(19) United States
(12) Patent Application Publication
(10) Pub. No.: US 2022/0388962 A1
(43) Pub. Date: Dec. 8, 2022

(54) MOLECULES THAT BIND TO TDP-43 FOR THE TREATMENT OF AMYOTROPHIC LATERAL SCLEROSIS AND RELATED DISORDERS

(71) Applicant: Biohaven Therapeutics Ltd., New Haven, CT (US)

(72) Inventors: Marianne CARLSEN, Yardley, PA (US); Garry R. SMITH, Elverson, PA (US); Randall J. BINDER, Conshohocken, PA (US); Camille REMEUR, Toulouse (FR); Allen B. REITZ, Lansdale, PA (US); Xiao JIN, King of Prussia, PA (US)

(21) Appl. No.: 17/635,421

(22) PCT Filed: Aug. 21, 2020
(86) PCT No.: PCT/US20/47287

§ 371 Date: Feb. 15, 2022

Related U.S. Application Data
(60) Provisional application No. 62/890,493, filed on Aug. 22, 2019.

(51) Int. Cl. C07D 215/42; C07D 401/12; C07D 215/46; C07D 487/04; C07D 401/10; C07D 471/04; C07D 487/10; C07D 401/14; C07D 413/12; C07D 405/12; C07D 401/04; C07D 405/04; C07D 413/14; C07D 491/056

(52) U.S. Cl. CPC C07D 215/42; C07D 401/12; C07D 215/46; C07D 487/04; C07D 401/10; C07D 471/04; C07D 487/10; C07D 401/14; C07D 413/12; C07D 405/12; C07D 401/04; C07D 405/04; C07D 413/14; C07D 491/056; C07B 2200/05

(57) ABSTRACT

Pharmaceutical compositions of the invention comprise TDP-43 binding agents having a disease-modifying action in the treatment of diseases associated with TDP-43 that include ALS, FTLD, CTE, hippocampal sclerosis of aging (CARTS), Alzheimer's disease, and Alzheimer's disease related disorder, and disease that involve excess amounts of TDP-43 in the cytosol.

### CROSS-REFERENCE TO RELATED APPLICATION

This application claims priority to and the benefit of U.S. Provisional Application No. 62/890,493 filed on Aug. 22, 2019, which is hereby incorporated in its entirety by reference.

### STATEMENT OF FEDERALLY FUNDED RESEARCH

This invention was made with government support under R44 AG059278-01 awarded by the National Institutes of Health. The government has certain rights in the invention.

### FIELD OF INVENTION

The present invention relates to the use of compounds that bind to the protein trans-activating response (TAR) DNA binding protein TDP-43 and block the binding of nucleic acid to TDP-43. Such compounds may be useful for the treatment of amyotrophic lateral sclerosis (ALS), frontotemporal lobar degeneration of the TDP-43 type (FTLD-TDP-43), chronic traumatic encephalopathy (CTE), inclusion body myositis (IBM) and certain forms of Alzheimer's disease and related disorders (ADRD). In addition, compounds binding to TDP-43 with high affinity and selectivity may be useful for as imaging agents, such as by positron emission tomography (PET).

### BACKGROUND OF THE INVENTION

ALS, FTLD-TDP-43, CTE and IBM are debilitating neurodegenerative disorders linked to TDP-43 and are considered as TDP-43 proteinopathies, associated with excess amounts of TDP-43 in the cytosol, typically C-terminal fragments after proteolysis followed by ubiquitylation and phosphorylation.

ALS is an orphan disease (1:1000 lifetime risk, ˜35,000 patients US) characterized by diminished motor function resulting in muscle wasting and death typically 3-5 years from diagnosis, often due to respiratory failure. The disease is separated into sporadic (˜90%) and familial (˜10%) patient populations. The primary genetic mutation comprising ˜40% of familial ALS (fALS) patients involves the repeat element (GGGGCC)_{600-1,000} in Orf92 on chromosome 9. A smaller subset (˜10-15%) of fALS patients have mutations in the earlier-discovered protein superoxide dismutase SOD1, with A4V SOD1 being the most prevalent in North America. Frontotemporal lobar degeneration (FTLD) is now recognized to be a leading cause in cognitive decline, making it the second largest cause of dementia after Alzheimer's disease accounting for 20% of all dementia. About half of FTLD is clearly associated with excess TDP-43 pathology, and is termed FTLD-TDP-43. CTE is a neurodegenerative disease caused by chronic traumatic head injuries, and is associated with abnormal depositions of TDP-43. IBM is a peripheral neurodegenerative disease affecting muscle control and motor function. It is also associated with abnormal deposits of TDP-43 among other aggregating proteins. TDP-43 is also associated with non-amnestic subsets of ADRD patients, and certain patients where the hippocampus undergoes scaring termed hippocampal sclerosis of aging (also known as cerebral age-related TDP-43 and sclerosis (CARTS)).

The extent and devastation of both ALS and FTLD are amplified by a broad impact upon caregivers and society with especially high costs in the terminal stages of the diseases. There are two drugs currently approved by the US FDA for the treatment of ALS: riluzole and edavarone. Unfortunately, there are no FDA-approved treatments for FTLD-TDP-43 or CTE. Although much research into applied drug discovery and development efforts have gone into understanding or modifying various pathways hypothesized to be important in ALS and FTLD-TDP-43 disease pathogenesis, there remains a large unmet medical need for disease-modifying therapeutic relief that will slow or halt disease progression, improve quality of life, and/or extend lifespan.

### BRIEF SUMMARY OF THE INVENTION

The present invention is directed toward novel small molecules capable of binding to TDP-43 (TDP-43 binders), a compound of formula (I),

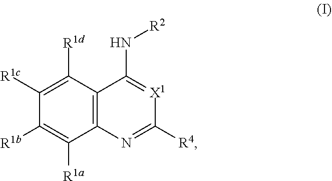

an enantiomer, a diastereomer, a hydrate, a solvate, a pharmaceutically acceptable salt, an isotopic analog, a prodrug or a complex thereof, wherein:
X¹ is selected from the group consisting of nitrogen and CH;
R^{1a} is selected from the group consisting of hydrogen, halogen, C₁₋₂₀ linear alkyl, C₃₋₂₀ branched alkyl, C₁₋₂₀ linear heteroalkyl, C₃₋₂₀ branched heteroalkyl, each of which except hydrogen and halogen are optionally substituted;
R^{1b} is selected from the group consisting of hydrogen, halogen, C₁₋₂₀ linear alkyl, C₃₋₂₀ branched alkyl, C₁₋₂₀ linear heteroalkyl, C₃₋₂₀ branched heteroalkyl, each of which except hydrogen and halogen are optionally substituted;
R^{1c} is selected from the group consisting of hydrogen, halogen, C₁₋₂₀ linear alkyl, C₃₋₂₀ branched alkyl, C₁₋₂₀ linear heteroalkyl, C₃₋₂₀ branched heteroalkyl, each of which except hydrogen and halogen are optionally substituted; and
R^{1d} is selected from the group consisting of hydrogen, halogen, C₁₋₂₀ linear alkyl, C₃₋₂₀ branched alkyl, C₁₋₂₀ linear heteroalkyl, C₃₋₂₀ branched heteroalkyl, each of which except hydrogen and halogen are optionally substituted.
R² is a substituted or unsubstituted C₁-C₂₀ linear, branched, or cyclic organic group including at least one nitrogen; and
R⁴ is a hydrogen, halogen, a hydroxyl group, a cyano group, a nitro group, a substituted or unsubstituted C₀-C₁₀ amino group, a substituted or unsubstituted C₁-C₁₀ alkyl group, a substituted or unsubstituted C₂-C₁₀ alkenyl group, a substituted or unsubstituted C₂-C₁₀ alkynyl group, and a substituted or unsubstituted C₁-C₁₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₂₀ aryl group, a substituted or unsubstituted C₆-C₂₀ aryloxy group, a substituted or unsubstituted C₆-C₂₀ arylthio group, a substituted or unsubstituted C₂-C₂₀ heteroaryl group, a substituted or unsubstituted C₂-C₂₀ heteroaryloxy group, or a substituted or unsubstituted C₂-C₂₀ heteroarylthio group.

The present invention is further directed toward novel small molecules capable of binding to TDP-43 (TDP-43 binders), a compound of formula (I),

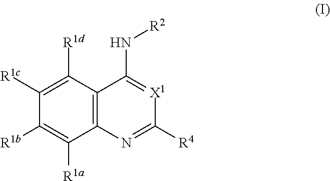

including an enantiomer, a diastereomer, a hydrate, a solvate, a pharmaceutically acceptable salt, an isotopic analog, a prodrug, and a complex thereof, wherein:
X¹ is selected from the group consisting of nitrogen and CH;
R^{1a} is selected from the group consisting of hydrogen, halogen, CF₃, OCF₃, C₁₋₄ linear alkyl, C₃₋₄ branched alkyl, (C₂₋₈ dialkylamino)(C₂₋₄ alkyl), (C₃₋₆ alkyleneamino)(C₂₋₄ alkyl), C₁₋₄ linear alkoxy, and C₃₋₄ branched alkoxy;
R^{1b} is selected from the group consisting of hydrogen, halogen, CF₃, OCF₃, C₁-4 linear alkyl, C₃₋₄ branched alkyl, (C₂₋₈ dialkylamino)(C₂₋₄ alkyl), (C₃₋₆ alkyleneamino)(C₂₋₄ alkyl), C₁₋₄ linear alkoxy, and C₃₋₄ branched alkoxy;
R^{1c} is selected from the group consisting of hydrogen, halogen, CF₃, OCF₃, C₁-4 linear alkyl, C₃₋₄ branched alkyl, (C₂₋₈ dialkylamino)(C₂₋₄ alkyl), (C₃₋₆ alkyleneamino)(C₂₋₄ alkyl), C₁₋₄ linear alkoxy, and C₃₋₄ branched alkoxy;
R^{1d} is selected from the group consisting of hydrogen, halogen, CF₃, OCF₃, C₁₋₄ linear alkyl, C₃₋₄ branched alkyl, (C₂₋₈ dialkylamino)(C₂₋₄ alkyl), (C₃₋₆ alkyleneamino)(C₂₋₄ alkyl), C₁₋₄ linear alkoxy, and C₃₋₄ branched alkoxy;
wherein any two selected from the group consisting of R^{1a}, R^{1b}, R^{1c}, and R^{1d} are optionally connected to form a ring;
R² is selected from the group consisting of —(CH₂)ₙ—NR⁵R⁶, —(CH₂)ₙC(O)—NR⁵R⁶,

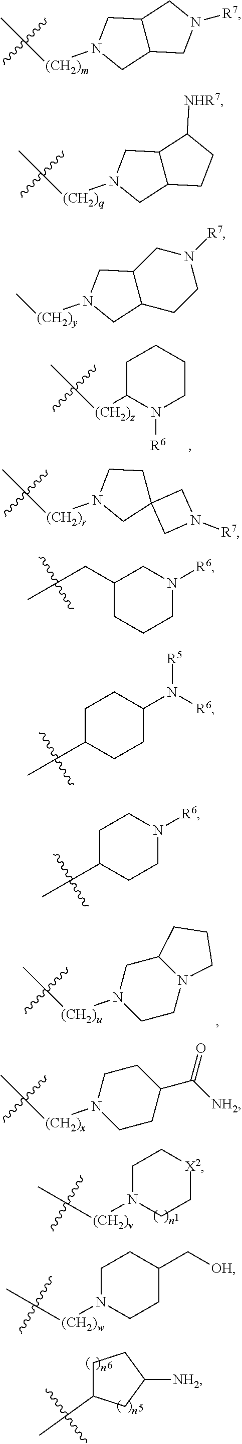

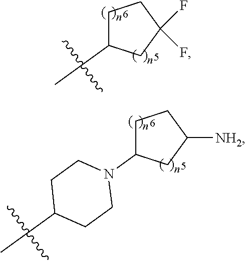 [SMILES: *C1CCC(F)(F)C1 ; *C1CCN(C2CCC(N)C2)CC1]

n¹ is 1 or 2;
R⁴ is hydrogen; CF₃; a five-membered monocyclic heteroaryl ring comprising at least one heteroatom selected from the group consisting from O, N, and S that is optionally substituted with up to 2 groups selected from C₁₋₄ linear alkyl, C₃₋₄ branched alkyl, C₁-4 linear alkoxy, C₃₋₄ branched alkoxy, CF₃, CF₃O, halogen,

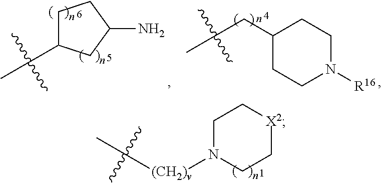

a five-membered monocyclic heteroaryl ring comprising at least one heteroatom selected from the group consisting from O, N, and S;

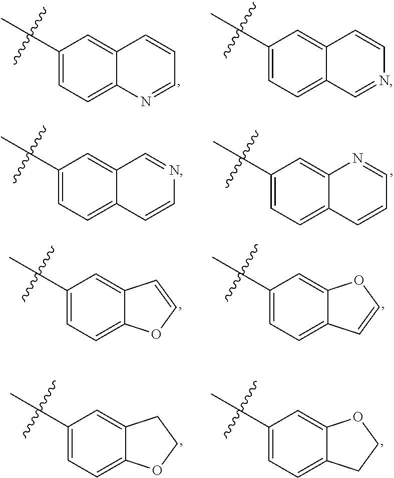

a phenyl ring that is optionally substituted with up to 2 groups selected from C₁₋₄ linear alkyl, C₃₋₄ branched alkyl, C₁₋₄ linear alkoxy, C₃₋₄ branched alkoxy, C₃₋₆ cycloalkyl, C₃₋₆ cycloalkoxy, CF₃, CF₃O, halogen, NR^{3a}R^{3b}, SO₂NR^{3a}R^{3b}, NHSO₂R^{3a},

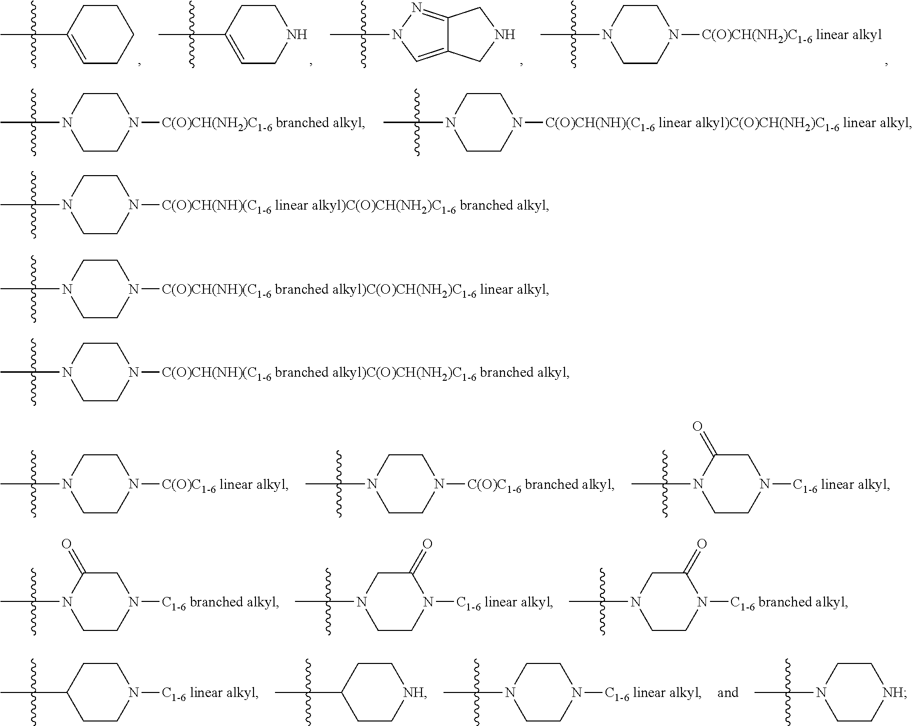

R^{3a} is at each occurrence independently selected from the group consisting of C₁₋₄ linear alkyl and C₃₋₄ branched alkyl;
R^{3b} is at each occurrence independently selected from the group consisting of C₁₋₄ linear alkyl and C₃₋₄ branched alkyl;
R⁵ is selected from the group consisting of hydrogen, C₁₋₄ linear alkyl, C₃₋₄ branched alkyl, —CH₂—(C₁₋₆ cycloalkyl), C(O)C₁₋₆ linear alkyl, C(O)C₃₋₆ branched alkyl, C(O)CH(NH₂)C₁₋₆ linear alkyl, C(O)CH(NH₂)C₃₋₆ branched alkyl,

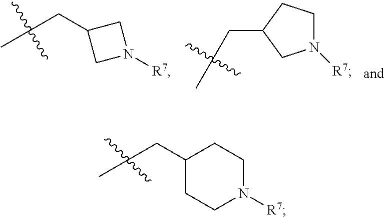

R⁶ is selected from the group consisting of C₁₋₄ linear alkyl, C₃₋₄ branched alkyl,

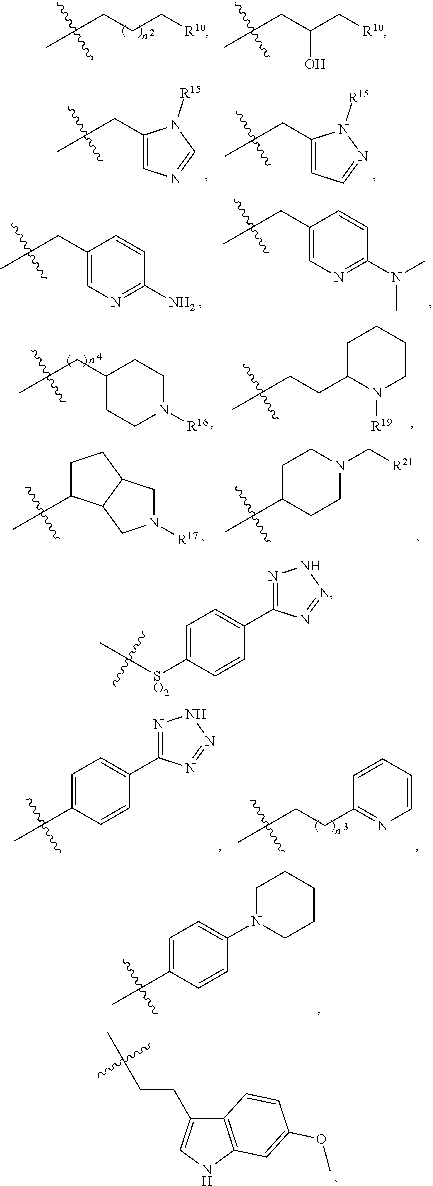

and an optionally substituted benzyl group wherein the optionally substituted benzyl group is substituted with 0 to 2 groups selected from the group consisting of halogen, —CN, —NO₂, —OH, —NH₂, C₁₋₆ alkyl, C₃₋₇ branched alkyl, C₁₋₆ linear haloalkyl, C₃₋₇ branched haloalkyl, C₁₋₆ linear alkoxy, C₃₋₇ branched alkoxy, C₁₋₆ linear haloalkoxy, C₃₋₇ branched haloalkoxy, C₃₋₇ cycloalkyl, aryl, heterocycle, and heteroaryl, provided that when R² is

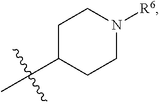

R⁶ is not hydrogen, C₁₋₄ linear alkyl, C₃₋₄ branched alkyl, or benzyl;
n² is 1 or 2;
n³ is 0 or 1;
n⁴ is 0 or 1;
n⁵ is 0, 1, or 2;
n⁶ is 0 or 1;
R⁷ is selected from the group consisting of hydrogen and C(O)OR⁸;
R⁸ is selected from the group consisting of C₁₋₄ linear alkyl and C₃₋₄ branched alkyl;
X² is selected from the group consisting of a single bond, oxygen, CH₂, CHOH, and NR⁹;
R⁹ is C₁₋₄ linear alkyl that is optionally substituted with an NH₂;
R¹⁰ is selected from the group consisting of OH, OR¹¹, NR¹²R¹³, NHSO₂R²², and

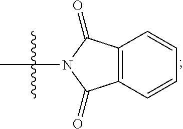 [SMILES: *N1C(=O)c2ccccc2C1=O]

R¹¹ is selected from the group consisting of C₁₋₄ linear alkyl and C₃₋₄ branched alkyl;
R¹² is selected from the group consisting of hydrogen, C₁₋₄ linear alkyl, C₃₋₄ branched alkyl, C₁₋₄ linear fluoroalkyl, C(O)R¹⁴;
R¹³ is selected from the group consisting of hydrogen, C₁₋₄ linear alkyl, C₃₋₄ branched alkyl, and heteroaryl;
R¹² and R¹³ are optionally taken together with the atoms to which they are connected to form a ring with 3 to 6 atoms;
R¹⁴ is selected from the group consisting of C₁₋₄ linear alkyl C₃₋₄ branched alkyl, and

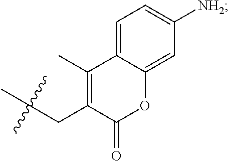 [SMILES: *Cc1c(C)c2ccc(N)cc2oc1=O]

R¹⁵ is selected from the group consisting of C₁₋₄ linear alkyl and C₃₋₄ branched alkyl;
R¹⁶ is selected from the group consisting of hydrogen, C₁₋₄ linear alkyl, C₃₋₄ branched alkyl, CH₂—(C₁₋₆ cycloalkyl), and C(O)R¹⁸;
R¹⁷ is selected from the group consisting of hydrogen, benzyl and C(O)R¹⁸;
R¹⁸ is selected from the group consisting of C₁₋₄ linear alkyl and C₃₋₄ branched alkyl;
R¹⁹ is selected from the group consisting of hydrogen and C(O)R²⁰;
R²⁰ is selected from the group consisting of C₁₋₄ linear alkyl and C₃₋₄ branched alkyl;
R²¹ is a benzene ring that is optionally substituted with 0 to 2 groups selected from the group consisting of halogen, —CN, —NO₂, —OH, —NH₂, C₁₋₆ alkyl, C₃₋₇ branched alkyl, C₁₋₆ linear haloalkyl, C₃₋₇ branched haloalkyl, C₁₋₆ linear alkoxy, C₃₋₇ branched alkoxy, C₁₋₆ linear haloalkoxy, C₃₋₇ branched haloalkoxy, C₃₋₇ cycloalkyl, aryl, heterocycle, and heteroaryl;
R²² is selected from the group consisting of C₁₋₄ linear alkyl, C₃₋₄ branched alkyl,

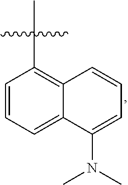 [SMILES: *c1cccc2c(N(C)C)cccc12]

n is 2, 3, or 4;
m is 2, 3, or 4;
q is 2, 3, or 4;
y is 2, 3, or 4;
u is 2, 3, or 4;
v is 2, 3, or 4;
w is 2, 3, or 4;
z is 1, 2 or 3;
r is 2, 3, or 4; and
x is 2, 3, or 4.

The present invention further relates to compositions comprising:

an effective amount of one or more compounds according to the present invention and an excipient.

The present invention also relates to a method for treating or preventing diseases that involve TDP-43, including, for example, ALS, FTLD, CTE, hippocampal sclerosis of aging (CARTS), Alzheimer's disease, or an Alzheimer's disease related disorder, said method comprising administering to a subject an effective amount of a compound or composition according to the present invention.

The present invention yet further relates to a method for treating or preventing diseases that involve TDP-43, including, for example, ALS, FTLD, CTE, hippocampal sclerosis of aging (CARTS), Alzheimer's disease, or an Alzheimer's disease related disorder, wherein said method comprises administering to a subject a composition comprising an effective amount of one or more compounds according to the present invention and an excipient.

The present invention yet further relates to a method for treating or preventing diseases that involve TDP-43, including, for example, ALS, FTLD, CTE, hippocampal sclerosis of aging (CARTS), Alzheimer's disease, or an Alzheimer's disease related disorder, wherein said method comprises administering to a subject a composition comprising an effective amount of one or more compounds according to the present invention and an excipient, and on or more compounds selected from the group consisting of riluzole, troriluzole, trigriluzole, and edavarone.

The present invention also relates to a method for treating or preventing disease or conditions associated with TDP-43 proteinopathies, and diseases that involve excess amounts of TDP-43 in the cytosol. Said methods comprise administering to a subject an effective amount of a compound or composition according to the present invention.

The present invention yet further relates to a method for treating or preventing disease or conditions associated with TDP-43 proteinopathies, and diseases that involve excess amounts of TDP-43 in the cytosol, wherein said method comprises administering to a subject a composition comprising an effective amount of one or more compounds according to the present invention and an excipient.

The present invention yet further relates to a method for treating or preventing disease or conditions associated with TDP-43 proteinopathies, and diseases that involve an excess amounts of TDP-43 in the cytosol, wherein said method comprises administering to a subject a composition comprising an effective amount of one or more compounds according to the present invention and an excipient, and on or more compounds selected from the group consisting of riluzole, troriluzole, trigriluzole, and edavarone.

The present invention also relates to a method for treating or preventing disease or conditions associated with TDP-43. Said methods comprise administering to a subject an effective amount of a compound or composition according to the present invention.

The present invention yet further relates to a method for treating or preventing disease or conditions associated with TDP-43, wherein said method comprises administering to a subject a composition comprising an effective amount of one or more compounds according to the present invention and an excipient.

The present invention yet further relates to a method for treating or preventing disease or conditions associated with TDP-43, wherein said method comprises administering to a subject a composition comprising an effective amount of one or more compounds according to the present invention and an excipient, and on or more compounds selected from the group consisting of riluzole, troriluzole, trigriluzole, and edavarone.

The present invention yet further relates to the method of use of the TDP-43 binders of the present invention as positron emission tomography (PET) imaging agents, wherein said method comprises administering to a subject an effective amount of an isotopically labeled compound or composition according to the present invention.

The present invention yet further relates to the method of use of the TDP-43 binders of the present invention as single-photon emission computed tomography (SPECT) imaging agents, wherein said method comprises administering to a subject an effective amount of an isotopically labeled compound or composition according to the present invention.

The present invention further relates to a process for preparing the TDP-43 binders of the present invention.

These and other objects, features, and advantages will become apparent to those of ordinary skill in the art from a reading of the following detailed description and the appended claims. All percentages, ratios and proportions herein are by weight, unless otherwise specified. All temperatures are in degrees Celsius (° C.) unless otherwise specified. All documents cited are in relevant part, incorporated herein by reference; the citation of any document is not to be construed as an admission that it is prior art with respect to the present invention.

### BRIEF DESCRIPTION OF THE DRAWINGS

These and/or other aspects will become apparent and more readily appreciated from the following description of the embodiments, taken in conjunction with the accompanying drawings, in which:

FIG. 1: SH-SY5Y and A172 cells were seeded in 6 well plates at 1×106 and 2×105 cells/well respectively. Approximately 18 hrs after seeding, MG132 and compounds were added at the indicated concentrations and incubation at 37° C. was continued for 20 hours. The cells were harvested and cytoplasmic fractions were prepared according to the supplier's protocol using the Thermo NE-PER kit (#78833). The samples were denatured in 0.1M DTT, Novex NuPAGE LDS Sample Buffer at 80° C. for 10 minutes, separated on 12% polyacrylamide Tris-Glycine SDS gels, transferred to PVDF membranes and probed with a C-terminal TDP-43-specific antibody (ProteinTech 12892-1-AP).

### DETAILED DESCRIPTION OF THE INVENTION

The TDP-43 binders of the present invention are capable of treating and preventing diseases associated with TDP-43, for example ALS, FTLD, CTE, hippocampal sclerosis of aging (CARTS), Alzheimer's disease, and Alzheimer's disease related disorders. In addition, the TDP-43 binders of the present invention are also useful as positron emission tomography (PET) imaging agents, useful for the diagnosis of diseases and conditions associated with TDP-43. Further, the TDP-43 binders of the present invention are also useful as single-photon emission computed tomography (SPECT) imaging agents, useful for the diagnosis of diseases and conditions associated with TDP-43.

Throughout the description, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited processing steps.

In the application, where an element or component is said to be included in and/or selected from a list of recited elements or components, it should be understood that the element or component can be any one of the recited elements or components and can be selected from a group consisting of two or more of the recited elements or components.

The use of the singular herein includes the plural (and vice versa) unless specifically stated otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions can be conducted simultaneously.

As used herein, the term “halogen” shall mean chlorine, bromine, fluorine and iodine.

As used herein, unless otherwise noted, “alkyl” and/or “aliphatic” whether used alone or as part of a substituent group refers to straight and branched carbon chains having 1 to 20 carbon atoms or any number within this range, for example 1 to 6 carbon atoms or 1 to 4 carbon atoms. Designated numbers of carbon atoms (e.g., C₁₋₆) shall refer independently to the number of carbon atoms in an alkyl moiety or to the alkyl portion of a larger alkyl-containing substituent. Non-limiting examples of alkyl groups include methyl, ethyl, n-propyl, iso-propyl, n-butyl, sec-butyl, iso-butyl, tert-butyl, and the like. Alkyl groups can be optionally substituted. Non-limiting examples of substituted alkyl groups include hydroxymethyl, chloromethyl, trifluoromethyl, aminomethyl, 1-chloroethyl, 2-hydroxyethyl, 1,2-difluoroethyl, 3-carboxypropyl, and the like. In substituent groups with multiple alkyl groups such as (C₁₋₆alkyl)₂ amino, the alkyl groups may be the same or different.

As used herein, unless otherwise noted, “heteroalkyl” whether used alone or as part of a substituent group refers to an “alkyl” group as defined above in which at least one carbon atom is replaced with a heteroatom selected from the group consisting of nitrogen (N), oxygen (O), sulfur (S), and phosphorus (P). Non-limiting examples of alkyl groups include methoxy, ethoxy, methoxyethyl, methoxyethoxy, dimethylaminoethyl, dimethylaminopropyl, diethylaminoethyl, diethylaminopropyl, isopropylaminopropyl, azetidinopropyl, pyrrolidinopropyl, piperidinopropyl, pyrrolidinopropoxy, piperidinopropoxy, and the like.

As used herein, the terms “alkenyl” and “alkynyl” groups, whether used alone or as part of a substituent group, refer to straight and branched carbon chains having 2 or more carbon atoms, preferably 2 to 20, wherein an alkenyl chain has at least one double bond in the chain and an alkynyl chain has at least one triple bond in the chain. Alkenyl and alkynyl groups can be optionally substituted. Non-limiting examples of alkenyl groups include ethenyl, 3-propenyl, 1-propenyl (also 2-methylethenyl), isopropenyl (also 2-methylethen-2-yl), buten-4-yl, and the like. Non-limiting examples of substituted alkenyl groups include 2-chloroethenyl (also 2-chlorovinyl), 4-hydroxybuten-1-yl, 7-hydroxy-7-methyloct-4-en-2-yl, 7-hydroxy-7-methyloct-3,5-dien-2-yl, and the like. Non-limiting examples of alkynyl groups include ethynyl, prop-2-ynyl (also propargyl), propyn-1-yl, and 2-methyl-hex-4-yn-1-yl. Non-limiting examples of substituted alkynyl groups include, 5-hydroxy-5-methylhex-3-ynyl, 6-hydroxy-6-methylhept-3-yn-2-yl, 5-hydroxy-5-ethylhept-3-ynyl, and the like.

As used herein, “cycloalkyl,” whether used alone or as part of another group, refers to a non-aromatic carbon-containing ring including cyclized alkyl, alkenyl, and alkynyl groups, e.g., having from 3 to 14 ring carbon atoms, preferably from 3 to 7 or 3 to 6 ring carbon atoms, or even 3 to 4 ring carbon atoms, and optionally containing one or more (e.g., 1, 2, or 3) double or triple bond. Cycloalkyl groups can be monocyclic (e.g., cyclohexyl) or polycyclic (e.g., containing fused, bridged, and/or spiro ring systems), wherein the carbon atoms are located inside or outside of the ring system. Any suitable ring position of the cycloalkyl group can be covalently linked to the defined chemical structure. Cycloalkyl rings can be optionally substituted. Non-limiting examples of cycloalkyl groups include: cyclopropyl, 2-methyl-cyclopropyl, cyclopropenyl, cyclobutyl, 2,3-dihydroxycyclobutyl, cyclobutenyl, cyclopentyl, cyclopentenyl, cyclopentadienyl, cyclohexyl, cyclohexenyl, cycloheptyl, cyclooctanyl, decalinyl, 2,5-dimethylcyclopentyl, 3,5-dichlorocyclohexyl, 4-hydroxycyclohexyl, 3,3,5-trimethylcyclohex-1-yl, octahydropentalenyl, octahydro-1H-indenyl, 3a,4,5,6,7,7a-hexahydro-3H-inden-4-yl, decahydroazulenyl; bicyclo[6.2.0]decanyl, decahydronaphthalenyl, and dodecahydro-1H-fluorenyl. The term “cycloalkyl” also includes carbocyclic rings which are bicyclic hydrocarbon rings, non-limiting examples of which include, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

“Haloalkyl” is intended to include both branched and straight-chain saturated aliphatic hydrocarbon groups having the specified number of carbon atoms, substituted with 1 or more halogen. Haloalkyl groups include perhaloalkyl groups, wherein all hydrogens of an alkyl group have been replaced with halogens (e.g., —CF₃, —CF₂CF₃). Haloalkyl groups can optionally be substituted with one or more substituents in addition to halogen. Examples of haloalkyl groups include, but are not limited to, fluoromethyl, dichloroethyl, trifluoromethyl, trichloromethyl, pentafluoroethyl, and pentachloroethyl groups.

The term “alkoxy” refers to the group —O-alkyl, wherein the alkyl group is as defined above. Alkoxy groups optionally may be substituted. The term C₃-C₆ cyclic alkoxy refers to a ring containing 3 to 6 carbon atoms and at least one oxygen atom (e.g., tetrahydrofuran, tetrahydro-2H-pyran). C₃-C₆ cyclic alkoxy groups optionally may be substituted.

The term “aryl,” wherein used alone or as part of another group, is defined herein as a an unsaturated, aromatic monocyclic ring of 6 carbon members or to an unsaturated, aromatic polycyclic ring of from 10 to 14 carbon members. Aryl rings can be, for example, phenyl or naphthyl ring each optionally substituted with one or more moieties capable of replacing one or more hydrogen atoms. Non-limiting examples of aryl groups include: phenyl, naphthylen-1-yl, naphthylen-2-yl, 4-fluorophenyl, 2-hydroxyphenyl, 3-methylphenyl, 2-amino-4-fluorophenyl, 2-(N,N-diethylamino)phenyl, 2-cyanophenyl, 2,6-di-tert-butylphenyl, 3-methoxyphenyl, 8-hydroxynaphthylen-2-yl 4,5-dimethoxynaphthylen-1-yl, and 6-cyano-naphthylen-1-yl. Aryl groups also include, for example, phenyl or naphthyl rings fused with one or more saturated or partially saturated carbon rings (e.g., bicyclo[4.2.0]octa-1,3,5-trienyl, indanyl), which can be substituted at one or more carbon atoms of the aromatic and/or saturated or partially saturated rings.

The term “arylalkyl” or “aralkyl” refers to the group -alkyl-aryl, where the alkyl and aryl groups are as defined herein. Aralkyl groups of the present invention are optionally substituted. Examples of arylalkyl groups include, for example, benzyl, 1-phenylethyl, 2-phenylethyl, 3-phenylpropyl, 2-phenylpropyl, fluorenylmethyl and the like.

The terms “heterocyclic” and/or “heterocycle” and/or “heterocyclyl,” whether used alone or as part of another group, are defined herein as one or more ring having from 3 to 20 atoms wherein at least one atom in at least one ring is a heteroatom selected from nitrogen (N), oxygen (O), or sulfur (S), and wherein further the ring that includes the heteroatom is non-aromatic. In heterocycle groups that include 2 or more fused rings, the non-heteroatom bearing ring may be aryl (e.g., indolinyl, tetrahydroquinolinyl, chromanyl). Exemplary heterocycle groups have from 3 to 14 ring atoms of which from 1 to 5 are heteroatoms independently selected from nitrogen (N), oxygen (O), or sulfur (S). One or more N or S atoms in a heterocycle group can be oxidized. Heterocycle groups can be optionally substituted.

Non-limiting examples of heterocyclic units having a single ring include: diazirinyl, aziridinyl, urazolyl, azetidinyl, pyrazolidinyl, imidazolidinyl, oxazolidinyl, isoxazolinyl, isoxazolyl, thiazolidinyl, isothiazolyl, isothiazolinyl oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl(valerolactam), 2,3,4,5-tetrahydro-1H-azepinyl, 2,3-dihydro-1H-indole, and 1,2,3,4-tetrahydro-quinoline. Non-limiting examples of heterocyclic units having 2 or more rings include: hexahydro-1H-pyrrolizinyl, 3a,4,5,6,7,7a-hexahydro-1H-benzo[d]imidazolyl, 3a,4,5,6,7,7a-hexahydro-1H-indolyl, 1,2,3,4-tetrahydroquinolinyl, chromanyl, isochromanyl, indolinyl, isoindolinyl, and decahydro-1H-cycloocta[b]pyrrolyl.

The term “heteroaryl,” whether used alone or as part of another group, is defined herein as one or more rings having from 5 to 20 atoms wherein at least one atom in at least one ring is a heteroatom chosen from nitrogen (N), oxygen (O), or sulfur (S), and wherein further at least one of the rings that includes a heteroatom is aromatic. In heteroaryl groups that include 2 or more fused rings, the non-heteroatom bearing ring may be a carbocycle (e.g., 6,7-Dihydro-5H-cyclopentapyrimidine) or aryl (e.g., benzofuranyl, benzothiophenyl, indolyl). Exemplary heteroaryl groups have from 5 to 14 ring atoms and contain from 1 to 5 ring heteroatoms independently selected from nitrogen (N), oxygen (O), or sulfur (S). One or more N or S atoms in a heteroaryl group can be oxidized. Heteroaryl groups can be substituted. Non-limiting examples of heteroaryl rings containing a single ring include: 1,2,3,4-tetrazolyl, [1,2,3]triazolyl, [1,2,4]triazolyl, triazinyl, thiazolyl, 1H-imidazolyl, oxazolyl, furanyl, thiopheneyl, pyrimidinyl, 2-phenylpyrimidinyl, pyridinyl, 3-methylpyridinyl, and 4-dimethylaminopyridinyl. Non-limiting examples of heteroaryl rings containing 2 or more fused rings include: benzofuranyl, benzothiophenyl, benzoxazolyl, benzthiazolyl, benztriazolyl, cinnolinyl, naphthyridinyl, phenanthridinyl, 7H-purinyl, 9H-purinyl, 6-amino-9H-purinyl, 5H-pyrrolo[3,2-d]pyrimidinyl, 7H-pyrrolo[2,3-d]pyrimidinyl, pyrido[2,3-d]pyrimidinyl, 2-phenylbenzo[d]thiazolyl, 1H-indolyl, 4,5,6,7-tetrahydro-1-H-indolyl, quinoxalinyl, 5-methylquinoxalinyl, quinazolinyl, quinolinyl, 8-hydroxy-quinolinyl, and isoquinolinyl.

One non-limiting example of a heteroaryl group as described above is C₁-C₅ heteroaryl, which has 1 to 5 carbon ring atoms and at least one additional ring atom that is a heteroatom (preferably 1 to 4 additional ring atoms that are heteroatoms) independently selected from nitrogen (N), oxygen (O), or sulfur (S). Examples of C₁-C₅ heteroaryl include, but are not limited to, triazinyl, thiazol-2-yl, thiazol-4-yl, imidazol-1-yl, 1H-imidazol-2-yl, 1H-imidazol-4-yl, isoxazolin-5-yl, furan-2-yl, furan-3-yl, thiophen-2-yl, thiophen-4-yl, pyrimidin-2-yl, pyrimidin-4-yl, pyrimidin-5-yl, pyridin-2-yl, pyridin-3-yl, and pyridin-4-yl.

Unless otherwise noted, when two substituents are taken together to form a ring having a specified number of ring atoms (e.g., R² and R³ taken together with the nitrogen (N) to which they are attached to form a ring having from 3 to 7 ring members), the ring can have carbon atoms and optionally one or more (e.g., 1 to 3) additional heteroatoms independently selected from nitrogen (N), oxygen (O), or sulfur (S). The ring can be saturated or partially saturated and can be optionally substituted.

For the purposed of the present invention fused ring units, as well as spirocyclic rings, bicyclic rings and the like, which comprise a single heteroatom will be considered to belong to the cyclic family corresponding to the heteroatom containing ring. For example, 1,2,3,4-tetrahydroquinoline having the formula:

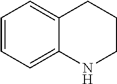

is, for the purposes of the present invention, considered a heterocyclic unit. 6,7-Dihydro-5H-cyclopentapyrimidine having the formula:

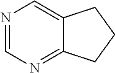

is, for the purposes of the present invention, considered a heteroaryl unit. When a fused ring unit contains heteroatoms in both a saturated and an aryl ring, the aryl ring will predominate and determine the type of category to which the ring is assigned. For example, 1,2,3,4-tetrahydro-[1,8]naphthyridine having the formula:

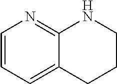

is, for the purposes of the present invention, considered a heteroaryl unit.

Whenever a term or either of their prefix roots appear in a name of a substituent the name is to be interpreted as including those limitations provided herein. For example, whenever the term “alkyl” or “aryl” or either of their prefix roots appear in a name of a substituent (e.g., arylalkyl, alkylamino) the name is to be interpreted as including those limitations given above for “alkyl” and “aryl.”

The term “substituted” is used throughout the specification. The term “substituted” is defined herein as a moiety, whether acyclic or cyclic, which has one or more hydrogen atoms replaced by a substituent or several (e.g., 1 to 10) substituents as defined herein below. The substituents are capable of replacing one or two hydrogen atoms of a single moiety at a time. In addition, these substituents can replace two hydrogen atoms on two adjacent carbons to form said substituent, new moiety or unit. For example, a substituted unit that requires a single hydrogen atom replacement includes halogen, hydroxyl, and the like. A two hydrogen atom replacement includes carbonyl, oximino, and the like. A two hydrogen atom replacement from adjacent carbon atoms includes epoxy, and the like. The term “substituted” is used throughout the present specification to indicate that a moiety can have one or more of the hydrogen atoms replaced by a substituent. When a moiety is described as “substituted” any number of the hydrogen atoms may be replaced. For example, difluoromethyl is a substituted C₁ alkyl; trifluoromethyl is a substituted C₁ alkyl; 4-hydroxyphenyl is a substituted aromatic ring; (N,N-dimethyl-5-amino)octanyl is a substituted C₈ alkyl; 3-guanidinopropyl is a substituted C₃ alkyl; and 2-carboxypyridinyl is a substituted heteroaryl.

The variable groups defined herein, e.g., alkyl, alkenyl, alkynyl, cycloalkyl, alkoxy, aryloxy, aryl, heterocycle and heteroaryl groups defined herein, whether used alone or as part of another group, can be optionally substituted. Optionally substituted groups will be so indicated.

The following are non-limiting examples of substituents which can substitute for hydrogen atoms on a moiety: halogen (chlorine (Cl), bromine (Br), fluorine (F) and iodine(I)), —CN, —NO₂, oxo (═O), —OR²³, —SR²³, —N(R²³)₂, —NR²³C(O)R²³, —SO₂R²³, —SO₂OR²³, —SO₂N(R²³)₂, —C(O)R²³, —C(O)OR²³, —C(O)N(R²³)₂, C₁₋₆ alkyl, C₁₋₆ haloalkyl, C₁₋₆ alkoxy, C₂₋₈ alkenyl, C₂₋₈ alkynyl, C₃₋₁₄ cycloalkyl, aryl, heterocycle, or heteroaryl, wherein each of the alkyl, haloalkyl, alkenyl, alkynyl, alkoxy, cycloalkyl, aryl, heterocycle, and heteroaryl groups is optionally substituted with 1-10 (e.g., 1-6 or 1-4) groups selected independently from halogen, —CN, —NO₂, oxo, and R²³; wherein R²³, at each occurrence, independently is hydrogen, —OR²⁴, —SR²⁴, —C(O)R²⁴, —C(O)OR²⁴, —C(O)N(R²⁴)₂, —SO₂R²⁴, —S(O)₂₀R²⁴, —N(R²⁴)₂, —NR²⁴C(O)R²⁴, C₁₋₆ alkyl, C₁₋₆ haloalkyl, C₂₋₈ alkenyl, C₂₋₈ alkynyl, cycloalkyl (e.g., C₃₋₆ cycloalkyl), aryl, heterocycle, or heteroaryl, or two R²³ units taken together with the atom(s) to which they are bound form an optionally substituted carbocycle or heterocycle wherein said carbocycle or heterocycle has 3 to 7 ring atoms; wherein R²⁴, at each occurrence, independently is hydrogen, C₁₋₆ alkyl, C₁₋₆ haloalkyl, C₂₋₈ alkenyl, C₂₋₈ alkynyl, cycloalkyl (e.g., C₃₋₆ cycloalkyl), aryl, heterocycle, or heteroaryl, or two R²⁴ units taken together with the atom(s) to which they are bound form an optionally substituted carbocycle or heterocycle wherein said carbocycle or heterocycle preferably has 3 to 7 ring atoms.

In some embodiments, the substituents are selected from
i) —OR²⁵; for example, —OH, —OCH₃, —OCH₂CH₃, —OCH₂CH₂CH₃;
ii) —C(O)R²⁵; for example, —COCH₃, —COCH₂CH₃, —COCH₂CH₂CH₃;
iii) —C(O)OR²⁵; for example, —CO₂CH₃, —CO₂CH₂CH₃, —CO₂CH₂CH₂CH₃;
iv) —C(O)N(R²⁵)₂; for example, —CONH₂, —CONHCH₃, —CON(CH₃)₂;
v) —N(R²⁵)₂; for example, —NH₂, —NHCH₃, —N(CH₃)₂, —NH(CH₂CH₃), pyrrolidinyl;
vi) halogen: —F, —Cl, —Br, and —I;
vii) —CHₑX_{g}; wherein X is halogen, e and g are independently selected from 0 to 2, provided that e+g=3; for example, —CH₂F, —CHF₂, —CF₃, —CCl₃, or —CBr₃;
viii) —SO₂R²⁵; for example, —SO₂H; —SO₂CH₃; —SO₂C₆H₅;
ix) C₁-C₆ linear, branched, or cyclic alkyl;
x) cyano
xi) nitro;
xii) N(R²⁵)C(O)R²⁵;
xiii) oxo (═O);
xiv) heterocycle; and
xv) heteroaryl,
wherein each R²⁵ is independently hydrogen, optionally substituted C₁-C₆ linear or branched alkyl (e.g., optionally substituted C₁-C₄ linear or branched alkyl), or optionally substituted C₃-C₆ cycloalkyl (e.g., optionally substituted C₃-C₄ cycloalkyl); or two R²⁵ units can be taken together to form a ring comprising 3-7 ring atoms, which may include a heteroatom selected from N, O, S, and P. In certain aspects, each R²⁵ is independently hydrogen, C₁-C₆ linear or branched alkyl optionally substituted with halogen or C₃-C₆ cycloalkyl or C₃-C₆ cycloalkyl.

At various places in the present specification, substituents of compounds are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆, alkyl.

For the purposes of the present invention the terms “compound,” “analog,” and “composition of matter” stand equally well for the TDP-43 binders described herein, including all enantiomeric forms, diastereomeric forms, salts, and the like, and the terms “compound,” “analog,” and “composition of matter” are used interchangeably throughout the present specification.

Compounds described herein can contain an asymmetric atom (also referred as a chiral center), and some of the compounds can contain one or more asymmetric atoms or centers, which can thus give rise to optical isomers (enantiomers) and diastereomers. The present teachings and compounds disclosed herein include such enantiomers and diastereomers, as well as the racemic and resolved, enantiomerically pure R and S stereoisomers, as well as other mixtures of the R and S stereoisomers and pharmaceutically acceptable salts thereof. Optical isomers can be obtained in pure form by standard procedures known to those skilled in the art, which include, but are not limited to, diastereomeric salt formation, kinetic resolution, and asymmetric synthesis. The present teachings also encompass cis and trans isomers of compounds containing alkenyl moieties (e.g., alkenes and imines). It is also understood that the present teachings encompass all possible regioisomers, and mixtures thereof, which can be obtained in pure form by standard separation procedures known to those skilled in the art, and include, but are not limited to, column chromatography, thin-layer chromatography, and high-performance liquid chromatography.

Pharmaceutically acceptable salts of compounds of the present teachings, which can have an acidic moiety, can be formed using organic and inorganic bases. Both mono and polyanionic salts are contemplated, depending on the number of acidic hydrogens available for deprotonation. Suitable salts formed with bases include metal salts, such as alkali metal or alkaline earth metal salts, for example sodium, potassium, or magnesium salts; ammonia salts and organic amine salts, such as those formed with morpholine, thiomorpholine, piperidine, pyrrolidine, a mono-, di- or tri-lower alkylamine (e.g., ethyl-tert-butyl-, diethyl-, diisopropyl-, triethyl-, tributyl- or dimethylpropylamine), or a mono-, di-, or trihydroxy lower alkylamine (e.g., mono-, di- or triethanolamine). Specific non-limiting examples of inorganic bases include NaHCO₃, Na₂CO₃, KHCO₃, K₂CO₃, Cs₂CO₃, LiOH, NaOH, KOH, NaH₂PO₄, Na₂HPO₄, and Na₃PO₄. Internal salts also can be formed. Similarly, when a compound disclosed herein contains a basic moiety, salts can be formed using organic and inorganic acids. For example, salts can be formed from the following acids: acetic, propionic, lactic, benzenesulfonic, benzoic, camphorsulfonic, citric, tartaric, succinic, dichloroacetic, ethenesulfonic, formic, fumaric, gluconic, glutamic, hippuric, hydrobromic, hydrochloric, isethionic, lactic, maleic, malic, malonic, mandelic, methanesulfonic, mucic, napthalenesulfonic, nitric, oxalic, pamoic, pantothenic, phosphoric, phthalic, propionic, succinic, sulfuric, tartaric, toluenesulfonic, and camphorsulfonic as well as other known pharmaceutically acceptable acids.

When any variable occurs more than one time in any constituent or in any formula, its definition in each occurrence is independent of its definition at every other occurrence (e.g., in N(R²⁴)₂, each R²⁴ may be the same or different than the other). Combinations of substituents and/or variables are permissible only if such combinations result in stable compounds.

The terms “treat” and “treating” and “treatment” as used herein, refer to partially or completely alleviating, inhibiting, ameliorating and/or relieving a condition from which a patient is suspected to suffer.

As used herein, “therapeutically effective” and “effective dose” refer to a substance or an amount that elicits a desirable biological activity or effect.

Except when noted, the terms “subject” or “patient” are used interchangeably and refer to mammals such as human patients and non-human primates, as well as experimental animals such as rabbits, rats, and mice, and other animals. Accordingly, the term “subject” or “patient” as used herein means any mammalian patient or subject to which the compounds of the invention can be administered. In an exemplary embodiment of the present invention, to identify subject patients for treatment according to the methods of the invention, accepted screening methods are employed to determine risk factors associated with a targeted or suspected disease or condition or to determine the status of an existing disease or condition in a subject. These screening methods include, for example, conventional work-ups to determine risk factors that may be associated with the targeted or suspected disease or condition. These and other routine methods allow the clinician to select patients in need of therapy using the methods and compounds of the present invention.

The TDP-43 binders of the present invention are 4-aminoquinoline compounds and 4-aminoquinazoline compounds, which include all enantiomeric and diastereomeric forms and pharmaceutically accepted salts thereof. The TDP-43 binders of the present invention may include a compound having the formula (I):

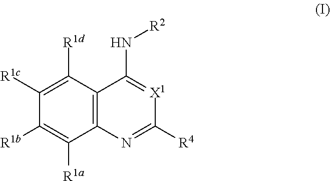

including an enantiomer, a diastereomer, a hydrate, a solvate, a pharmaceutically acceptable salt, an isotopic analog, a prodrug, and a complex thereof.

In formula (I),
R^{1a} is selected from the group consisting of hydrogen, halogen, C₁₋₂₀ linear alkyl, C₃₋₂₀ branched alkyl, C₁₋₂₀ linear heteroalkyl, C₃₋₂₀ branched heteroalkyl, each of which except hydrogen and halogen are optionally substituted;
R^{1b} is selected from the group consisting of hydrogen, halogen, C₁₋₂₀ linear alkyl, C₃₋₂₀ branched alkyl, C₁₋₂₀ linear heteroalkyl, C₃₋₂₀ branched heteroalkyl, each of which except hydrogen and halogen are optionally substituted;
R^{1c} is selected from the group consisting of hydrogen, halogen, C₁₋₂₀ linear alkyl, C₃₋₂₀ branched alkyl, C₁₋₂₀ linear heteroalkyl, C₃₋₂₀ branched heteroalkyl, each of which except hydrogen and halogen are optionally substituted; and
R^{1d} is selected from the group consisting of hydrogen, halogen, C₁₋₂₀ linear alkyl, C₃₋₂₀ branched alkyl, C₁₋₂₀ linear heteroalkyl, C₃₋₂₀ branched heteroalkyl, each of which except hydrogen and halogen are optionally substituted.

In an embodiment of formula (I),
R^{1a} is selected from the group consisting of hydrogen, halogen, C₁₋₂₀ linear alkyl, C₃₋₂₀ branched alkyl, C₁₋₂₀ linear alkoxy, C₃₋₂₀ branched alkoxy, C₁₋₂₀ linear aminoalkyl, C₃₋₂₀ branched aminoalkyl, C₁₋₂₀ linear aminoalkoxy, and C₃₋₂₀ branched aminoalkoxy, each of which except hydrogen and halogen are optionally substituted;
R^{1b} is selected from the group consisting of hydrogen, halogen, C₁₋₂₀ linear alkyl, C₃₋₂₀ branched alkyl, C₁₋₂₀ linear alkoxy, C₃₋₂₀ branched alkoxy, C₁₋₂₀ linear aminoalkyl, C₃₋₂₀ branched aminoalkyl, C₁₋₂₀ linear aminoalkoxy, and C₃₋₂₀ branched aminoalkoxy, each of which except hydrogen and halogen are optionally substituted;
R^{1c} is selected from the group consisting of hydrogen, halogen, C₁₋₂₀ linear alkyl, C₃₋₂₀ branched alkyl, C₁₋₂₀ linear alkoxy, C₃₋₂₀ branched alkoxy, C₁₋₂₀ linear aminoalkyl, C₃₋₂₀ branched aminoalkyl, C₁₋₂₀ linear aminoalkoxy, and C₃₋₂₀ branched aminoalkoxy, each of which except hydrogen and halogen are optionally substituted; and
R^{1d} is selected from the group consisting of hydrogen, halogen, C₁₋₂₀ linear alkyl, C₃₋₂₀ branched alkyl, C₁₋₂₀ linear alkoxy, C₃₋₂₀ branched alkoxy, C₁₋₂₀ linear aminoalkyl, C₃₋₂₀ branched aminoalkyl, C₁₋₂₀ linear aminoalkoxy, and C₃₋₂₀ branched aminoalkoxy, each of which except hydrogen and halogen are optionally substituted.

In an embodiment of formula (I), at least one selected from the group consisting of R^{1a}, R^{1b}, R^{1c}, and R^{1d} is a substituted or unsubstituted C₁₋₂₀ aminoalkoxy group. The term “amino” may refer to an unsubstituted amino group (—NH₂) or a substituted amino group (—NR₂), wherein groups R are the same or different and are independently selected from hydrogen or a C₁-C₁₀ alkyl group, and wherein groups R may be connected to form a ring. An example of the C₁₋₂₀ aminoalkoxy group is a pyrrolidino(C₃-C₈)alkoxy group.

In an embodiment of formula (I), each of the C₁₋₂₀ linear alkyl, C₃₋₂₀ branched alkyl, C₁₋₂₀ linear alkoxy, and C₃₋₂₀ branched alkoxy is optionally substituted with at least one fluorine.

In another embodiment of formula (I), any two neighboring groups selected from the group consisting of R^{1a}, R^{1b}, R^{1c}, and R^{1d} are optionally connected to form a ring. Such a connection can be formed by an imaginary abstraction of any atom or a group of atoms from each of the neighboring groups to form a free bond and then connecting the free bonds to form a ring. For example, when R^{1b} and R^{1c} are each an ethyl group (—CH₂CH₃), an imaginary abstraction of a hydrogen atom (H) from CH₃ of R^{1b} and R^{1c} followed by connection of the free bonds could produce —CH₂CH₂—CH₂CH₂—, wherein the carbon atoms in bold are the connected atoms. In another example, when R^{1b} an ethyl group (—CH₂CH₃) and R^{1c} is a methoxy group (—OCH₃), imaginary abstraction of a hydrogen atom (H) from CH₃ of R^{1b} and R^{1c} followed by connection of the free bonds could produce —CH₂CH₂—CH₂O—, wherein the carbon atoms in bold are the connected atoms. In yet another example, when R^{1b} and R^{1c} are each a trifluoromethoxy group (—OCF₃), imaginary abstraction of a fluorine atom (F) from R^{1b} and a trifluoromethyl group (CF₃) from R^{1c} followed by connection of the free bonds could produce —OCF₂—O—, wherein the carbon and oxygen atoms in bold are the connected atoms.

In formula (I), R² is a C₁-C₂₀ organic group including at least one nitrogen atom. The C₁-C₂₀ organic group may be linear, branched, or cyclic, and may be either substituted or unsubstituted. The C₁-C₂₀ organic group may be a substituted or unsubstituted C₁-C₂₀ hydrocarbon group, in which at least one carbon atom is replaced by nitrogen. The C₁-C₂₀ organic group including at least one nitrogen atom may include at least one C₀-C₁₀ amino group, which may be primary, secondary, or tertiary, and which may be monocyclic, bicyclic, or tricyclic. The C₁-C₂₀ organic group may include a C₄-C₈ monocyclic amino group, a C₄-C₁₀ bicyclic amino group, or a C₆-C₁₂ tricyclic amino group.

In an embodiment, at least one of the substituents of the C₁-C₂₀ heteroalkyl group may be selected from:
—F, —Cl, —Br, —I, a hydroxyl group, a cyano group, a nitro group, a C₀-C₁₀ amino group, a C₁-C₁₀ alkyl group, a C₂-C₁₀ alkenyl group, a C₂-C₁₀ alkynyl group, and a C₁-C₁₀ alkoxy group;
a C₁-C₁₀ alkyl group, a C₂-C₁₀ alkenyl group, a C₂-C₁₀ alkynyl group, and a C₁-C₁₀ alkoxy group, each substituted with at least one selected from —F, —Cl, —Br, —I, a hydroxyl group, a cyano group, a nitro group, a C₀-C₁₀ amino group, a C₃-C₁₀ cycloalkyl group, a C₂-C₁₀ heterocycloalkyl group, a C₃-C₁ cycloalkenyl group, a C₂-C₁₀ heterocycloalkenyl group, a C₆-C₂₀ aryl group, a C₆-C₂₀ aryloxy group, a C₆-C₂₀ arylthio group, a C₂-C₂₀ heteroaryl group,
a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₂₀ aryl group, a C₆-C₂₀ aryloxy group, a C₆-C₂₀ arylthio group, a C₂-C₂₀ heteroaryl group, and
a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₂₀ aryl group, a C₆-C₂₀ aryloxy group, a C₆-C₂₀ arylthio group, a C₂-C₂₀ heteroaryl group, each substituted with at least one selected from —F, —Cl, —Br, —I, a hydroxyl group, a cyano group, a nitro group, a C₀-C₁₀ amino group, a C₁-C₁₀ alkyl group, a C₂-C₁₀ alkenyl group, a C₂-C₁₀ alkynyl group, a C₁-C₁₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₂₀ aryl group, a C₆-C₂₀ aryloxy group, a C₆-C₂₀ arylthio group, a C₂-C₂₀ heteroaryl group.

In an embodiment, R² is not

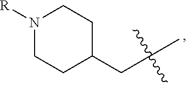

wherein R is hydrogen, C₁-C₆ alkyl, hydroxy(C₁-C₆)alkyl, amino(C₁-C₆)alkyl, mono- or di(C₁-C₆)alkylamino(C₁-C₆)alkyl, alkoxy(C₁-C₆)alkyl, (C₁-C₆)alkoxy, aryl(C₁-C₆)alkyl, or C(O)NR′R″ wherein R′ and R″ are the same or different and are independently hydrogen, lower alkyl, or NR″′, wherein R′ and R″ optionally form a ring having 3-7 members.

In formula (I), R⁴ is hydrogen, —F, —Cl, —Br, —I, a cyano group, a nitro group, a substituted or unsubstituted C₁-C₂₀ alkyl group, a substituted or unsubstituted C₂-C₂₀ alkenyl group, a substituted or unsubstituted C₂-C₂₀ alkynyl group, a substituted or unsubstituted C₁-C₂₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₂₀ aryl group, a substituted or unsubstituted C₆-C₂₀ aryloxy group, a substituted or unsubstituted C₆-C₂₀ arylthio group, or a substituted or unsubstituted C₂-C₂₀ heteroaryl group.

In an embodiment, at least one of substituents of the substituted C₁-C₂₀ alkyl group, the substituted C₂-C₂₀ alkenyl group, the substituted C₂-C₂₀ alkynyl group, the substituted C₁-C₂₀ alkoxy group, the substituted C₃-C₁₀ cycloalkyl group, the substituted C₂-C₁₀ heterocycloalkyl group, the substituted C₃-C₁₀ cycloalkenyl group, the substituted C₂-C₁₀ heterocycloalkenyl group, the substituted C₆-C₂₀ aryl group, the substituted C₆-C₂₀ aryloxy group, the substituted C₆-C₂₀ arylthio group, and the substituted C₂-C₂₀ heteroaryl group may be selected from:
—F, —Cl, —Br, —I, a hydroxyl group, a cyano group, a nitro group, a C₀-C₁₀ amino group, a C₁-C₁₀ alkyl group, a C₂-C₁₀ alkenyl group, a C₂-C₁₀ alkynyl group, and a C₁-C₁₀ alkoxy group;
a C₁-C₁₀ alkyl group, a C₂-C₁₀ alkenyl group, a C₂-C₁₀ alkynyl group, and a C₁-C₁₀ alkoxy group, each substituted with at least one selected from —F, —Cl, —Br, —I, a hydroxyl group, a cyano group, a nitro group, a C₀-C₁₀ amino group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₂₀ aryl group, a C₆-C₂₀ aryloxy group, a C₆-C₂₀ arylthio group, a C₂-C₂₀ heteroaryl group,
a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₂₀ aryl group, a C₆-C₂₀ aryloxy group, a C₆-C₂₀ arylthio group, a C₂-C₂₀ heteroaryl group, and
a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₂₀ aryl group, a C₆-C₂₀ aryloxy group, a C₆-C₂₀ arylthio group, a C₂-C₂₀ heteroaryl group, each substituted with at least one selected from —F, —Cl, —Br, —I, a hydroxyl group, a cyano group, a nitro group, a C₀-C₁₀ amino group, a C₁-C₁₀ alkyl group, a C₂-C₁₀ alkenyl group, a C₂-C₁₀ alkynyl group, a C₁-C₁₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₂₀ aryl group, a C₆-C₂₀ aryloxy group, a C₆-C₂₀ arylthio group, a C₂-C₂₀ heteroaryl group.

In an embodiment of formula (I), R⁴ is
a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isooxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzooxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group;
a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isooxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, phenanthridinyl, acridinyl, phenanthrolinyl, phenazinyl, a benzoimidazolyl group, a benzofuranyl group, a benzothiophenyl group, an isobenzothiazolyl group, a benzooxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group, each substituted with at least one selected from —F, —Cl, —Br, —I, a hydroxyl group, a cyano group, a nitro group, a C₀-C₁₀ amino group, a C₁-C₁₀ alkyl group, a C₁-C₁₀ alkoxy group, a phenyl group, a phenyl group substituted with a C₁-C₁₀ alkyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isooxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzooxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, a biphenyl group.

In an embodiment of formula (I):
X¹ is selected from the group consisting of nitrogen and CH;
R^{1a} is selected from the group consisting of hydrogen, halogen, CF₃, OCF₃, C₁₋₄ linear alkyl, C₃₋₄ branched alkyl, (C₂₋₈ dialkylamino)(C₂₋₄ alkyl), (C₃₋₆ alkyleneamino)(C₂₋₄ alkyl), C₁₋₄ linear alkoxy, and C₃₋₄ branched alkoxy;
R^{1b} is selected from the group consisting of hydrogen, halogen, CF₃, OCF₃, C₁₋₄ linear alkyl, C₃₋₄ branched alkyl, (C₂₋₈ dialkylamino)(C₂₋₄ alkyl), (C₃₋₆ alkyleneamino)(C₂₋₄ alkyl), C₁₋₄ linear alkoxy, and C₃₋₄ branched alkoxy;
R^{1c} is selected from the group consisting of hydrogen, halogen, CF₃, OCF₃, C₁₋₄ linear alkyl, C₃₋₄ branched alkyl, (C₂₋₈ dialkylamino)(C₂₋₄ alkyl), (C₃₋₆ alkyleneamino)(C₂₋₄ alkyl), C₁₋₄ linear alkoxy, and C₃₋₄ branched alkoxy;
R^{1d} is selected from the group consisting of hydrogen, halogen, CF₃, OCF₃, C₁₋₄ linear alkyl, C₃₋₄ branched alkyl, (C₂₋₈ dialkylamino)(C₂₋₄ alkyl), (C₃₋₆ alkyleneamino)(C₂₋₄ alkyl), C₁₋₄ linear alkoxy, and C₃₋₄ branched alkoxy;
wherein any two selected from the group consisting of R^{1a}, R^{1b}, R^{1c}, and R^{1d} are optionally connected to each other to form a ring;
R² is selected from the group consisting of —(CH₂)ₙ—NR⁵R⁶, —(CH₂)ₙC(O)—NR⁵R⁶,

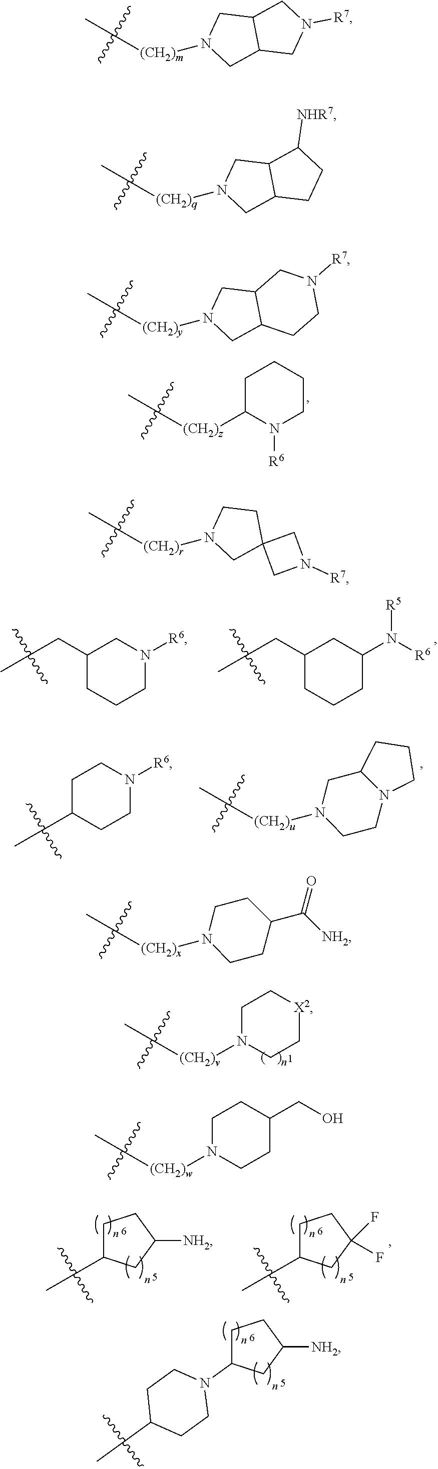

n¹ is 1 or 2;
R⁴ is hydrogen; CF₃; a five-membered monocyclic heteroaryl ring comprising at least one heteroatom selected from the group consisting from O, N, and S that is optionally substituted with up to 2 groups selected from C₁₋₄ linear alkyl, C₃₋₄ branched alkyl, C₁₋₄ linear alkoxy, C₃₋₄ branched alkoxy, CF₃, CF₃O, halogen,

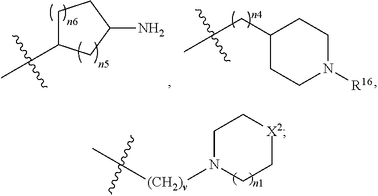

a five-membered monocyclic heteroaryl ring comprising at least one heteroatom selected from the group consisting from O, N, and S;

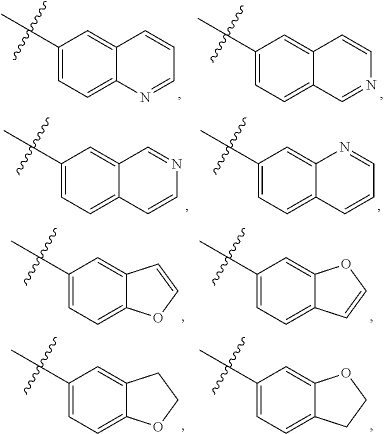

a phenyl ring that is optionally substituted with up to 2 groups selected from C₁₋₄ linear alkyl, C₃₋₄ branched alkyl, C₁₋₄ linear alkoxy, C₃₋₄ branched alkoxy, C₃₋₆ cycloalkyl, C₃₋₆ cycloalkoxy, CF₃, CF₃O, halogen, NR^{3a}R^{3b}, SO₂NR^{3a}R^{3b}, NSO₂R^{3a}

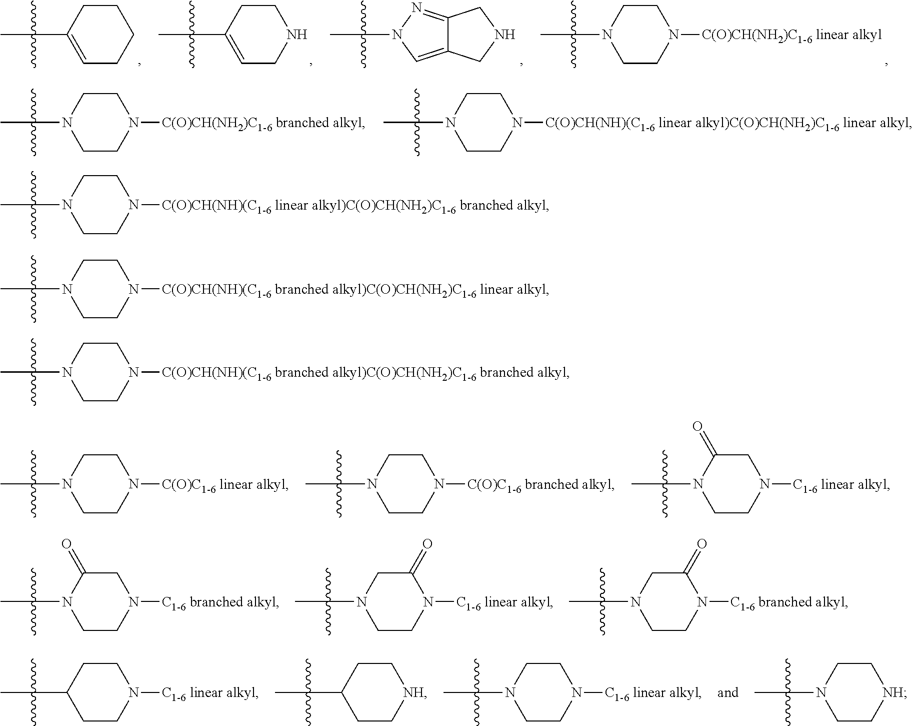

R^{3a} is at each occurrence independently selected from the group consisting of C₁₋₄ linear alkyl and C₃₋₄ branched alkyl;
R^{3b} is at each occurrence independently selected from the group consisting of C₁₋₄ linear alkyl and C₃₋₄ branched alkyl;
R⁵ is selected from the group consisting of hydrogen, C₁₋₄ linear alkyl, C₃₋₄ branched alkyl, —CH₂—(C₁₋₆ cycloalkyl), C(O)C₁₋₆ linear alkyl, C(O)C₃₋₆ branched alkyl, C(O)CH(NH₂)C₁₋₆ linear alkyl, C(O)CH(NH₂)C₃₋₆ branched alkyl,

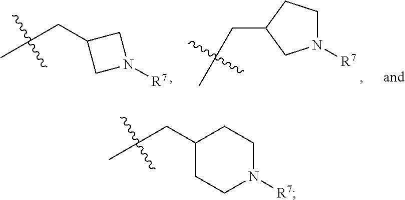

R⁶ is selected from the group consisting of C₁₋₄ linear alkyl, C₃₋₄ branched alkyl

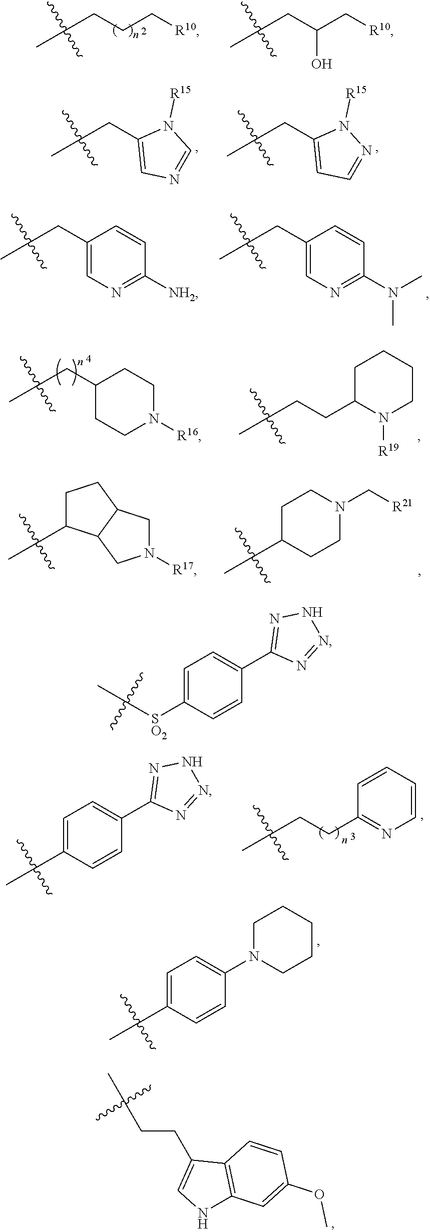

and an optionally substituted benzyl group wherein the optionally substituted benzyl group is substituted with 0 to 2 groups selected from the group consisting of halogen, —CN, —NO₂, —OH, —NH₂, C₁₋₆ alkyl, C₃₋₇ branched alkyl, C₁₋₆ linear haloalkyl, C₃₋₇ branched haloalkyl, C₁₋₆ linear alkoxy, C₃₋₇ branched alkoxy, C₁₋₆ linear haloalkoxy, C₃₋₇ branched haloalkoxy, C₃₋₇ cycloalkyl, aryl, heterocycle, and heteroaryl;
n² is 1 or 2;
n³ is 0 or 1;
n⁴ is 0 or 1;
n⁵ is 0, 1, or 2;
n⁶ is 0 or 1;
R⁷ is selected from the group consisting of hydrogen and C(O)OR⁸;
R⁸ is selected from the group consisting of C₁₋₄ linear alkyl and C₃₋₄ branched alkyl;
X² is selected from the group consisting of a single bond, oxygen, CH₂, CHOH, and NR⁹;
R⁹ is C₁₋₄ linear alkyl that is optionally substituted with an NH₂;
R¹⁰ is selected from the group consisting of OH, OR¹¹, NR¹²R¹³, NHSO₂R²², and

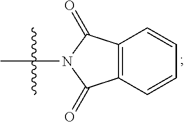 [SMILES: *N1C(=O)c2ccccc2C1=O]

R¹¹ is selected from the group consisting of C₁₋₄ linear alkyl and C₃₋₄ branched alkyl;
R¹² is selected from the group consisting of hydrogen, C₁₋₄ linear alkyl, C₃₋₄ branched alkyl, C₁₋₄ linear fluoroalkyl, C(O)R¹⁴;
R¹³ is selected from the group consisting of hydrogen, C₁₋₄ linear alkyl, C₃₋₄ branched alkyl, and heteroaryl;
R¹² and R¹³ are optionally taken together with the atoms to which they are connected to form a ring with 3 to 6 atoms;
R¹⁴ is selected from the group consisting of C₁₋₄ linear alkyl C₃₋₄ branched alkyl, and

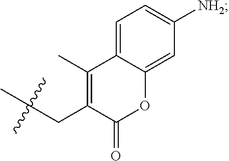 [SMILES: *Cc1c(C)c2ccc(N)cc2oc1=O]

R¹⁵ is selected from the group consisting of C₁₋₄ linear alkyl and C₃₋₄ branched alkyl;
R¹⁶ is selected from the group consisting of hydrogen, C₁₋₄ linear alkyl, C₃₋₄ branched alkyl, CH₂—(C₁₋₆ cycloalkyl), and C(O)R¹⁸;
R¹⁷ is selected from the group consisting of hydrogen, benzyl and C(O)R¹⁸;
R¹⁸ is selected from the group consisting of C₁₋₄ linear alkyl and C₃₋₄ branched alkyl;
R¹⁹ is selected from the group consisting of hydrogen and C(O)R²⁰;
R²⁰ is selected from the group consisting of C₁₋₄ linear alkyl and C₃₋₄ branched alkyl;
R²¹ is a benzene ring that is optionally substituted with 0 to 2 groups selected from the group consisting of halogen, —CN, —NO₂, —OH, —NH₂, C₁₋₆ alkyl, C₃₋₇ branched alkyl, C₁₋₆ linear haloalkyl, C₃₋₇ branched haloalkyl, C₁₋₆ linear alkoxy, C₃₋₇ branched alkoxy, C₁₋₆ linear haloalkoxy, C₃₋₇ branched haloalkoxy, C₃₋₇ cycloalkyl, aryl, heterocycle, and heteroaryl;
R²² is selected from the group consisting of C₁₋₄ linear alkyl, C₃₋₄ branched alkyl,

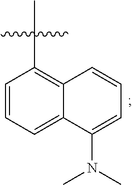 [SMILES: *c1cccc2c(N(C)C)cccc12]

n is 2, 3, or 4;
m is 2, 3, or 4;
q is 2, 3, or 4;
y is 2, 3, or 4;
u is 2, 3, or 4;
v is 2, 3, or 4;
w is 2, 3, or 4;
z is 1, 2 or 3;
r is 2, 3, or 4; and
x is 2, 3, or 4.

The compounds of the present invention include a compound having formula (II):

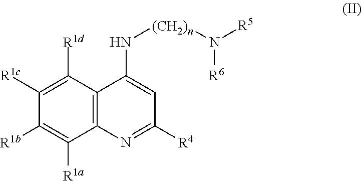

including an enantiomer, a diastereomer, a hydrate, a solvate, a pharmaceutically acceptable salt, an isotopic analog, a prodrug, and a complex thereof.

The compounds of the present invention include a compound having formula (III):

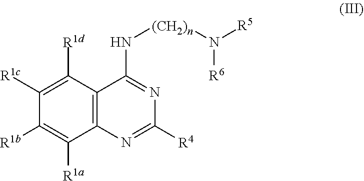

including an enantiomer, a diastereomer, a hydrate, a solvate, a pharmaceutically acceptable salt, an isotopic analog, a prodrug, and a complex thereof.

The compounds of the present invention include a compound having formula (IV):

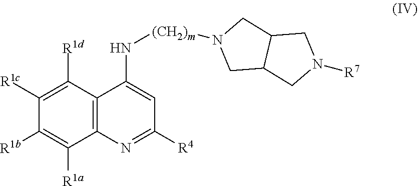

including an enantiomer, a diastereomer, a hydrate, a solvate, a pharmaceutically acceptable salt, an isotopic analog, a prodrug, and a complex thereof.

The compounds of the present invention include a compound having formula (V):

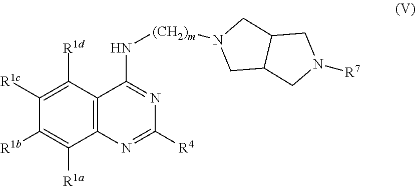

including an enantiomer, a diastereomer, a hydrate, a solvate, a pharmaceutically acceptable salt, an isotopic analog, a prodrug, and a complex thereof.

The compounds of the present invention include a compound having formula (VI):

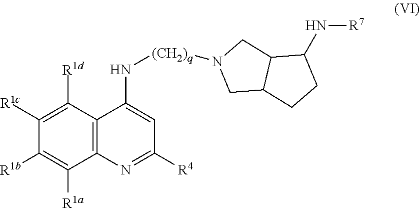

including an enantiomer, a diastereomer, a hydrate, a solvate, a pharmaceutically acceptable salt, an isotopic analog, a prodrug, and a complex thereof.

The compounds of the present invention include a compound having formula (VII):

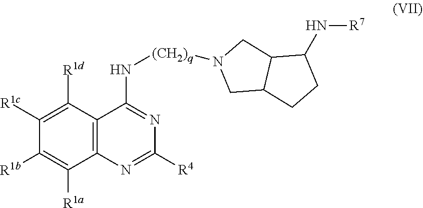

including an enantiomer, a diastereomer, a hydrate, a solvate, a pharmaceutically acceptable salt, an isotopic analog, a prodrug, and a complex thereof.

The compounds of the present invention include a compound having formula (VIII):

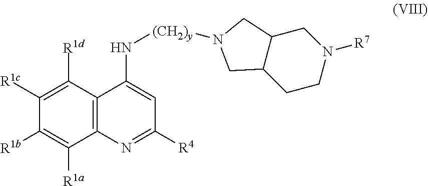

including an enantiomer, a diastereomer, a hydrate, a solvate, a pharmaceutically acceptable salt, an isotopic analog, a prodrug, and a complex thereof.

The compounds of the present invention include a compound having formula (IX):

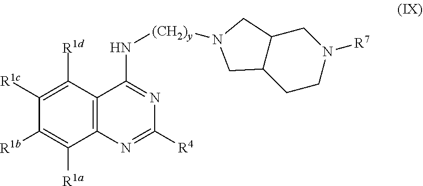

including an enantiomer, a diastereomer, a hydrate, a solvate, a pharmaceutically acceptable salt, an isotopic analog, a prodrug, and a complex thereof.

The compounds of the present invention include a compound having formula (X):

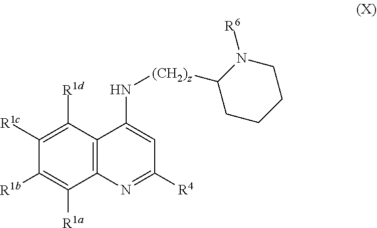

including an enantiomer, a diastereomer, a hydrate, a solvate, a pharmaceutically acceptable salt, an isotopic analog, a prodrug, and a complex thereof.

The compounds of the present invention include a compound having formula (XI):

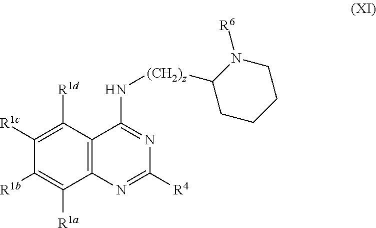

including an enantiomer, a diastereomer, a hydrate, a solvate, a pharmaceutically acceptable salt, an isotopic analog, a prodrugs, and a complex thereof.

The compounds of the present invention include a compound having formula (XII):

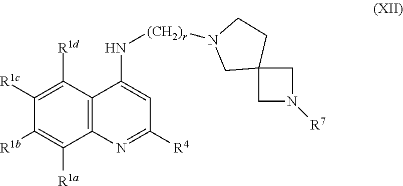

including an enantiomer, a diastereomer, a hydrate, a solvate, a pharmaceutically acceptable salt, an isotopic analog, a prodrug, and a complex thereof.

The compounds of the present invention include a compound having formula (XIII):

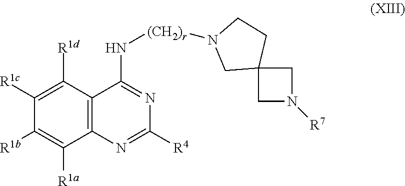

including an enantiomer, a diastereomer, a hydrate, a solvate, a pharmaceutically acceptable salt, an isotopic analog, a prodrug, and a complex thereof.

The compounds of the present invention include a compound having formula (XIV):

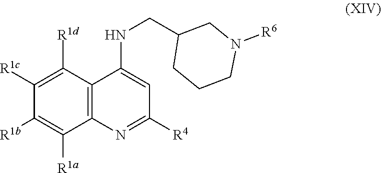

including an enantiomer, a diastereomer, a hydrate, a solvate, a pharmaceutically acceptable salt, an isotopic analog, a prodrug, and a complex thereof.

The compounds of the present invention include a compound having formula (XV):

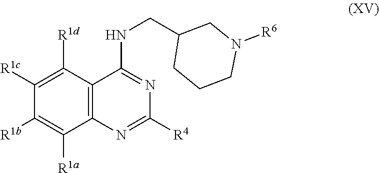

including an enantiomer, a diastereomer, a hydrate, a solvate, a pharmaceutically acceptable salt, an isotopic analog, a prodrug, and a complex thereof.

The compounds of the present invention include a compound having formula (XVI):

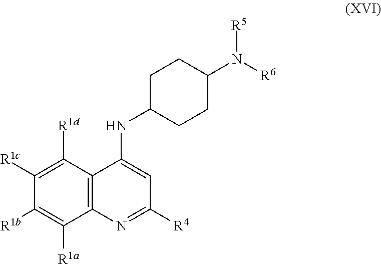

including an enantiomer, a diastereomer, a hydrate, a solvate, a pharmaceutically acceptable salt, an isotopic analog, a prodrug, and a complex thereof.

The compounds of the present invention include a compound having formula (XVII):

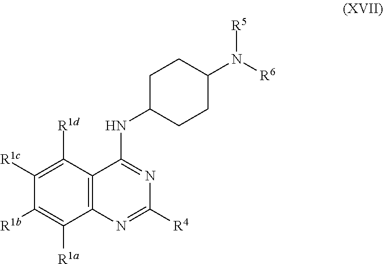

including an enantiomer, a diastereomer, a hydrate, a solvate, a pharmaceutically acceptable salt, an isotopic analog, a prodrug, and a complex thereof.

The compounds of the present invention include a compound having formula (XVIII):

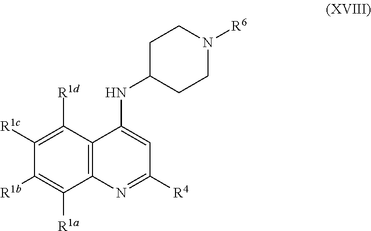

including an enantiomer, a diastereomer, a hydrate, a solvate, a pharmaceutically acceptable salt, an isotopic analog, a prodrug, and a complex thereof.

In some embodiments, a compound having formula (XVIII), wherein R6 is selected from the group consisting of hydrogen, C₁₋₄ linear alkyl, C₃₋₄ branched alkyl, and a benzyl group are excluded. In other embodiments, a compound having formula (XVIII), wherein R⁶ is selected from the group consisting of hydrogen, C₁₋₄ linear alkyl, C₃₋₄ branched alkyl and benzyl, and wherein R^{1b} is selected from the group consisting of CH₃O and

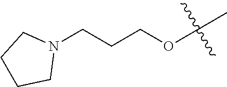 [SMILES: *OCCCN1CCCC1]

are excluded.

The compounds of the present invention include a compound having formula (XIX):

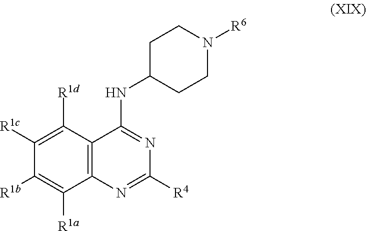

including an enantiomer, a diastereomer, a hydrate, a solvate, a pharmaceutically acceptable salt, an isotopic analog, a prodrug, and a complex thereof. In some embodiments, a compound having formula (XIX), wherein R⁶ is selected from the group consisting of hydrogen, C₁₋₄ linear alkyl, C₃₋₄ branched alkyl, and a benzyl group are excluded. In other embodiments, a compound having formula (XIX), wherein R⁶ is selected from the group consisting of hydrogen, C₁₋₄ linear alkyl, C₃₋₄ branched alkyl and benzyl, and wherein R^{1b} is selected from the group consisting of CH₃O

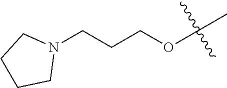 [SMILES: *OCCCN1CCCC1]

are excluded.

The compounds of the present invention include a compound having formula (XX):

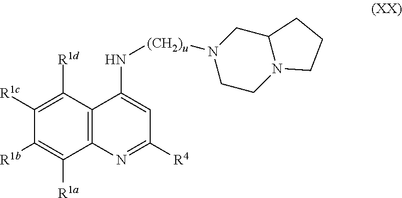

including an enantiomer, a diastereomer, a hydrate, a solvate, a pharmaceutically acceptable salt, an isotopic analog, a prodrug, and a complex thereof.

The compounds of the present invention include a compound having formula (XXI):

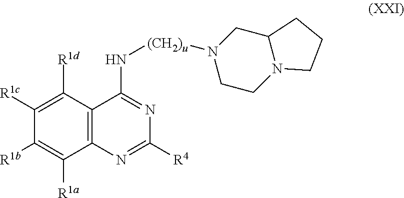

including an enantiomers, a diastereomer, a hydrate, a solvate, a pharmaceutically acceptable salt, an isotopic analog, a prodrug, and a complex thereof.

The compounds of the present invention include a compound having formula (XXII):

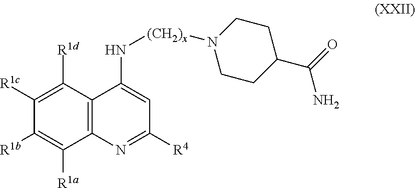

including an enantiomer, a diastereomer, a hydrate, a solvate, a pharmaceutically acceptable salt, an isotopic analog, a prodrug, and a complex thereof.

The compounds of the present invention include a compound having formula (XXIII):

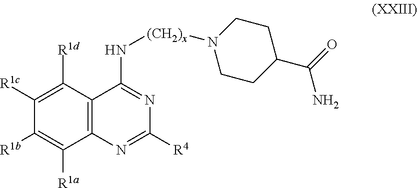

including an enantiomer, a diastereomer, a hydrate, a solvate, a pharmaceutically acceptable salt, an isotopic analog, a prodrug, and a complex thereof.

The compounds of the present invention include a compound having formula (XXIV):

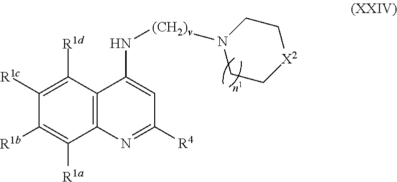

including an enantiomer, a diastereomer, a hydrate, a solvate, a pharmaceutically acceptable salt, an isotopic analog, a prodrug, and a complex thereof.

The compounds of the present invention include a compound having formula (XXV):

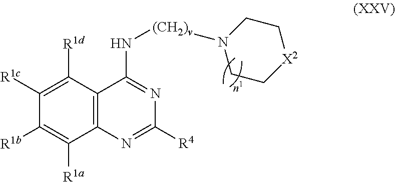

including an enantiomer, a diastereomer, a hydrate, a solvate, a pharmaceutically acceptable salt, an isotopic analog, a prodrug, and a complex thereof.

The compounds of the present invention include a compound having formula (XXVI):

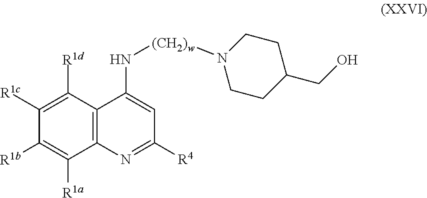

including an enantiomer, a diastereomer, a hydrate, a solvate, a pharmaceutically acceptable salt, an isotopic analog, a prodrug, and a complex thereof.

The compounds of the present invention include a compound having formula (XXVII):

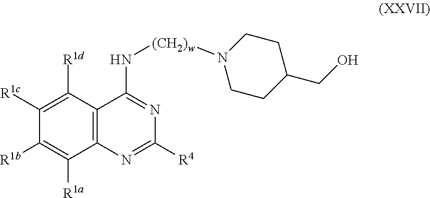

including an enantiomer, a diastereomer, a hydrate, a solvate, a pharmaceutically acceptable salt, an isotopic analog, a prodrug, and a complex thereof.

In some embodiments X¹ is nitrogen.

In some embodiments X¹ is CH.

In some embodiments R^{1a} is hydrogen.

In some embodiments R^{1a} is halogen.

In some embodiments R^{1a} is CF₃.

In some embodiments R^{1a} is OCF₃.

In some embodiments R^{1a} is C₁₋₄ linear alkyl.

In some embodiments R^{1a} is C₃₋₄ branched alkyl.

In some embodiments R^{1a} is (C₂₋₈ dialkylamino)(C₂₋₄ alkyl).

In some embodiments R^{1a} is (C₃₋₆ alkyleneamino)(C₂₋₄ alkyl).

In some embodiments R^{1a} is C₁₋₄ linear alkoxy.

In some embodiments R^{1a} is C₃₋₄ branched alkoxy.

In some embodiments R^{1b} is hydrogen.

In some embodiments R^{1b} is halogen.

In some embodiments R^{1b} is CF₃.

In some embodiments R^{1b} is OCF₃.

In some embodiments R^{1b} is C₁₋₄ linear alkyl.

In some embodiments R^{1b} is C₃₋₄ branched alkyl.

In some embodiments R^{1b} is (C₂₋₈ dialkylamino)(C₂₋₄ alkyl).

In some embodiments R^{1b} is (C₃₋₆ alkyleneamino)(C₂₋₄ alkyl).

In some embodiments R^{1b} is C₁₋₄ linear alkoxy.

In some embodiments R^{1b} is C₃₋₄ branched alkoxy.

In some embodiments R^{1c} is hydrogen.

In some embodiments R^{1c} is halogen.

In some embodiments R^{1c} is CF₃.

In some embodiments R^{1c} is OCF₃.

In some embodiments R^{1c} is C₁₋₄ linear alkyl.

In some embodiments R^{1c} is C₃₋₄ branched alkyl.

In some embodiments R^{1c} is (C₂₋₈ dialkylamino)(C₂₋₄ alkyl).

In some embodiments R^{1c} is (C₃₋₆ alkyleneamino)(C₂₋₄ alkyl).

In some embodiments R^{1c} is C₁₋₄ linear alkoxy.

In some embodiments R^{1c} is C₃₋₄ branched alkoxy.

In some embodiments R^{1a} is hydrogen.

In some embodiments R^{1d} is halogen.

In some embodiments R^{1d} is CF₃.

In some embodiments R^{1d} is OCF₃.

In some embodiments R^{1d} is C₁₋₄ linear alkyl.

In some embodiments R^{1d} is C₃₋₄ branched alkyl.

In some embodiments R^{1d} is (C₂₋₈ dialkylamino)(C₂₋₄ alkyl).

In some embodiments R^{1d} is (C₃₋₆ alkyleneamino)(C₂₋₄ alkyl).

In some embodiments R^{1d} is C₁₋₄ linear alkoxy.

In some embodiments R^{1d} is C₃₋₄ branched alkoxy.

In some embodiment, any two selected from the group consisting of R^{1a}, R^{1b}, R^{1c}, and R^{1d} are optionally connected to each other to form a ring.

In some embodiments R² is —(CH₂)ₙ—NR⁵R⁶.

In some embodiments R² is —(CH₂)ₙC(O)—NR⁵R⁶.

In some embodiments R² is

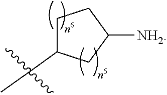

In some embodiments R² is

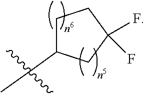

In some embodiments R² is

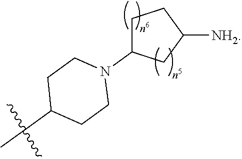

In some embodiments R² is

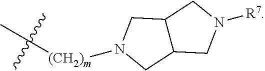

In some embodiments R² is

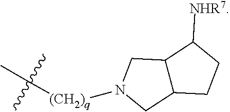

In some embodiments R² is

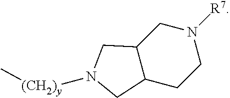

In some embodiments R² is

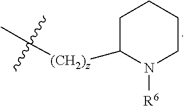

In some embodiments R² is

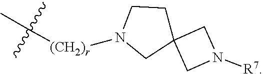

In some embodiments R² is

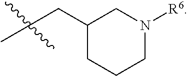

In some embodiments R² is

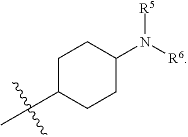

In some embodiments R² is

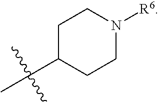

In some embodiments R² is

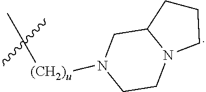

In some embodiments R² is

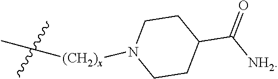

In some embodiments R² is

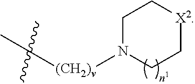

In some embodiments R² is

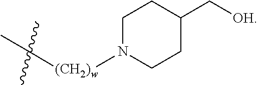

In some embodiments n¹ is 1.

In some embodiments n¹ is 2.

In some embodiments R⁴ is hydrogen.

In some embodiments R⁴ is CF₃.

In some embodiments R⁴ is a five-membered monocyclic heteroaryl ring comprising at least one heteroatom selected from the group consisting from O, N, and S.

In some embodiments R⁴ is a five-membered monocyclic heteroaryl ring comprising at least one heteroatom selected from the group consisting from O, N, and S that is optionally substituted with up to 2 groups selected from C₁₋₄ linear alkyl, C₃₋₄ branched alkyl, C₁₋₄ linear alkoxy, C₃₋₄ branched alkoxy, CF₃, CF₃O, halogen,

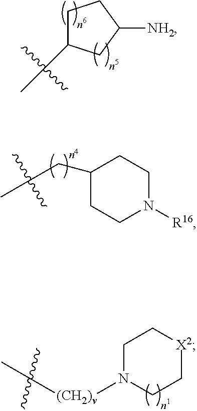

a five-membered monocyclic heteroaryl ring comprising at least one heteroatom selected from the group consisting from O, N, and S.

In some embodiments R⁴ is

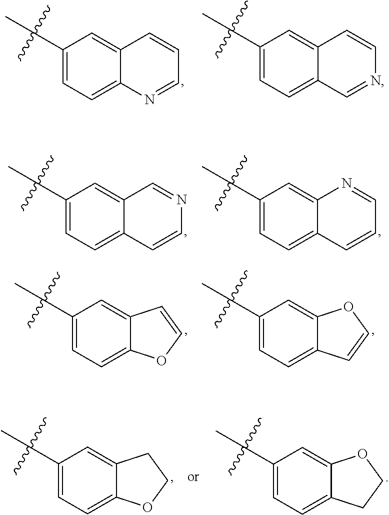

In some embodiments R⁴ is a phenyl ring.

In some embodiments R⁴ is a phenyl ring that is substituted with one group selected from C₁₋₄ linear alkyl, C₃₋₄ branched alkyl, C₁₋₄ linear alkoxy, C₃₋₄ branched alkoxy, C₃₋₆ cycloalkyl, C₃₋₆ cycloalkoxy, CF₃, CF₃O, halogen, NR^{3a}R^{3b}, SO₂NR^{3a}R^{3b}, NHSO₂R^{3a}, and

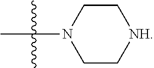 [SMILES: *N1CCNCC1]

In some embodiments R⁴ is a phenyl ring that is substituted with two groups selected from C₁₋₄ linear alkyl, C₃₋₄ branched alkyl, C₁₋₄ linear alkoxy, C₃₋₄ branched alkoxy, CF₃, CF₃O, halogen, NR^{3a}R^{3b}, SO₂NR^{3a}R^{3b}, NHSO₂R^{3a}, and

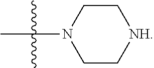 [SMILES: *N1CCNCC1]

In some embodiments R^{3a} is C₁₋₄ linear alkyl.

In some embodiments R^{3a} is C₃₋₄ branched alkyl.

In some embodiments R^{3b} is C₁₋₄ linear alkyl.

In some embodiments R^{3b} is C₃₋₄ branched alkyl.

In some embodiments R⁵ is hydrogen.

In some embodiments R⁵ is C₁₋₄ linear alkyl.

In some embodiments R⁵ is C₃₋₄ branched alkyl.

In some embodiments R⁵ is —CH₂—(C₁₋₆ cycloalkyl).

In some embodiments R⁵ is C(O)C₁₋₆ linear alkyl.

In some embodiments R⁵ is C(O)C₃₋₆ branched alkyl.

In some embodiments R⁵ is C(O)CH(NH₂)C₁₋₆ linear alkyl.

In some embodiments R⁵ is C(O)CH(NH₂)C₃₋₆ branched alkyl.

In some embodiments R⁵ is

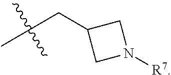

In some embodiments R⁵ is

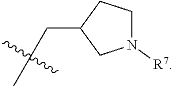

In some embodiments R⁵ is

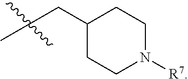

In some embodiments R⁶ is C₁₋₄ linear alkyl.

In some embodiments R⁶ is C₃₋₄ branched alkyl.

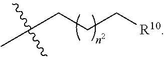

In some embodiments R⁶ is

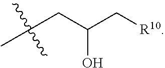

In some embodiments R⁶ is

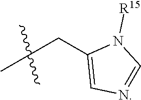

In some embodiments R⁶ is

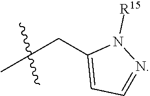

In some embodiments R⁶ is

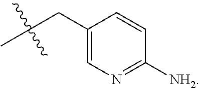 [SMILES: *Cc1ccc(N)nc1]

In some embodiments R⁶ is

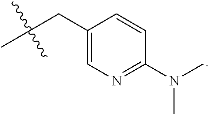 [SMILES: *Cc1ccc(N(C)C)nc1]

In some embodiments R⁶ is

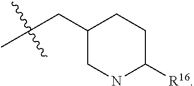

In some embodiments R⁶ is

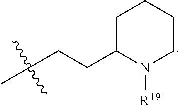

In some embodiments R⁶ is

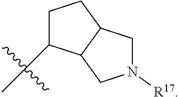

In some embodiments R⁶ is

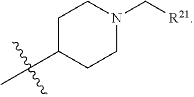

In some embodiments R⁶ is

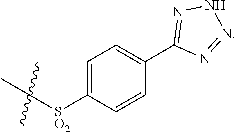 [SMILES: *S(=O)(=O)c1ccc(-c2nn[nH]n2)cc1]

In some embodiments R⁶ is

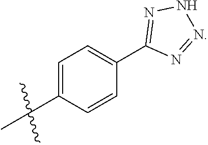 [SMILES: *c1ccc(-c2nn[nH]n2)cc1]

In some embodiments R⁶ is

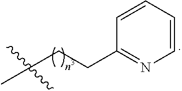

In some embodiments R⁶ is

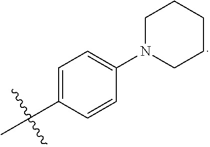 [SMILES: *c1ccc(N2CCCCC2)cc1]

In some embodiments R⁶ is

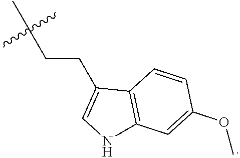 [SMILES: *CCc1c[nH]c2cc(OC)ccc12]

In some embodiments R⁶ is benzyl.

In some embodiments R⁶ is a benzyl group that is substituted with 1 group selected from the group consisting of halogen, —CN, —NO₂, —OH, —NH₂, C₁₋₆ alkyl, C₃₋₇ branched alkyl, C₁₋₆ linear haloalkyl, C₃₋₇ branched haloalkyl, C₁₋₆ linear alkoxy, C₃₋₇ branched alkoxy, C₁₋₆ linear haloalkoxy, C₃₋₇ branched haloalkoxy, C₃₋₇ cycloalkyl, aryl, heterocycle, and heteroaryl.

In some embodiments R⁶ is a benzyl group that is substituted with 2 groups selected from the group consisting of halogen, —CN, —NO₂, —OH, —NH₂, C₁₋₆ alkyl, C₃₋₇ branched alkyl, C₁₋₆ linear haloalkyl, C₃₋₇ branched haloalkyl, C₁₋₆ linear alkoxy, C₃₋₇ branched alkoxy, C₁₋₆ linear haloalkoxy, C₃₋₇ branched haloalkoxy, C₃₋₇ cycloalkyl, aryl, heterocycle, and heteroaryl.

In some embodiments n² is 1.

In some embodiments n² is 2.

In some embodiments n³ is 0.

In some embodiments n³ is 1.

In some embodiments n⁴ is 0.

In some embodiments n⁴ is 1.

In some embodiments n⁵ is 0.

In some embodiments n⁵ is 1.

In some embodiments n⁵ is 2.

In some embodiments n⁶ is 0.

In some embodiments n⁶ is 1.

In some embodiments R⁷ is hydrogen.

In some embodiments R⁷ is C(O)OR⁸.

In some embodiments R⁸ is C₁₋₄ linear alkyl.

In some embodiments R⁸ is C₃₋₄ branched alkyl.

In some embodiments X² is a single bond.

In some embodiments X² is CH₂.

In some embodiments X² is oxygen.

In some embodiments X² is CHOH.

In some embodiments X² is NR⁹.

In some embodiments R⁹ is C₁₋₄ linear alkyl.

In some embodiments R⁹ is C₁₋₄ linear alkyl that is substituted with an NH₂.

In some embodiments R¹⁰ is OH.

In some embodiments R¹⁰ is OR¹¹.

In some embodiments R¹⁰ is NR¹²R¹³.

In some embodiments R¹⁰ is NHSO₂R²².

In some embodiments R¹⁰ is

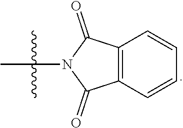 [SMILES: *N1C(=O)c2ccccc2C1=O]

In some embodiments R¹¹ is C₁₋₄ linear alkyl.

In some embodiments R¹¹ is C₃₋₄ branched alkyl.

In some embodiments R¹² is hydrogen.

In some embodiments R¹² is C₁₋₄ linear alkyl.

In some embodiments R¹² is C₃₋₄ branched alkyl.

In some embodiments R¹² is C₁₋₄ linear fluoroalkyl.

In some embodiments R¹² is C(O)R¹⁴.

In some embodiments R¹³ is hydrogen.

In some embodiments R¹³ is C₁₋₄ linear alkyl.

In some embodiments R¹³ is C₃₋₄ branched alkyl

In some embodiments R¹³ is heteroaryl.

In some embodiments R¹² and R¹³ are taken together with the atoms to which they are connected to form a ring with 3 atoms.

In some embodiments R¹² and R¹³ are taken together with the atoms to which they are connected to form a ring with 4 atoms.

In some embodiments R¹² and R¹³ are taken together with the atoms to which they are connected to form a ring with 5 atoms.

In some embodiments R¹² and R¹³ are taken together with the atoms to which they are connected to form a ring with 6 atoms.

In some embodiments R¹⁴ is C₁₋₄ linear alkyl.

In some embodiments R¹⁴ is C₃₋₄ branched alkyl.

In some embodiments R¹⁴ is

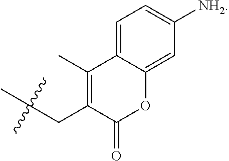 [SMILES: *Cc1c(C)c2ccc(N)cc2oc1=O]

In some embodiments R¹⁵ is C₁₋₄ linear alkyl.

In some embodiments R¹⁵ is C₃₋₄ branched alkyl.

In some embodiments R¹⁶ is hydrogen.

In some embodiments R¹⁶ is C₁₋₄ linear alkyl.

In some embodiments R¹⁶ is C₃₋₄ branched alkyl.

In some embodiments R¹⁶ is CH₂—(C₁₋₆ cycloalkyl).

In some embodiments R¹⁶ is C(O)R¹⁸.

In some embodiments R¹⁷ is hydrogen.

In some embodiments R¹⁷ is benzyl.

In some embodiments R¹⁷ is C(O)R¹⁸.

In some embodiments R¹⁸ is C₁₋₄ linear alkyl.

In some embodiments R¹⁸ is C₃₋₄ branched alkyl.

In some embodiments R¹⁹ is hydrogen.

In some embodiments R¹⁹ is C(O)R²⁰.

In some embodiments R²⁰ is C₁₋₄ linear alkyl.

In some embodiments R²⁰ is C₃₋₄ branched alkyl.

In some embodiments R²¹ is a benzene ring.

In some embodiments R²¹ is a benzene ring that is substituted with 1 group selected from the group consisting of halogen, —CN, —NO₂, —OH, —NH₂, C₁₋₆ alkyl, C₃₋₇ branched alkyl, C₁₋₆ linear haloalkyl, C₃₋₇ branched haloalkyl, C₁₋₆ linear alkoxy, C₃₋₇ branched alkoxy, C₁₋₆ linear haloalkoxy, C₃₋₇ branched haloalkoxy, C₃₋₇ cycloalkyl, aryl, heterocycle, and heteroaryl.

In some embodiments R²¹ is a benzene ring that is substituted with 2 groups selected from the group consisting of halogen, —CN, —NO₂, —OH, —NH₂, C₁₋₆ alkyl, C₃₋₇ branched alkyl, C₁₋₆ linear haloalkyl, C₃₋₇ branched haloalkyl, C₁₋₆ linear alkoxy, C₃₋₇ branched alkoxy, C₁₋₆ linear haloalkoxy, C₃₋₇ branched haloalkoxy, C₃₋₇ cycloalkyl, aryl, heterocycle, and heteroaryl.

In some embodiments R²² is C₁₋₄ linear alkyl.

In some embodiments R²² is C₃₋₄ branched alkyl.

In some embodiments R²² is

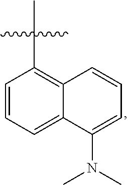 [SMILES: *c1cccc2c(N(C)C)cccc12]

In some embodiments n is 2.

In some embodiments n is 3.

In some embodiments n is 4.

In some embodiments m is 2.

In some embodiments m is 3.

In some embodiments m is 4.

In some embodiments q is 2.

In some embodiments q is 3.

In some embodiments q is 4.

In some embodiments y is 2.

In some embodiments y is 3.

In some embodiments y is 4.

In some embodiments u is 2.

In some embodiments u is 3.

In some embodiments u is 4.

In some embodiments v is 2.

In some embodiments v is 3.

In some embodiments v is 4.

In some embodiments w is 2.

In some embodiments w is 3.

In some embodiments w is 4.

In some embodiments z is 2.

In some embodiments z is 3.

In some embodiments z is 4.

In some embodiments r is 2.

In some embodiments r is 3.

In some embodiments r is 4.

In some embodiments x is 2.

In some embodiments x is 3.

In some embodiments x is 4.

The following compounds in which each hydrogen is a proton may be excluded:

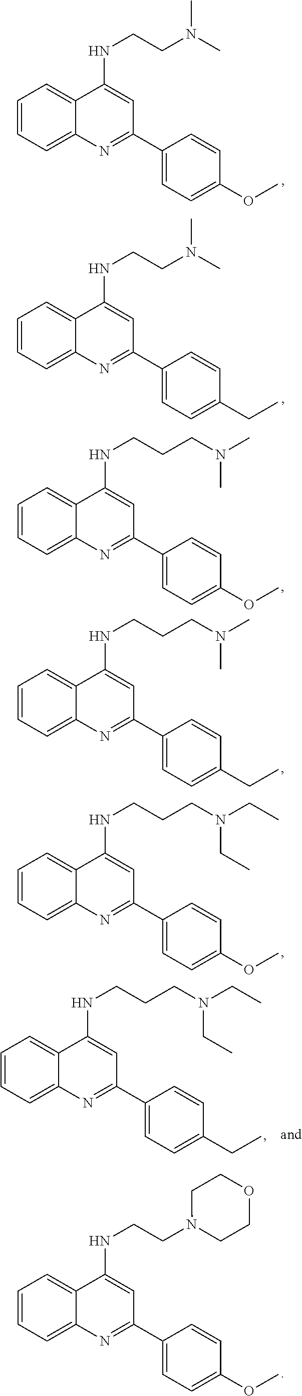

For the purposes of demonstrating the manner in which the compounds of the present invention are named and referred to herein, the compound having the formula:

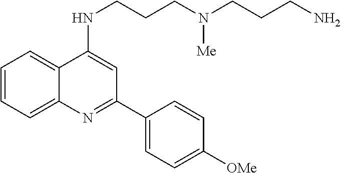 [SMILES: COc1ccc(-c2cc(NCCCN(C)CCCN)c3ccccc3n2)cc1]

has the chemical name N¹-(3-aminopropyl)-N³-(2-(4-methoxyphenyl)quinolin-4-yl)-N¹-methylpropane-1,3-diamine.

For the purposes of demonstrating the manner in which the compounds of the present invention are named and referred to herein, the compound having the formula:

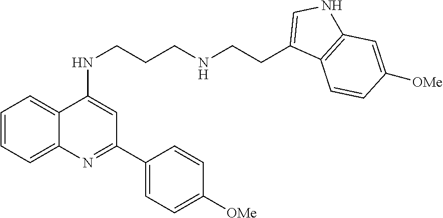 [SMILES: COc1ccc(-c2cc(NCCCNCCc3c[nH]c4cc(OC)ccc34)c3ccccc3n2)cc1]

has the chemical name N-(3-(2-(6-methoxy-1H-indol-3-yl)ethylamino)propyl)-2-(4-methoxyphenyl) quinolin-4-amine.

For the purposes of the present invention, a compound depicted by the racemic formula will stand equally well for either of the two enantiomers or mixtures thereof, or in the case where a second chiral center is present, all diastereomers.

For the purposes of the present invention, a compound depicted by the racemic formula will stand equally well for either of the two enantiomers or mixtures thereof, or in the case where a second chiral center is present, all diastereomers.

In all of the embodiments provided herein, examples of suitable optional substituents are not intended to limit the scope of the claimed invention. The compounds of the invention may contain any of the substituents, or combinations of substituents, provided herein.

### PROCESS OF PREPARATION

The present invention further relates to a process for preparing the TDP-43 binding agents of the present invention.

Compounds of the present teachings can be prepared in accordance with the procedures outlined herein, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given, other process conditions can also be used unless otherwise stated. Optimum reaction conditions can vary with the particular reactants or solvent used, but such conditions can be determined by one skilled in the art by routine optimization procedures. Those skilled in the art of organic synthesis will recognize that the nature and order of the synthetic steps presented can be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatography such as high pressure liquid chromatography (HPLC), gas chromatography (GC), gel-permeation chromatography (GPC), or thin layer chromatography (TLC).

Preparation of the compounds can involve protection and deprotection of various chemical groups. The need for protection and deprotection and the selection of appropriate protecting groups can be readily determined by one skilled in the art. The chemistry of protecting groups can be found, for example, in Greene et al., Protective Groups in Organic Synthesis, 2d. Ed. (Wiley & Sons, 1991), the entire disclosure of which is incorporated by reference herein for all purposes.

The reactions or the processes described herein can be carried out in suitable solvents which can be readily selected by one skilled in the art of organic synthesis. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

The compounds of these teachings can be prepared by methods known in the art of organic chemistry. The reagents used in the preparation of the compounds of these teachings can be either commercially obtained or can be prepared by standard procedures described in the literature. For example, compounds of the present invention can be prepared according to the method illustrated in the General Synthetic Schemes:

### GENERAL SYNTHETIC SCHEMES FOR PREPARATION OF COMPOUNDS

The reagents used in the preparation of the compounds of this invention can be either commercially obtained or can be prepared by standard procedures described in the literature. In accordance with this invention, compounds in the genus may be produced by one of the following reaction schemes.

Compounds of formula (I) may be prepared according to the process outlined in schemes 1-41.

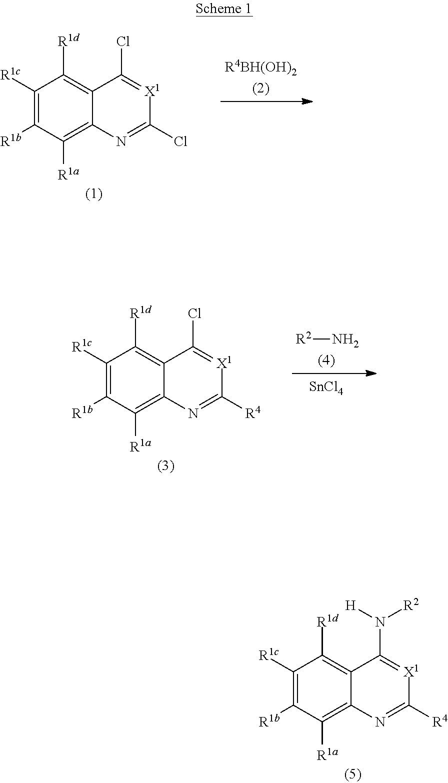

A compound of the formula (1), a known compound or a compound prepared by known methods, is reacted with a compound of the formula (2), a known compound or a compound prepared by known methods, in the presence of a palladium catalyst such as palladium (II) acetate, tetrakis(triphenylphosphine)palladium(0), dichlorobis(triphenylphosphine)palladium(II), palladium on carbon, bis(acetonitrile)dichloropalladium(II), and the like, in a solvent such as methanol, ethanol, tetrahydrofuran, 1,4-dioxane, acetonitrile, methylene chloride, 1,2-dichloroethane, 1,2-dimethoxyethane, and the like, optionally in the presence of toluene, optionally in the present of water, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, optionally with heating, optionally with microwave irradiation to provide a compound of the formula (3). A compound of the formula (3) is reacted with a compound of the formula (4), a known compound or a compound prepared by known methods, in the presence of tin tetrachloride, optionally in the presence of a solvent N,N-dimethylformamide, dimethyl sulfoxide, N,N-dimethylacetamide, and the like, optionally with heating, optionally, with microwave irradiation to provide a compound of the formula (5).

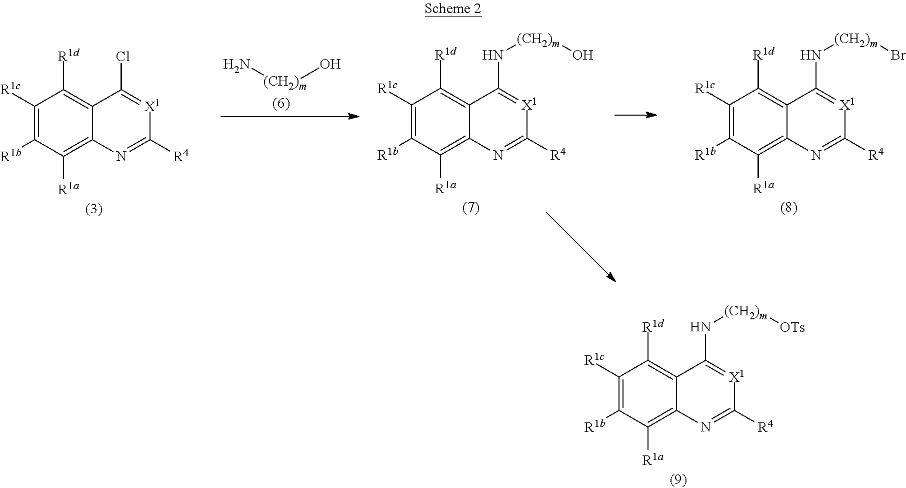

Alternatively, a compound of the formula (3) is reacted with a compound of the formula (6), a known compound or a compound prepared by known methods, in the presence of tin chloride, optionally in the presence of a solvent N,N-dimethylformamide, dimethyl sulfoxide, N,N-dimethylacetamide, and the like, optionally with heating, optionally with microwave irradiation to provide a compound of the formula (7). A compound of the formula (7) is reacted with carbon tetrabromide in the presence of triphenyl phosphine, in the presence of a solvent such as methylene chloride, 1,2-dichloroethane, tetrahydrofuran, 1,4-dioxane, and the like, optionally with heating, optionally with microwave irradiation to provide a compound of the formula (8). Alternatively, a compound of the formula (7) is reacted with 4-methylbenzenesulfonyl chloride in the presence of a base such as triethylamine, N,N-diisopropylethylamine, pyridine, 2,6-dimethylpyridine, and the like, in the presence of a solvent such as methylene chloride, 1,2-dichloroethane, tetrahydrofuran, 1,4-dioxane, and the like, optionally with heating, optionally with microwave irradiation to provide a compound of the formula (9).

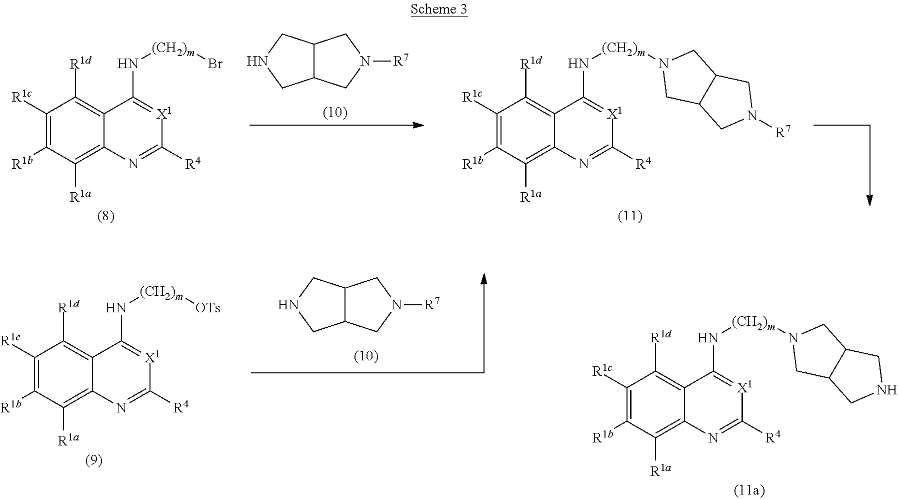

A compound of the formula (8) is reacted with a compound of the formula (10), a known compound or a prepared by known methods, optionally in the presence of solvent such as acetonitrile, tetrahydrofuran, 1,4-dioxane, methylene chloride, 1,2-dichloroethane, N,N-dimethylformamide, dimethyl sulfoxide, N,N-dimethylacetamide, and the like, optionally in the presence of a base such as sodium carbonate, lithium carbonate, potassium carbonate, triethylamine, N,N-diisopropylethylamine, pyridine, 2,6-dimethylpyridine, and the like, optionally with heating, optionally with microwave irradiation to provide a compound of the formula (11). Alternatively, a compound of the formula (9) is reacted with a compound of the formula (10), a known compound or a prepared by known methods, optionally in the presence of solvent such as acetonitrile, tetrahydrofuran, 1,4-dioxane, methylene chloride, 1,2-dichloroethane, N,N-dimethylformamide, dimethyl sulfoxide, N,N-dimethylacetamide, and the like, optionally in the presence of a base such as sodium carbonate, lithium carbonate, potassium carbonate, triethylamine, N,N-diisopropylethylamine, pyridine, 2,6-dimethylpyridine, and the like, optionally with heating, optionally with microwave irradiation to provide a compound of the formula (11). A compound of the formula (11) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethyl sulfoxide, N,N-dimethylacetamide, and the like, optionally with heating, optionally with microwave irradiation to provide a compound of the formula (11a).

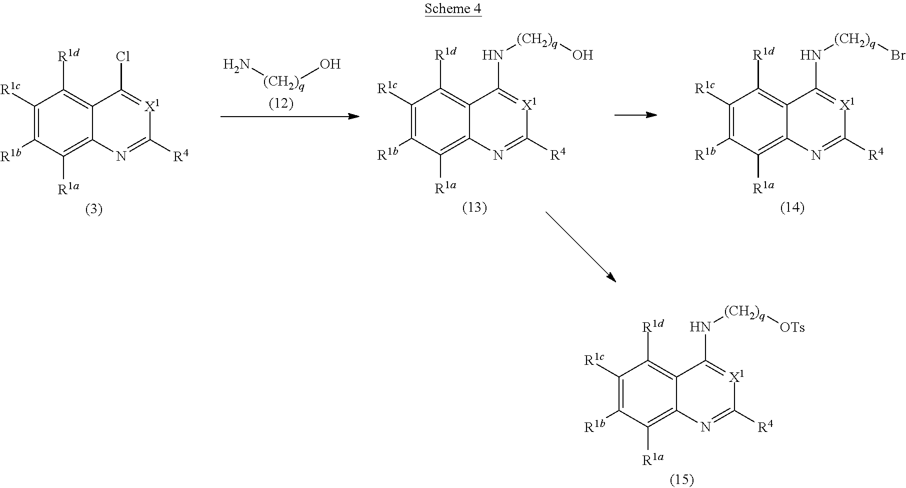

Alternatively, a compound of the formula (3) is reacted with a compound of the formula (12), a known compound or a compound prepared by known methods, in the presence of tin tetrachloride, optionally in the presence of a solvent N,N-dimethylformamide, dimethyl sulfoxide, N,N-dimethylacetamide, and the like, optionally with heating, optionally, with microwave irradiation to provide a compound of the formula (13). A compound of the formula (13) is reacted with carbon tetrabromide in the presence of triphenyl phosphine, in the presence of a solvent such as methylene chloride, 1,2-dichloroethane, tetrahydrofuran, 1,4-dioxane, and the like, optionally with heating, optionally with microwave irradiation to provide a compound of the formula (14). Alternatively, a compound of the formula (13) is reacted with 4-methylbenzenesulfonyl chloride in the presence of a base such as triethylamine, N,N-diisopropylethylamine, pyridine, 2,6-dimethylpyridine, and the like, in the presence of a solvent such as methylene chloride, 1,2-dichloroethane, tetrahydrofuran, 1,4-dioxane, and the like, optionally with heating, optionally with microwave irradiation to provide a compound of the formula (15).

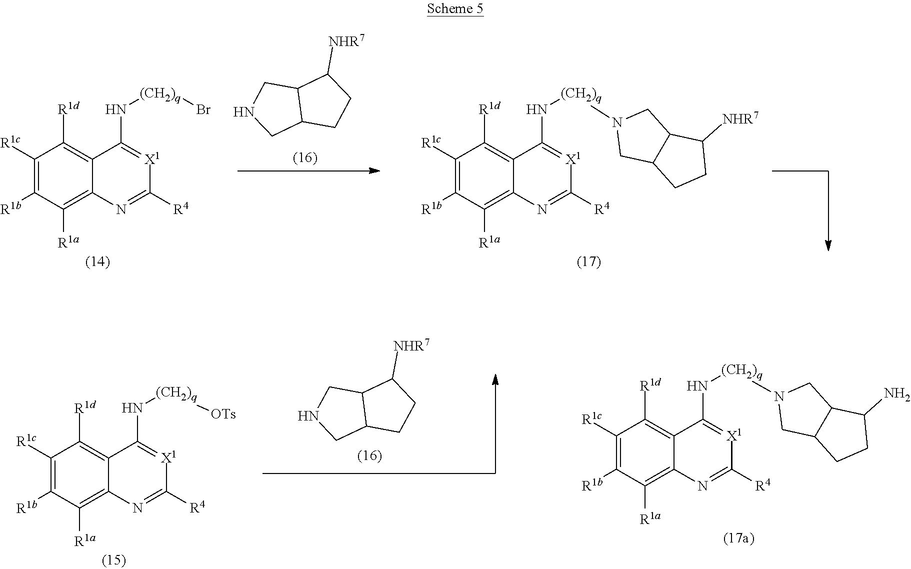

A compound of the formula (14) is reacted with a compound of the formula (16), a known compound or a prepared by known methods, optionally in the presence of solvent such as acetonitrile, tetrahydrofuran, 1,4-dioxane, methylene chloride, 1,2-dichloroethane, N,N-dimethylformamide, dimethyl sulfoxide, N,N-dimethylacetamide, and the like, optionally in the presence of a base such as sodium carbonate, lithium carbonate, potassium carbonate, triethylamine, N,N-diisopropylethylamine, pyridine, 2,6-dimethylpyridine, and the like, optionally with heating, optionally with microwave irradiation to provide a compound of the formula (17). Alternatively, a compound of the formula (15) is reacted with a compound of the formula (16), a known compound or a prepared by known methods, optionally in the presence of solvent such as acetonitrile, tetrahydrofuran, 1,4-dioxane, methylene chloride, 1,2-dichloroethane, N,N-dimethylformamide, dimethyl sulfoxide, N,N-dimethylacetamide, and the like, optionally in the presence of a base such as sodium carbonate, lithium carbonate, potassium carbonate, triethylamine, N,N-diisopropylethylamine, pyridine, 2,6-dimethylpyridine, and the like, optionally with heating, optionally with microwave irradiation to provide a compound of the formula (17). A compound of the formula (17) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethyl sulfoxide, N,N-dimethylacetamide, and the like, optionally with heating, optionally with microwave irradiation to provide a compound of the formula (17a).

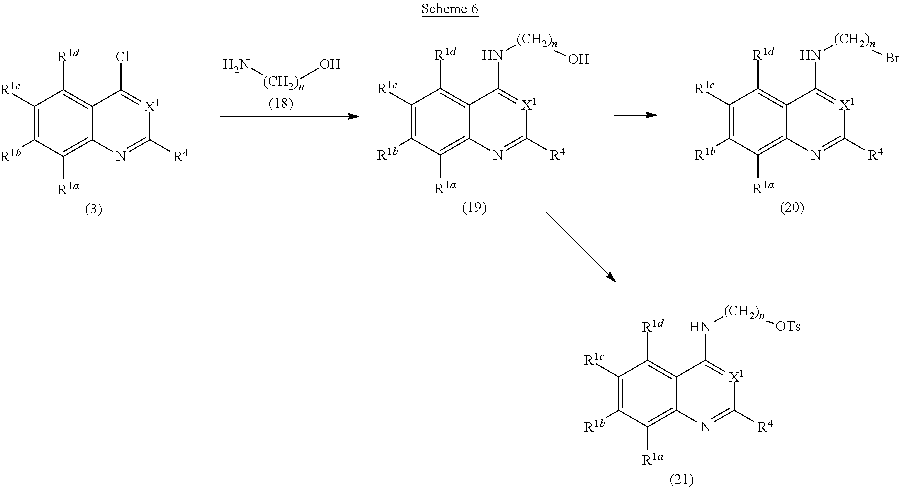

Alternatively, a compound of the formula (3) is reacted with a compound of the formula (18), a known compound or a compound prepared by known methods, in the presence of tin chloride, optionally in the presence of a solvent N,N-dimethylformamide, dimethyl sulfoxide, N,N-dimethylacetamide, and the like, optionally with heating, optionally, with microwave irradiation to provide a compound of the formula (19). A compound of the formula (19) is reacted with carbon tetrabromide in the presence of triphenyl phosphine, in the presence of a solvent such as methylene chloride, 1,2-dichloroethane, tetrahydrofuran, 1,4-dioxane, and the like, optionally with heating, optionally with microwave irradiation to provide a compound of the formula (20). Alternatively, a compound of the formula (19) is reacted with 4-methylbenzenesulfonyl chloride in the presence of a base such as triethylamine, N,N-diisopropylethylamine, pyridine, 2,6-dimethylpyridine, and the like, in the presence of a solvent such as methylene chloride, 1,2-dichloroethane, tetrahydrofuran, 1,4-dioxane, and the like, optionally with heating, optionally with microwave irradiation to provide a compound of the formula (21).

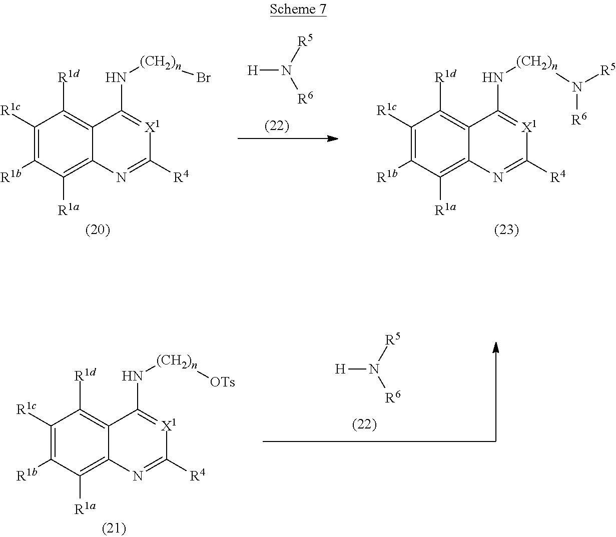

A compound of the formula (20) is reacted with a compound of the formula (22), a known compound or a prepared by known methods, optionally in the presence of solvent such as acetonitrile, tetrahydrofuran, 1,4-dioxane, methylene chloride, 1,2-dichloroethane, N,N-dimethylformamide, dimethyl sulfoxide, N,N-dimethylacetamide, and the like, optionally in the presence of a base such as sodium carbonate, lithium carbonate, potassium carbonate, triethylamine, N,N-diisopropylethylamine, pyridine, 2,6-dimethylpyridine, and the like, optionally with heating, optionally with microwave irradiation to provide a compound of the formula (23). Alternatively, a compound of the formula (21) is reacted with a compound of the formula (22), a known compound or a prepared by known methods, optionally in the presence of solvent such as acetonitrile, tetrahydrofuran, 1,4-dioxane, methylene chloride, 1,2-dichloroethane, N,N-dimethylformamide, dimethyl sulfoxide, N,N-dimethylacetamide, and the like, optionally in the presence of a base such as sodium carbonate, lithium carbonate, potassium carbonate, triethylamine, N,N-diisopropylethylamine, pyridine, 2,6-dimethylpyridine, and the like, optionally with heating, optionally with microwave irradiation to provide a compound of the formula (23).

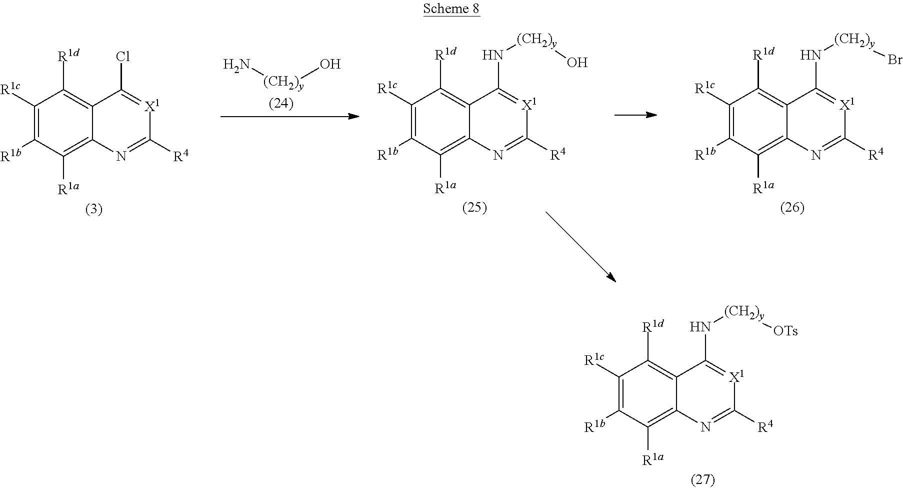

Alternatively, a compound of the formula (3) is reacted with a compound of the formula (24), a known compound or a compound prepared by known methods, in the presence of tin chloride, optionally in the presence of a solvent N,N-dimethylformamide, dimethyl sulfoxide, N,N-dimethylacetamide, and the like, optionally with heating, optionally, with microwave irradiation to provide a compound of the formula (25). A compound of the formula (25) is reacted with carbon tetrabromide in the presence of triphenyl phosphine, in the presence of a solvent such as methylene chloride, 1,2-dichloroethane, tetrahydrofuran, 1,4-dioxane, and the like, optionally with heating, optionally with microwave irradiation to provide a compound of the formula (26). Alternatively, a compound of the formula (25) is reacted with 4-methylbenzenesulfonyl chloride in the presence of a base such as triethylamine, N,N-diisopropylethylamine, pyridine, 2,6-dimethylpyridine, and the like, in the presence of a solvent such as methylene chloride, 1,2-dichloroethane, tetrahydrofuran, 1,4-dioxane, and the like, optionally with heating, optionally with microwave irradiation to provide a compound of the formula (27).

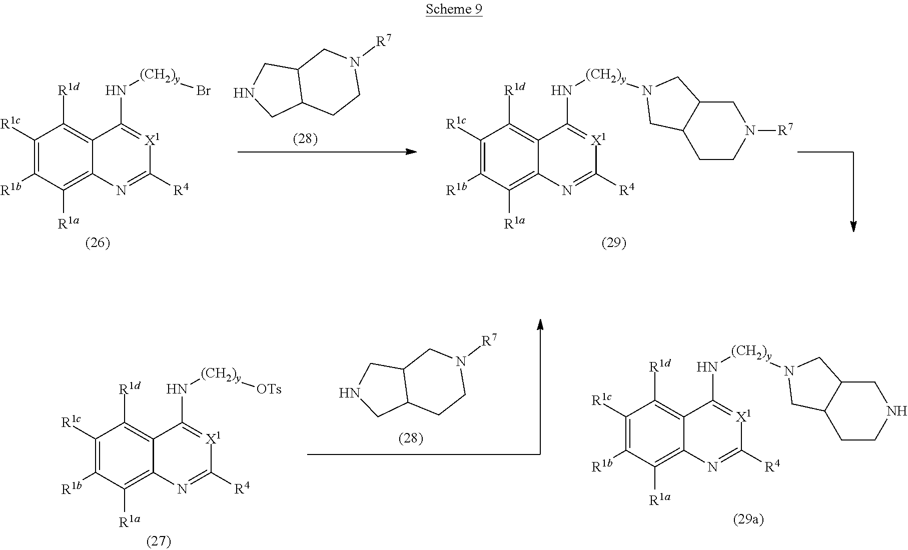

A compound of the formula (26) is reacted with a compound of the formula (28), a known compound or a prepared by known methods, optionally in the presence of solvent such as acetonitrile, tetrahydrofuran, 1,4-dioxane, methylene chloride, 1,2-dichloroethane, N,N-dimethylformamide, dimethyl sulfoxide, N,N-dimethylacetamide, and the like, optionally in the presence of a base such as sodium carbonate, lithium carbonate, potassium carbonate, triethylamine, N,N-diisopropylethylamine, pyridine, 2,6-dimethylpyridine, and the like, optionally with heating, optionally with microwave irradiation to provide a compound of the formula (29). Alternatively, a compound of the formula (27) is reacted with a compound of the formula (28), a known compound or a prepared by known methods, optionally in the presence of solvent such as acetonitrile, tetrahydrofuran, 1,4-dioxane, methylene chloride, 1,2-dichloroethane, N,N-dimethylformamide, dimethyl sulfoxide, N,N-dimethylacetamide, and the like, optionally in the presence of a base such as sodium carbonate, lithium carbonate, potassium carbonate, triethylamine, N,N-diisopropylethylamine, pyridine, 2,6-dimethylpyridine, and the like, optionally with heating, optionally with microwave irradiation to provide a compound of the formula (29). A compound of the formula (29) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethyl sulfoxide, N,N-dimethylacetamide, and the like, optionally with heating, optionally with microwave irradiation to provide a compound of the formula (29a).

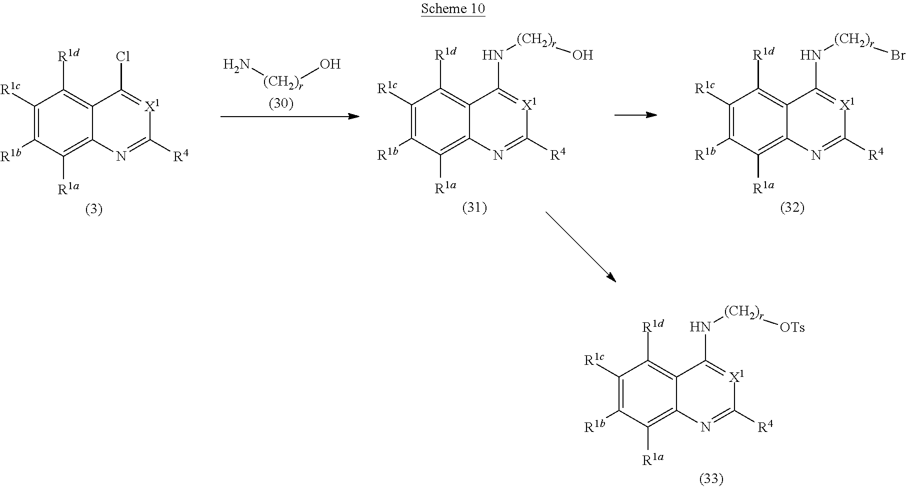

Alternatively, a compound of the formula (3) is reacted with a compound of the formula (30), a known compound or a compound prepared by known methods, in the presence of tin chloride, optionally in the presence of a solvent N,N-dimethylformamide, dimethyl sulfoxide, N,N-dimethylacetamide, and the like, optionally with heating, optionally, with microwave irradiation to provide a compound of the formula (31). A compound of the formula (31) is reacted with carbon tetrabromide in the presence of triphenyl phosphine, in the presence of a solvent such as methylene chloride, 1,2-dichloroethane, tetrahydrofuran, 1,4-dioxane, and the like, optionally with heating, optionally with microwave irradiation to provide a compound of the formula (32). Alternatively, a compound of the formula (31) is reacted with 4-methylbenzenesulfonyl chloride in the presence of a base such as triethylamine, N,N-diisopropylethylamine, pyridine, 2,6-dimethylpyridine, and the like, in the presence of a solvent such as methylene chloride, 1,2-dichloroethane, tetrahydrofuran, 1,4-dioxane, and the like, optionally with heating, optionally with microwave irradiation to provide a compound of the formula (33).

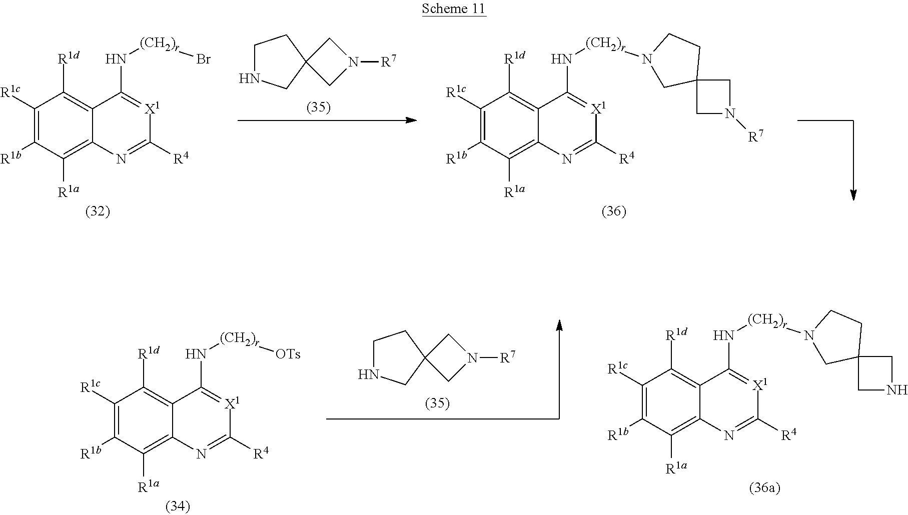

A compound of the formula (32) is reacted with a compound of the formula (35), a known compound or a prepared by known methods, optionally in the presence of solvent such as acetonitrile, tetrahydrofuran, 1,4-dioxane, methylene chloride, 1,2-dichloroethane, N,N-dimethylformamide, dimethyl sulfoxide, N,N-dimethylacetamide, and the like, optionally in the presence of a base such as sodium carbonate, lithium carbonate, potassium carbonate, triethylamine, N,N-diisopropylethylamine, pyridine, 2,6-dimethylpyridine, and the like, optionally with heating, optionally with microwave irradiation to provide a compound of the formula (36). Alternatively, a compound of the formula (34) is reacted with a compound of the formula (35), a known compound or a prepared by known methods, optionally in the presence of solvent such as acetonitrile, tetrahydrofuran, 1,4-dioxane, methylene chloride, 1,2-dichloroethane, N,N-dimethylformamide, dimethyl sulfoxide, N,N-dimethylacetamide, and the like, optionally in the presence of a base such as sodium carbonate, lithium carbonate, potassium carbonate, triethylamine, N,N-diisopropylethylamine, pyridine, 2,6-dimethylpyridine, and the like, optionally with heating, optionally with microwave irradiation to provide a compound of the formula (36). A compound of the formula (36) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethyl sulfoxide, N,N-dimethylacetamide, and the like, optionally with heating, optionally with microwave irradiation to provide a compound of the formula (36a).

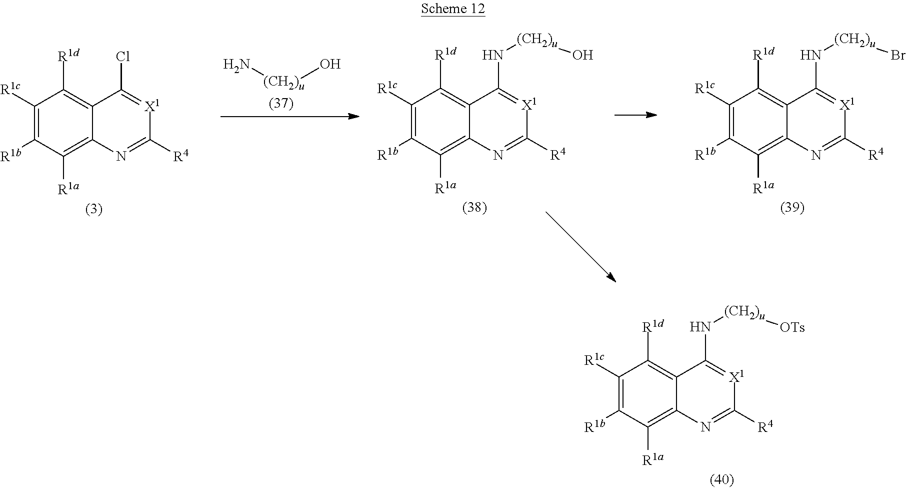

Alternatively, a compound of the formula (3) is reacted with a compound of the formula (37), a known compound or a compound prepared by known methods, in the presence of tin chloride, optionally in the presence of a solvent N,N-dimethylformamide, dimethyl sulfoxide, N,N-dimethylacetamide, and the like, optionally with heating, optionally, with microwave irradiation to provide a compound of the formula (38). A compound of the formula (38) is reacted with carbon tetrabromide in the presence of triphenyl phosphine, in the presence of a solvent such as methylene chloride, 1,2-dichloroethane, tetrahydrofuran, 1,4-dioxane, and the like, optionally with heating, optionally with microwave irradiation to provide a compound of the formula (39). Alternatively, a compound of the formula (38) is reacted with 4-methylbenzenesulfonyl chloride in the presence of a base such as triethylamine, N,N-diisopropylethylamine, pyridine, 2,6-dimethylpyridine, and the like, in the presence of a solvent such as methylene chloride, 1,2-dichloroethane, tetrahydrofuran, 1,4-dioxane, and the like, optionally with heating, optionally with microwave irradiation to provide a compound of the formula (40).

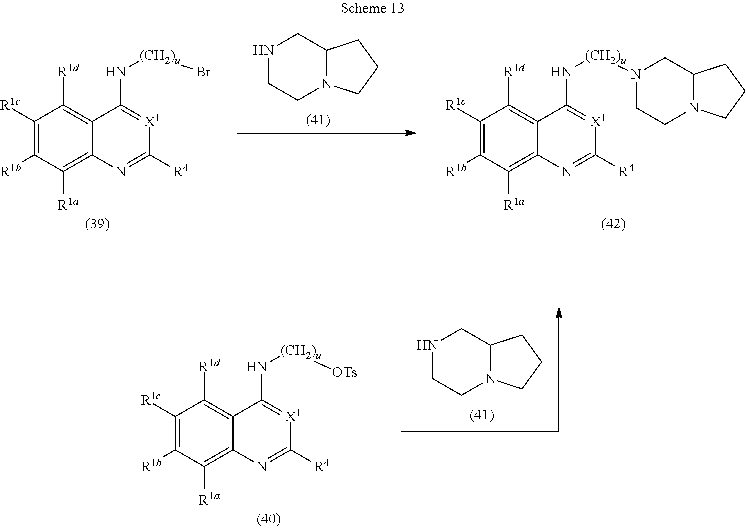

A compound of the formula (39) is reacted with a compound of the formula (41), a known compound or a prepared by known methods, optionally in the presence of solvent such as acetonitrile, tetrahydrofuran, 1,4-dioxane, methylene chloride, 1,2-dichloroethane, N,N-dimethylformamide, dimethyl sulfoxide, N,N-dimethylacetamide, and the like, optionally in the presence of a base such as sodium carbonate, lithium carbonate, potassium carbonate, triethylamine, N,N-diisopropylethylamine, pyridine, 2,6-dimethylpyridine, and the like, optionally with heating, optionally with microwave irradiation to provide a compound of the formula (42). Alternatively, a compound of the formula (40) is reacted with a compound of the formula (41), a known compound or a prepared by known methods, optionally in the presence of solvent such as acetonitrile, tetrahydrofuran, 1,4-dioxane, methylene chloride, 1,2-dichloroethane, N,N-dimethylformamide, dimethyl sulfoxide, N,N-dimethylacetamide, and the like, optionally in the presence of a base such as sodium carbonate, lithium carbonate, potassium carbonate, triethylamine, N,N-diisopropylethylamine, pyridine, 2,6-dimethylpyridine, and the like, optionally with heating, optionally with microwave irradiation to provide a compound of the formula (42).

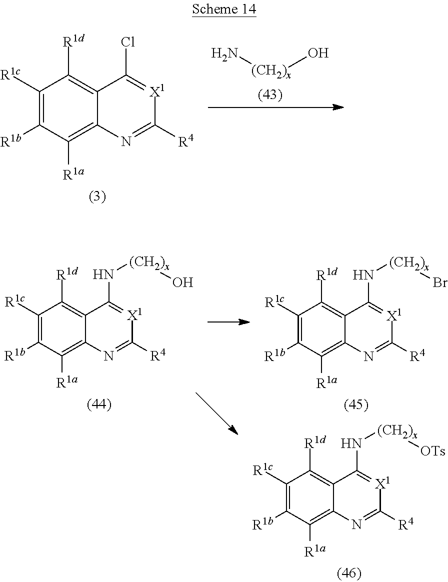

Alternatively, a compound of the formula (3) is reacted with a compound of the formula (43), a known compound or a compound prepared by known methods, in the presence of tin chloride, optionally in the presence of a solvent N,N-dimethylformamide, dimethyl sulfoxide, N,N-dimethylacetamide, and the like, optionally with heating, optionally, with microwave irradiation to provide a compound of the formula (44). A compound of the formula (44) is reacted with carbon tetrabromide in the presence of triphenyl phosphine, in the presence of a solvent such as methylene chloride, 1,2-dichloroethane, tetrahydrofuran, 1,4-dioxane, and the like, optionally with heating, optionally with microwave irradiation to provide a compound of the formula (45). Alternatively, a compound of the formula (44) is reacted with 4-methylbenzenesulfonyl chloride in the presence of a base such as triethylamine, N,N-diisopropylethylamine, pyridine, 2,6-dimethylpyridine, and the like, in the presence of a solvent such as methylene chloride, 1,2-dichloroethane, tetrahydrofuran, 1,4-dioxane, and the like, optionally with heating, optionally with microwave irradiation to provide a compound of the formula (46).

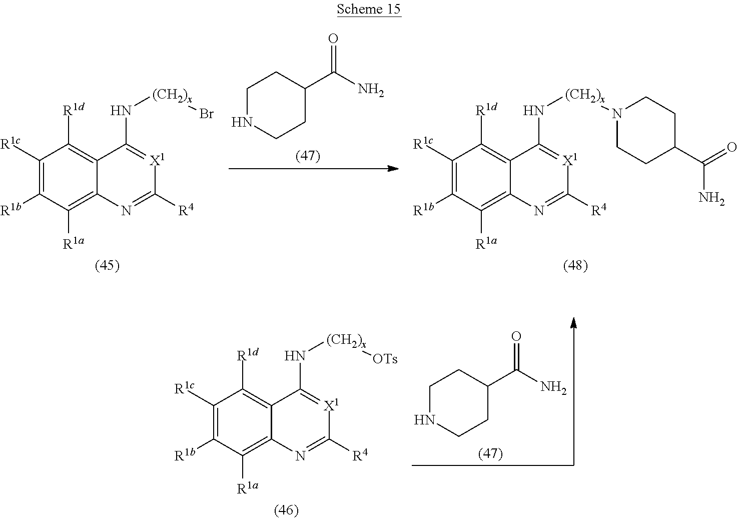

A compound of the formula (45) is reacted with a compound of the formula (47), a known compound or a prepared by known methods, optionally in the presence of solvent such as acetonitrile, tetrahydrofuran, 1,4-dioxane, methylene chloride, 1,2-dichloroethane, N,N-dimethylformamide, dimethyl sulfoxide, N,N-dimethylacetamide, and the like, optionally in the presence of a base such as sodium carbonate, lithium carbonate, potassium carbonate, triethylamine, N,N-diisopropylethylamine, pyridine, 2,6-dimethylpyridine, and the like, optionally with heating, optionally with microwave irradiation to provide a compound of the formula (48). Alternatively, a compound of the formula (46) is reacted with a compound of the formula (47), a known compound or a prepared by known methods, optionally in the presence of solvent such as acetonitrile, tetrahydrofuran, 1,4-dioxane, methylene chloride, 1,2-dichloroethane, N,N-dimethylformamide, dimethyl sulfoxide, N,N-dimethylacetamide, and the like, optionally in the presence of a base such as sodium carbonate, lithium carbonate, potassium carbonate, triethylamine, N,N-diisopropylethylamine, pyridine, 2,6-dimethylpyridine, and the like, optionally with heating, optionally with microwave irradiation to provide a compound of the formula (48).

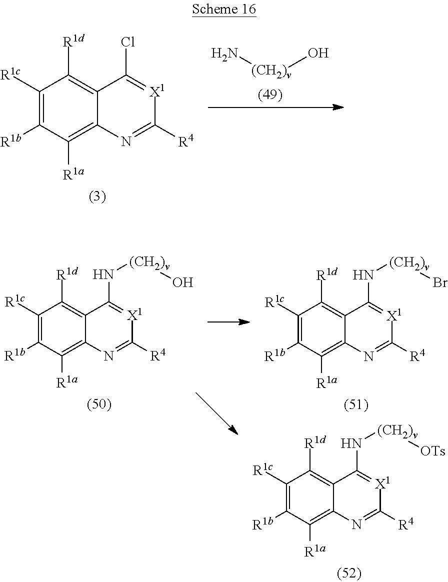

Alternatively, a compound of the formula (3) is reacted with a compound of the formula (49), a known compound or a compound prepared by known methods, in the presence of tin chloride, optionally in the presence of a solvent N,N-dimethylformamide, dimethyl sulfoxide, N,N-dimethylacetamide, and the like, optionally with heating, optionally, with microwave irradiation to provide a compound of the formula (50). A compound of the formula (50) is reacted with carbon tetrabromide in the presence of triphenyl phosphine, in the presence of a solvent such as methylene chloride, 1,2-dichloroethane, tetrahydrofuran, 1,4-dioxane, and the like, optionally with heating, optionally with microwave irradiation to provide a compound of the formula (51). Alternatively, a compound of the formula (50) is reacted with 4-methylbenzenesulfonyl chloride in the presence of a base such as triethylamine, N,N-diisopropylethylamine, pyridine, 2,6-dimethylpyridine, and the like, in the presence of a solvent such as methylene chloride, 1,2-dichloroethane, tetrahydrofuran, 1,4-dioxane, and the like, optionally with heating, optionally with microwave irradiation to provide a compound of the formula (52).

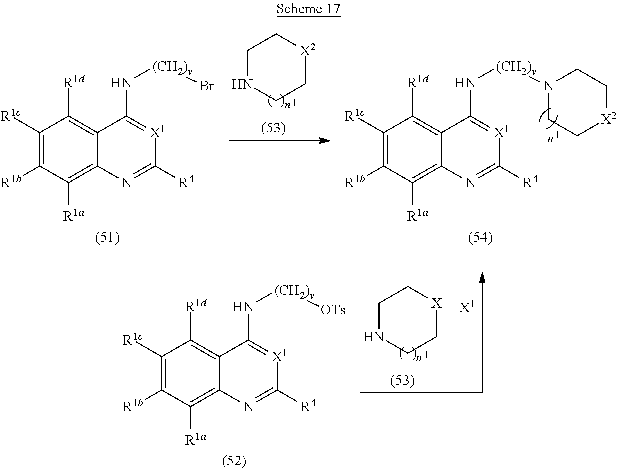

A compound of the formula (51) is reacted with a compound of the formula (53), a known compound or a prepared by known methods, optionally in the presence of solvent such as acetonitrile, tetrahydrofuran, 1,4-dioxane, methylene chloride, 1,2-dichloroethane, N,N-dimethylformamide, dimethyl sulfoxide, N,N-dimethylacetamide, and the like, optionally in the presence of a base such as sodium carbonate, lithium carbonate, potassium carbonate, triethylamine, N,N-diisopropylethylamine, pyridine, 2,6-dimethylpyridine, and the like, optionally with heating, optionally with microwave irradiation to provide a compound of the formula (54). Alternatively, a compound of the formula (52) is reacted with a compound of the formula (53), a known compound or a prepared by known methods, optionally in the presence of solvent such as acetonitrile, tetrahydrofuran, 1,4-dioxane, methylene chloride, 1,2-dichloroethane, N,N-dimethylformamide, dimethyl sulfoxide, N,N-dimethylacetamide, and the like, optionally in the presence of a base such as sodium carbonate, lithium carbonate, potassium carbonate, triethylamine, N,N-diisopropylethylamine, pyridine, 2,6-dimethylpyridine, and the like, optionally with heating, optionally with microwave irradiation to provide a compound of the formula (54).

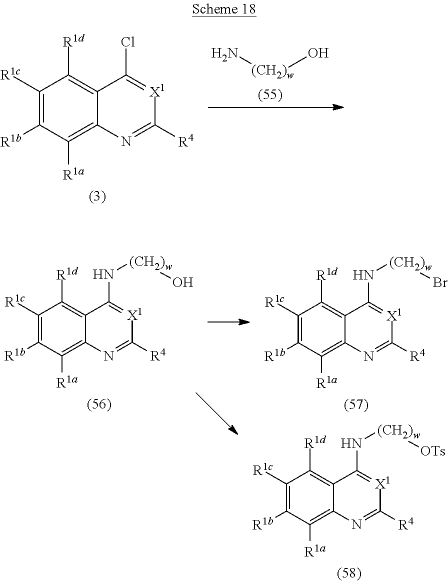

Alternatively, a compound of the formula (3) is reacted with a compound of the formula (55), a known compound or a compound prepared by known methods, in the presence of tin chloride, optionally in the presence of a solvent N,N-dimethylformamide, dimethyl sulfoxide, N,N-dimethylacetamide, and the like, optionally with heating, optionally, with microwave irradiation to provide a compound of the formula (56). A compound of the formula (56) is reacted with carbon tetrabromide in the presence of triphenyl phosphine, in the presence of a solvent such as methylene chloride, 1,2-dichloroethane, tetrahydrofuran, 1,4-dioxane, and the like, optionally with heating, optionally with microwave irradiation to provide a compound of the formula (57). Alternatively, a compound of the formula (56) is reacted with 4-methylbenzenesulfonyl chloride in the presence of a base such as triethylamine, N,N-diisopropylethylamine, pyridine, 2,6-dimethylpyridine, and the like, in the presence of a solvent such as methylene chloride, 1,2-dichloroethane, tetrahydrofuran, 1,4-dioxane, and the like, optionally with heating, optionally with microwave irradiation to provide a compound of the formula (58).

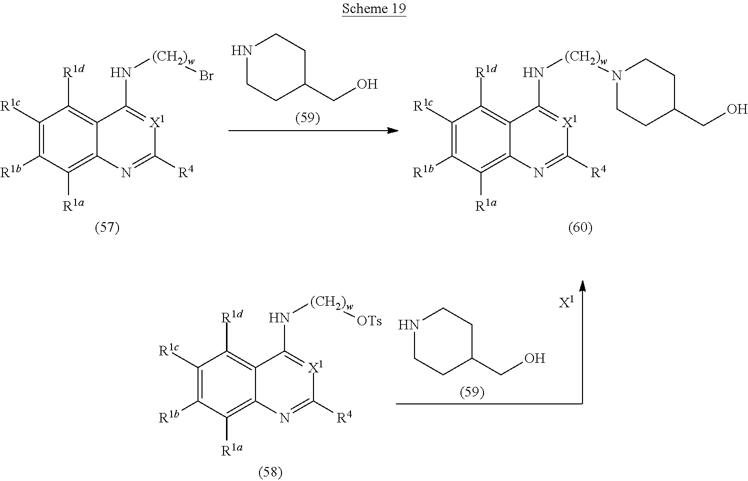

A compound of the formula (57) is reacted with a compound of the formula (59), a known compound or a prepared by known methods, optionally in the presence of solvent such as acetonitrile, tetrahydrofuran, 1,4-dioxane, methylene chloride, 1,2-dichloroethane, N,N-dimethylformamide, dimethyl sulfoxide, N,N-dimethylacetamide, and the like, optionally in the presence of a base such as sodium carbonate, lithium carbonate, potassium carbonate, triethylamine, N,N-diisopropylethylamine, pyridine, 2,6-dimethylpyridine, and the like, optionally with heating, optionally with microwave irradiation to provide a compound of the formula (60). Alternatively, a compound of the formula (58) is reacted with a compound of the formula (59), a known compound or a prepared by known methods, in the presence of solvent such as acetonitrile, tetrahydrofuran, 1,4-dioxane, methylene chloride, 1,2-dichloroethane, N,N-dimethylformamide, dimethyl sulfoxide, N,N-dimethylacetamide, and the like, optionally in the presence of a base such as sodium carbonate, lithium carbonate, potassium carbonate, triethylamine, N,N-diisopropylethylamine, pyridine, 2,6-dimethylpyridine, and the like, optionally with heating, optionally with microwave irradiation to provide a compound of the formula (60).

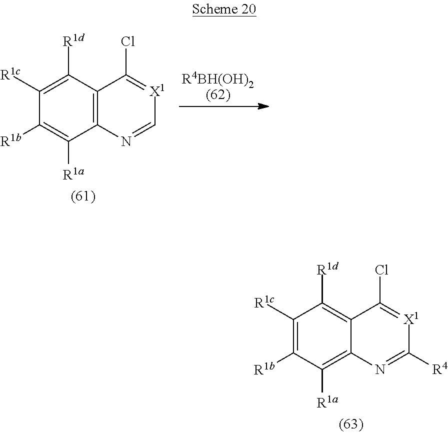

A compound of the formula (61), a known compound or a compound prepared by known methods, is reacted with a compound of the formula (62), a known compound or a compound prepared by known methods, in the presence of an acid such as a trifluoroacetic acid, acetic acid, formic acid, hydrochloric acid, and the like, in the presence of silver nitrate, in the presence of potassium persulfate, optionally in the presence of water, optionally in the presence of a solvent such as methanol, ethanol, tetrahydrofuran, 1,4-dioxane, acetonitrile, methylene chloride, 1,2-dichloroethane, 1,2-dimethoxyethane, and the like, optionally with heating, optionally with microwave irradiation to provide a compound of the formula (63).

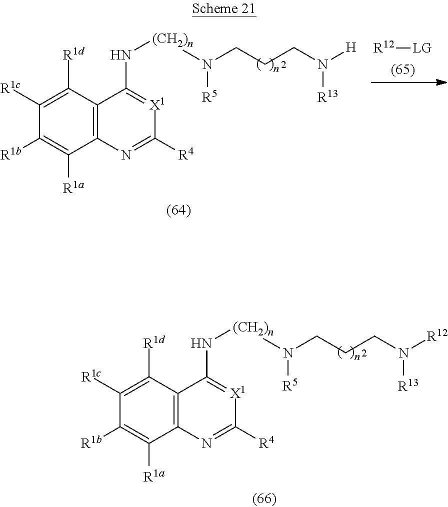

A compound of the formula (64) is reacted with a compound of the formula (65), a known compound or a compound prepared by known methods wherein LG is selected from the group consisting of iodine, bromine, mesylate, and tosylate, in the presence of a base such as sodium carbonate, lithium carbonate, potassium carbonate, cesium carbonate, and the like, in a solvent such as methylene chloride, tetrahydrofuran, 1,4-dioxane, acetonitrile, 1,2-dimethoxyethane, N,N-dimethylformamide, dimethyl sulfoxide, N,N-dimethylacetamide and the like, optionally with heating, optionally with microwave irradiation to provide a compound of the formula (66).

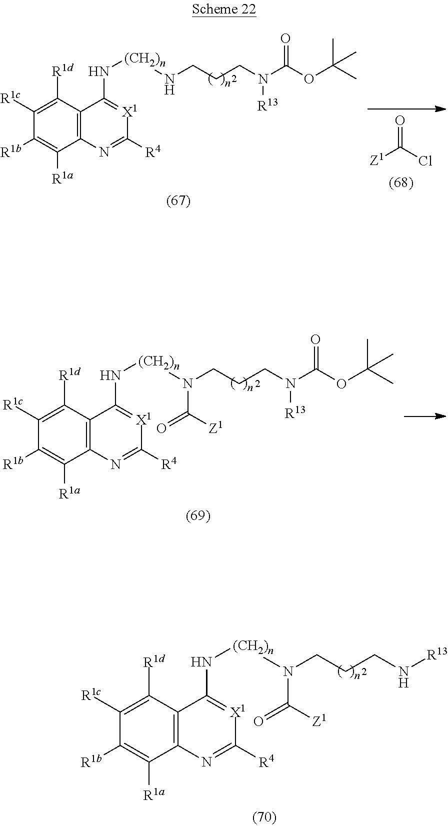

A compound of the formula (67) is reacted with a compound of the formula (68), a known compound or a compound prepared by known methods wherein Z¹ is selected from the group consisting of C₁₋₆ linear alkyl and C₃₋₆ branched alkyl, in the presence of a base such as triethylamine, N,N-diisopropylethylamine, pyridine, 2,6-dimethylpyridine, and the like, in a solvent such as tetrahydrofuran, 1,4-dioxane, acetonitrile, methylene chloride, 1,2-dichloroethane, 1,2-dimethoxyethane, and the like, optionally with heating, optionally with microwave irradiation to provide a compound of the formula (69). A compound of the formula (69) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethyl sulfoxide, N,N-dimethylacetamide, and the like, optionally with heating, optionally with microwave irradiation to provide a compound of the formula (70).

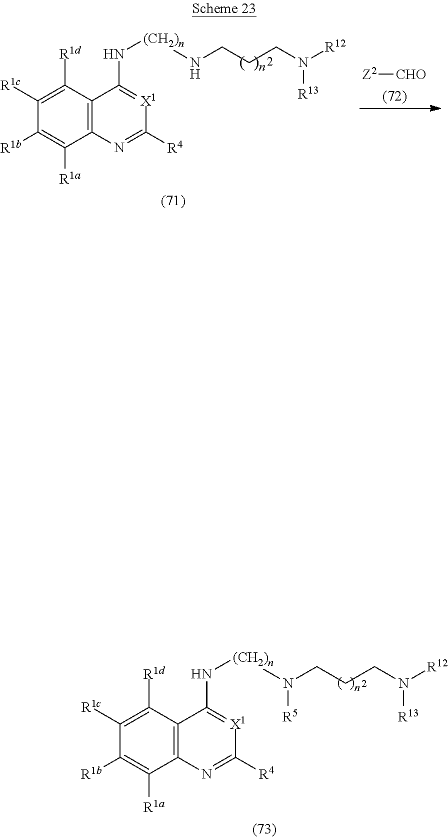

A compound of the formula (71) is reacted with a compound of the formula (72), a known compound or a compound prepared by known methods in which Z² is selected from the group consisting of hydrogen, C₁₋₄ linear alkyl, C₃₋₄ branched alkyl, —CH₂—(C₁₋₆ cycloalkyl), C(O)C₁₋₆ linear alkyl, C(O)C₃₋₆ branched alkyl,

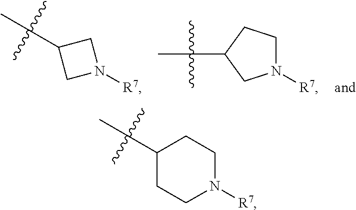

in the presence of a reducing agent such as sodium triacetoxyborohydride, lithium triacetoxyborohydride, sodium borohydride, lithium borohydride, and the like, in the presence of an acid such as acetic acid, trifluoroacetic acid, formic acid, and the like, in a solvent such as methylene chloride, 1,2-dichloroethane, tetrahydrofuran, 1,4-dioxane, methanol, ethanol, and the like, optionally with heating, optionally with microwave irradiation to provide a compound of the formula (73).

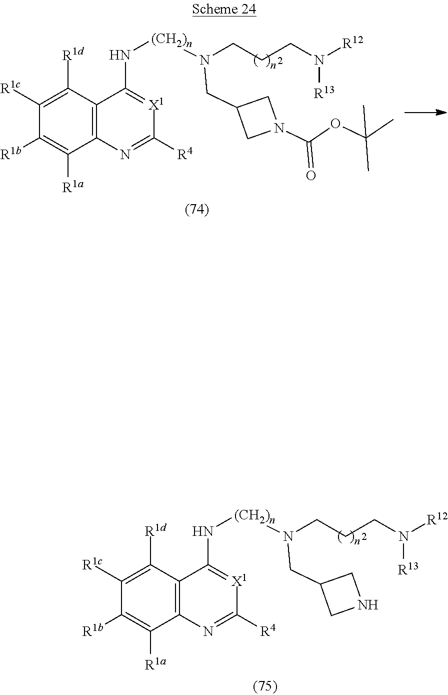

A compound of the formula (74) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethyl sulfoxide, N,N-dimethylacetamide, and the like, optionally with heating, optionally with microwave irradiation to provide a compound of the formula (75).

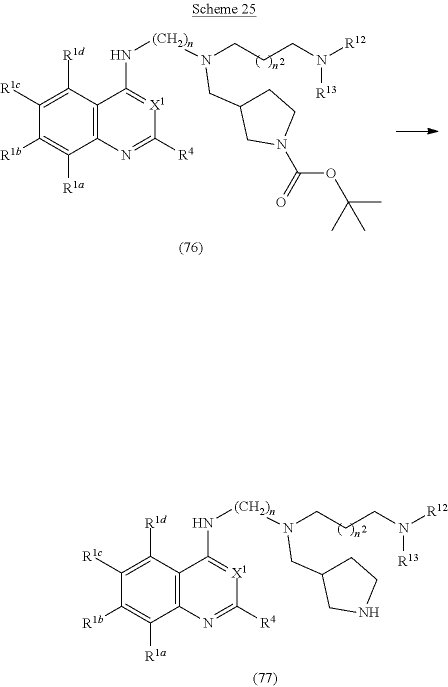

A compound of the formula (76) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethyl sulfoxide, N,N-dimethylacetamide, and the like, optionally with heating, optionally with microwave irradiation to provide a compound of the formula (77).

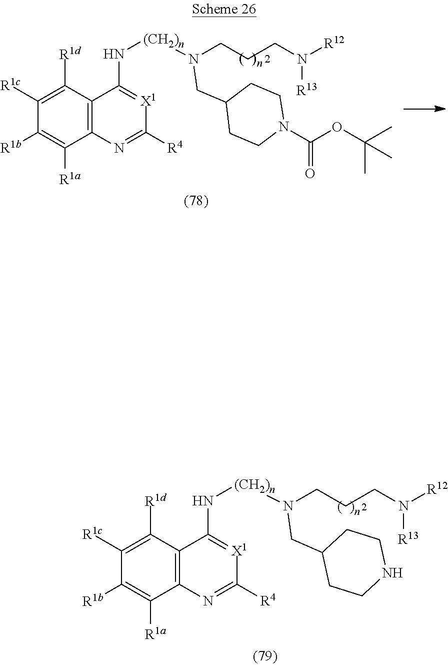

A compound of the formula (78) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethyl sulfoxide, N,N-dimethylacetamide, and the like, optionally with heating, optionally with microwave irradiation to provide a compound of the formula (79).

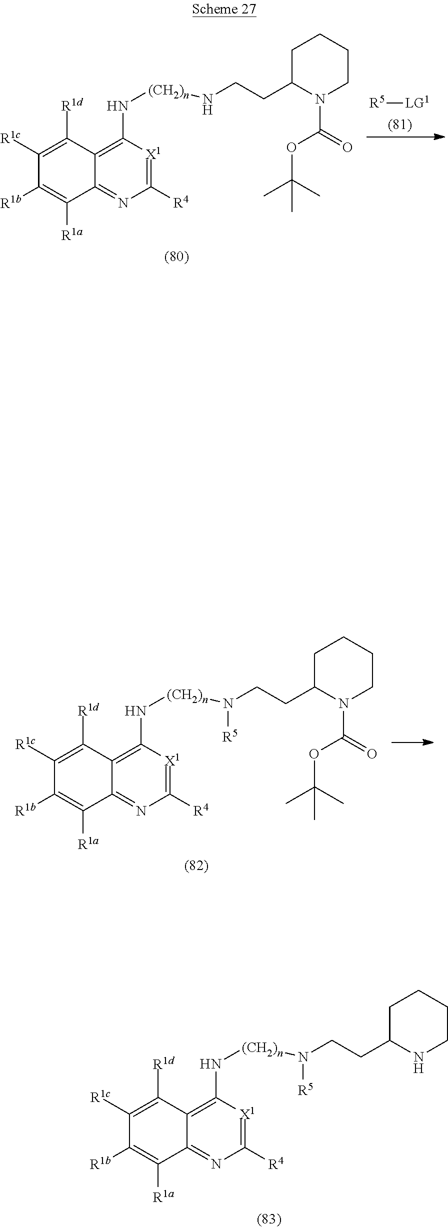

A compound of the formula (80) is reacted with a compound of the formula (81), a known compound or a compounds prepared by known methods wherein LG¹ is selected from the group consisting of iodine, bromine, mesylate, and tosylate, in the presence of a base such as sodium carbonate, lithium carbonate, potassium carbonate, cesium carbonate, and the like, in a solvent such as methylene chloride, tetrahydrofuran, 1,4-dioxane, acetonitrile, 1,2-dimethoxyethane, N,N-dimethylformamide, dimethyl sulfoxide, N,N-dimethylacetamide and the like, optionally with heating, optionally with microwave irradiation to provide a compound of the formula (82). A compound of the formula (82) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethyl sulfoxide, N,N-dimethylacetamide, and the like, optionally with heating, optionally with microwave irradiation to provide a compound of the formula (83).

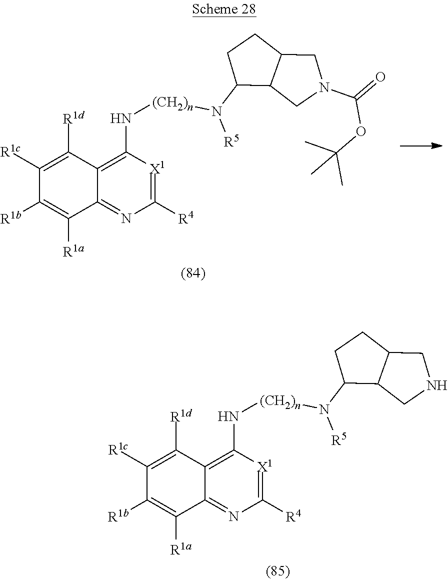

A compound of the formula (84) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethyl sulfoxide, N,N-dimethylacetamide, and the like, optionally with heating, optionally with microwave irradiation to provide a compound of the formula (85).

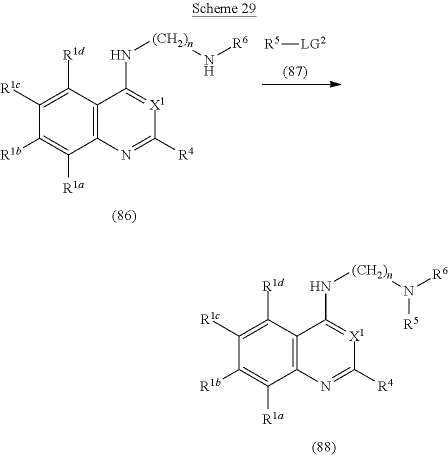

A compound of the formula (86) is reacted with a compound of the formula (87), a known compound or a compound prepared by known methods wherein LG² is selected from the group consisting of iodine, bromine, mesylate, and tosylate, in the presence of a base such as sodium carbonate, lithium carbonate, potassium carbonate, cesium carbonate, and the like, in a solvent such as methylene chloride, tetrahydrofuran, 1,4-dioxane, acetonitrile, 1,2-dimethoxyethane, N,N-dimethylformamide, dimethyl sulfoxide, N,N-dimethylacetamide and the like, optionally with heating, optionally with microwave irradiation to provide a compound of the formula (88).

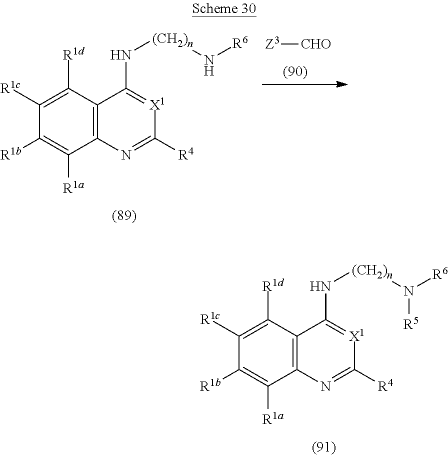

A compound of the formula (89) is reacted with a compound of the formula (90), a known compound or a compound prepared by known methods in which Z³ is selected from the group consisting of hydrogen, C₁₋₄ linear alkyl, C₃₋₄ branched alkyl, —CH₂—(C₁₋₆ cycloalkyl), C(O)C₁₋₆ linear alkyl, C(O)C₃₋₆ branched alkyl,

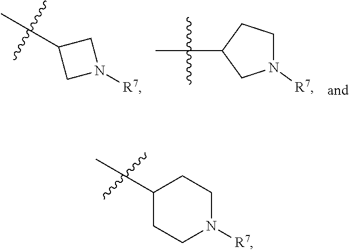

in the presence of a reducing agent such as sodium triacetoxyborohydride, lithium triacetoxyborohydride, sodium borohydride, lithium borohydride, and the like, in the presence of an acid such as acetic acid, trifluoroacetic acid, formic acid, and the like, in a solvent such as methylene chloride, 1,2-dichloroethane, tetrahydrofuran, 1,4-dioxane, methanol, ethanol, and the like, optionally with heating, optionally with microwave irradiation to provide a compound of the formula (91).

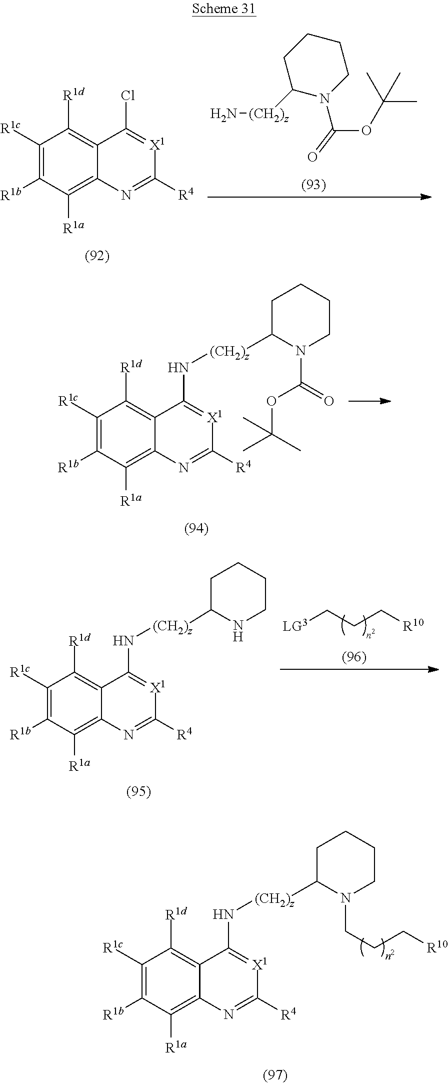

A compound of the formula (92) is reacted with a compound of the formula (93), a known compound or a compound prepared by known methods, in the presence of tin tetrachloride, optionally in the presence of a solvent N,N-dimethylformamide, dimethyl sulfoxide, N,N-dimethylacetamide, and the like, optionally with heating, optionally, with microwave irradiation to provide a compound of the formula (94). A compound of the formula (94) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethyl sulfoxide, N,N-dimethylacetamide, and the like, optionally with heating, optionally with microwave irradiation to provide a compound of the formula (95). A compound of the formula (95) is reacted with a compound of the formula (96), a known compound or a compound prepared by known methods wherein LG³ is selected from the group consisting of iodine, bromine, mesylate, and tosylate, in the presence of a base such as sodium carbonate, lithium carbonate, potassium carbonate, cesium carbonate, and the like, in a solvent such as methylene chloride, tetrahydrofuran, 1,4-dioxane, acetonitrile, 1,2-dimethoxyethane, N,N-dimethylformamide, dimethyl sulfoxide, N,N-dimethylacetamide and the like, optionally with heating, optionally with microwave irradiation to provide a compound of the formula (97).

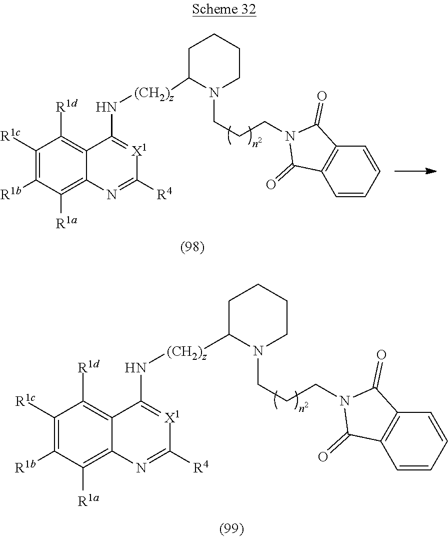

A compound of the formula (98) is reacted with hydrazine in the presence of a solvent such as methanol, ethanol, isopropanol, tetrahydrofuran, 1,4-dioxane, 1,2-dimethoxyethane, N,N-dimethylformamide, dimethyl sulfoxide, N,N-dimethylacetamide and the like, optionally with heating, optionally with microwave irradiation to provide a compound of the formula (99).

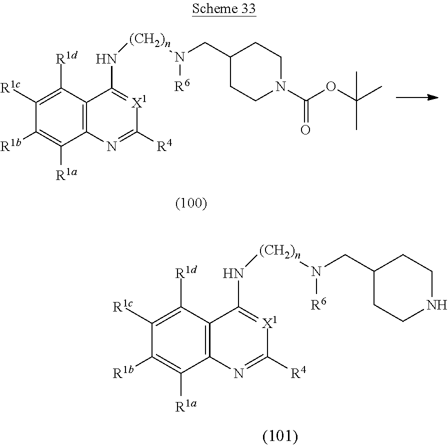

A compound of the formula (100) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethyl sulfoxide, N,N-dimethylacetamide, and the like, optionally with heating, optionally with microwave irradiation to provide a compound of the formula (101).

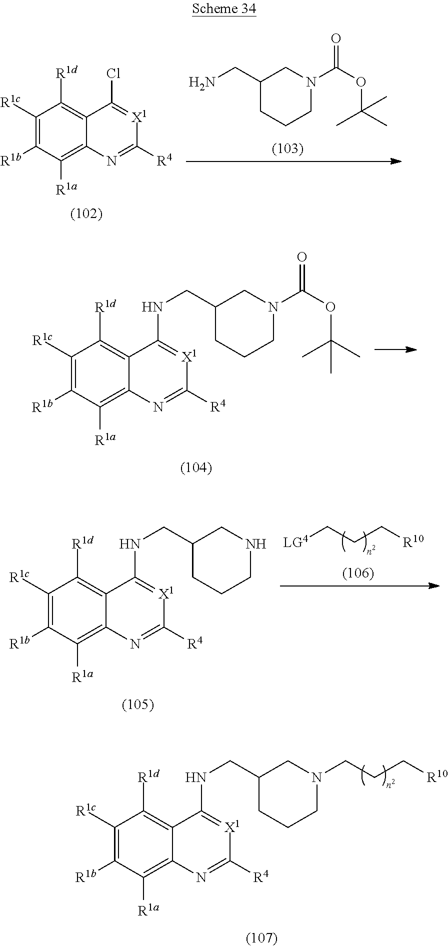

A compound of the formula (102) is reacted with a compound of the formula (103), a known compound or a compound prepared by known methods, in the presence of tin tetrachloride, optionally in the presence of a solvent N,N-dimethylformamide, dimethyl sulfoxide, N,N-dimethylacetamide, and the like, optionally with heating, optionally, with microwave irradiation to provide a compound of the formula (104). A compound of the formula (104) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethyl sulfoxide, N,N-dimethylacetamide, and the like, optionally with heating, optionally with microwave irradiation to provide a compound of the formula (105). A compound of the formula (105) is reacted with a compound of the formula (106), a known compound or a compound prepared by known methods wherein LG⁴ is selected from the group consisting of iodine, bromine, mesylate, and tosylate, in the presence of a base such as sodium carbonate, lithium carbonate, potassium carbonate, cesium carbonate, and the like, in a solvent such as methylene chloride, tetrahydrofuran, 1,4-dioxane, acetonitrile, 1,2-dimethoxyethane, N,N-dimethylformamide, dimethyl sulfoxide, N,N-dimethylacetamide and the like, optionally with heating, optionally with microwave irradiation to provide a compound of the formula (107).

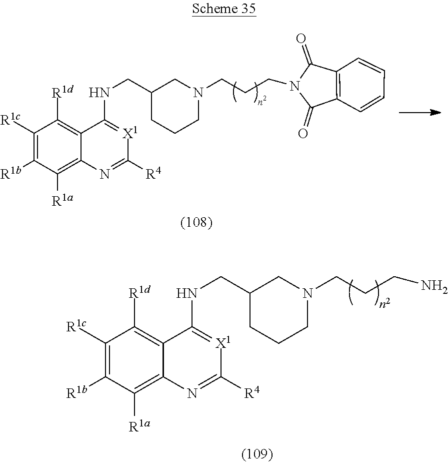

A compound of the formula (108) is reacted with hydrazine in the presence of a solvent such as methanol, ethanol, isopropanol, tetrahydrofuran, 1,4-dioxane, 1,2-dimethoxyethane, N,N-dimethylformamide, dimethyl sulfoxide, N,N-dimethylacetamide and the like, optionally with heating, optionally with microwave irradiation to provide a compound of the formula (109).

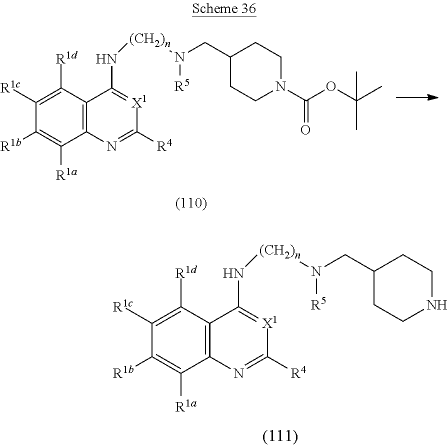

A compound of the formula (110) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethyl sulfoxide, N,N-dimethylacetamide, and the like, optionally with heating, optionally with microwave irradiation to provide a compound of the formula (111).

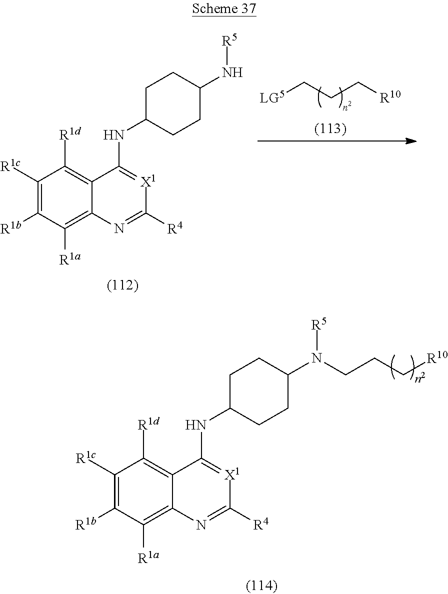

A compound of the formula (112) is reacted with a compound of the formula (113), a known compound or a compound prepared by known methods wherein LG⁵ is selected from the group consisting of iodine, bromine, mesylate, and tosylate, in the presence of a base such as sodium carbonate, lithium carbonate, potassium carbonate, cesium carbonate, and the like, in a solvent such as methylene chloride, tetrahydrofuran, 1,4-dioxane, acetonitrile, 1,2-dimethoxyethane, N,N-dimethylformamide, dimethyl sulfoxide, N,N-dimethylacetamide and the like, optionally with heating, optionally with microwave irradiation to provide a compound of the formula (114).

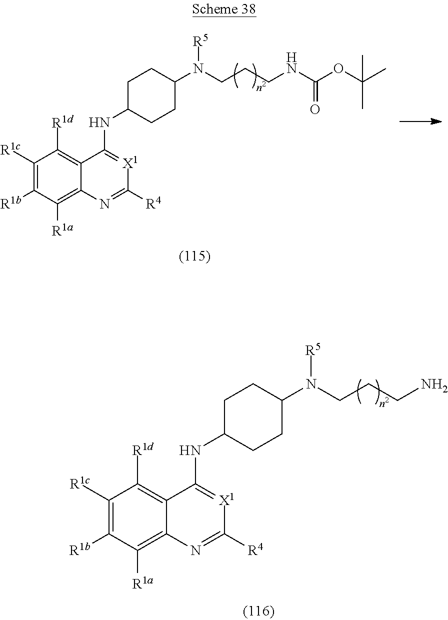

A compound of the formula (115) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethyl sulfoxide, N,N-dimethylacetamide, and the like, optionally with heating, optionally with microwave irradiation to provide a compound of the formula (116).

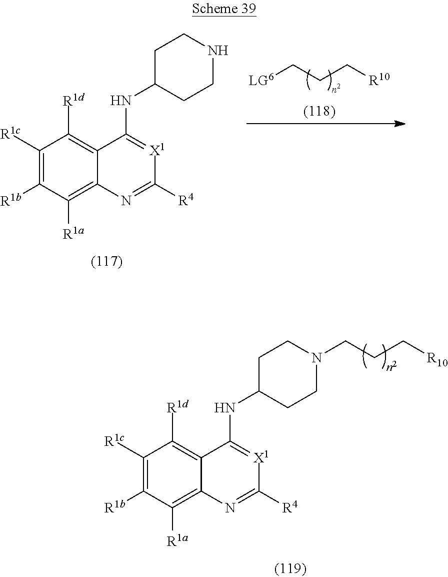

A compound of the formula (117) is reacted with a compound of the formula (118), a known compound or a compound prepared by known methods wherein LG⁶ is selected from the group consisting of iodine, bromine, mesylate, and tosylate, in the presence of a base such as sodium carbonate, lithium carbonate, potassium carbonate, cesium carbonate, and the like, in a solvent such as methylene chloride, tetrahydrofuran, 1,4-dioxane, acetonitrile, 1,2-dimethoxyethane, N,N-dimethylformamide, dimethyl sulfoxide, N,N-dimethylacetamide and the like, optionally with heating, optionally with microwave irradiation to provide a compound of the formula (119).

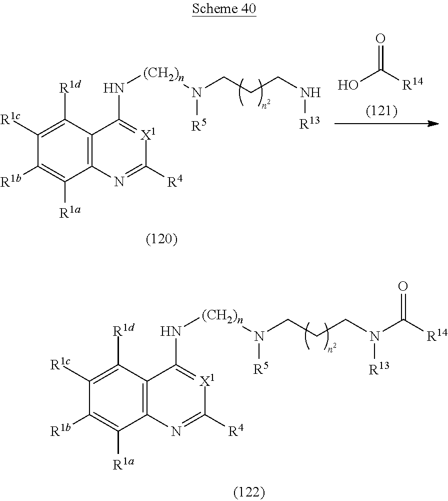

A compound of the formula (120) is reacted with a compound of the formula (121), a known compound or a compound made by known methods, in the presence of a coupling agent such as O-(benzotriazol-1-yl)-N,N,N′,N′-tetramethyluronium hexafluorophosphate, N,N′-dicyclohexyl carbodiimide, 1-ethyl-3-(3-dimethylaminopropyl)carbodiimide, 1-[bis(dimethylamino)methylene]-1H-1,2,3-triazolo[4,5-b]pyridinium 3-oxid hexafluorophosphate, and the like, optionally in the presence of 1-hydroxy-7-azabenzotriazole, optionally in the presence of a base such as triethylamine, diisopropylethylamine, N-methylmorpholine and the like, in a solvent such as N,N-dimethylformamide, 1,4-dioxane, tetrahydrofuran, methylene chloride and the like, optionally with heating, optionally with microwave irradiation to provide a compound of the formula (122).

The TDP-43 binding agents of the disclosure may be present as isotopically labeled forms of compounds detailed herein. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine and chlorine, such as, but not limited to ²H (deuterium, D), ³H (tritium), ¹³C, ¹⁴C, ¹⁵N, ¹⁸F, ³¹P, ³²p, ³⁵S, ³⁶CI, and ¹²⁹I. Various isotopically labeled compounds of the present disclosure, for example those into which radioactive isotopes such as ³H, ¹³C, and ¹⁴C are incorporated, are provided. Such isotopically labeled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of subjects (e.g., humans). Also provided for isotopically labeled compounds described herein are any pharmaceutically acceptable salts, or hydrates, as the case may be.

In some variations, the compounds disclosed herein may be varied such that from 1 to “n” hydrogens attached to a carbon atom is/are replaced by deuterium, in which “n” is the number of hydrogens in the molecule. Such compounds may exhibit increased resistance to metabolism and are thus useful for increasing the half-life of the compound when administered to a subject. See, for example, Foster, “Deuterium Isotope Effects in Studies of Drug Metabolism”, Trends Pharmacol. Sci. 5(12):524-527 (1984). Such compounds are synthesized by means well known in the art, for example by employing starting materials in which one or more hydrogens have been replaced by deuterium.

Deuterium labeled or substituted therapeutic compounds of the disclosure may have improved drug metabolism and pharmacokinetics (DMPK) properties, relating to absorption, distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F labeled compound may be useful for PET or SPECT studies. Isotopically labeled compounds of this disclosure can generally be prepared by carrying out the procedures known to those skilled in the art by substituting a readily available isotopically labeled reagent for a non-isotopically labeled reagent. It is understood that deuterium in this context is regarded as a substituent in the compounds provided herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen either exclusively or at its natural abundance isotopic composition, or “deuterium”. For example, when the position is designated “CH₃”, the position encompasses “CH₃”, “CDH₂”, “CD₂H”, or “CD₃”.

The Examples provided below provide representative methods for preparing exemplary compounds of the present invention. The skilled practitioner will know how to substitute the appropriate reagents, starting materials and purification methods known to those skilled in the art, in order to prepare the compounds of the present invention.

¹H-NMR spectra were obtained on a Varian Mercury 300-MHz NMR. Purity (%) and mass spectral data were determined with a Waters Alliance 2695 HPLC/MS (Waters Symmetry C18, 4.6×75 mm, 3.5 μm) with a 2996 diode array detector from 210-400 nm.

### EXAMPLES

The examples provide methods for preparing representative compound of formula (I). The skilled practitioner will know how to substitute the appropriate reagents, starting materials and purification methods known to those skilled in the art, in order to prepare additional compounds of the present invention.

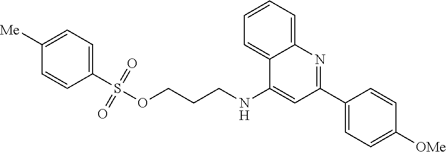 [SMILES: COc1ccc(-c2cc(NCCCOS(=O)(=O)c3ccc(C)cc3)c3ccccc3n2)cc1]

### Synthesis of intermediate A (IA): 3-(2-(4-methoxyphenyl)quinolin-4-ylamino)propyl-4-methylbenzenesulfonate

Step 1: To a stirring solution of 2,4-dichloroquinoline (6.16 g, 31.10 mmol) in toluene/ethanol (1:1 10 mL) was added 4-methoxyphenylboronic acid (5.2 g, 34.21 mmol), followed by sodium carbonate (1 M, 62 mL, 62.20 mmol). The reaction mixture was degassed over a slow flow of nitrogen for 15 minutes, then tetrakis(triphenylphosphine)-palladium(0) was added, the reaction flask flushed with nitrogen and placed in a heating bath to 90° C. for 6 hours. The cooled reaction mixture was diluted with ethyl acetate (50 mL), washed with water (25 mL), the aqueous washed with brine (20 mL), dried over sodium sulfate and filtered. The product was purified on silica gel (220 gm, 60-120 mesh) and concentrated under reduced pressure to give 4-chloro-2-(4-methoxyphenyl)quinoline as a white solid (5.0 g, 59%). ¹H NMR (CDCl₃) δ: 9.06 (d, J=8.2 Hz, 1H), 8.96-9.02 (m, 3H), 8.78-8.79 (m, 1H), 8.59-8.64 (m, 1H), 8.42-8.47 (m, 1H), 8.11-8.12 (m, 1H), 7.90 (d, J=7.6 Hz, 2H), 4.75 (s, 3H). MS m/z (M+) 270.03.

Step 2: A solution of 4-chloro-2-(4-methoxyphenyl)quinoline (1.0 g, 3.71 mmol) in 3-aminopropanol (567 μL, 7.41 mmol) was treated with tin tetrachloride (10 drops) and heated overnight at 130° C. The resulting hot reaction mixture was poured into ice water and extracted with ethyl acetate, whereby a semi solid formed which was filtered and dried to give 3-(2-(4-methoxyphenyl)quinolin-4-ylamino)propan-1-ol as an off white solid (0.840 g, 73%). ¹H NMR (CDCl₃) δ: 8.05 (d, J=8.8 Hz, 2H), 7.63-7.69 (m, 1H), 7.56-7.63 (m, 1H), 7.29-7.38 (m, 1H), 7.26 (s, 1H), 7.01 (d, J=8.8 Hz, 2H), 6.76 (s, 1H), 3.86 (s, 3H), 3.47 (q, J=5.5 Hz, 2H), 2.75 (br d, J=11.2 Hz, 2H), 2.54-2.61 (m, 2H), 2.34 (s, 4H), 2.16-2.31 (m, 5H), 1.70-2.02 (m, 6H), 1.40-1.40 (m, 1H), 1.12-1.34 (m, 2H). MS m/z (M+) 309.03.

Step 3: To a solution of 3-(2-(4-methoxyphenyl)quinolin-4-ylamino)propan-1-ol (2.58 g, 8.37 mmol) in anhydrous tetrahydrofuran (40 mL), was added triethylamine (2.5 ml, 17.9 mmol) and 4-methylbenzene-1-sulfonyl chloride (1.75 g, 9.2 mmol in 10 mL tetrahydrofuran) over a period of 10 minutes. The reaction mixture was stirred at 23° C. overnight, at which time additional 4-methylbenzene-1-sulfonyl chloride (0.75 g, 3.93 mmol in 5 mL tetrahydrofuran) was added. The reaction mixture was stirred for an additional 2 hours. The reaction mixture was partitioned between ethyl acetate and 5% sodium bicarbonate, washed with sodium bicarbonate (2×20 mL) and brine (25 mL), dried over sodium sulfate, concentrated under reduced pressure and purified on silica gel (120 g, 60-120 mesh) to afford 3-(2-(4-methoxyphenyl)quinolin-4-ylamino)propyl 4-methylbenzenesulfonate (IA, 2.21 g, 57%) as a white foam. ¹H NMR (CDCl₃) δ: 7.96-8.10 (m, 3H), 7.75-7.77 (m, 1H), 7.74 (s, 1H), 7.57-7.68 (m, 2H), 7.33-7.43 (m, 1H), 7.17-7.29 (m, 2H), 7.01 (d, J=8.8 Hz, 2H), 6.75 (s, 1H), 4.21 (t, J=5.6 Hz, 2H), 3.87 (s, 3H), 3.51 (d, J=5.9 Hz, 2H), 2.33 (s, 3H), 2.04-2.17 (m, 2H). MS m/z (M+) 463.2.

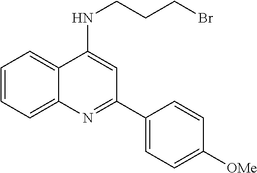 [SMILES: COc1ccc(-c2cc(NCCCBr)c3ccccc3n2)cc1]

Synthesis of intermediate B (IB). N-(3-bromopropyl)-2-(4-methoxyphenyl)-quinolin-4-amine: To a solution of 3-(2-(4-methoxyphenyl)quinolin-4-ylamino)propan-1-ol (200 mg, 0.65 mmol) and triphenylphosphine (187 mg, 0.71 mmol) in anhydrous dichloromethane (6 mL) was slowly added carbon tetrabromide (237 mg, 0.71 mmol) dissolved in anhydrous dichloromethane (10 mL) over a period of 15 minutes. The reaction mixture was stirred at 23° C. while monitoring for product formation. At 1 hour 0.2 equivalent of each triphenylphosphine and carbon tetrabromide was added and stirring resumed at 23° C. overnight. The resultant reaction mixture was quenched with 15% sodium hydroxide and extracted three times with dichloromethane (15 mL). Combined organic layers were washed with brine and dried over sodium sulfate, decanted and concentrated in vacuo. Purified on 20 g silica gel, eluting with ethyl acetate:hexane 0-50% to give N-(3-bromopropyl)-2-(4-methoxyphenyl)quinolin-4-amine as a white fluffy solid (IB, 197 mg, 83%). ¹H NMR (CDCl₃) δ: 8.04 (d, J=1.0 Hz, 3H), 7.69 (d, J=8.3 Hz, 1H), 7.58 (t, J=1.0 Hz, 1H), 7.32 (t, J=1.0 Hz, 1H), 6.98 (d, J=1.0 Hz, 2H), 6.82 (s, 1H), 6.82 (s, 1H), 5.28 (t, J=5.6 Hz, 1H), 3.82 (s, 3H), 3.43-3.54 (m, 4H), 2.09-2.35 (m, 1H). MS m/z (M+H)=371.05.

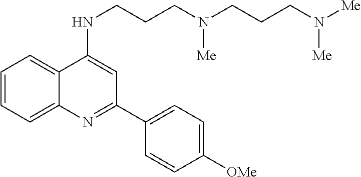 [SMILES: COc1ccc(-c2cc(NCCCN(C)CCCN(C)C)c3ccccc3n2)cc1]

Example 1: Synthesis of N¹-(3-((2-(4-Methoxyphenyl)quinolin-4-yl)amino)propyl)-N¹,N³,N³-trimethyl propane-1,3-diamine. To a solution of N,N,N′-trimethylpropane-1,3-diamine (0.031 g, 0.26 mmol) in acetonitrile (0.5 mL) was added triethylamine (36 μL, 0.26 mmol) followed by IA (0.08 g, 0.17 mmol). The resulting reaction mixture was stirred at 23° C. over a period of 16 hours. The reaction mixture was then diluted with ethyl acetate (5 mL), washed with sodium bicarbonate (5 mL). Aqueous layer was extracted three times with ethyl acetate, dried over magnesium sulfate and filtered. The resultant crude product was purified through silica gel cartridge with dichloromethane/methanol with 5% ammonium hydroxide to give N¹-(3-((2-(4-methoxyphenyl)quinolin-4-yl)amino)propyl)-N¹,N³,N³-trimethylpropane-1,3-diamine as a pale yellow powder (12 mg, 18%). ¹H NMR (300 MHz, CDCl₃) δ: 8.12 (d, J=8.5 Hz, 1H), 8.05 (d, J=8.7 Hz, 2H), 7.73 (d, J=8.6 Hz, 1H), 7.61 (t, J=7.6 Hz, 1H), 7.32-7.45 (m, 1H), 7.00 (d, J=8.7 Hz, 2H), 6.72 (s, 1H), 3.86 (s, 3H), 3.50 (q, J=5.1 Hz, 2H), 2.63-2.70 (m, 2H), 2.46-2.56 (m, 2H), 2.39 (s, 3H), 2.31 (t, J=7.3 Hz, 2H), 2.15 (s, 6H), 1.97 (quin, J=5.8 Hz, 2H), 1.75 (quin, J=7.4 Hz, 2H). MS m/z (M+H)=407.1.

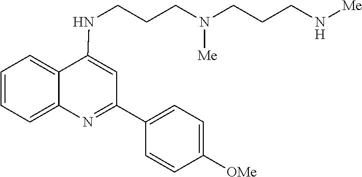 [SMILES: CNCCCN(C)CCCNc1cc(-c2ccc(OC)cc2)nc2ccccc12]

Example 2: Synthesis of 2-(4-Methoxyphenyl)-N-(3-{methyl[3-(methylamino)propyl]-amino}propyl) quinolin-4-amine. A solution of IB (197 mg, 0.53 mmol) in N¹,N³-dimethylpropane-1,3-diamine (1.00 mL) was stirred for 60 minutes. The resultant crude product was purified through silica gel cartridge eluting with dichloromethane: 5% ammonium hydroxide in 10% MeOH 0-100% to give 2-(4-methoxyphenyl)-N-(3-{methyl[3(methylamino)propyl]amino}propyl)quinolin-4-amine as a milky glass product (90 mg, 43%). ¹H NMR (DMSO-d₆) δ: 8.62-9.02 (m, 2H), 8.52 (br d, J=8.5 Hz, 1H), 8.10 (d, J=8.7 Hz, 3H), 7.85 (br t, J=7.7 Hz, 1H), 7.60 (br t, J=7.6 Hz, 1H), 7.17 (d, J=8.6 Hz, 2H), 7.03 (s, 1H), 3.87 (s, 3H), 3.68 (br d, J=6.1 Hz, 2H), 2.94 (br t, J=7.4 Hz, 3H), 2.56-2.78 (m, 2H), 1.71-2.21 (m, 2H). MS m/z (M+H)=393.33.

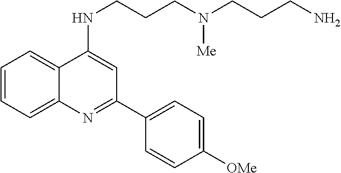 [SMILES: COc1ccc(-c2cc(NCCCN(C)CCCN)c3ccccc3n2)cc1]

Example 3: Synthesis of N¹-(3-Aminopropyl)-N³-(2-(4-methoxyphenyl)quinolin-4-yl)-N¹-methylpropane-1,3-diamine. To a solution of 4-chloro-2-(4-methoxyphenyl)quinoline (0.2 g, 0.74 mmol) in 3,3′-diamino-N-methyldipropylamine (1.43 mL, 8.88 mmol) was added two drops of tin tetrachloride. The reaction was carried out following the same procedure as step 2 of the synthesis of intermediate A (IA) N¹-(3-aminopropyl)-N³-(2-(4-methoxyphenyl)quinolin-4-yl)-N1-methylpropane-1,3-diamine was obtained as a yellow powder (177 mg, 64%). ¹H NMR (300 MHz, DMSO-d₆) δ 8.24-8.01 (m, 3H), 7.81 (d, J=8.4 Hz, 1H), 7.59 (t, J=7.6 Hz, 1H), 7.44-7.24 (m, 2H), 7.05 (d, J=8.6 Hz, 2H), 6.91 (s, 1H), 3.82 (s, 3H), 3.42 (br d, J=5.9 Hz, 2H), 3.11 (br s, 2H), 2.59 (br t, J=6.6 Hz, 2H), 2.45 (br t, J=7.0 Hz, 2H), 2.36 (t, J=7.2 Hz, 2H), 2.18 (s, 3H), 1.94-1.78 (m, 2H), 1.54 (quin, J=7.0 Hz, 2H). MS m/z (M+H)=379.3.

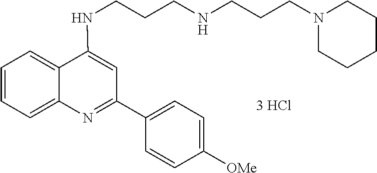 [SMILES: COc1ccc(-c2cc(NCCCNCCCN3CCCCC3)c3ccccc3n2)cc1]

Example 4: Synthesis of N¹-(2-(4-Methoxyphenyl)quinolin-4-yl)-N³-(3-(piperidin-1-yl)propyl) propane-1,3-diamine trihydrochloride. Into a vial with IB (0.1 g, 0.27 mmol) dissolved in dichloromethane (0.5 mL) was added N-(3-aminopropyl)piperidine (0.214 mL, 1.35 mmol). The reaction was carried out following the same procedure as Example 2. The obtained oil was dissolved in diethyl ether and excess of hydrogen chloride solution in diethyl ether was added. The solvent was evaporated and the resulting powder was dried under vacuum to give N¹-(2-(4-methoxyphenyl)quinolin-4-yl)-N³-(3-(piperidin-1-yl)propyl)propane-1,3-diamine trihydrochloride as a pale yellow powder (74 mg, 63%). ¹H NMR (300 MHz, DMSO-d₆) δ 13.62 (s, 1H), 10.51 (s, 1H), 9.62 (br t, J=5.8 Hz, 1H), 9.46 (s, 2H), 8.71 (d, J=8.5 Hz, 1H), 8.25 (d, J=8.5 Hz, 1H), 8.18-8.10 (m, 2H), 7.97-7.86 (m, 1H), 7.71-7.59 (m, 1H), 7.19 (d, J=9.0 Hz, 2H), 7.07 (s, 1H), 3.87 (s, 3H), 3.20-3.09 (m, 2H), 3.08-2.93 (m, 4H), 2.90-2.71 (m, 3H), 2.22-2.07 (m, 4H), 1.91-1.58 (m, 8H), 1.47-1.27 (m, 1H). MS m/z (M+H)=433.2.

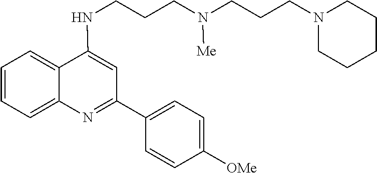 [SMILES: COc1ccc(-c2cc(NCCCN(C)CCCN3CCCCC3)c3ccccc3n2)cc1]

Example 5: Synthesis of N¹-(2-(4-methoxyphenyl)quinolin-4-yl)-N³-methyl-N³-(3-(piperidin-1-yl)propyl)propane-1,3-diamine trihydrochloride. To a solution of N-methyl-3-(piperidin-1-yl)propan-1-amine (0.135 mg, 0.86 mmol) in dimethylformamide (0.5 mL) was added potassium carbonate (0.059 g, 0.43 mmol) followed by IA (0.08 g, 0.17 mmol). The resulting reaction mixture was stirred at 23° C. over a period of 16 hours. The reaction mixture was then diluted with ethyl acetate (5 mL), washed with sodium bicarbonate (5 mL). Aqueous layer was extracted three times with ethyl acetate, dried over magnesium sulfate and filtered. The resultant crude product was purified through silica gel cartridge with dichloromethane/methanol with 5% ammonium hydroxide to give an oil. The obtained oil was dissolved in diethyl ether and excess of hydrogen chloride solution in diethyl ether was added. The solvent was evaporated and the resulting powder was dried under vacuum to give N¹-(2-(4-methoxyphenyl)quinolin-4-yl)-N³-methyl-N³-(3-(piperidin-1-yl)propyl)propane-1,3-diamine trihydrochloride as a pale yellow powder (12 mg, 17%). ¹H NMR (300 MHz, DMSO-d₆) δ 13.65 (s, 1H), 11.00 (s, 1H), 10.66 (s, 1H), 9.68-9.55 (m, 1H), 8.74 (d, J=8.5 Hz, 1H), 8.25 (d, J=8.5 Hz, 1H), 8.14 (d, J=8.6 Hz, 2H), 7.92 (t, J=7.7 Hz, 1H), 7.66 (t, J=7.6 Hz, 1H), 7.19 (d, J=8.7 Hz, 2H), 7.07 (s, 1H), 3.87 (s, 3H), 3.78-3.77 (m, 2H), 3.43-3.30 (m, 3H), 3.29-3.00 (m, 5H), 2.81 (br d, J=10.8 Hz, 2H), 2.74 (d, J=4.6 Hz, 3H), 2.29-2.07 (m, 4H), 1.82-1.65 (m, 5H), 1.21 (s, 1H). MS m/z (M+H)=447.5.

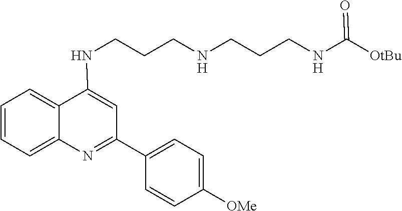 [SMILES: COc1ccc(-c2cc(NCCCNCCCNC(=O)OC(C)(C)C)c3ccccc3n2)cc1]

Example 6: Synthesis of tert-Butyl N-{3-[(3-{[2-(4-methoxyphenyl)quinolin-4yl]amino}propyl)amino] propyl}carbamate. To a solution of IA (100 mg, 0.27 mmol) in dichloromethane (1.0 mL) was added tert-butyl 3-aminopropylcarbamate (94 uL, 0.54 mmol) and the solution was stirred for 6 hours at 23° C. The reaction mixture was concentrated. The crude product was purified through silica gel cartridge (4 g, 60-120 mesh) eluting with dichloromethane/methanol with 5% ammonium hydroxide 0-10% to give the tert-butyl 3-aminopropylcarbamate as a white foam (75%). ¹H NMR (CDCl₃) δ: 7.95-8.14 (m, 3H), 7.76 (d, J=8.3 Hz, 1H), 7.60 (ddd, J=8.4, 6.9, 1.3 Hz, 1H), 7.30-7.45 (m, 1H), 7.25 (s, 2H), 7.01 (d, J=8.8 Hz, 2H), 6.77 (s, 1H), 4.83 (s, 1H), 3.87 (s, 3H), 3.39-3.59 (m, 2H), 3.25 (q, J=6.5 Hz, 2H), 2.88 (t, J=5.7 Hz, 2H), 2.73 (t, J=6.8 Hz, 2H), 1.96 (quin, J=5.9 Hz, 2H), 1.75 (quin, J=6.7 Hz, 5H), 1.43 (s, 7H). MS m/z (M+H)=465.28.

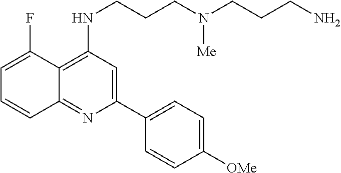 [SMILES: COc1ccc(-c2cc(NCCCN(C)CCCN)c3c(F)cccc3n2)cc1]

### Example 7: N¹-(3-aminopropyl)-N³-(5-fluoro-2-(4-methoxyphenyl)quinolin-4-yl)-N¹-methyl propane-1,3-diamine

Step 1: 2-Bromo-5-fluorobenzenamine (10.2 g, 53.8 mmol) and malonic acid (8.4 g, 80.7 mmol) were charged to a round bottom flask with phosphorous V oxychloride (50 mL). The reaction mixture was heated to 100° C. for 20 hours then heated to 140° C. for 90 minutes. The mixture was cooled to 23° C. and phosphorous V oxychloride was removed under vacuum. The resulting mixture was slowly dripped into water(400 mL) under rapid stirring. The dark solution was extracted with dichloromethane (2×300 mL), combined organics were washed with water (2×100 mL) dried over magnesium sulfate and concentrated under vacuum. Resulting mixture was purified on silica gel, eluted with dichloromethane. Combined pure fractions to obtain 8-bromo-2,4-dichloro-5-fluoroquinoline as a pale yellow solid (2.8 g, 17.6%). ¹H NMR (CDCl₃) δ: 8.04 (dd, J=8.5, 4.9 Hz, 1H), 7.57 (s, 1H), 7.20 (dd, J=11.3, 8.4 Hz, 1H).

Step 2: 8-bromo-2,4-dichloro-5-fluoroquinoline (1.5 g, 5.09 mmol) was dissolved in diethyl ether (90 mL) and tetrahydrofuran (18 mL) and cooled to −60° C. Butyllithium was added and the mixture stirred for 20 minutes, quenched with water, diluted with ethyl acetate and washed with sodium bicarbonate. The organic layer was dried over magnesium sulfate and concentrated under vacuum. The resulting 2,4-dichloro-5-fluoroquinoline (900 mg) was used without further purification.

Step 3: 2,4-Dichloro-5-fluoroquinoline (850 mg, 3.93 mmol) was dissolved in 1,4-dioxane (5.0 mL) and 4-methoxyphenylboronic acid (600 mg, 3.93 mmol) was added, followed by cesium carbonate in water (5.0 mL) (709 mg, 2.0 mmol), nitrogen was bubbled through for 10 minutes, then palladium tetrakistriphenyl phosphine (Pd 0) (77 mg, 0.067 mmol) was added and the solution heated to 80° C. for 3 hours, followed by cooling to 23° C. and treatment with saturated sodium bicarbonate (30 mL) and extracted into ethyl acetate (2×30 mL). The combined organics were dried over sodium sulfate and concentrated under vacuum, followed by purification on silica gel in 0-15% ethyl acetate/hexanes to afford 4-chloro-5-fluoro-2-(4-methoxyphenyl)quinoline as a solid (820 mg, 73%). ¹H NMR (CDCl₃) δ: 8.06-8.18 (m, 2H), 7.95 (d, J=8.6 Hz, 1H), 7.90 (s, 1H), 7.64 (td, J=8.2, 5.4 Hz, 1H), 7.21 (dd, J=11.9, 7.8 Hz, 1H).

Step 3: 4-Chloro-5-fluoro-2-(4-methoxyphenyl)quinoline (100 mg, 0.348 mmol), was dissolved in N¹-(3-aminopropyl)-N¹-methylpropane-1,3-diamine and 2 drops of tin tetrachloride was added, the reaction was carried out following the same procedure as step 2 of the synthesis of intermediate A (IA) [(3-Aminopropyl)(methyl)amino]propyl}-5-fluoro-2-(4-methoxyphenyl) quinolin-4-amine was obtained as a light yellow solid (130 mg, 94%). ¹H NMR (DMSO-d₆) δ: 13.82 (br s, 1H), 11.01-11.17 (m, 1H), 8.64-8.85 (m, 1H), 8.07-8.25 (m, 5H), 7.86-7.98 (m, 1H), 7.50 (dd, J=13.4, 8.0 Hz, 1H), 7.20 (d, J=8.9 Hz, 2H), 7.08 (s, 1H), 3.88 (s, 3H), 3.76-3.86 (m, 2H), 3.03-3.32 (m, 4H), 2.81-2.96 (m, 2H), 2.70 (d, J=4.7 Hz, 3H), 1.94-2.23 (m, 2H). MS m/z (M+H)=397.

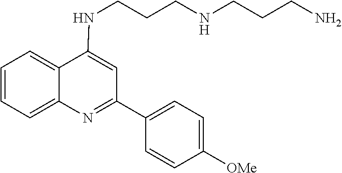 [SMILES: COc1ccc(-c2cc(NCCCNCCCN)c3ccccc3n2)cc1]

Example 8: N-{3-[(3-Aminopropyl)amino]propyl}-2-(4-methoxyphenyl)quinolin-4-amine trihydrochloride. To a solution of tert-butyl N-{3-[(3-{[2-(4-methoxyphenyl)quinoline-4-yl]amino}propyl)amino]propyl}carbamate in 1,4-dioxane (1 mL) was added excess of 4N hydrochloric acid in 1,4-dioxane, and the reaction stirred at 23° C. for 4 hours. The reaction mixture was filtered and the precipitate was washed with 1,4-dioxane and dried to give the product as a white solid (66.7 mg, 76%). ¹H NMR (CDCl₃) δ: 8.85 (br d, J=4.3 Hz, 1H), 7.91-8.24 (m, 3H), 7.76 (s, 1H), 6.99-7.21 (m, 1H), 6.92 (br d, J=8.4 Hz, 2H), 6.71 (s, 1H), 3.79 (s, 3H), 3.10-3.44 (m, 2H), 2.69 (br t, J=6.8 Hz, 2H), 2.07-2.57 (m, 7H), 1.34-1.96 (m, 4H). MS m/z (M+H)=365.36.

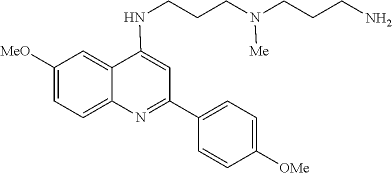 [SMILES: COc1ccc(-c2cc(NCCCN(C)CCCN)c3cc(OC)ccc3n2)cc1]

### Example 9: N¹-(3-Aminopropyl)-N³-(6-methoxy-2-(4-methoxyphenyl)quinolin-4-yl)-N¹-methylpropane-1,3-diamine

Step 1: To a stirring solution of 4-chloro-6-methoxyquinoline (5.0 g, 25.8 mmol) in dichloromethane (150 mL) was added trifluoroacetic acid (1.99 mL, 25.8 mmol) followed by (4-methoxyphenyl)boronic acid (5.88 g, 38.7 mmol). Water (150 mL) was then added, followed by silver nitrate (0.208 g, 1.22 mmol) and potassium persulfate (4.96 g, 18.3 mmol). The reaction mixture was stirred at 23° C. over a period of 3 hours. If not complete after 3 hours, a second addition of silver nitrate (0.104 g, 0.61 mmol) and potassium persulfate (2.48 g, 9.15 mmol) was added. After 24 hours, the resulting reaction mixture was diluted with dichloromethane (200 mL) and washed with sodium bicarbonate (250 mL). Aqueous layer was extracted three times with dichloromethane, dried over magnesium sulfate and filtered. The resultant crude product was purified through silica gel cartridge eluting with ethyl acetate/hexanes to give 4-chloro-6-methoxy-2-(4-methoxyphenyl)quinoline as a yellow powder (1.52 g, 20%). ¹H NMR (300 MHz, DMSO-d₆) δ 8.07 (d, J=9.4 Hz, 2H), 8.05 (s, 1H), 7.89 (s, 1H), 7.53-7.33 (m, 2H), 7.01 (d, J=9.96 Hz, 2H), 3.98 (s, 3H), 3.88 (s, 3H). MS m/z (M+H)=300.08.

Step 2: To a solution of 4-chloro-6-methoxy-2-(4-methoxyphenyl)quinoline (0.1 g, 0.37 mmol) with 3,3′-diamino-N-methyldipropylamine (0.717 mL, 4.45 mmol) was added two drops of tin tetrachloride. The reaction mixture was stirred at 130° C. for 12 hour. The reaction mixture was directly purified through silica gel cartridge eluting with dichloromethane/methanol with 5% ammonium hydroxide to give N¹-(3-aminopropyl)-N³-(6-methoxy-2-(4-methoxyphenyl)quinolin-4-yl)-N¹-methylpropane-1,3-diamine as a pale brown oil (71 mg, 47%). ¹H NMR (300 MHz, CDCl₃) δ 8.08-7.98 (m, 2H), 7.95 (d, J=9.1 Hz, 1H), 7.32-7.26 (m, 1H), 7.16 (br s, 1H), 7.05-6.95 (m, 3H), 6.75 (s, 1H), 3.92 (s, 3H), 3.86 (s, 3H), 3.49-3.44 (m, 2H), 2.72 (t, J=6.9 Hz, 2H), 3.66-2.57 (m, 2H), 2.56-2.46 (m, 2H), 2.39 (s, 3H), 1.97 (quin, J=5.8 Hz, 2H), 1.69 (quin, J=7.2 Hz, 2H), 1.37 (br s, 2H). MS m/z (M+H)=409.3.

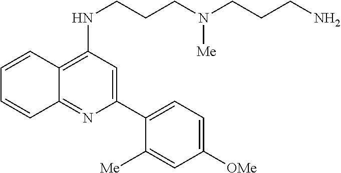 [SMILES: COc1ccc(-c2cc(NCCCN(C)CCCN)c3ccccc3n2)c(C)c1]

Example 10: N¹-(3-Aminopropyl)-N³-(2-(2-methyl-4-methoxyphenyl)quinolin-4-yl)-N¹-methylpropane-1,3-diamine. 2,4-dichloroquinoline (1.0 g, 5.05 mmol) and 4-methoxy-2-methylphenylboronic acid (838 mg, 5.05 mmol) in ethanol/toluene/water were reacted according to step 1 of the procedure for the synthesis of intermediate (IA) to provide 4-chloro-2-(4-methoxy-2-methylphenyl)quinoline. (820 mg, 57%). MS m/z (M+H)=284.02. 4-Chloro-2-(4-methoxy-2-methylphenyl)quinoline (100 mg, 0.35 mmol) and excess N¹-(3-aminopropyl)-N¹-methylpropane-1,3-diamine and tin tetrachloride (3 drops) were reacted according to the procedure of step 2 of the synthesis of intermediate (IA) to afford the product as a pale yellow solid (55 mg, 40%). ¹H NMR (DMSO-d₆) δ: 13.80 (s, 1H), 10.92-11.18 (m, 1H), 9.75 (br t, J=5.6 Hz, 1H), 8.79 (d, J=8.5 Hz, 1H), 8.24 (br s, 4H), 8.06 (d, J=8.4 Hz, 1H), 7.85-8.00 (m, 1H), 7.61-7.76 (m, 1H), 6.85 (s, 1H), 3.82 (s, 3H), 3.66 (s, 3H), 3.00-3.35 (m, 5H), 2.80-2.97 (m, 3H), 2.70 (d, J=4.5 Hz, 4H), 2.47 (dt, J=3.7, 1.8 Hz, 6H), 2.36 (s, 3H), 1.95-2.23 (m, 5H). MS m/z (M+H)=393.33.

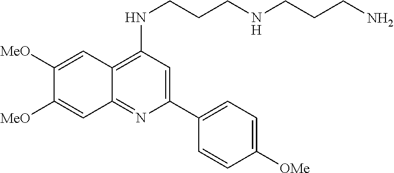 [SMILES: COc1ccc(-c2cc(NCCCNCCCN)c3cc(OC)c(OC)cc3n2)cc1]

### Example 11: N¹-(3-Aminopropyl)-N³-(6,7-dimethoxy-2-(4-methoxyphenyl)quinolin-4-yl)-N¹-methylpropane-1,3-diamine

Step 1: To a stirred solution of 4-chloro-6,7-dimethoxyquinoline (1.0 g, 4.47 mmol) in dichloromethane (30 mL) was added trifluoroacetic acid (0.34 mL, 4.47 mmol) followed by (4-methoxyphenyl)boronic acid (1.02 g, 6.71 mmol). Water (30 mL) was then added followed by silver nitrate (0.15 g, 0.89 mmol) and potassium persulfate (3.62 g, 13.4 mmol). The reaction mixture was stirred at 23° C. over a period of 3 hours. After monitoring by TLC, a large amount of heterocycle still remains after 3 hours, a second batch of silver nitrate (0.076 g, 0.44 mmol) and potassium persulfate (1.81 g, 6.7 mmol) was added. After 24 hours, the resulting reaction mixture was diluted with dichloromethane (50 mL) and washed with sodium bicarbonate (50 mL). Aqueous layer was extracted three times with dichloromethane, dried over magnesium sulfate and filtered. The resultant crude product was purified through silica gel cartridge eluting with ethyl acetate/hexanes to give 4-chloro-6,7-dimethoxy-2-(4-methoxyphenyl)quinoline as a yellow powder (419 mg, 29%). ¹H NMR (300 MHz, CDCl₃) δ 8.08 (dd, J=8.79, 2.93 Hz, 2H), 7.78 (s, 1H), 7.48 (s, 1H), 7.44-7.37 (m, 1H), 7.03 (dd, J=8.8, 1.8 Hz, 2H), 4.09 (s, 3H), 4.07 (s, 3H), 3.89 (s, 3H). MS m/z (M+H)=330.08.

Step 2: To a solution of 4-chloro-6,7-dimethoxy-2-(4-methoxyphenyl)quinoline (0.1 g, 0.3 mmol) with bis-(3-aminopropyl)-N-methylamine (0.587 mL, 3.64 mmol) was added two drops of tin tetrachloride. The reaction was carried out following the same procedure as step 2 of the synthesis of intermediate A (IA). The obtained oil was dissolved in diethyl ether and excess of hydrogen chloride solution in diethyl ether was added. The solvent was evaporated and the resulting powder was dried under vacuum to give N¹-(3-aminopropyl)-N³-(6,7-dimethoxy-2-(4-methoxyphenyl)quinolin-4-yl)-N¹-methylpropane-1,3-diamine trihydrochloride as a yellow powder (14 mg, 11%). ¹H NMR (300 MHz, DMSO-d₆) δ. 13.51-13.17 (m, 1H), 11.14-10.60 (m, 1H), 9.48-9.16 (m, 1H), 8.29-7.93 (m, 6H), 7.70 (s, 1H), 7.20 (d, J=8.8 Hz, 2H), 6.99 (s, 1H), 3.99 (s, 3H), 3.95 (s, 3H), 3.89 (s, 3H), 3.82-3.68 (m, 2H), 3.26 (s, 4H), 3.00-2.83 (m, 2H), 2.74 (s, 3H), 2.31-2.13 (m, 2H), 2.12-1.93 (m, 2H). MS m/z (M+H)=439.3.

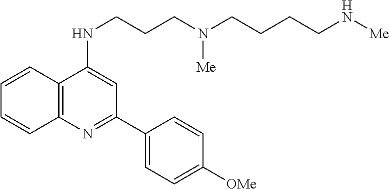 [SMILES: CNCCCCN(C)CCCNc1cc(-c2ccc(OC)cc2)nc2ccccc12]

Example 12: N¹-(3-((2-(4-Methoxyphenyl)quinolin-4-yl)amino)propyl)-N¹,N⁴-dimethyl butane-1,4-diamine trihydrochloride. To a solution of N,N′-dimethyl-1,4-butanediamine (0.100 mg, 0.86 mmol) in dimethylformamide (0.5 mL) was added potassium carbonate (0.059 g, 0.43 mmol) followed by IA (0.08 g, 0.17 mmol). The reaction was carried out following the same procedure as Example 5 The obtained oil was dissolved in diethyl ether and excess of hydrogen chloride solution in diethyl ether was added. The solvent was evaporated and the resulting powder was dried under vacuum to give N¹-(3-((2-(4-methoxyphenyl)quinolin-4-yl)amino)propyl)-N¹,N⁴-dimethylbutane-1,4-diamine trihydrochloride as a white powder (51 mg, 74%). ¹H NMR (300 MHz, DMSO-d₆) δ 13.68 (s, 1H), 10.65 (br s, 1H), 9.75-9.60 (m, 1H), 9.41 (br s, 2H), 8.73 (d, J=8.5 Hz, 1H), 8.29 (d, J=8.5 Hz, 1H), 8.15 (d, J=8.9 Hz, 2H), 7.97-7.80 (m, 1H), 7.71-7.56 (m, 1H), 7.17 (d, J=8.9 Hz, 2H), 7.06 (s, 1H), 3.87 (s, 3H), 3.84-3.73 (m, 2H), 3.11-2.97 (m, 4H), 2.96-2.83 (m, 2H), 2.70 (s, 3H), 2.68 (s, 3H), 2.15 (br t, J=6.8 Hz, 2H), 1.73 (br d, J=6.9 Hz, 4H). MS m/z (M+H)=407.5.

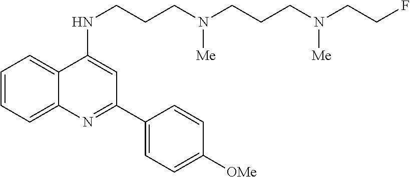 [SMILES: COc1ccc(-c2cc(NCCCN(C)CCCN(C)CCF)c3ccccc3n2)cc1]

Example 13: N¹-(2-fluoroethyl)-N³-(3-((2-(4-methoxyphenyl)quinolin-4-yl)amino)propyl)-N¹,N³-dimethylpropane-1,3-diamine. To a stirring solution of 2-(4-methoxyphenyl)-N-(3-{methyl[3(methylamino)propyl]amino}propyl)quinolin-4-amine (90 mg, 0.23 mmol) in N-methylpyrrolidine (1.0 mL) was added potassium carbonate (48 mg, 0.35 mmol) and 2-iodoethylfluoride (20 uL, 0.23 mmol). The reaction mixture was heated to 85° C. overnight. The reaction mixture was then allowed to cool to 23° C. and diluted with ethyl acetate (10 mL) and the organic layer was washed with water (2×20 mL), brine (20 mL), dried (sodium sulfate), filtered and concentrated under reduced pressure. The resultant crude product was purified through silica gel cartridge (4 g, 60-120 mesh) eluting with dichloromethane/methanol with 5% ammonium hydroxide to give the product as pale yellow foam in 41% yield. ¹H NMR (CDCl₃) δ: 8.95 (s, 1H), 8.75 (d, J=8.5 Hz, 1H), 8.23 (br d, J=8.4 Hz, 1H), 7.96-8.16 (m, 2H), 7.59-7.76 (m, 1H), 7.36-7.53 (m, 1H), 6.80 (d, J=8.9 Hz, 2H), 6.57 (s, 1H), 4.60 (t, J=4.8 Hz, 1H), 4.44 (t, J=4.8 Hz, 1H), 3.63-3.89 (m, 5H), 2.90-3.24 (m, 3H), 2.78 (t, J=4.8 Hz, 1H), 2.68 (br d, J=14.5 Hz, 1H), 2.63 (t, J=6.5 Hz, 1H), 2.33 (s, 3H), 2.26 (br t, J=6.4 Hz, 2H), 1.89 (br t, J=6.8 Hz, 2H). MS m/z (M+H)=439.26.

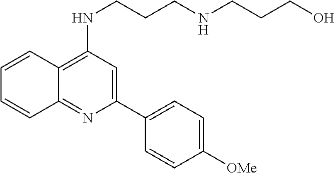 [SMILES: COc1ccc(-c2cc(NCCCNCCCO)c3ccccc3n2)cc1]

Example 14: 3-((3-((2-(4-Methoxyphenyl)quinolin-4-yl)amino)propyl)amino)-propan-1-ol. To a solution of 3-aminopropan-1-ol (0.016 g, 0.21 mmol) in dimethylformamide (0.5 mL) was added pyridine (20 μL, 0.25 mmol) followed by IA (0.08 g, 0.17 mmol). The resulting reaction mixture was stirred at 23° C. over a period of 16 hours. The reaction mixture was then diluted with dichloromethane (5 mL), washed with water (5 mL). Aqueous layer was extracted three times with dichloromethane, dried over magnesium sulfate and filtered. The resultant crude product was purified through silica gel cartridge with dichloromethane/methanol with 5% ammonium hydroxide to give 3-((3-((2-(4-methoxyphenyl)quinolin-4-yl)amino)propyl)amino)propan-1-ol as pale yellow powder (33 mg, 54%). ¹H NMR (DMSO-d₆) δ: 13.59 (s, 1H), 9.59 (br t, J=5.8 Hz, 1H), 9.10 (br s, 1H), 8.68 (d, J=8.5 Hz, 1H), 8.19-8.27 (m, 1H), 8.11 (d, J=8.9 Hz, 2H), 7.87-7.97 (m, 1H), 7.61-7.72 (m, 1H), 7.14-7.27 (m, 2H), 7.06 (s, 1H), 3.87 (s, 3H), 3.79 (q, J=6.4 Hz, 2H), 3.46 (t, J=6.0 Hz, 3H), 2.84-3.11 (m, 4H), 2.02-2.19 (m, 2H), 1.70-1.89 (m, 2H). MS m/z (M+H)=366.4.

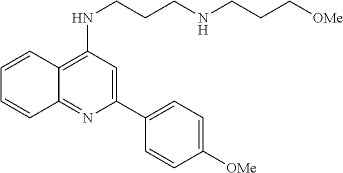 [SMILES: COCCCNCCCNc1cc(-c2ccc(OC)cc2)nc2ccccc12]

Example 15: N-(3-(3-methoxypropylamino)propyl)-2-(4-methoxyphenyl)quinolin-4-amine. Intermediate IA (100 mg, 0.22 mmol) was dissolved in 3-methoxypropan-1-amine (1.0 mL) and heated to 110° C. for 14 hours. The cooled reaction mixture was poured into ice water and the resulting white solid collected by filtration. Washed with water and dichloromethane and dried under vacuum to afford N-(3-(3-methoxypropylamino)propyl)-2-(4-methoxyphenyl)quinolin-4-amine as a white solid (26 mg, 31%). ¹H NMR (CDCl₃) Shift: 8.05 (d, J=8.8 Hz, 2H), 7.76-7.82 (m, 1H), 7.61 (ddd, J=8.4, 6.9, 1.3 Hz, 1H), 7.28-7.40 (m, 2H), 7.02 (q, J=5.1 Hz, 1H), 6.75 (s, 1H), 3.88 (s, 3H), 3.45-3.53 (m, 4H), 3.34 (s, 3H), 2.88-2.95 (m, 2H), 2.79 (t, J=7.0 Hz, 2H), 1.72-2.02 (m, 9H). MS m/z (M+H)=380.

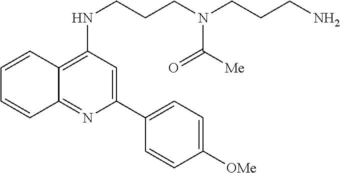 [SMILES: COc1ccc(-c2cc(NCCCN(CCCN)C(C)=O)c3ccccc3n2)cc1]

### Example 16: N-(3-(2-(4-Methoxyphenyl)quinolin-4-ylamino)propyl)-N-(3-aminopropyl) acetamide

Step 1: Synthesis of Tert-butyl N-{3-[(3-{[2-(4-methoxyphenyl)quinolin-4yl]amino}propyl)amino] propyl}carbamate. IB (100 mg, 0.27 mmol) in dichloromethane (1.0 mL) was added tert-butyl 3-aminopropylcarbamate (94 uL, 0.54 mmol) and the solution was stirred for 6 hours at 23° C. The reaction mixture was concentrated. The crude product was purified through silica gel cartridge (4 g, 60-120 mesh) eluting with dichloromethane/methanol with 5% ammonium hydroxide 0-10% to give the tert-butyl 3-aminopropylcarbamate as a white foam (75%). ¹H NMR (CHLOROFORM-d) δ: 7.95-8.14 (m, 3H), 7.76 (d, J=8.3 Hz, 1H), 7.60 (ddd, J=8.4, 6.9, 1.3 Hz, 1H), 7.30-7.45 (m, 1H), 7.25 (s, 2H), 7.01 (d, J=8.8 Hz, 2H), 6.77 (s, 1H), 4.83 (s, 1H), 3.87 (s, 3H), 3.39-3.59 (m, 2H), 3.25 (q, J=6.5 Hz, 2H), 2.88 (t, J=5.7 Hz, 2H), 2.73 (t, J=6.8 Hz, 2H), 1.96 (quin, J=5.9 Hz, 2H), 1.75 (quin, J=6.7 Hz, 5H), 1.43 (s, 7H). MS m/z (M+H)=465.28.

Step 2: To a cooled solution of tert-butyl N-{3-[(3-{[2-(4-methoxyphenyl)quinolin-4yl]amino}propyl)amino]propyl}carbamate (132 mg, 0.28 mmol) and triethylamine (60 uL, 0.43 mmol) in anhydrous dichloromethane and triethylamine was added acetyl chloride (20 uL, 0.28 mmol). The cooling bath was removed and the reaction was allowed to reach 23° C. The reaction was quenched with water and partitioned between dichloromethane and water. The layers were separated and the organic layer was washed with saturated sodium bicarbonate and brine, dried over sodium sulfate, decanted and concentrated under vacuum. The crude product was purified on silica gel cartridge (4 g, 60-120 mesh)), eluting with dichloromethane/methanol with 5% ammonium hydroxide to give tert-butyl 3-(N-(3-(2-(4-methoxyphenyl)quinolin-4-ylamino)-propyl)acetamido)propyl carbamate as a pale foam (105 mg, 74%). ¹H NMR (CDCl₃) δ: 7.96-8.17 (m, 5H), 7.55-7.64 (m, 1H), 7.27-7.45 (m, 1H), 7.26 (s, 1H), 6.86-7.03 (m, 3H), 6.63-6.72 (m, 1H), 4.88 (s, 1H), 3.82 (d, J=1.3 Hz, 3H), 2.99-3.18 (m, 1H), 2.92-3.57 (m, 9H), 1.95-2.21 (m, 4H), 1.59-1.94 (m, 4H), 1.30-1.50 (m, 9H). MS m/z (M+H)=507.36. To a solution of tert-butyl 3-(N-(3-(2-(4-methoxyphenyl)quinolin-4-ylamino)propyl)acetamido) (100 mg, 0.20 mmol) in dichloromethane was added hydrochloric acid (0.20 mL, 4N) in 1,4-dioxane. Stirred at 23° C. for 30 minutes. Concentrated under vacuum and dissolved in water, and dried on lyophilizer overnight. Isolated N-(3-(2-(4-methoxyphenyl)quinolin-4-ylamino)propyl)-N-(3-aminopropyl)acetamide as a pale yellow solid (75 mg, 93%). ¹H NMR (DMSO-d₆) δ: 13.64 (br d, J=14.3 Hz, 1H), 9.37-9.65 (m, 1H), 8.61-8.84 (m, 1H), 8.17-8.34 (m, 3H), 8.11 (d, J=8.7 Hz, 4H), 7.91 (t, J=7.7 Hz, 1H), 7.56-7.72 (m, 1H), 7.19 (d, J=8.5 Hz, 3H), 6.92-7.09 (m, 1H), 3.87 (s, 4H), 3.60-3.72 (m, 3H), 3.31-3.47 (m, 6H), 2.62-2.86 (m, 2H), 2.48 (quin, J=1.9 Hz, 6H), 2.00 (d, J=2.5 Hz, 5H), 1.66-1.97 (m, 3H). MS m/z (M+H)=407.17.

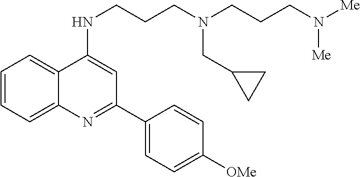 [SMILES: COc1ccc(-c2cc(NCCCN(CCCN(C)C)CC3CC3)c3ccccc3n2)cc1]

### Example 17: N¹-(Cyclopropylmethyl)-N¹-(3-((2-(4-methoxyphenyl)quinolin-4-yl)amino) propyl)-N³,N³-dimethylpropane-1,3-diamine

Step 1: Synthesis of N¹-(3-((2-(4-methoxyphenyl)quinolin-4-yl)amino)propyl)-N³,N³-dimethylpropane-1,3-diamine. To a solution of IA (800 mg, 1.73 mmol) in acetonitrile (0.5 mL) was added N¹,N¹-dimtehylpropane-1,3-diamine (265 mg, 2.6 mmol) and triethylamine (262 mg, 2.6 mmol). The reaction was stirred at 23° C. for 72 hours. Partitioned between ethyl acetate and saturated sodium bicarbonate. Extracted with ethyl acetate (3×5 mL) washed combined organics with magnesium sulfate, decanted and concentrated under vacuum. The resultant crude product was purified through silica gel cartridge with dichloromethane/methanol with 5% ammonium hydroxide to provide N¹-(3-((2-(4-methoxyphenyl)quinolin-4-yl)amino)propyl)-N³,N³-dimethylpropane-1,3-diamine.

Step 2: To a solution of N¹-(3-((2-(4-methoxyphenyl)quinolin-4-yl)amino)propyl)-N³,N³-dimethylpropane-1,3-diamine (0.080 g, 0.20 mmol) in dichloroethane (0.5 mL) was added cyclopropane carboxaldehyde (0.028 g, 0.40 mmol), a few drops of acetic acid followed by sodium triacetoxyborohydride (0.112 g, 0.53 mmol). The resulting reaction mixture was stirred at 23° C. over a period of 16 hours. The reaction mixture was then diluted with ethyl acetate (5 mL), washed with sodium bicarbonate (5 mL). Aqueous layer was extracted three times with ethyl acetate, dried over magnesium sulfate and filtered. The resultant crude product was purified through silica gel cartridge with dichloromethane/methanol with 5% ammonium hydroxide to give N¹-(cyclopropylmethyl)-N¹-(3-((2-(4-methoxyphenyl)quinolin-4-yl)amino)propyl)-N³,N³-dimethyl propane-1,3-diamine as a yellow oil (54 mg, 61%). ¹H NMR (300 MHz, CDCl₃) δ 8.06 (d, J=8.7 Hz, 2H), 8.01 (d, J=8.6 Hz, 1H), 7.82 (d, J=8.3 Hz, 1H), 7.70 (br s, 1H), 7.54-7.63 (m, 1H), 7.28-7.38 (m, 1H), 7.00 (d, J=8.7 Hz, 2H), 6.73 (s, 1H), 3.85 (s, 3H), 3.47 (q, J=5.2 Hz, 2H), 2.72-2.81 (m, 2H), 2.61-2.71 (m, 2H), 2.45 (d, J=6.6 Hz, 2H), 2.24 (t, J=7.3 Hz, 2H), 2.06-2.15 (m, 6H), 1.86-1.99 (m, 2H), 1.70 (quin, J=7.5 Hz, 2H), 0.88-1.03 (m, 1H), 0.44-0.56 (m, 2H), 0.14 (d, J=5.0 Hz, 1H). MS m/z (M+H)=447.1.

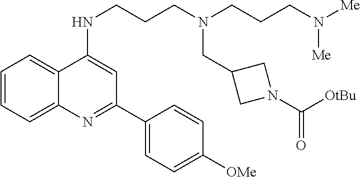 [SMILES: COc1ccc(-c2cc(NCCCN(CCCN(C)C)CC3CN(C(=O)OC(C)(C)C)C3)c3ccccc3n2)cc1]

Example 18: tert-Butyl 3-(((3-(dimethylamino)propyl)(3-((2-(4-methoxyphenyl)quinolin-4-yl)amino)propyl)amino)methyl)azetidine-1-carboxylate. To a solution of N¹-(3-((2-(4-methoxy phenyl)quinolin-4-yl)amino)propyl)-N³,N³-dimethylpropane-1,3-diamine (0.080 g, 0.20 mmol) in dichloroethane (0.5 mL) was added tert-butyl 3-formylazetidine-1-carboxylate (0.076 g, 0.41 mmol), a few drops of acetic acid followed by sodium triacetoxyborohydride (0.112 g, 0.53 mmol). The resulting reaction mixture was stirred at 23° C. over a period of 16 hours. The reaction mixture was then diluted with ethyl acetate (5 mL), washed with sodium bicarbonate (5 mL). Aqueous layer was extracted three times with ethyl acetate, dried over magnesium sulfate and filtered. The resultant crude product was purified through silica gel cartridge with dichloromethane/methanol with 5% ammonium hydroxide to give tert-butyl 3-(((3-(dimethylamino)propyl)(3-((2-(4-methoxyphenyl)quinolin-4-yl)amino)propyl)amino)methyl)-azetidine-1-carboxylate as a pale yellow oil (42 mg, 38%). ¹H NMR (300 MHz, CDCl₃) δ: 7.97-8.09 (m, 3H), 7.66 (d, J=8.3 Hz, 1H), 7.59 (t, J=7.6 Hz, 1H), 7.30-7.40 (m, 1H), 7.01 (d, J=8.5 Hz, 2H), 6.75 (s, 1H), 6.62 (br t, J=4.3 Hz, 1H), 3.92-4.02 (m, 2H), 3.86 (s, 3H), 3.58 (dd, J=8.5, 4.2 Hz, 2H), 3.44 (q, J=5.4 Hz, 2H), 2.72 (s, 3H), 2.62 (br t, J=5.8 Hz, 2H), 2.53 (br t, J=7.5 Hz, 2H), 2.24 (t, J=7.2 Hz, 2H), 2.14 (s, 6H), 1.85-1.99 (m, 2H), 1.66 (br t, J=7.4 Hz, 2H), 1.39 (d, J=0.9 Hz, 9H). MS m/z (M+H)=562.4.

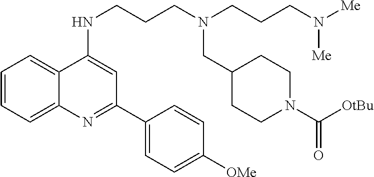 [SMILES: COc1ccc(-c2cc(NCCCN(CCCN(C)C)CC3CCN(C(=O)OC(C)(C)C)CC3)c3ccccc3n2)cc1]

Example 19: tert-Butyl 4-(((3-(dimethylamino)propyl)(3-((2-(4-methoxyphenyl)quinolin-4-yl)amino)propyl)amino)methyl)piperidine-1-carboxylate. To a solution of N¹-(3-((2-(4-methoxyphenyl)quinolin-4-yl)amino)propyl)-N³,N³-dimethylpropane-1,3-diamine (0.080 g, 0.20 mmol) in dichloroethane (0.5 mL) was added tert-butyl 4-formylpiperidine-1-carboxylate (0.087 g, 0.40 mmol), a few drops of acetic acid followed by sodium triacetoxyborohydride (0.112 g, 0.53 mmol). The resulting reaction mixture was stirred at 23° C. over a period of 16 hours. The reaction mixture was then diluted with ethyl acetate (5 mL), washed with sodium bicarbonate (5 mL). Aqueous layer was extracted three times with ethyl acetate, dried over magnesium sulfate and filtered. The resultant crude product was purified through silica gel cartridge with dichloromethane/methanol with 5% ammonium hydroxide to give tert-butyl 4-(((3-(dimethylamino)propyl)(3-((2-(4-methoxyphenyl)quinolin-4-yl)amino)propyl)amino)methyl)-piperidine-1-carboxylate as a pale yellow oil (63 mg, 54%). ¹H NMR (300 MHz, CDCl₃) δ 7.97-8.10 (m, 3H), 7.72 (d, J=8.3 Hz, 1H), 7.59 (t, J=7.6 Hz, 1H), 7.29-7.39 (m, 1H), 7.00 (d, J=8.8 Hz, 2H), 6.76 (s, 1H), 6.39 (t, J=4.4 Hz, 1H), 4.01 (s, 2H), 3.85 (s, 3H), 3.45 (q, J=5.6 Hz, 2H), 2.44-2.70 (m, 6H), 2.19-2.33 (m, 4H), 2.14 (s, 6H), 1.83-1.97 (m, 2H), 1.74 (br d, J=13.2 Hz, 2H), 1.54-1.68 (m, 3H), 1.41 (s, 9H), 0.92-1.15 (m, 2H). MS m/z (M+H)=590.2.

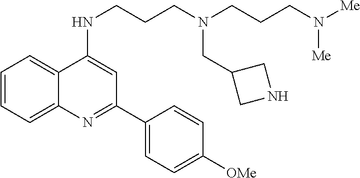 [SMILES: COc1ccc(-c2cc(NCCCN(CCCN(C)C)CC3CNC3)c3ccccc3n2)cc1]

Example 20: N¹-(Azetidin-3-ylmethyl)-N¹-(3-((2-(4-methoxyphenyl)quinolin-4-yl)amino)propyl)-N³,N³-dimethylpropane-1,3-diamine. To a solution of tert-butyl 3-(((3-(dimethyl amino)propyl)(3-((2-(4-methoxyphenyl)quinolin-4-yl)amino)propyl)amino)methyl)azetidine-1-carboxylate (80 mg, 0.020 mmol) in diethyl ether (1 mL) was added excess of hydrogen chloride solution in diethyl ether. The reaction mixture was stirred overnight at 23° C. The solvent was evaporated and the resulting powder was dried under vacuum to give N¹-(azetidin-3-ylmethyl)-N¹-(3-((2-(4-methoxyphenyl)quinolin-4-yl)amino)propyl)-N³,N³-dimethylpropane-1,3-diamine was dried under high vacuum, and obtained as a white powder (34 mg, 98%). ¹H NMR (DMSO-d₆) δ: 13.67 (s, 1H), 11.14-11.72 (m, 1H), 10.56-11.05 (m, 1H), 9.54-9.86 (m, 1H), 9.21-9.51 (m, 1H), 8.79 (br d, J=8.1 Hz, 1H), 8.61 (br d, J=5.3 Hz, 1H), 8.27 (br d, J=8.5 Hz, 1H), 8.11-8.21 (m, 2H), 7.92 (t, J=7.7 Hz, 1H), 7.60-7.71 (m, 1H), 7.15-7.25 (m, 2H), 7.04-7.14 (m, 1H), 3.95-4.17 (m, 2H), 3.88 (s, 3H), 3.71-3.87 (m, 4H), 3.44-3.60 (m, 2H), 3.06-3.36 (m, 6H), 2.84-3.05 (m, 1H), 2.67-2.80 (m, 6H), 2.23 (br d, J=12.3 Hz, 4H). MS m/z (M+H)=462.1.

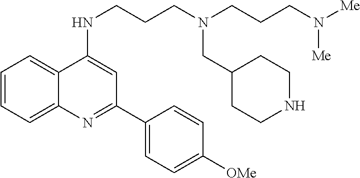 [SMILES: COc1ccc(-c2cc(NCCCN(CCCN(C)C)CC3CCNCC3)c3ccccc3n2)cc1]

Example 21: N¹-(3-((2-(4-Methoxyphenyl)quinolin-4-yl)amino)propyl)-N³,N³-dimethyl-N¹-(piperidin-4-ylmethyl)propane-1,3-diamine. To a solution of tert-butyl 4-(((3-(dimethylamino) propyl)(3-((2-(4-methoxyphenyl)quinolin-4-yl)amino)propyl)amino)-methyl)piperidine-1-carboxylate (63 mg, 0.011 mmol) in diethyl ether (1 mL) was added excess of hydrogen chloride solution in diethyl ether. The reaction mixture was stirred overnight at 23° C. The solvent was evaporated and the resulting powder was dried under vacuum to give N¹-(3-((2-(4-Methoxyphenyl)quinolin-4-yl)amino)propyl)-N³,N³-dimethyl-N¹-(piperidin-4-ylmethyl)propane-1,3-diamine was obtained as a white powder (56 mg, 100%). ¹H NMR (DMSO-d₆) δ: 13.64 (s, 1H), 10.94 (s, 2H), 9.73 (s, 1H), 8.90-9.20 (m, 2H), 8.82 (d, J=8.6 Hz, 1H), 8.25 (d, J=8.5 Hz, 1H), 8.15 (d, J=8.8 Hz, 2H), 7.93 (t, J=7.8 Hz, 1H), 7.61-7.70 (m, 1H), 7.20 (d, J=8.8 Hz, 2H), 7.08 (s, 1H), 3.88 (s, 3H), 3.78 (br d, J=6.6 Hz, 2H), 3.16-3.31 (m, 6H), 2.97-3.15 (m, 4H), 2.76-2.89 (m, 2H), 2.72 (br d, J=4.3 Hz, 6H), 2.10-2.36 (m, 5H), 2.03 (br t, J=12.4 Hz, 2H), 1.42 (q, J=11.9 Hz, 2H). MS m/z (M+H)=490.2.

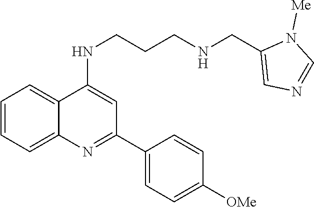 [SMILES: COc1ccc(-c2cc(NCCCNCc3cncn3C)c3ccccc3n2)cc1]

Example 22: N¹-(2-(4-Methoxyphenyl)quinolin-4-yl)-N³-((1-methyl-1H-imidazol-5-yl)methyl)propane-1,3-diamine. To a solution of (1-methyl-1H-imidazol-5-yl)methanamine (0.096 mg, 0.86 mmol) in dimethylformamide (0.5 mL) was added potassium carbonate (0.059 g, 0.43 mmol) followed by IA (0.08 g, 0.17 mmol). The reaction was carried out following the same procedure as Example 3. N¹-(2-(4-methoxyphenyl)quinolin-4-yl)-N³-((1-methyl-1H-imidazol-5-yl)methyl)propane-1,3-diamine was obtained as pale yellow powder (3 mg, 5%). ¹H NMR (CDCl₃) δ: 7.99-8.08 (m, 3H), 7.95 (q, J=4.3 Hz, 2H), 7.68-7.75 (m, 1H), 7.62 (ddd, J=8.4, 7.0, 1.3 Hz, 1H), 7.31-7.40 (m, 1H), 6.98 (d, J=8.8 Hz, 2H), 6.82 (s, 1H), 6.76 (d, J=8.6 Hz, 2H), 5.32 (br t, J=5.4 Hz, 1H), 4.74-4.87 (m, 2H), 3.93 (br s, 1H), 3.86 (s, 3H), 3.55 (q, J=6.3 Hz, 2H), 2.50 (quin, J=6.4 Hz, 2H).

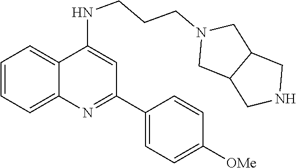 [SMILES: COc1ccc(-c2cc(NCCCN3CC4CNCC4C3)c3ccccc3n2)cc1]

Example 23: N-(3-(Hexahydropyrrolo[3,4-c]pyrrol-2(1H)-yl)propyl)-2-(4-methoxyphenyl) quinolin-4-amine. To a solution of tert-butyl 5-(3-((2-(4-methoxyphenyl)quinolin-4-yl)amino) propyl)hexahydropyrrolo[3,4-c]pyrrole-2(1H)-carboxylate (45 mg, 0.013 mmol) in diethyl ether (1 mL) was added excess of hydrogen chloride solution in diethyl ether. The reaction mixture was stirred overnight at 23° C. The solvent was evaporated and the resulting powder was dried under vacuum to give N-(3-(hexahydropyrrolo[3,4-c]pyrrol-2(1H)-yl)propyl)-2-(4-methoxyphenyl) quinolin-4-amine was obtained as a pale yellow powder (27 mg, 75%). ¹H NMR (DMSO-d₆) δ: 13.51-13.74 (m, 1H), 11.21-11.63 (m, 1H), 9.21-9.74 (m, 2H), 8.66 (dd, J=11.4, 8.5 Hz, 1H), 8.23 (dd, J=8.5, 5.7 Hz, 1H), 8.13 (d, J=8.5 Hz, 2H), 7.88-7.98 (m, 1H), 7.62-7.72 (m, 1H), 7.20 (d, J=8.8 Hz, 2H), 7.07 (d, J=4.5 Hz, 1H), 3.88 (s, 3H), 3.72-3.85 (m, 3H), 3.58-3.70 (m, 1H), 3.05-3.36 (m, 10H), 2.06-2.30 (m, 2H). MS m/z (M+H)=403.1.

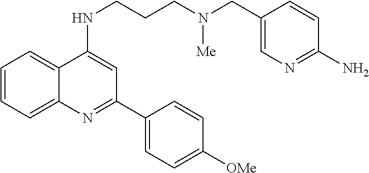 [SMILES: COc1ccc(-c2cc(NCCCN(C)Cc3ccc(N)nc3)c3ccccc3n2)cc1]

Example 24: N¹-((6-Aminopyridin-3-yl)methyl)-N³-(2-(4-methoxyphenyl)-quinolin-4-yl)-N¹-methylpropane-1,3-diamine. To a solution of N¹-(2-(4-methoxyphenyl)quinolin-4-yl)-N³-methylpropane-1,3-diamine (0.060 g, 0.19 mmol) in dichloroethane (0.5 mL) was added 6-aminonicotinaldehyde (0.046 g, 0.37 mmol), a few drops of acetic acid followed by sodium triacetoxyborohydride (0.103 g, 0.48 mmol). The resulting reaction mixture was stirred at 23° C. over a period of 16 hours. The reaction mixture was then diluted with ethyl acetate (5 mL), washed with sodium bicarbonate (5 mL). Aqueous layer was extracted three times with ethyl acetate, dried over magnesium sulfate and filtered. The resultant crude product was purified through silica gel cartridge with dichloromethane/methanol with 5% ammonium hydroxide to give N¹-((6-aminopyridin-3-yl)methyl)-N³-(2-(4-methoxyphenyl)quinolin-4-yl)-N¹-methylpropane-1,3-diamine as pale yellow powder (6 mg, 8%). ¹H NMR (CDCl₃) δ: 8.06 (d, J=8.9 Hz, 3H), 7.98 (br d, J=16.9 Hz, 1H), 7.60 (ddd, J=8.4, 6.9, 1.3 Hz, 1H), 7.52 (d, J=8.3 Hz, 1H), 7.38 (dd, J=8.4, 2.3 Hz, 1H), 7.26 (br dd, J=15.1, 1.3 Hz, 2H), 7.01 (d, J=8.8 Hz, 2H), 6.75 (s, 1H), 6.33 (d, J=8.4 Hz, 1H), 4.45 (s, 2H), 3.87 (s, 3H), 3.44 (s, 4H), 2.58-2.70 (m, 2H), 2.34 (s, 3H), 1.92-2.02 (m, 2H). MS m/z (M+H)=428.1.

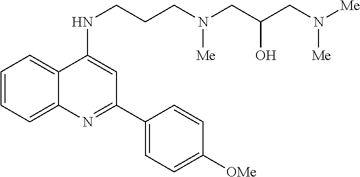 [SMILES: COc1ccc(-c2cc(NCCCN(C)CC(O)CN(C)C)c3ccccc3n2)cc1]

Example 25: 1-(Dimethylamino)-3-((3-((2-(4-methoxyphenyl)quinolin-4-yl)-amino) propyl)(methyl)amino)propan-2-ol. To a solution of 1-amino-3-(dimethylamino)propan-2-ol (31 mg, 0.26 mmol) in acetonitrile (0.5 mL) was added triethylamine (36 μL, 0.26 mmol) followed by 3-((2-(4-methoxyphenyl)quinolin-4-yl)amino)propyl 4-methylbenzenesulfonate (80 mg, 0.17 mmol). The reaction mixture was stirred overnight at 23° C. The reaction mixture was diluted in ethyl acetate, washed with saturated aqueous sodium bicarbonate. Aqueous layer was extracted three times with ethyl acetate, dried over magnesium sulfate and filtered. The resultant crude product was purified through silica gel cartridge eluting with dichloromethane/methanol with 5% ammonium hydroxide to give 1-(dimethylamino)-3-((3-((2-(4-methoxyphenyl)quinolin-4-yl)amino)propyl)(methyl)amino)propan-2-ol as pale yellow powder (27 mg, 39%). ¹H NMR (CDCl₃) δ: 8.00-8.11 (m, 3H), 7.83 (d, J=8.3 Hz, 1H), 7.59 (t, J=7.6 Hz, 1H), 7.32 (t, J=7.6 Hz, 1H), 7.00 (d, J=8.6 Hz, 2H), 6.73 (s, 1H), 3.88-3.97 (m, 1H), 3.83-3.87 (m, 3H), 3.48 (br d, J=5.9 Hz, 2H), 2.82-3.03 (m, 3H), 2.56-2.77 (m, 3H), 2.34-2.47 (m, 1H), 2.29 (s, 6H), 2.19 (dd, J=12.1, 3.4 Hz, 1H), 1.96 (br t, J=5.9 Hz, 2H). MS m/z (M+H)=409.2.

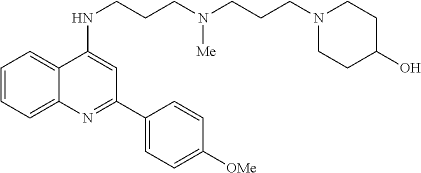 [SMILES: COc1ccc(-c2cc(NCCCN(C)CCCN3CCC(O)CC3)c3ccccc3n2)cc1]

Example 26: 1-(3-((3-((2-(4-Methoxyphenyl)quinolin-4-yl)amino)propyl)-methylamino) propyl) piperidin-4-ol. To a solution of 1-(3-aminopropyl)piperidin-4-ol (41 mg, 0.26 mmol) in acetonitrile (0.5 mL) was added triethylamine (36 μL, 0.26 mmol) followed by 3-((2-(4-methoxyphenyl)quinolin-4-yl)amino)propyl 4-methylbenzenesulfonate (80 mg, 0.17 mmol). The reaction mixture was stirred overnight at 23° C. The reaction mixture was diluted in ethyl acetate, washed with saturated aqueous sodium bicarbonate. Aqueous layer was extracted three times with ethyl acetate, dried over magnesium sulfate and filtered. The resultant crude product was purified through silica gel cartridge eluting with dichloromethane/methanol with 5% ammonium hydroxide to give 1-(3-((3-((2-(4-methoxyphenyl)quinolin-4-yl)amino)propyl)(methyl)amino)propyl)piperidin-4-ol as a white powder (20 mg, 27%). ¹H NMR (CDCl₃) δ: 7.91-8.06 (m, 4H), 7.78 (d, J=7.9 Hz, 1H), 7.51-7.60 (m, 1H), 7.26-7.32 (m, 1H), 7.15 (d, J=7.9 Hz, 1H), 6.95 (d, J=8.6 Hz, 2H), 6.65 (s, 1H), 3.80-3.85 (m, 3H), 3.55-3.67 (m, 1H), 3.50 (br t, J=6.0 Hz, 2H), 2.97 (t, J=6.1 Hz, 2H), 2.85 (t, J=6.5 Hz, 2H), 2.77 (br d, J=12.3 Hz, 2H), 2.43 (t, J=6.6 Hz, 2H), 2.32 (s, 1H), 1.99-2.17 (m, 4H), 1.73-1.91 (m, 4H), 1.42-1.61 (m, 2H). MS m/z (M+H)=449.4.

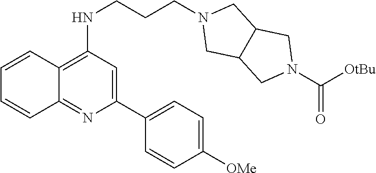 [SMILES: COc1ccc(-c2cc(NCCCN3CC4CN(C(=O)OC(C)(C)C)CC4C3)c3ccccc3n2)cc1]

Example 27: tert-butyl 5-(3-((2-(4-methoxyphenyl)quinolin-4-yl)amino)propyl)hexahydro-pyrrolo[3,4-c]pyrrole-2(1H)-carboxylate. To a solution of 2-Boc-hexahydro-pyrrolo [3,4-c]pyrrole (0.055 g, 0.26 mmol) in acetonitrile (0.5 mL) was added triethylamine (0.036 μL, 0.26 mmol) followed by IA (0.08 g, 0.17 mmol). The resulting reaction mixture was stirred at 23° C. over a period of 16 hours. The reaction mixture was then diluted with ethyl acetate (5 mL), washed with sodium bicarbonate (5 mL). Aqueous layer was extracted three times with ethyl acetate, dried over magnesium sulfate and filtered. The resultant crude product was purified through silica gel cartridge with dichloromethane/methanol with 5% ammonium hydroxide to give tert-butyl 5-(3-((2-(4-methoxyphenyl)quinolin-4-yl)amino)propyl)hexahydro-pyrrolo[3,4-c]pyrrole-2(1H)-carboxylate (45 mg, 53%). ¹H NMR (CDCl₃) δ: 10.08-10.29 (m, 1H), 8.55 (br d, J=8.4 Hz, 1H), 8.12 (s, 1H), 7.80-8.06 (m, 4H), 7.59 (t, J=7.6 Hz, 1H), 7.39-7.52 (m, 1H), 7.27-7.38 (m, 2H), 7.19 (dd, J=12.2, 8.1 Hz, 3H), 6.97 (d, J=8.5 Hz, 2H), 6.73 (d, J=8.2 Hz, 2H), 6.51 (s, 1H), 6.25 (s, 1H), 4.61-4.76 (m, 1H), 3.84 (s, 3H), 3.71-3.79 (m, 1H), 3.65 (s, 3H), 3.34-3.59 (m, 6H), 3.14 (br d, J=11.7 Hz, 2H), 2.58-2.85 (m, 2H), 2.52 (br t, J=6.2 Hz, 2H), 2.35 (s, 2H), 2.02-2.26 (m, 3H), 1.79-1.96 (m, 4H), 1.43 (s, 9H). MS m/z (M+H)=503.3.

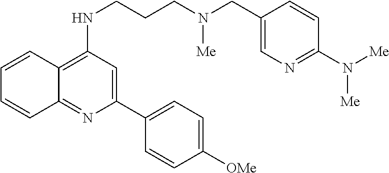 [SMILES: COc1ccc(-c2cc(NCCCN(C)Cc3ccc(N(C)C)nc3)c3ccccc3n2)cc1]

Example 28: N¹-((6-(dimethylamino)pyridin-3-yl)methyl)-N³-(2-(4-methoxyphenyl) quinolin-4-yl)-N¹-methylpropane-1,3-diamine. To a solution of N¹-(2-(4-methoxyphenyl)quinolin-4-yl)-N³-methylpropane-1,3-diamine (0.080 g, 0.25 mmol) in dichloroethane (0.5 mL) was added 6-(dimethylamino)nicotine-3-carboxaldehyde (0.075 g, 0.50 mmol), a few drops of acetic acid followed by sodium triacetoxyborohydride (0.137 g, 0.65 mmol). The resulting reaction mixture was stirred at 23° C. over a period of 16 hours. The reaction mixture was then diluted with ethyl acetate (5 mL), washed with sodium bicarbonate (5 mL). Aqueous layer was extracted three times with ethyl acetate, dried over magnesium sulfate and filtered. The resultant crude product was purified through silica gel cartridge with dichloromethane/methanol with 5% ammonium hydroxide to give N¹-((6-(dimethylamino)pyridin-3-yl)methyl)-N³-(2-(4-methoxyphenyl)quinolin-4-yl)-N¹-methylpropane-1,3-diamine as a white powder (13 mg, 12%). ¹H NMR (CDCl₃) δ: 7.98-8.12 (m, 4H), 7.59 (t, J=7.6 Hz, 1H), 7.52 (d, J=8.3 Hz, 1H), 7.38 (dd, J=8.7, 2.4 Hz, 1H), 7.17-7.29 (m, 2H), 7.01 (d, J=8.5 Hz, 2H), 6.74 (s, 1H), 6.34 (d, J=8.7 Hz, 1H), 3.87 (s, 3H), 3.45 (s, 4H), 3.05 (s, 6H), 2.65 (t, J=5.5 Hz, 2H), 2.34 (s, 3H), 1.92-2.04 (m, 2H). MS m/z (M+H)=456.3.

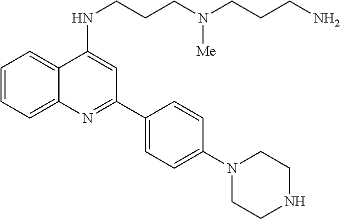 [SMILES: CN(CCCN)CCCNc1cc(-c2ccc(N3CCNCC3)cc2)nc2ccccc12]

### Example 29: Synthesis of N¹-(3-aminopropyl)-N¹-methyl-N³-(2-(4-(piperazin-1-yl)phenyl)quinolin-4-yl)propane-1,3-diamine

Step 1: To a stirring solution of 2,4-dichloroquinoline (0.5 g, 2.5 mmol) in toluene/ethanol (1:1 10 mL) was added 4-(4-tert-butoxycarbonylpiperazinyl)phenylboronic acid, pinacol ester (1.03 g, 2.65 mmol), followed by sodium carbonate (2 M, 2.5 mL, 5.0 mmol). The reaction mixture was degassed over a slow flow of nitrogen for 15 minutes, then tetrakis(triphenylphosphine)-palladium(0) (87 mg, 0.07 mmol) was added. The reaction flask was flushed with nitrogen and heated to 90° C. for 18 hours. The cooled reaction mixture was diluted with ethyl acetate (50 mL), washed with water (25 mL), the aqueous washed with brine (20 mL), dried over sodium sulfate and filtered. The filtrate was concentrated under reduced pressure to give crude tert-butyl 4-(4-(4-chloroquinolin-2-yl)phenyl)piperazine-1-carboxylate as a dark oil and used in the next step without further purification. MS m/z (M+H)=424.33.

Step 2: A solution of crude tert-butyl 4-(4-(4-chloroquinolin-2-yl)phenyl)piperazine-1-carboxylate (1.06 g, 2.5 mmol) in 3-aminopropanol (2.02 mL, 12.5 mmol) was treated with tin tetrachloride (4 drops) and heated overnight at 130° C. The resulting hot reaction mixture was poured into saturated aqueous sodium bicarbonate and extracted with dichloromethane, dried over sodium sulfate, and filtered. The product was purified on silica gel (220 gm, 60-120 mesh) and concentrated under reduced pressure to give tert-butyl 4-(4-(4-((3-((3-aminopropyl)(methyl)amino)propyl)amino)quinolin-2-yl)phenyl)piperazine-1-carboxylate as a yellow solid (0.432 g, 32% over two steps). ¹H NMR (CDCl₃) δ: 8.07 (br d, J=10.0 Hz, 1H), 8.01 (s, 4H), 7.81 (d, J=8.1 Hz, 1H), 7.56 (t, J=7.5 Hz, 1H), 7.35 (t, J=7.5 Hz, 1H), 7.01 (br d, J=8.3 Hz, 2H), 6.66 (s, 1H), 3.54-3.67 (m, 4H), 3.42-3.54 (m, 2H), 3.16-3.30 (m, 4H), 2.81 (t, J=7.5 Hz, 1H), 2.58-2.66 (m, 2H), 2.53 (t, J=7.5 Hz, 2H), 2.38 (s, 4H), 1.89-2.01 (m, 3H), 1.75 (br t, J=6.0 Hz, 1H), 1.49 (s, 9H). MS m/z (M+H)=533.60.

Step 3: Neat tert-butyl 4-(4-(4-((3-((3-aminopropyl)(methyl)amino)propyl)amino) quinolin-2-yl)phenyl)piperazine-1-carboxylate (410 mg, 0.77 mmol) was treated with 4N HCl/Dioxane (2.0 mL, 8 mmol) and stirred at ambient temperature until removal of the tert-butyl carbamate was complete by LC-MS. The reaction mixture was concentrated and purified using reversed-phase HPLC to provide N¹-(3-aminopropyl)-N¹-methyl-N³-(2-(4-(piperazin-1-yl)phenyl)quinolin-4-yl)propane-1,3-diamine trifluoroacetic acid salt as a brown oil upon lyophilization (638 mg). ¹H NMR (DMSO-d₆) δ: 13.28 (br s, 1H), 10.13 (br s, 1H), 9.11 (br t, J=4.8 Hz, 1H), 8.91-9.07 (m, 2H), 8.45 (d, J=8.4 Hz, 1H), 8.07 (d, J=8.8 Hz, 1H), 8.00 (d, J=8.5 Hz, 2H), 7.83-7.97 (m, 4H), 7.67 (t, J=7.7 Hz, 1H), 7.22 (d, J=8.6 Hz, 2H), 7.02 (s, 1H), 3.72 (q, J=5.8 Hz, 2H), 3.57 (br t, J=4.0 Hz, 4H), 3.15-3.34 (m, 8H), 2.85 (q, J=6.5 Hz, 2H), 2.77 (d, J=3.2 Hz, 3H), 2.69-2.72 (m, 1H), 2.61-2.68 (m, 1H), 2.05 (d, J=1.3 Hz, 4H). MS m/z (M+H)=433.50.

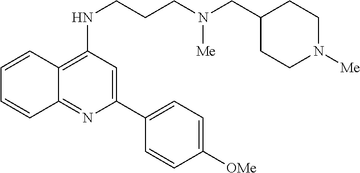 [SMILES: COc1ccc(-c2cc(NCCCN(C)CC3CCN(C)CC3)c3ccccc3n2)cc1]

Example 30: N¹-(2-(4-methoxyphenyl)quinolin-4-yl)-N³-methyl-N³-((1-methylpiperidin-4-yl)methyl)propane-1,3-diamine. To a suspension of N-(3-((piperidin-4-yl)methylamino)propyl)-2-(4-methoxyphenyl)quinolin-4-amine hydrochloride in dichloroethane was added excess of formaldehyde 37% w/v. After stirring for 30 minutes, to the clear solution was added sodium triacetoxyborohydride (50 mg, 0.23 mmol) in one portion. After 4 hours, the reaction was quenched with saturated ammonium chloride and partitioned between water and dichloromethane and the layers were separated. The aqueous layer was extracted with dichloromethane (2×10 mL) and the combined organic layers were washed with brine, dried over sodium sulfate decanted and concentrated under vacuum. The crude product was purified on silica gel cartridge (4 g, 60-120 mesh), eluting with dichloromethane/methanol with 5% ammonium hydroxide to give N-(3-((1-methylpiperidin-4-yl)methylamino)propyl)-2-(4-methoxyphenyl)quinolin-4-amine as a pale milky glass (15 mg, 20%). ¹H NMR (CDCl₃) δ: 8.05 (d, J=8.8 Hz, 2H), 7.63-7.69 (m, 1H), 7.56-7.63 (m, 1H), 7.29-7.38 (m, 1H), 7.26 (s, 1H), 7.01 (d, J=8.8 Hz, 2H), 6.76 (s, 1H), 3.86 (s, 3H), 3.47 (q, J=5.5 Hz, 2H), 2.75 (br d, J=11.2 Hz, 2H), 2.54-2.61 (m, 2H), 2.34 (s, 4H), 2.16-2.31 (m, 5H), 1.70-2.02 (m, 6H), 1.40-1.40 (m, 1H), 1.12-1.34 (m, 2H). MS m/z (M+H)=433.41.

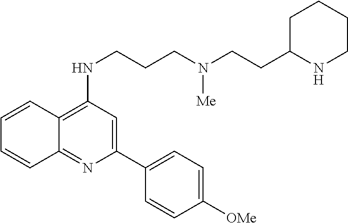 [SMILES: COc1ccc(-c2cc(NCCCN(C)CCC3CCCCN3)c3ccccc3n2)cc1]

### Example 31: N¹-(2-(4-methoxyphenyl)quinolin-4-yl)-N³-methyl-N³-(2-(piperidin-2-yl)ethyl)propane-1,3-diamine

Step 1: To a solution of 2-(aminoethyl)-1-N-boc-piperidine (0.080 g, 0.17 mmol) in acetonitrile (0.5 mL) was added triethylamine (0.036 mL, 0.26 mmol) followed by IA (0.08 g, 0.17 mmol). The reaction was carried out following the same procedure as Example 1. tert-butyl 2-(2-((3-((2-(4-methoxyphenyl)quinolin-4-yl)amino)propyl)amino)ethyl)piperidine-1-carboxylate was obtained as a pale brown oil (52 mg, 59%) and used without further purification.

Step 2: tert-Butyl 2-(2-((3-((2-(4-methoxyphenyl)quinolin-4-yl)amino)propyl)(methyl)amino)ethyl) piperidine-1-carboxylate: To a solution of tert-butyl 2-(2-((3-((2-(4-methoxyphenyl)quinolin-4-yl)amino)propyl)amino)ethyl) piperidine-1-carboxylate (0.052 g, 0.1 mmol) in dimethylformamide (0.5 mL) was added potassium carbonate (0.021 mg, 0.15 mmol). Methyl iodide (0.006 μL, 0.1 mmol) was then added and the reaction was stirred at 23° C. overnight. Water was added to the resulting reaction mixture and aqueous layer was extracted three times with dichloromethane, dried over magnesium sulfate and filtered. The reaction mixture was purified through silica gel cartridge eluting with dichloromethane/methanol with 5% ammonium hydroxide to give tert-butyl 2-(2-((3-((2-(4-methoxyphenyl)quinolin-4-yl)amino)propyl)(methyl)amino)ethyl)piperidine-1-carboxylate as a colorless oil (13 mg, 24%). ¹H NMR (300 MHz, CDCl₃) δ 8.05 (d, J=9.0 Hz, 2H), 8.03-7.99 (m, 1H), 7.80 (d, J=8.3 Hz, 1H), 7.65-7.56 (m, 1H), 7.54-7.39 (m, 1H), 7.39-7.27 (m, 1H), 7.00 (d, J=8.8 Hz, 2H), 6.74 (s, 1H), 3.86 (s, 3H), 3.57-3.41 (m, 2H), 2.94-2.82 (m, 1H), 2.74 (br d, J=2.4 Hz, 1H), 2.61 (br t, J=6.1 Hz, 2H), 2.36 (s, 3H), 2.14-1.85 (m, 5H), 1.76-1.53 (m, 6H), 1.45 (s, 9H), 1.43 (s, 2H). MS m/z (M+H)=533.3.
Step 3: To a solution of tert-butyl 2-(2-((3-((2-(4-methoxyphenyl)quinolin-4-yl)amino)propyl)(methyl)amino)ethyl)piperidine-1-carboxylate (13 mg, 0.024 mmol) in diethyl ether (1 mL) was added excess of hydrogen chloride solution in diethyl ether. The reaction mixture was stirred overnight at 23° C. The solvent was evaporated and the resulting powder was dried under vacuum to give N¹-(2-(4-methoxyphenyl)quinolin-4-yl)-N³-methyl-N³-(2-(piperidin-2-yl)ethyl)propane-1,3-diamine dihydrochloride as a white powder (10 mg, 97%). ¹H NMR (300 MHz, DMSO-d₆) δ 13.59 (s, 1H), 9.63-9.48 (m, 1H), 9.44-9.22 (m, 2H), 8.74-8.61 (m, 1H), 8.19 (d, J=8.5 Hz, 1H), 8.11 (d, J=8.8 Hz, 2H), 7.99-7.88 (m, 1H), 7.78-7.59 (m, 1H), 7.20 (d, J=8.9 Hz, 1H), 7.08 (s, 1H), 3.88 (s, 3H), 3.85-3.74 (m, 2H), 3.58 (s, 1H), 3.25-3.11 (m, 4H), 3.09-2.97 (m, 3H), 2.83-2.67 (m, 2H), 2.23-2.03 (m, 3H), 2.01-1.88 (m, 1H), 1.86-1.78 (m, 1H), 1.73-1.71 (m, 3H), 1.47-1.33 (m, 2H), 1.21 (s, 1H). MS m/z (M+H)=433.2.

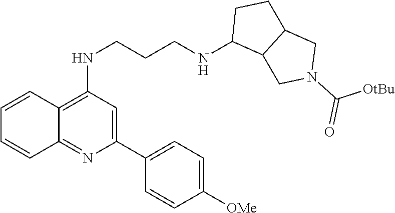 [SMILES: COc1ccc(-c2cc(NCCCNC3CCC4CN(C(=O)OC(C)(C)C)CC43)c3ccccc3n2)cc1]

Example 32: tert-Butyl(2-(3-((2-(4-methoxyphenyl)quinolin-4-yl)amino)propyl)octahydrocyclopenta[c]pyrrol-4-yl)carbamate. To a solution of tert-butyl octahydrocyclopenta[c]pyrrol-4-yl carbamate (0.059 g, 0.26 mmol) in acetonitrile (0.5 mL) was added triethylamine (0.036 μL, 0.26 mmol) followed by IA (0.08 g, 0.17 mmol). The reaction was carried out following the same procedure as Example 1. tert-Butyl(2-(3-((2-(4-methoxyphenyl)quinolin-4-yl)amino)propyl)octahydrocyclopenta[c]pyrrol-4-yl)carbamate was obtained as pale yellow powder (40 mg, 46%). ¹H NMR (300 MHz, CDCl₃) δ 8.05 (d, J=8.8 Hz, 2H), 7.71 (br d, J=8.4 Hz, 1H), 7.66-7.54 (m, 1H), 7.44-7.30 (m, 1H), 7.01 (d, J=8.8 Hz, 1H), 6.97-6.87 (m, 1H), 6.84-6.72 (m, 1H), 6.26 (s, 1H), 5.63 (br d, J=7.7 Hz, 1H), 3.87 (s, 3H), 3.53-3.48 (m, 3H), 2.81 (br s, 1H), 2.73-2.55 (m, 4H), 2.49 (br d, J=5.7 Hz, 2H), 2.37-2.25 (m, 1H), 1.96 (br t, J=6.1 Hz, 2H), 1.88-1.79 (m, 1H), 1.75-1.61 (m, 3H), 1.51 (s, 1H), 1.48-1.32 (m, 9H). MS m/z (M+H)=517.3.

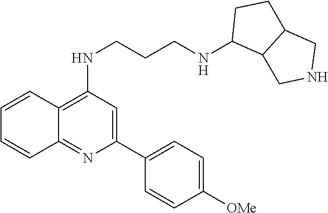 [SMILES: COc1ccc(-c2cc(NCCCNC3CCC4CNCC43)c3ccccc3n2)cc1]

Example 33: N¹-(2-(4-Methoxyphenyl)quinolin-4-yl)-N³-(octahydrocyclopenta[c]pyrrol-4-yl)propane-1,3-diamine dihydrochloride. To a solution of tert-butyl 4-((3-((2-(4-methoxyphenyl) quinolin-4-yl)amino)propyl)amino)hexahydrocyclopenta[c]pyrrole-2(1H)-carboxylate (49 mg, 0.094 mmol) in diethyl ether (1 mL) was added excess of hydrogen chloride solution in diethyl ether. The reaction mixture was stirred overnight at 23° C. The solvent was evaporated and the resulting powder was dried under vacuum to give N¹-(2-(4-methoxyphenyl)quinolin-4-yl)-N³-(octahydrocyclopenta[c]pyrrol-4-yl)propane-1,3-diamine dihydrochloride was obtained as a yellow powder (32 mg, 82%). ¹H NMR (300 MHz, DMSO-d₆) δ 13.66 (s, 1H), 9.59 (br d, J=6.2 Hz, 1H), 8.76-8.66 (m, 1H), 8.59 (br s, 2H), 8.44 (br d, J=5.6 Hz, 1H), 8.26 (d, J=8.7 Hz, 1H), 8.14 (d, J=8.8 Hz, 2H), 7.92 (t, J=7.7 Hz, 1H), 7.66 (d, J=12.3 Hz, 1H), 7.19 (d, J=8.7 Hz, 2H), 7.06 (d, J=3.3 Hz, 1H), 3.87 (s, 3H), 3.78 (br s, 3H), 3.63-3.44 (m, 2H), 3.30-3.17 (m, 2H), 3.16-2.63 (m, 4H), 2.32-2.07 (m, 2H), 2.05-1.42 (m, 4H). MS m/z (M+H)=417.2.

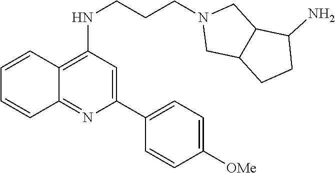 [SMILES: COc1ccc(-c2cc(NCCCN3CC4CCC(N)C4C3)c3ccccc3n2)cc1]

Example 34: N-(3-(4-Aminohexahydrocyclopenta[c]pyrrol-2(1H)-yl)propyl)-2-(4-methoxyphenyl)quinolin-4-amine trihydrochloride. To a solution of tert-butyl(2-(3-((2-(4-methoxy phenyl)quinolin-4-yl)amino)propyl)octahydrocyclopenta[c]pyrrol-4-yl)carbamate (40 mg, 0.077 mmol) in diethyl ether (1 mL) was added excess of hydrogen chloride solution in diethyl ether. The reaction mixture was stirred overnight at 23° C. The solvent was evaporated and the resulting powder was dried under vacuum to give N-(3-(4-aminohexahydrocyclopenta[c]pyrrol-2(1H)-yl)propyl)-2-(4-methoxyphenyl)quinolin-4-amine trihydrochloride as a pale yellow powder (39 mg, 100%). ¹H NMR (300 MHz, DMSO-d₆) δ 13.66 (s, 1H), 11.46-11.17 (m, 1H), 9.58 (br s, 1H), 8.69 (br d, J=8.1 Hz, 1H), 8.59 (s, 2H), 8.48-8.37 (m, 1H), 8.27 (d, J=8.6 Hz, 1H), 8.14 (d, J=8.7 Hz, 2H), 7.92 (t, J=7.7 Hz, 1H), 7.65 (t, J=7.7 Hz, 1H), 7.19 (d, J=8.7 Hz, 2H), 7.06 (d, J=3.2 Hz, 1H), 3.87 (s, 3H), 3.79 (br s, 3H), 3.63-3.41 (m, 2H), 3.28-3.25 (m, 3H), 3.13-2.60 (m, 3H), 2.21-2.15 (m, 2H), 2.07-1.40 (m, 4H). MS m/z (M+H)=417.48.

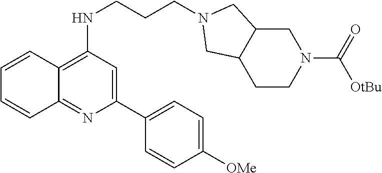 [SMILES: COc1ccc(-c2cc(NCCCN3CC4CCN(C(=O)OC(C)(C)C)CC4C3)c3ccccc3n2)cc1]

Example 35: tert-Butyl 2-(3-((2-(4-methoxyphenyl)quinolin-4-yl)amino)propyl) hexahydro-1H-pyrrolo[3,4-c]pyridine-5(6H)-carboxylate. To a solution of tert-butyl hexahydro-1H-pyrrolo[3,4-c]pyridine-2(3H)-carboxylate (0.059 g, 0.26 mmol) in acetonitrile (0.5 mL) was added triethylamine (0.036 mL, 0.26 mmol) followed by IA (0.08 g, 0.17 mmol). The reaction mixture was stirred overnight at 23° C. The reaction mixture was diluted in ethyl acetate, washed with saturated aqueous sodium bicarbonate. Aqueous layer was extracted three times with ethyl acetate, dried over magnesium sulfate and filtered. The resultant crude product was purified through silica gel cartridge eluting with dichloromethane/methanol with 5% ammonium hydroxide to give tert-butyl 2-(3-((2-(4-methoxyphenyl)quinolin-4-yl)amino)propyl)hexahydro-1H-pyrrolo[3,4-c]pyridine-5(6H)-carboxylate (45 mg, 52%). ¹H NMR (300 MHz, CDCl₃) δ 8.05 (d, J=8.8 Hz, 2H), 8.01 (s, 1H), 7.67 (d, J=8.3 Hz, 1H), 7.60 (t, J=8.4 Hz, 1H), 7.41-7.30 (m, 1H), 7.06-6.96 (m, 2H), 6.76 (s, 1H), 3.87 (s, 3H), 3.67-3.41 (m, 4H), 3.37-3.16 (m, 2H), 3.10-3.07 (m, 2H), 2.82-2.70 (m, 2H), 2.62-2.40 (m, 2H), 2.26 (br s, 1H), 1.97 (quin, J=5.8 Hz, 2H), 1.90-1.57 (m, 4H), 1.47 (s, 9H). MS m/z (M+H)=517.2.

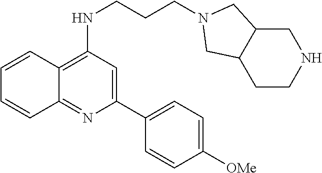 [SMILES: COc1ccc(-c2cc(NCCCN3CC4CCNCC4C3)c3ccccc3n2)cc1]

Example 36: N-(3-(Hexahydro-1H-pyrrolo[3,4-c]pyridin-2(3H)-yl)propyl)-2-(4-methoxy phenyl)quinolin-4-amine dihydrochloride. To a solution of tert-butyl 2-(3-((2-(4-methoxyphenyl)quinolin-4-yl)amino)propyl)hexahydro-1H-pyrrolo[3,4-c]pyridine-5(6H)-carboxylate (45 mg, 0.087 mmol) in diethyl ether (1 mL) was added excess of hydrogen chloride solution in diethyl ether. The reaction mixture was stirred overnight at 23° C. The solvent was evaporated and the resulting powder was dried under vacuum to give N-(3-(hexahydro-1H-pyrrolo[3,4-c]pyridin-2(3H)-yl)propyl)-2-(4-methoxyphenyl)quinolin-4-amine dihydrochloride as a pale yellow powder (35 mg, 97%). ¹H NMR (300 MHz, DMSO-d₆) δ 13.67 (s, 1H), 11.48 (s, 1H), 9.63-9.44 (m, 2H), 8.79-8.63 (m, 1H), 8.33-8.18 (m, 1H), 8.13 (d, J=8.8 Hz, 2H), 8.01-7.88 (m, 1H), 7.66 (s, 1H), 7.19 (d, J=8.8 Hz, 2H), 7.07 (s, 1H), 3.87 (s, 3H), 3.84-3.73 (m, 2H), 3.58 (br s, 2H), 3.51-3.38 (m, 1H), 3.14 (br s, 4H), 3.04-2.91 (m, 2H), 2.83-2.69 (m, 1H), 2.68-2.57 (m, 1H), 2.27-2.09 (m, 2H), 1.90-1.73 (m, 3H). MS m/z (M+H)=417.48.

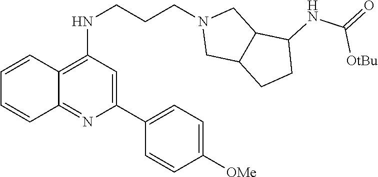 [SMILES: COc1ccc(-c2cc(NCCCN3CC4CCC(NC(=O)OC(C)(C)C)C4C3)c3ccccc3n2)cc1]

Example 37: tert-Butyl 4-((3-((2-(4-methoxyphenyl)quinolin-4-yl)amino)propyl)amino)hexahydrocyclopenta[c]pyrrole-2(1H)-carboxylate. To a solution of 4-amino(hexahydrocyclopenta[c]pyrrole)-2-carboxylic acid tert-butyl ester (0.111 g, 0.49 mmol) in acetonitrile (1 mL) was added triethylamine (0.069 mL, 0.49 mmol) followed by IA (0.15 g, 0.32 mmol). The reaction was carried out following the same procedure as Example 1. tert-Butyl 4-((3-((2-(4-methoxyphenyl)quinolin-4-yl)amino)propyl)amino)hexahydrocyclopenta[c]pyrrole-2(1H)-carboxylate was obtained as a clear oil (49 mg, 30%). ¹H NMR (300 MHz, CDCl₃) δ 8.05 (d, J=8.7 Hz, 1H), 7.71 (br d, J=8.4 Hz, 1H), 7.66-7.55 (m, 1H), 7.44-7.31 (m, 1H), 7.01 (d, J=8.5 Hz, 2H), 6.92 (s, 1H), 6.85-6.73 (m, 1H), 6.26 (br s, 1H), 3.87 (s, 3H), 3.54-3.47 (m, 2H), 2.81-2.80 (m, 2H), 2.72-2.56 (m, 4H), 2.54-2.43 (m, 2H), 2.04-1.89 (m, 3H), 1.90-1.80 (m, 1H), 1.79-1.62 (m, 3H), 1.51 (br s, 2H), 1.44-1.39 (m, 9H). MS m/z (M+H)=517.3.

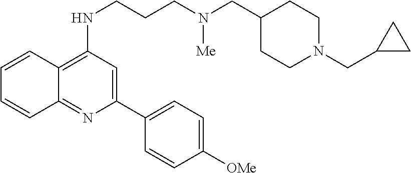 [SMILES: COc1ccc(-c2cc(NCCCN(C)CC3CCN(CC4CC4)CC3)c3ccccc3n2)cc1]

Example 38: N¹-((1-(Cyclopropylmethyl)piperidin-4-yl)methyl)-N³-(2-(4-methoxy phenyl)quinolin-4-yl)-N¹-methylpropane-1,3-diamine. To a solution of N¹-((1-(cyclopropyl methyl)piperidin-4-yl)methyl)-N³-(2-(4-methoxyphenyl)quinolin-4-yl)propane-1,3-diamine (0.038 g, 0.083 mmol) in dimethylformamide (0.5 mL) was added potassium carbonate (0.018 mg, 0.12 mmol). Methyl iodide (0.005 μL, 0.083 mmol) was then added and the reaction was stirred at 23° C. overnight. Water was added to the resulting reaction mixture and aqueous layer was extracted three times with dichloromethane, dried over magnesium sulfate and filtered. The reaction mixture was purified through silica gel cartridge eluting with dichloromethane/methanol with 5% ammonium hydroxide to give N¹-((1-(cyclopropylmethyl)piperidin-4-yl)methyl)-N³-(2-(4-methoxyphenyl) quinolin-4-yl)-N¹-methylpropane-1,3-diamine. ¹H NMR (300 MHz, CDCl₃) δ 8.06 (d, J=8.7 Hz, 2H), 8.03-7.98 (m, 1H), 7.71-7.64 (m, 1H), 7.60 (t, J=8.4 Hz, 1H), 7.39-7.30 (m, 1H), 7.08 (br s, 1H), 7.01 (d, J=8.7 Hz, 2H), 6.77 (s, 1H), 3.87 (s, 3H), 3.49 (q, J=5.5 Hz, 2H), 2.97 (br d, J=11.2 Hz, 2H), 2.65-2.54 (m, 2H), 2.35 (s, 3H), 2.28 (d, J=6.8 Hz, 2H), 2.18 (d, J=6.5 Hz, 2H), 2.01-1.90 (m, 2H), 1.87-1.71 (m, 4H), 1.34-1.18 (m, 3H), 0.89-0.69 (m, 1H), 0.50-0.42 (m, 2H), 0.11-0.02 (m, 2H). MS m/z (M+H) 473.27.

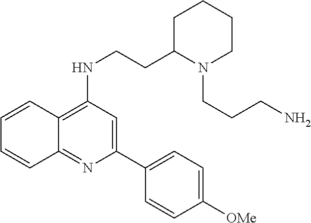 [SMILES: COc1ccc(-c2cc(NCCC3CCCCN3CCCN)c3ccccc3n2)cc1]

Example 39: N-(2-(1-(3-Aminopropyl)piperidin-2-yl)ethyl)-2-(4-methoxyphenyl) quinolin-4-amine dihydrochloride. To a solution of 2-(3-(2-(2-((2-(4-methoxyphenyl)quinolin-4-yl)amino)ethyl)piperidin-1-yl)propyl)isoindoline-1,3-dione (0.020 g, 0.036 mmol) in ethanol (1 mL) was added hydrazine hydrate (2 μL, 0.044 mmol). The reaction was stirred at 23° C. overnight. Water was added to the resulting reaction mixture and aqueous layer was extracted three times with dichloromethane, dried over magnesium sulfate and filtered. The reaction mixture was purified through silica gel cartridge eluting with dichloromethane/methanol with 5% ammonium hydroxide to give N-(2-(1-(3-aminopropyl)piperidin-2-yl)ethyl)-2-(4-methoxyphenyl)quinolin-4-amine. The obtained oil was dissolved in diethyl ether and excess of hydrogen chloride solution in diethyl ether was added. The solvent was evaporated and the resulting powder was dried under vacuum to give N-(2-(1-(3-aminopropyl)piperidin-2-yl)ethyl)-2-(4-methoxyphenyl)quinolin-4-amine dihydrochloride as a white powder (16 mg, 100%). ¹H NMR (300 MHz, DMSO-d₆) δ 13.67 (br d, J=8.3 Hz, 1H), 10.95 (s, 1H), 9.72-9.53 (m, 1H), 8.78 (t, J=8.4 Hz, 1H), 8.33-8.18 (m, 3H), 8.15 (dd, J=8.9, 2.3 Hz, 2H), 7.92 (t, J=7.7 Hz, 1H), 7.71-7.59 (m, 1H), 7.20 (d, J=8.8 Hz, 2H), 7.06 (d, J=9.0 Hz, 1H), 3.88 (s, 3H), 3.63-3.46 (m, 2H), 3.45-3.17 (m, 4H), 2.98-2.80 (m, 3H), 2.17-1.98 (m, 4H), 1.85-1.62 (m, 5H), 1.58-1.40 (m, 1H). MS m/z (M+H)=419.3.

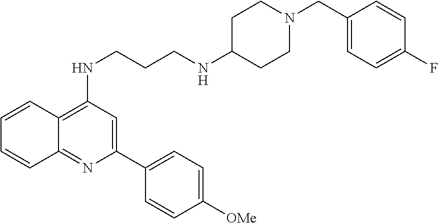 [SMILES: COc1ccc(-c2cc(NCCCNC3CCN(Cc4ccc(F)cc4)CC3)c3ccccc3n2)cc1]

Example 40: N¹-(1-(4-Fluorobenzyl)piperidin-4-yl)-N³-(2-(4-methoxyphenyl)quinolin-4-yl)propane-1,3-diamine. To a solution of 1-[(4-Fluorophenyl)methyl]piperidin-4-amine (0.054 g, 0.26 mmol) in acetonitrile (0.5 mL) was added triethylamine (0.036 μL, 0.26 mmol) followed by IA (0.08 g, 0.17 mmol). The resulting reaction mixture was stirred at 23° C. over a period of 16 hours. The reaction mixture was then diluted with ethyl acetate (5 mL), washed with sodium bicarbonate (5 mL). Aqueous layer was extracted three times with ethyl acetate, dried over magnesium sulfate and filtered. The resultant crude product was purified through silica gel cartridge with dichloromethane/methanol with 5% ammonium hydroxide to give N¹-(1-(4-fluorobenzyl)piperidin-4-yl)-N³-(2-(4-methoxyphenyl)quinolin-4-yl)propane-1,3-diamine as a white powder (37 mg, 44%). ¹H NMR (CDCl₃) δ: 7.99-8.08 (m, 3H), 7.79 (d, J=8.3 Hz, 1H), 7.55-7.65 (m, 1H), 7.31-7.40 (m, 2H), 7.21-7.30 (m, 2H), 7.01 (d, J=8.7 Hz, 4H), 6.73 (s, 1H), 3.86 (s, 3H), 3.43-3.46 (m, 4H), 2.92 (t, J=5.5 Hz, 2H), 2.84 (br d, J=11.6 Hz, 2H), 2.42-2.55 (m, 1H), 1.89-2.07 (m, 6H), 1.74 (s, 1H), 1.37-1.55 (m, 2H). MS m/z (M+H)=499.2.

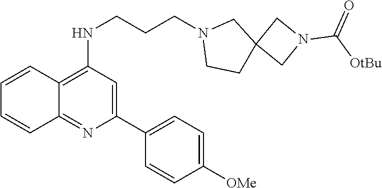 [SMILES: COc1ccc(-c2cc(NCCCN3CCC4(C3)CN(C(=O)OC(C)(C)C)C4)c3ccccc3n2)cc1]

Example 41: tert-Butyl 6-(3-((2-(4-methoxyphenyl)quinolin-4-yl)amino)propyl)-2,6-diaza spiro[3.4]octane-2-carboxylate. To a solution of tert-butyl 2,6-diazaspiro[3.4]octane-2-carboxylate (0.055 g, 0.26 mmol) in acetonitrile (0.5 mL) was added triethylamine (0.036 μL, 0.26 mmol) followed by IA (0.08 g, 0.17 mmol). The resulting reaction mixture was stirred at 23° C. over a period of 16 hours. The reaction mixture was then diluted with ethyl acetate (5 mL), washed with sodium bicarbonate (5 mL). Aqueous layer was extracted three times with ethyl acetate, dried over magnesium sulfate and filtered. The resultant crude product was purified through silica gel cartridge with dichloromethane/methanol with 5% ammonium hydroxide to give tert-butyl 6-(3-((2-(4-methoxyphenyl)quinolin-4-yl)amino)propyl)-2,6-diazaspiro[3.4]octane-2-carboxylate as a yellow oil (59 mg, 69%). ¹H NMR (DMSO-d₆) δ: 13.38-13.75 (m, 1H), 11.19-11.56 (m, 1H), 9.44-9.53 (m, 1H), 8.66 (d, J=8.4 Hz, 1H), 8.22 (d, J=8.5 Hz, 1H), 8.13 (d, J=8.9 Hz, 2H), 7.92 (s, 1H), 7.66 (s, 1H), 7.19 (d, J=8.8 Hz, 2H), 7.07 (s, 1H), 3.87 (s, 3H), 3.66-3.83 (m, 6H), 3.46-3.61 (m, 1H), 3.17-3.29 (m, 4H), 2.99-3.14 (m, 1H), 2.15 (br d, J=6.6 Hz, 4H), 1.35 (s, 9H). MS m/z (M+H)=503.3.

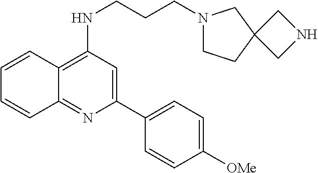 [SMILES: COc1ccc(-c2cc(NCCCN3CCC4(CNC4)C3)c3ccccc3n2)cc1]

Example 42: N-(3-(2,6-Diazaspiro[3.4]octan-6-yl)propyl)-2-(4-methoxyphenyl)quinolin-4-amine. To a solution of tert-butyl 6-(3-((2-(4-methoxyphenyl)quinolin-4-yl)amino)propyl)-2,6-diazaspiro[3.4]octane-2-carboxylate (59 mg, 0.011 mmol) in diethyl ether (1 mL) was added excess of hydrogen chloride solution in diethyl ether. The reaction mixture was stirred overnight at 23° C. The solvent was evaporated and the resulting powder was dried under vacuum to give N-(3-(2,6-diazaspiro[3.4]octan-6-yl)propyl)-2-(4-methoxyphenyl)quinolin-4-amine was obtained as a pale yellow powder (25 mg, 57%). ¹H NMR (DMSO-d₆) δ: 13.68 (s, 1H), 11.53 (s, 1H), 9.58 (br t, J=5.6 Hz, 1H), 9.38 (br s, 1H), 8.71 (d, J=8.5 Hz, 1H), 8.27 (d, J=8.5 Hz, 1H), 8.14 (d, J=8.7 Hz, 2H), 7.92 (t, J=7.7 Hz, 1H), 7.60-7.70 (m, 1H), 7.19 (d, J=8.7 Hz, 2H), 7.06 (s, 1H), 4.05-4.17 (m, 1H), 3.91-4.03 (m, 2H), 3.87 (s, 3H), 3.72-3.84 (m, 3H), 3.54 (s, 3H), 3.17-3.36 (m, 3H), 3.00-3.15 (m, 1H), 2.22-2.34 (m, 1H), 2.06-2.20 (m, 2H). MS m/z (M+H)=403.2.

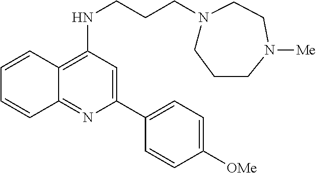 [SMILES: COc1ccc(-c2cc(NCCCN3CCCN(C)CC3)c3ccccc3n2)cc1]

Example 43: 2-(4-Methoxyphenyl)-N-(3-(4-methyl-1,4-diazepan-1-yl)propyl)quinolin-4-amine. To a solution of 1-methylhomopiperazine (0.033 μL, 0.26 mmol) in acetonitrile (0.5 mL) was added triethylamine (0.036 μL, 0.26 mmol) followed by IA (0.08 g, 0.17 mmol). The resulting reaction mixture was stirred at 23° C. over a period of 16 hours. The reaction mixture was then diluted with ethyl acetate (5 mL), washed with sodium bicarbonate (5 mL). Aqueous layer was extracted three times with ethyl acetate, dried over magnesium sulfate and filtered. The resultant crude product was purified through silica gel cartridge with dichloromethane/methanol with 5% ammonium hydroxide to give 2-(4-methoxyphenyl)-N-(3-(4-methyl-1,4-diazepan-1-yl)propyl)quinolin-4-amine as a white powder (32 mg, 47%). ¹H NMR (CDCl₃) δ: 7.99-8.09 (m, 3H), 7.87 (d, J=8.3 Hz, 1H), 7.57-7.65 (m, 1H), 7.36 (dd, J=8.5, 7.0 Hz, 1H), 7.32 (s, 1H), 7.01 (d, J=8.5 Hz, 2H), 6.73 (s, 1H), 3.86 (d, J=1.0 Hz, 3H), 3.44 (d, J=0.9 Hz, 3H), 2.79-2.90 (m, 4H), 2.67-2.77 (m, 6H), 2.45 (br d, J=4.9 Hz, 3H), 1.91 (sxt, J=6.0 Hz, 3H). MS m/z (M+H)=405.5.

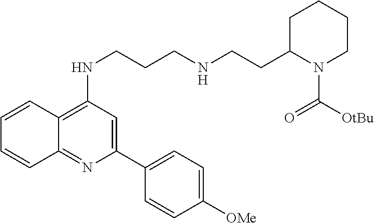 [SMILES: COc1ccc(-c2cc(NCCCNCCC3CCCCN3C(=O)OC(C)(C)C)c3ccccc3n2)cc1]

Example 44: tert-Butyl 2-(2-((3-((2-(4-methoxyphenyl)quinolin-4-yl)amino)propyl)amino)ethyl)piperidine-1-carboxylate. To a solution of 2-(aminoethyl)-1-N-boc-piperidine (0.060 mg, 0.26 mmol) in acetonitrile (0.5 mL) was added triethylamine (0.036 μL, 0.26 mmol) followed by IA (0.08 g, 0.17 mmol). The resulting reaction mixture was stirred at 23° C. over a period of 16 hours. The reaction mixture was then diluted with ethyl acetate (5 mL), washed with sodium bicarbonate (5 mL). Aqueous layer was extracted three times with ethyl acetate, dried over magnesium sulfate and filtered. The resultant crude product was purified through silica gel cartridge with dichloromethane/methanol with 5% ammonium hydroxide to give tert-butyl 2-(2-((3-((2-(4-methoxyphenyl)quinolin-4-yl)amino)propyl)amino)ethyl)piperidine-1-carboxylate as a white oil (66 mg, 75%). ¹H NMR (CDCl₃) δ: 8.04-8.09 (m, 2H), 8.01 (d, J=8.5 Hz, 1H), 7.80 (d, J=8.3 Hz, 1H), 7.59 (t, J=7.7 Hz, 1H), 7.32 (t, J=7.5 Hz, 1H), 7.01 (d, J=8.7 Hz, 2H), 6.75 (s, 1H), 4.25-4.48 (m, 2H), 3.92-4.09 (m, 2H), 3.87 (s, 3H), 3.69-3.81 (m, 2H), 3.49 (s, 2H), 2.88 (s, 2H), 2.55-2.69 (m, 4H), 1.90-2.04 (m, 3H), 1.79-1.88 (m, 2H), 1.45 (s, 9H). MS m/z (M+H)=519.3.

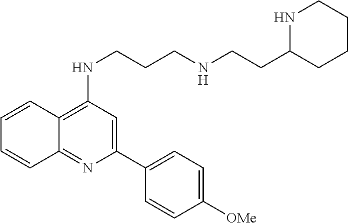 [SMILES: COc1ccc(-c2cc(NCCCNCCC3CCCCN3)c3ccccc3n2)cc1]

Example 45: N¹-(2-(4-methoxyphenyl)quinolin-4-yl)-N³-(2-(piperidin-2-yl)ethyl)propane-1,3-diamine To a solution of tert-butyl 2-(2-((3-((2-(4-methoxyphenyl)quinolin-4-yl)amino)propyl)amino)ethyl)piperidine-1-carboxylate (66 mg, 0.13 mmol) in diethyl ether (1 mL) was added excess of hydrogen chloride solution in diethyl ether. The reaction mixture was stirred overnight at 23° C. The solvent was evaporated and the resulting powder was dried under vacuum to give N¹-(2-(4-methoxyphenyl)quinolin-4-yl)-N³-(2-(piperidin-2-yl)ethyl)propane-1,3-diamine as a white powder (59 mg, 100%). ¹H NMR (MeOH-d₄) δ: 8.46-8.55 (m, 1H), 8.02 (d, J=8.4 Hz, 3H), 7.89-7.98 (m, 1H), 7.65-7.75 (m, 1H), 7.21 (d, J=8.2 Hz, 2H), 7.07 (s, 1H), 3.92 (s, 3H), 3.82-3.90 (m, 2H), 3.34-3.46 (m, 2H), 3.24 (br d, J=9.9 Hz, 3H), 3.08 (br d, J=8.8 Hz, 3H), 2.95-3.04 (m, 2H), 2.24-2.36 (m, 1H), 1.98-2.11 (m, 4H), 1.88 (s, 5H). MS m/z (M+H)=419.2.

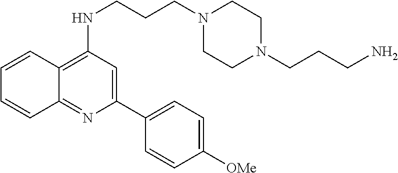 [SMILES: COc1ccc(-c2cc(NCCCN3CCN(CCCN)CC3)c3ccccc3n2)cc1]

Example 46: N-(3-(4-(3-Aminopropyl)piperazin-1-yl)propyl)-2-(4-methoxyphenyl)quinolin-4-amine hydrochloride. To a solution of 4-chloro-2-(4-methoxyphenyl)quinoline (0.1 g, 0.37 mmol) with 1,4-bis(3-aminopropyl)piperazine (0.914 mL, 4.44 mmol) was added two drops of tin tetrachloride. The reaction was carried out following the same procedure as step 2 of the synthesis of intermediate A (IA). The obtained oil was dissolved in diethyl ether and excess of hydrogen chloride solution in diethyl ether was added. The solvent was evaporated and the resulting powder was dried under vacuum to give N-(3-(4-(3-aminopropyl)piperazin-1-yl)propyl)-2-(4-methoxyphenyl)quinolin-4-amine hydrochloride as a pale yellow powder (48 mg, 30%). ¹H NMR (300 MHz, DMSO-d₆) δ 13.66 (s, 1H), 9.52 (s, 1H), 8.67 (br d, J=8.4 Hz, 1H), 8.26 (d, J=8.5 Hz, 1H), 8.13 (br d, J=8.4 Hz, 2H), 7.93 (t, J=7.7 Hz, 1H), 7.75-7.60 (m, 1H), 7.20 (d, J=8.7 Hz, 2H), 7.07 (s, 1H), 3.87 (s, 3H), 3.83-3.71 (m, 4H), 3.70-3.52 (m, 4H), 3.30-3.05 (m, 4H), 2.89 (br d, J=7.7 Hz, 3H), 2.32-2.14 (m, 2H), 2.01 (br s, 2H), 1.80-1.69 (m, 1H), 1.21 (s, 2H). MS m/z (M+H)=434.4.

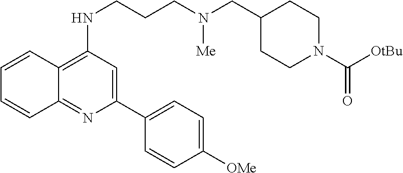 [SMILES: COc1ccc(-c2cc(NCCCN(C)CC3CCN(C(=O)OC(C)(C)C)CC3)c3ccccc3n2)cc1]

Example 47: tert-Butyl 4-{[(3-{[2-(4-methoxyphenyl)quinolin-4-yl]amino}propyl)(methyl)amino]methyl}piperidine-1-carboxylate. To a suspension of 2-(4-methoxyphenyl)-N-(3-(methylamino)propyl)quinolin-4-amine hydrochloride (200 mg, 0.56 mmol) in dichloroethane (2 mL), was added tert-butyl 4-formylpiperidine-1-carboxylate (119 mg, 0.56 mmol) and acetic acid (32 uL, 0.56 mmol). The mixture was stirred for 30 min at 23° C., then sodium triacetoxyborohydride (154 mg, 0.73 mmol) was added in one portion. Upon completion the reaction was quenched with aqueous sodium bicarbonate (5%) and extracted with dichloromethane (3×20 mL) and the combined organics were dried over sodium sulfate, decanted and concentrated under vacuum. The crude product was purified on silica gel cartridge (12 g, 60-120 mesh), eluting with dichloromethane/methanol with 5% ammonium hydroxide to give tert-butyl 4-{[(3-{[2-(4-methoxyphenyl)quinolin-4-yl]amino}propyl)(methyl)amino]methyl}piperidine-1-carboxylate as a clear film (99 mg, 34%). ¹H NMR (CDCl₃) δ: 8.32 (br d, J=8.6 Hz, 2H), 7.98 (d, J=8.6 Hz, 3H), 7.54-7.65 (m, 1H), 7.31-7.41 (m, 1H), 7.00 (d, J=8.7 Hz, 2H), 6.52 (s, 1H), 3.85 (s, 3H), 3.72 (q, J=7.0 Hz, 1H), 3.53 (br t, J=6.1 Hz, 2H), 2.77 (br t, J=6.3 Hz, 2H), 2.65 (br t, J=12.8 Hz, 3H), 2.44 (s, 5H), 2.07 (br d, J=6.0 Hz, 2H), 1.76 (br d, J=12.1 Hz, 3H), 1.42 (s, 9H), 1.24 (t, J=7.0 Hz, 2H), 1.02-1.19 (m, 2H). MS m/z (M+H)=519.07.

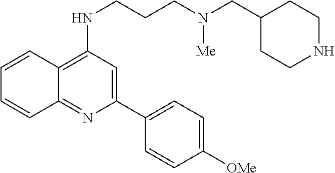 [SMILES: COc1ccc(-c2cc(NCCCN(C)CC3CCNCC3)c3ccccc3n2)cc1]

Example 48: N-(3-(N-Methyl-N-((piperidin-4-yl)methyl)amino)propyl)-2-(4-methoxy phenyl)quinolin-4-amine. To a solution of tert-butyl 4-{[(3-{[2-(4-methoxyphenyl)quinolin-4-yl]amino}propyl)(methyl)amino] methyl}piperidine-1-carboxylate (99 mg, 0.24 mmol) in 1,4-dioxane was added 4 N hydrogen chloride in 1,4-dioxane (0.5 mL) and the reaction was stirred at 23° C. for 2 hrs. The reaction mixture was concentrated under vacuum, dissolved in water, and dried on lyophilizer to give N-(3-(N-methyl-N-((piperidin-4-yl)methyl)amino)propyl)-2-(4-methoxyphenyl) quinolin-4-amine hydrochloride as a white solid (90 mg, 75%). ¹H NMR (MeOH-d₄) δ: 8.48-8.54 (m, 1H), 8.02 (d, J=8.9 Hz, 3H), 7.92-7.99 (m, 1H), 7.67-7.75 (m, 1H), 7.22 (d, J=8.9 Hz, 2H), 7.08 (s, 1H), 3.93 (s, 3H), 3.79-3.87 (m, 2H), 3.36-3.49 (m, 2H), 2.98-3.16 (m, 2H), 2.95 (s, 3H), 2.29-2.42 (m, 2H), 1.99-2.23 (m, 4H), 1.59 (m, 4H). MS m/z (M+H)=419.

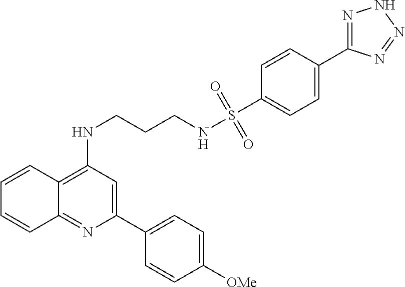 [SMILES: COc1ccc(-c2cc(NCCCNS(=O)(=O)c3ccc(-c4nn[nH]n4)cc3)c3ccccc3n2)cc1]

Example 49: N-(3-((2-(4-Methoxyphenyl)quinolin-4-yl)amino)propyl)-4-(2H-tetrazol-5-yl)benzene sulfonamide. To a solution of 4-(2H-tetrazol-5-yl)benzenesulfonamide (0.195 g, 0.86 mmol) in dimethylformamide (0.5 mL) was added pyridine (0.035 μL, 0.43 mmol) followed by IA (0.08 g, 0.17 mmol). The resulting reaction mixture was stirred at 23° C. over a period of 16 hours. The reaction mixture was then diluted with dichloromethane (5 mL), washed with water (5 mL). Aqueous layer was extracted three times with dichloromethane, dried over magnesium sulfate and filtered. The resultant crude product was purified through silica gel cartridge with dichloromethane/methanol with 5% ammonium hydroxide to give N-(3-((2-(4-methoxyphenyl)quinolin-4-yl)amino)propyl)-4-(2H-tetrazol-5-yl)benzenesulfonamide (3 mg, 4%). ¹H NMR (CDCl₃) δ: 8.16-8.22 (m, 2H), 7.99-8.07 (m, 2H), 7.92-7.98 (m, 4H), 7.57-7.68 (m, 3H), 7.34-7.41 (m, 1H), 6.96-7.04 (m, 1H), 6.92 (d, J=8.7 Hz, 2H), 6.77 (s, 1H), 4.85-4.96 (m, 2H), 3.84-3.90 (m, 3H), 3.56-3.66 (m, 2H), 2.49-2.63 (m, 2H). MS m/z (M+H)=516.2.

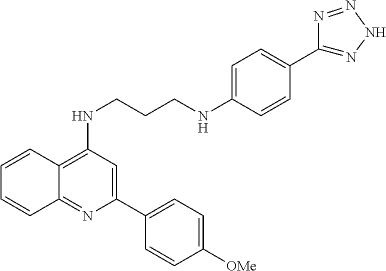 [SMILES: COc1ccc(-c2cc(NCCCNc3ccc(-c4nn[nH]n4)cc3)c3ccccc3n2)cc1]

Example 50: N¹-(4-(2H-Tetrazol-5-yl)phenyl)-N³-(2-(4-methoxyphenyl)quinolin-4-yl) propane-1,3-diamine. To a solution of 4-(2H-tetrazol-5-y)aniline (0.140 g, 0.86 mmol) in dimethylformamide (0.5 mL) was added pyridine (0.035 μL, 0.43 mmol) followed by IA (0.08 g, 0.17 mmol). The reaction was carried out following the same procedure as Example 1. The obtained oil was dissolved in diethyl ether and excess of hydrogen chloride solution in diethyl ether was added. The solvent was evaporated and the resulting powder was dried under vacuum. N¹-(4-(2H-tetrazol-5-yl)phenyl)-N³-(2-(4-methoxyphenyl)quinolin-4-yl)propane-1,3-diamine was obtained as a pale yellow powder (9 mg, 12%). MS m/z (M+H)=452.4.

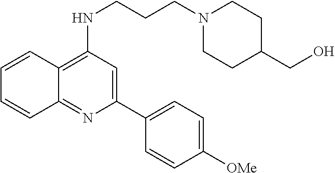 [SMILES: COc1ccc(-c2cc(NCCCN3CCC(CO)CC3)c3ccccc3n2)cc1]

Example 51: (1-(3-((2-(4-Methoxyphenyl)quinolin-4-yl)amino)propyl)piperidin-4-yl) methanol. To a solution of 1-amino-3-(diethylamino)propan-2-ol (0.127 g, 0.86 mmol) in dimethylformamide (0.5 mL) was added pyridine (0.035 μL, 0.43 mmol) followed by IA (0.08 g, 0.17 mmol). The resulting reaction mixture was stirred at 23° C. over a period of 16 hours. The reaction mixture was then diluted with dichloromethane (5 mL), washed with water (5 mL). Aqueous layer was extracted three times with dichloromethane, dried over magnesium sulfate and filtered. The resultant crude product was purified through silica gel cartridge with dichloromethane/methanol with 5% ammonium hydroxide to give (1-(3-((2-(4-methoxyphenyl)quinolin-4-yl)amino)propyl)piperidin-4-yl)methanol (14 mg, 21%). ¹H NMR (CDCl₃) δ: 8.03 (d, J=8.7 Hz, 2H), 7.86 (d, J=8.3 Hz, 1H), 7.54-7.64 (m, 2H), 7.26-7.34 (m, 1H), 6.95-7.04 (m, 2H), 6.70 (s, 1H), 3.85 (s, 3H), 3.56 (d, J=5.6 Hz, 2H), 3.37-3.47 (m, 2H), 3.09 (br d, J=11.1 Hz, 2H), 2.53-2.63 (m, 2H), 2.28 (br s, 1H), 1.88-2.01 (m, 4H), 1.79 (br d, J=12.0 Hz, 2H), 1.37-1.61 (m, 3H). MS m/z (M+H)=406.3.

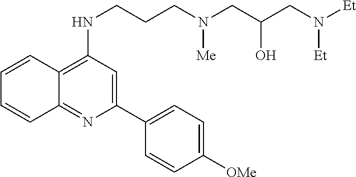 [SMILES: CCN(CC)CC(O)CN(C)CCCNc1cc(-c2ccc(OC)cc2)nc2ccccc12]

Example 52: 1-(Diethylamino)-3-((3-((2-(4-methoxyphenyl)quinolin-4-yl)amino)propyl)amino)propan-2-ol dihydrochloride. To a solution of 1-amino-3-(diethylamino)propan-2-ol (0.127 g, 0.86 mmol) in dimethylformamide (0.5 mL) was added pyridine (0.035 μL, 0.43 mmol) followed by IA (0.08 g, 0.17 mmol). The reaction was carried out following the same procedure as Example 1. The obtained oil was dissolved in diethyl ether and excess of hydrogen chloride solution in diethyl ether was added. The solvent was evaporated and the resulting powder was dried under vacuum to give 1-(diethylamino)-3-((3-((2-(4-methoxyphenyl)quinolin-4-yl)amino)propyl)amino)propan-2-ol dihydrochloride as a white powder (34 mg, 46%). ¹H NMR (300 MHz, DMSO-d₆) δ 13.85-13.57 (m, 1H), 10.03-9.85 (m, 1H), 9.69-9.53 (m, 1H), 9.44-9.23 (m, 1H), 8.78-8.63 (m, 1H), 8.23 (s, 1H), 8.13 (d, J=8.9 Hz, 2H), 7.99-7.87 (m, 1H), 7.72-7.60 (m, 1H), 7.19 (d, J=8.9 Hz, 2H), 7.07 (s, 1H), 6.47-6.28 (m, 1H), 4.58-4.28 (m, 1H), 3.87 (s, 3H), 3.84-3.71 (m, 1H), 3.24-3.02 (m, 10H), 3.00-2.88 (m, 1H), 2.27-2.05 (m, 2H), 1.22 (t, J=7.2 Hz, 6H). MS m/z (M+H)=437.4.

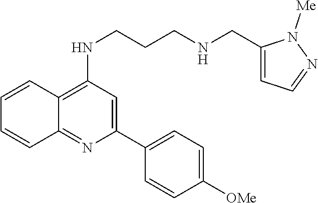 [SMILES: COc1ccc(-c2cc(NCCCNCc3ccnn3C)c3ccccc3n2)cc1]

Example 53: N¹-(2-(4-Methoxyphenyl)quinolin-4-yl)-N³-((1-methyl-1H-pyrazol-5-yl)methyl) propane-1,3-diamine dihydrochloride. To a solution of (1-methyl-1H-pyrazol-5-yl)methylamine (0.096 μL, 0.86 mmol) in dimethylformamide (0.5 mL) was added potassium carbonate (0.059 mg, 0.43 mmol) followed by IA (0.08 g, 0.17 mmol). The reaction was then conducted according to the procedure of Example 14. The obtained oil was dissolved in diethyl ether and excess of hydrogen chloride solution in diethyl ether was added. The solvent was evaporated and the resulting powder was dried under vacuum to give N¹-(2-(4-methoxyphenyl)quinolin-4-yl)-N³-((1-methyl-1H-pyrazol-5-yl)methyl)propane-1,3-diamine dihydrochloride as a pale yellow powder (14 mg, 21%). ¹H NMR (300 MHz, DMSO-d₆) δ 13.69 (s, 1H), 9.95-9.63 (m, 3H), 8.72 (d, J=8.5 Hz, 1H), 8.29 (d, J=8.5 Hz, 1H), 8.14 (d, J=8.7 Hz, 2H), 7.91 (t, J=7.7 Hz, 1H), 7.65 (t, J=7.7 Hz, 1H), 7.38 (d, J=1.9 Hz, 1H), 7.18 (d, J=8.7 Hz, 2H), 7.06 (s, 1H), 6.54 (d, J=1.9 Hz, 1H), 4.25 (br t, J=5.5H, 2H), 3.89 (s, 3H), 3.87 (s, 3H), 3.84-3.73 (m, 2H), 3.19-2.94 (m, 2H), 2.18 (br t, J=6.9 Hz, 2H). MS m/z (M+H)=402.2.

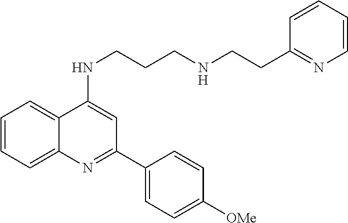 [SMILES: COc1ccc(-c2cc(NCCCNCCc3ccccn3)c3ccccc3n2)cc1]

Example 54: Synthesis of N¹-(2-(4-methoxyphenyl)quinolin-4-yl)-N³-(2-(pyridin-2-yl)ethyl)propane-1,3-diamine hydrochloride. To a solution of 2-(2-pyridyl)ethylamine (0.103 μL, 0.86 mmol) in dimethylformamide (0.5 mL) was added pyridine (35 μL, 0.43 mmol) followed by 3-((2-(4-methoxyphenyl)quinolin-4-yl)amino)propyl 4-methylbenzenesulfonate (0.08 g, 0.17 mmol). The reaction was carried out following the same procedure as Example 14. The obtained oil was dissolved in diethyl ether and excess of hydrogen chloride solution in diethyl ether was added. The solvent was evaporated and the resulting powder was dried under vacuum to give N¹-(2-(4-methoxyphenyl)quinolin-4-yl)-N³-(2-(pyridin-2-yl)ethyl)propane-1,3-diamine as a pale yellow powder (33 mg, 47%). ¹H NMR (300 MHz, DMSO-d6) δ 13.66 (s, 1H), 9.64 (br s, 2H), 8.79 (d, J=5.7 Hz, 1H), 8.75-8.67 (m, 1H), 8.48 (td, J=7.9, 1.6 Hz, 1H), 8.39-8.22 (m, 3H), 8.14 (d, J=8.9 Hz, 2H), 7.82-7.72 (m, 1H), 7.71-7.59 (m, 1H), 7.24-7.14 (m, 2H), 7.07 (s, 1H), 3.87 (s, 3H), 3.61-3.40 (m, 4H), 3.18 (t, J=6.5 Hz, 2H), 3.14-3.02 (m, 2H), 2.15 (br t, J=6.9 Hz, 2H). MS m/z (M+H)=413.2.

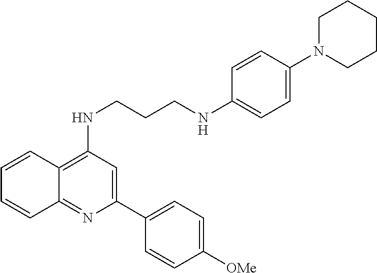 [SMILES: COc1ccc(-c2cc(NCCCNc3ccc(N4CCCCC4)cc3)c3ccccc3n2)cc1]

Example 55: N¹-(2-(4-Methoxyphenyl)quinolin-4-yl)-N³-(4-(piperidin-1-yl)phenyl) propane-1,3-diamine dihydrochloride. To a solution of 4-(N-piperidino)aniline (0.151 mg, 0.86 mmol) in dimethylformamide (0.5 mL) was added potassium carbonate (0.059 g, 0.43 mmol) followed by IA (0.08 g, 0.17 mmol). The reaction was carried out following the same procedure as Example 14. The obtained oil was dissolved in diethyl ether and excess of hydrogen chloride solution in diethyl ether was added. The solvent was evaporated and the resulting powder was dried under vacuum to give N¹-(2-(4-methoxyphenyl)quinolin-4-yl)-N³-(4-(piperidin-1-yl)phenyl)propane-1,3-diamine dihydrochloride as a brown powder (20 mg, 26%). ¹H NMR (300 MHz, DMSO-d₆) δ 13.52 (s, 1H), 9.69-9.28 (m, 1H), 8.65 (d, J=8.5 Hz, 1H), 8.20 (d, J=8.4 Hz, 1H), 8.02 (d, J=8.8 Hz, 2H), 7.92 (t, J=7.7 Hz, 1H), 7.73-7.60 (m, 1H), 7.52 (br d, J=8.4 Hz, 2H), 7.18 (d, J=8.8 Hz, 2H), 7.01 (s, 1H), 6.85-6.65 (m, 2H), 3.88 (s, 3H), 3.61-3.49 (m, 2H), 3.45-3.27 (m, 5H), 3.20 (br t, J=6.6 Hz, 2H), 2.11-1.94 (m, 2H), 1.94-1.74 (m, 3H), 1.72-1.44 (m, 4H). MS m/z (M+H)=467.4.

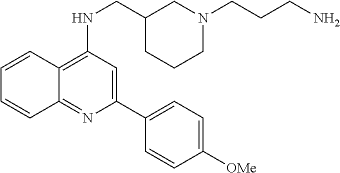 [SMILES: COc1ccc(-c2cc(NCC3CCCN(CCCN)C3)c3ccccc3n2)cc1]

Example 56: N-((1-(3-Aminopropyl)piperidin-3-yl)methyl)-2-(4-methoxyphenyl)quinolin-4-amine. To a solution of 2-(3-(3-(((2-(4-methoxyphenyl)quinolin-4-yl)amino)methyl)piperidin-1-yl)propyl)isoindoline-1,3-dione (0.044 g, 0.082 mmol) in ethanol (1 mL) was added hydrazine hydrate (5 μL, 0.099 mmol). The reaction was stirred at 23° C. overnight. Water was added to the resulting reaction mixture and aqueous layer was extracted three times with dichloromethane, dried over magnesium sulfate and filtered. The reaction mixture was purified through silica gel cartridge eluting with dichloromethane/methanol with 5% ammonium hydroxide to give N-((1-(3-aminopropyl)piperidin-3-yl)methyl)-2-(4-methoxyphenyl)quinolin-4-amine as a pale yellow powder (8 mg, 24%). MS m/z (M+H)=405.26.

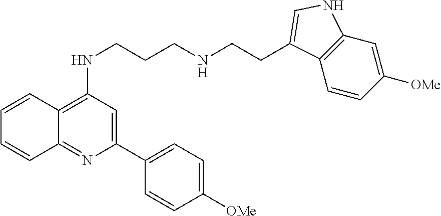 [SMILES: COc1ccc(-c2cc(NCCCNCCc3c[nH]c4cc(OC)ccc34)c3ccccc3n2)cc1]

Example 57: N-(3-(2-(6-Methoxy-1H-indol-3-yl)ethylamino)propyl)-2-(4-methoxyphenyl) quinolin-4-amine. To a solution of 2-(6-methoxy-1H-indol-3-yl)ethanamine (84 mg, 0.44 mmol) and triethylamine (100 uL, 0.66 mmol) in tetrahydrofuran (1 mL), was added IA dissolved in tetrahydrofuran (1 mL). The reaction was stirred at 23° C. overnight. Partitioned between ethyl acetate and water, extracted with ethyl acetate and washed combined organics with brine, dried over sodium sulfate, decanted and concentrated under vacuum. The crude product was purified on silica gel cartridge (4 g, 60-120 mesh), eluting with dichloromethane/methanol with 5% ammonium hydroxide to give N-(3-(2-(6-methoxy-1H-indol-3-yl)ethylamino)propyl)-2-(4-methoxyphenyl)quinolin-4-amine as a bright yellow foam (28 mg, 27%). ¹H NMR (CDCl₃) δ: 8.04 (d, J=8.8 Hz, 2H), 7.81 (d, J=8.3 Hz, 1H), 7.52-7.65 (m, 2H), 7.48 (d, J=8.5 Hz, 1H), 7.28-7.36 (m, 1H), 7.26 (s, 1H), 7.00 (d, J=8.8 Hz, 1H), 6.90 (d, J=2.2 Hz, 1H), 6.75-6.83 (m, 2H), 6.69 (s, 1H), 3.86 (s, 2H), 3.80-3.84 (m, 2H), 3.80-3.82 (m, 1H), 3.39-3.49 (m, 2H), 3.00-3.07 (m, 3H), 2.90 (t, J=5.5 Hz, 2H), 1.70-2.16 (m, 7H). MS m/z (M+H)=481.08.

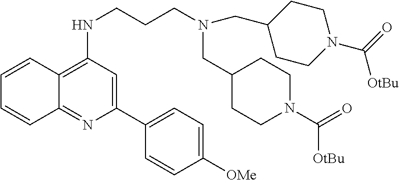 [SMILES: COc1ccc(-c2cc(NCCCN(CC3CCN(C(=O)OC(C)(C)C)CC3)CC3CCN(C(=O)OC(C)(C)C)CC3)c3ccccc3n2)cc1]

Example 58: tert-Butyl 4-{[({1-[(tert-butoxy)carbonyl]piperidin-4-yl}methyl)(3-{[2-(4-methoxyphenyl)-quinolin-4-yl]amino}propyl)amino]methyl}piperidine-1-carboxylate. To a solution of N-(3-aminopropyl)-2-(4-methoxyphenyl)quinolin-4-amine (500 mg, 1.63 mmol) in tetrahydrofuran was added tert-butyl 4-formylpiperidine-1-carboxylate (350 mg, 1.63 mmol). The mixture was stirred for 20 minutes, then sodium triacetoxyborohydride (345 mg, 1.63 mmol) was added and the reaction stirred at 23° C. overnight. The reaction was quenched with saturated ammonium chloride, and extracted with ethyl acetate (3 times 20 mL). The combined organic layers were washed with brine (25 mL), dried over sodium sulfate, decanted and concentrated under vacuum. The resulting crude product was purified on silica gel cartridge (24 g, 60-120 mesh) eluting with dichloromethane/methanol with 5% ammonium hydroxide to give tert-butyl 4-{[({1-[(tert-butoxy)carbonyl]piperidin-4-yl}methyl)(3-{[2-(4-methoxyphenyl)quinolin-4-yl]amino}propyl)amino]methyl}piperidine-1-carboxylate as a pale foam (132 mg, 11%). ¹H NMR (CDCl₃) δ: 8.04 (d, J=8.8 Hz, 2H), 7.59-7.73 (m, 2H), 7.38 (s, 1H), 7.03 (d, J=8.8 Hz, 2H), 6.80 (s, 1H), 3.88 (s, 3H), 3.39-3.49 (m, 2H), 2.86-2.98 (m, 1H), 2.50-2.67 (m, 6H), 2.23 (d, J=6.8 Hz, 4H), 1.84-1.97 (m, 3H), 1.62-1.79 (m, 10H), 1.42-1.44 (m, 18H), 0.92-1.12 (m, 4H). MS m/z (M+H)=702.80.

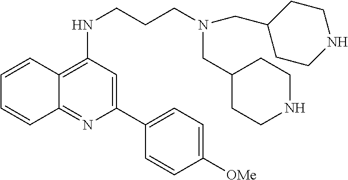 [SMILES: COc1ccc(-c2cc(NCCCN(CC3CCNCC3)CC3CCNCC3)c3ccccc3n2)cc1]

Example 59: N-(3-(Bis((piperidin-4-yl)methyl)amino)propyl)-2-(4-methoxyphenyl) quinolin-4-amine. To a solution of tert-butyl 4-{[({1-[(tert-butoxy)carbonyl]piperidin-4-yl}methyl)(3-{[2-(4-methoxy-phenyl)quinolin-4-yl]amino}propyl)amino]methyl}piperidine-1-carboxylate in tetrahydrofuran (1 mL) was added 4 N hydrogen chloride in 1,4-dioxane (800 uL, excess). The reaction was stirred overnight at 23° C. The reaction was concentrated under vacuum and the residue dissolved in water, washed with ethyl acetate and dried on a lyophilizer to afford a white solid. ¹H NMR (CDCl₃) δ: 7.97-8.09 (m, 3H), 7.70-7.81 (m, 1H), 7.55-7.66 (m, 1H), 7.28-7.39 (m, 1H), 7.26 (s, 1H), 7.11 (br s, 1H), 7.01 (d, J=8.8 Hz, 2H), 6.75 (s, 1H), 4.11 (s, 3H), 3.86 (br d, J=2.2 Hz, 1H), 3.47 (q, J=5.2 Hz, 2H), 2.63-2.96 (m, 4H), 2.56 (d, J=6.2 Hz, 2H), 1.84-2.03 (m, 4H), 1.61-1.80 (m, 5H), 1.46 (s, 9H), 1.05-1.27 (m, 2H). MS m/z (M+H)=502.16.

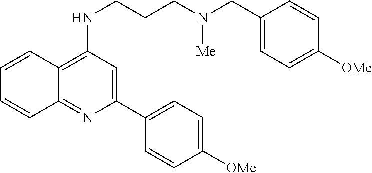 [SMILES: COc1ccc(CN(C)CCCNc2cc(-c3ccc(OC)cc3)nc3ccccc23)cc1]

Example 60: N¹-(4-Methoxybenzyl)-N³-(2-(4-methoxyphenyl)quinolin-4-yl)-N¹-methyl propane-1,3-diamine. To a solution of [(4-methoxyphenyl)methyl](methyl)amine (0.131 g, 0.86 mmol) in dimethylformamide (0.5 mL) was added potassium carbonate (0.059 g, 0.43 mmol) followed by IA (0.08 g, 0.17 mmol). The resulting reaction mixture was stirred at 23° C. over a period of 16 hours. The reaction mixture was then diluted with dichloromethane (5 mL), washed with water (5 mL). Aqueous layer was extracted three times with dichloromethane, dried over magnesium sulfate and filtered. The resultant crude product was purified through silica gel cartridge with dichloromethane/methanol with 5% ammonium hydroxide to give N¹-(4-methoxybenzyl)-N³-(2-(4-methoxyphenyl)quinolin-4-yl)-N¹-methylpropane-1,3-diamine as a white powder (30 mg, 40%). ¹H NMR (CDCl₃) δ: 8.06 (d, J=8.8 Hz, 3H), 7.55-7.64 (m, 1H), 7.47-7.54 (m, 1H), 7.16-7.29 (m, 4H), 6.96-7.06 (m, 2H), 6.77-6.83 (m, 2H), 6.75 (s, 1H), 3.86 (s, 3H), 3.77 (s, 3H), 3.51 (s, 2H), 3.40-3.49 (m, 2H), 2.60-2.71 (m, 2H), 2.32 (s, 3H), 1.98 (br t, J=5.6 Hz, 2H). MS m/z (M+H)=442.3.

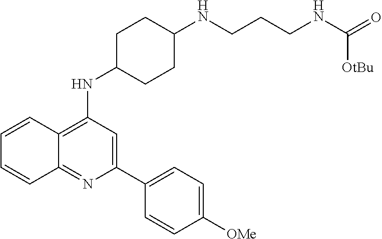 [SMILES: COc1ccc(-c2cc(NC3CCC(NCCCNC(=O)OC(C)(C)C)CC3)c3ccccc3n2)cc1]

### Example 61: tert-Butyl(3-((4-((2-(4-methoxyphenyl)quinolin-4-yl)amino)cyclohexyl)amino) propyl)carbamate

Step 1: To a solution of 4-chloro-2-(4-methoxyphenyl)quinoline (0.5 g, 1.85 mmol) with 3,3′-diamino-N-methyldipropylamine (1.06 g mL, 9.27 mmol) was added two drops of tin tetrachloride. The reaction was carried out following the same procedure as step 2 intermediate IA. The crude reaction was purified on silica gel eluting with dichloromethane methanol with ammonium hydroxide to provide N¹-(2-(4-methoxyphenyl)quinolin-4-yl)cyclohexane-1,4-diamine (336 mg, 53%).

Step 2: To a solution of tert-butyl(3-bromopropyl)carbamate (0.205 g, 0.88 mmol) in dimethylformamide (1 mL) was added potassium carbonate (0.048 g, 0.34 mmol) followed by N¹-(2-(4-methoxyphenyl)quinolin-4-yl)cyclohexane-1,4-diamine (0.06 g, 0.17 mmol). The resulting reaction mixture was stirred at 23° C. over a period of 16 hours. The reaction mixture was then diluted with dichloromethane (5 mL), washed with water (5 mL). Aqueous layer was extracted three times with dichloromethane, dried over magnesium sulfate and filtered. The resultant crude product was purified through silica gel cartridge with dichloromethane/methanol with 5% ammonium hydroxide to give tert-butyl(3-((4-((2-(4-methoxyphenyl)quinolin-4-yl)amino)cyclohexyl)amino)propyl) carbamate as a white powder (15 mg, 18%). ¹H NMR (DMSO-d₆) δ: 8.27 (d, J=8.4 Hz, 1H), 8.11 (d, J=8.9 Hz, 2H), 7.78 (dd, J=8.4, 1.3 Hz, 1H), 7.53-7.63 (m, 1H), 7.28-7.39 (m, 1H), 7.02 (d, J=8.9 Hz, 2H), 6.91 (s, 1H), 6.78-6.87 (m, 1H), 6.61 (br d, J=7.1 Hz, 1H), 3.81 (s, 3H), 2.97 (br d, J=6.5 Hz, 2H), 2.66-2.80 (m, 1H), 2.51 (s, 2H), 1.86 (s, 2H), 1.61-1.79 (m, 7H), 1.47-1.57 (m, 2H), 1.35 (s, 9H). MS m/z (M+H)=505.4.

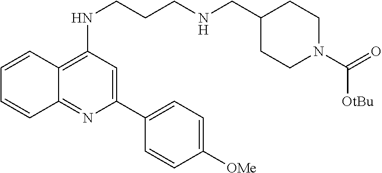 [SMILES: COc1ccc(-c2cc(NCCCNCC3CCN(C(=O)OC(C)(C)C)CC3)c3ccccc3n2)cc1]

Example 62: tert-Butyl 4-{[(3-{[2-(4-methoxyphenyl)quinolin-4-yl]amino}propyl)amino] methyl}piperidine-1-carboxylate. To a solution of N-(3-aminopropyl)-2-(4-methoxyphenyl) quinolin-4-amine (500 mg, 1.63 mmol) in tetrahydrofuran (2 mL) was added tert-butyl 4-formylpiperidine-1-carboxylate (350 mg, 1.63 mmol), the mixture was stirred at 23° C. for 20 minutes, then sodium tri-acetoxy borohydride (345 mg, 1.63 mmol) was added in one portion. The reaction was stirred at 23° C. for 14 hours. The reaction was quenched with saturated ammonium chloride and extracted into ethyl acetate, dried over sodium sulfate and concentrated under vacuum. The residue was purified on silica gel (24 g, 0-10% methanol with 5% ammonium hydroxide). Isolated tert-butyl 4-{[(3-{[2-(4-methoxyphenyl)quinolin-4yl]amino}propyl)amino]methyl}-piperidine-1-carboxylate (65.8 mg, 8%). ¹H NMR (CDCl₃) δ: 7.97-8.10 (m, 3H), 7.71-7.80 (m, 1H), 7.53-7.66 (m, 1H), 7.28-7.38 (m, 1H), 6.93-7.17 (m, 3H), 6.75 (s, 1H), 4.11 (s, 2H), 3.86 (br d, J=2.2 Hz, 1H), 3.47 (q, J=5.0 Hz, 2H), 2.83-2.97 (m, 2H), 2.71 (br t, J=12.7 Hz, 2H), 2.56 (d, J=6.2 Hz, 2H), 1.87-2.05 (m, 2H), 1.64-1.80 (m, 4H), 1.46 (s, 9H), 1.05-1.24 (m, 2H). MS m/z (M+H)=505.

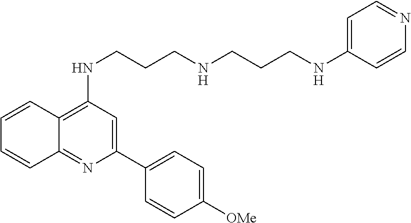 [SMILES: COc1ccc(-c2cc(NCCCNCCCNc3ccncc3)c3ccccc3n2)cc1]

Example 63: N¹-(2-(4-methoxyphenyl)quinolin-4-yl)-N³-(3-(pyridin-4-ylamino)propyl)propane-1,3-diamine dihydrochloride. To a solution of N-(3-aminopropyl)pyridine-4-amine (0.131 mg, 0.86 mmol) in dimethylformamide (0.5 mL) was added potassium carbonate (0.059 g, 0.43 mmol) followed by IA (0.08 g, 0.17 mmol). The reaction was then conducted according to the procedure of Example 14. The obtained oil was dissolved in diethyl ether and excess of hydrogen chloride solution in diethyl ether was added. The solvent was evaporated and the resulting powder was dried under vacuum to give N¹-(3-(Pyridine-4-yl)aminopropyl)-N³-(2-(4-methoxyphenyl)quinolin-4-yl)-N¹-propane-1,3-diamine dihydrochloride as a white powder (25 mg, 34%). ¹H NMR (300 MHz, DMSO-d₆) δ 13.64 (s, 1H), 13.52 (br s, 1H), 9.65 (br t, J=5.8 Hz, 1H), 9.57-9.38 (m, 2H), 9.10-8.91 (m, 1H), 8.72 (d, J=8.5 Hz, 1H), 8.32-8.24 (m, 1H), 8.23-8.18 (m, 1H), 8.14 (d, J=8.9 Hz, 2H), 8.05 (t, J=6.2 Hz, 1H), 7.92 (t, J=7.6 Hz, 1H), 7.65 (t, J=8.2 Hz, 1H), 7.18 (d, J=5.0 Hz, 1H), 7.07 (s, 1H), 6.92 (br t, J=5.2 Hz, 2H), 3.87 (s, 3H), 3.82 (d, J=5.9 Hz, 2H), 3.40 (q, J=6.6 Hz, 2H), 3.02 (br d, J=7.8 Hz, 4H), 2.15 (br t, J=6.8 Hz, 2H), 1.99 (q, J=7.1 Hz, 2H). MS m/z (M+H)=442.3.

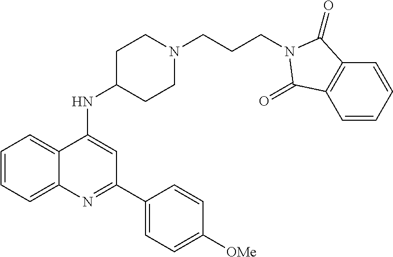 [SMILES: COc1ccc(-c2cc(NC3CCN(CCCN4C(=O)c5ccccc5C4=O)CC3)c3ccccc3n2)cc1]

### Example 64: 2-(3-(4-((2-(4-Methoxyphenyl)quinolin-4-yl)amino)piperidin-1-yl)propyl) isoindoline-1,3-dione dihydrochloride

Step 1: Synthesis of N-(1-benzylpiperidin-4-yl)-2-(4-methoxyphenyl)quinolin-4-amine. To a solution of 4-chloro-2-(4-methoxyphenyl)quinoline (0.500 g, 1.85 mmol) in 4-amino-1-benzyl-piperidine (4.23 g, 22.2 mmol) was added tin tetrachloride (3 drops) and the reactions was stirred at 135° C. for 72 hours and then 90 minutes under microwave irradiation at 220° C. The reaction was cooled to room temperature and the resulting material was purified on silica gel cartridge eluting with ethyl acetate in hexanes to provide 375 mg of N-(1-benzylpiperidin-4-yl)-2-(4-methoxyphenyl)quinolin-4-amine.

Step 2: Synthesis of 2-(4-methoxyphenyl)-N-(piperidin-4-yl)quinolin-4-amine. N-(1-benzylpiperidin-4-yl)-2-(4-methoxyphenyl)quinolin-4-amine (375 mg, 0.88 mmol) was placed in a Parr bottle and dissolved in methanol (3 mL), palladium on carbon (942 mg) was added and the flask charged to 50 psi H₂, stirred over night at 23° C. The reaction mixture was filtered through a plug of celite and concentrated in vacuum to provide the title compound which was used without further purification.

Step 3: To a solution of 2-(4-methoxyphenyl)-N-(piperidin-4-yl)quinolin-4-amine (0.06 g, 0.18 mmol) in dimethylformamide (1 mL) was added potassium carbonate (0.05 g, 0.36 mmol) followed by N-(3-bromopropyl)phthalimide (0.241 g, 0.89 mmol). The obtained oil was dissolved in diethyl ether and excess of hydrogen chloride solution in diethyl ether was added. The solvent was evaporated and the resulting powder was dried under vacuum to give 2-(3-(4-((2-(4-methoxyphenyl)quinolin-4-yl)amino)piperidin-1-yl)propyl)isoindoline-1,3-dione dihydrochloride as pale brown powder (34 mg, 37%). ¹H NMR (300 MHz, DMSO-d₆) δ 13.71 (s, 1H), 11.09-10.76 (m, 1H), 9.07 (br d, J=7.9 Hz, 1H), 8.74 (d, J=8.5 Hz, 1H), 8.26-8.18 (m, 1H), 8.10-8.02 (m, 2H), 7.97-7.90 (m, 1H), 7.89-7.79 (m, 4H), 7.70-7.60 (m, 1H), 7.20 (d, J=8.8 Hz, 2H), 7.08 (s, 1H), 4.41-4.17 (m, 1H), 3.87 (s, 3H), 3.66 (br t, J=6.3 Hz, 2H), 3.60-3.48 (m, 2H), 3.18-2.98 (m, 4H), 2.32-1.99 (m, 6H). MS m/z (M+H)=521.4.

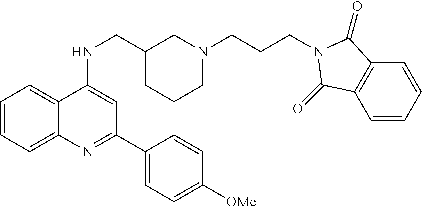 [SMILES: COc1ccc(-c2cc(NCC3CCCN(CCCN4C(=O)c5ccccc5C4=O)C3)c3ccccc3n2)cc1]

### Example 65: 2-(3-(3-(((2-(4-Methoxyphenyl)quinolin-4-yl)amino)methyl)piperidin-1-yl) propyl)isoindoline-1,3-dione

Step 1: To a solution of 4-chloro-2-(4-methoxyphenyl)quinoline (0.2 g, 0.74 mmol) with 2-(aminoethyl)-1-N-Boc-piperidine (0.797 mL, 3.7 mmol) was added two drops of tin tetrachloride. 2-(4-Methoxyphenyl)-N-(piperidin-3-ylmethyl)quinolin-4-amine was obtained as a pale brown oil (79 mg, 31%). ¹H NMR (300 MHz, CDCl₃) δ 8.04 (d, J=8.3 Hz, 3H), 7.71 (d, J=8.3 Hz, 1H), 7.62 (t, J=8.4 Hz, 1H), 7.38 (t, J=8.3 Hz, 1H), 7.09-6.97 (m, 2H), 6.80 (s, 1H), 5.11 (br t, J=5.3 Hz, 1H), 3.87 (s, 3H), 3.26 (t, J=5.9 Hz, 2H), 3.13 (br d, J=12.3 Hz, 2H), 2.63 (br t, J=12.0 Hz, 2H), 2.34 (s, 1H), 1.84 (br d, J=12.6 Hz, 3H), 1.40-1.19 (m, 2H). MS m/z (M+H)=348.3.

Step 2: To a solution of 2-(4-methoxyphenyl)-N-(piperidin-3-ylmethyl)quinolin-4-amine (0.06 mg, 0.14 mmol) in dimethylformamide (0.5 mL) was added potassium carbonate (0.079 g, 0.57 mmol) followed by N-(3-bromopropyl)phthalimide (0.038 g, 0.14 mmol). The reaction was carried out following the same procedure as Example 3 to give 2-(3-(3-(((2-(4-methoxyphenyl)quinolin-4-yl)amino)methyl)piperidin-1-yl)propyl)isoindoline-1,3-dione as a pale yellow powder (44 mg, 49%). ¹H NMR (300 MHz, CDCl₃) δ 8.11-7.95 (m, 3H), 7.87-7.77 (m, 2H), 7.74-7.64 (m, 3H), 7.59 (t, J=7.9 Hz, 1H), 7.57-7.54 (m, 1H), 7.42-7.30 (m, 1H), 7.01 (d, J=8.5 Hz, 2H), 6.73 (s, 1H), 5.19 (s, 1H), 3.85 (s, 3H), 3.74 (t, J=6.9 Hz, 2H), 3.43 (s, 1H), 3.17 (br t, J=5.8 Hz, 2H), 2.90 (br d, J=11.1 Hz, 2H), 2.40 (br t, J=7.0 Hz, 3H), 1.99-1.81 (m, 3H), 1.81-1.68 (m, 1H), 1.36-1.07 (m, 2H). MS m/z (M+H)=535.4.

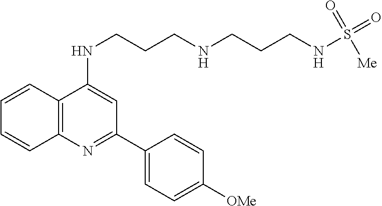 [SMILES: COc1ccc(-c2cc(NCCCNCCCNS(C)(=O)=O)c3ccccc3n2)cc1]

Example 66: N-(3-((3-((2-(4-Methoxyphenyl)quinolin-4-yl)amino)propyl)amino)propyl) methanesulfonamide hydrochloride. To a solution of N-(3-aminopropyl)methanesulfonamide (0.131 mg, 0.86 mmol) in dimethylformamide (0.5 mL) was added potassium carbonate (0.059 g, 0.43 mmol) followed by IA (0.08 g, 0.17 mmol). The reaction was carried out following the same procedure as Example 14. The obtained oil was dissolved in diethyl ether and excess of hydrogen chloride solution in diethyl ether was added. The solvent was evaporated and the resulting powder was dried under vacuum to give N-(3-((3-((2-(4-methoxyphenyl)quinolin-4-yl)amino)propyl)amino)propyl)methane sulfonamide hydrochloride as a white powder (49 mg, 66%). ¹H NMR (300 MHz, DMSO-d₆) δ 13.64 (s, 1H), 9.77-9.57 (m, 1H), 9.29 (br s, 2H), 8.70 (d, J=8.5 Hz, 1H), 8.26 (d, J=8.5 Hz, 1H), 8.13 (d, J=8.9 Hz, 1H), 7.99-7.79 (m, 1H), 7.74-7.58 (m, 1H), 7.18 (d, J=8.8 Hz, 3H), 7.06 (s, 1H), 3.87 (s, 3H), 3.80 (br d, J=6.4 Hz, 2H), 3.02 (br d, J=6.5 Hz, 4H), 2.97-2.90 (m, 2H), 2.89 (s, 3H), 2.12 (br t, J=6.9 Hz, 2H), 1.86 (quin, J=7.2 Hz, 2H). MS m/z (M+H)=443.4.

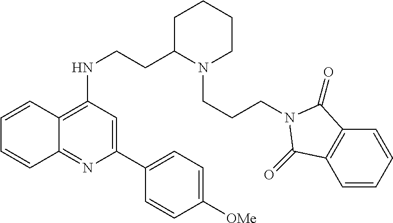 [SMILES: COc1ccc(-c2cc(NCCC3CCCCN3CCCN3C(=O)c4ccccc4C3=O)c3ccccc3n2)cc1]

### Example 67: 2-(3-(2-(2-((2-(4-Methoxyphenyl)quinolin-4-yl)amino)ethyl)piperidin-1-yl)propyl) isoindoline-1,3-dione

Step 1: To a solution of 4-chloro-2-(4-methoxyphenyl)quinoline (0.2 g, 0.74 mmol) with 2-(aminoethyl)-1-N-Boc-piperidine (0.84 g, 3.7 mmol) was added two drops of tin tetrachloride. 2-(4-Methoxyphenyl)-N-(2-(piperidin-2-yl)ethyl)quinolin-4-amine was obtained as a pale brown oil (158 mg, 60%). ¹H NMR (300 MHz, CDCl₃) δ 8.03 (d, J=8.4 Hz, 3H), 7.80 (d, J=8.3 Hz, 1H), 7.59 (t, J=8.5 Hz, 1H), 7.33 (d, J=8.4 Hz, 1H), 7.00 (d, J=8.4 Hz, 2H), 6.88 (s, 1H), 3.84 (s, 3H), 3.49-3.35 (m, 2H), 3.16 (br d, J=12.8 Hz, 1H), 3.04 (br s, 2H), 2.83-2.71 (m, 1H), 2.71-2.56 (m, 1H), 1.89-1.71 (m, 3H), 1.71-1.54 (m, 2H), 1.51-1.35 (m, 2H), 1.34-1.15 (m, 1H). MS m/z (M+H)=362.3.

Step 2: To a solution of 2-(4-methoxyphenyl)-N-(2-(piperidin-2-yl)ethyl)quinolin-4-amine (0.06 mg, 0.16 mmol) in dimethylformamide (0.5 mL) was added potassium carbonate (0.046 g, 0.33 mmol) followed by N-(3-bromopropyl)phthalimide (0.044 g, 0.17 mmol). The reaction was conducted according to the procedure of Example 14 and 2-(3-(2-(2-((2-(4-Methoxyphenyl)quinolin-4-yl)amino)ethyl)piperidin-1-yl)propyl)isoindoline-1,3-dione was obtained as pale yellow powder (33 mg, 38%). ¹H NMR (300 MHz, CDCl₃) δ 8.10-7.98 (m, 3H), 7.75-7.64 (m, 3H), 7.63-7.53 (m, 3H), 7.41-7.30 (m, 1H), 7.01 (d, J=8.4 Hz, 2H), 6.78 (s, 1H), 6.68 (s, 1H), 3.86 (s, 3H), 3.70 (t, J=6.8 Hz, 2H), 3.52-3.32 (m, 2H), 3.14-3.02 (m, 1H), 3.01-2.84 (m, 1H), 2.67-2.51 (m, 2H), 2.38-2.02 (m, 3H), 2.01-1.83 (m, 3H), 1.76-1.56 (m, 3H), 1.54-1.27 (m, 2H). MS m/z (M+H)=549.4.

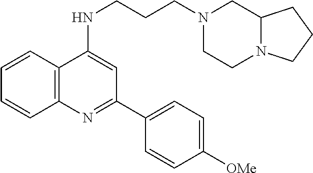 [SMILES: COc1ccc(-c2cc(NCCCN3CCN4CCCC4C3)c3ccccc3n2)cc1]

Example 68: N-(3-(Hexahydropyrrolo[1,2-a]pyrazin-2(1H)-yl)propyl)-2-(4-methoxy phenyl)quinolin-4-amine dihydrochloride. To a solution of octahydropyrrolo[1,2-a]pyrazine (0.113 mL, 0.86 mmol) in dimethylformamide (0.5 mL) was added cesium carbonate (0.141 g, 0.43 mmol) followed by IA (0.08 g, 0.17 mmol). The reaction was carried out following the same procedure as Example 14. The obtained oil was dissolved in diethyl ether and excess of hydrogen chloride solution in diethyl ether was added. The solvent was evaporated and the resulting powder was dried under vacuum to give N-(3-(hexahydropyrrolo[1,2-a]pyrazin-2(1H)-yl)propyl)-2-(4-methoxyphenyl) quinolin-4-amine as a pale orange powder (38 mg, 54%). ¹H NMR (300 MHz, DMSO-d₆) δ 13.63 (s, 1H), 12.50-11.73 (m, 1H), 9.85-9.35 (m, 1H), 8.66 (s, 1H), 8.24 (d, J=8.5 Hz, 1H), 8.13 (d, J=8.5 Hz, 2H), 7.93 (t, J=7.7 Hz, 1H), 7.67 (t, J=7.7 Hz, 1H), 7.20 (d, J=8.8 Hz, 2H), 7.08 (s, 1H), 3.88 (s, 3H), 3.79 (s, 3H), 3.71-3.50 (m, 4H), 3.21-3.16 (m, 4H), 3.16-2.94 (m, 1H), 2.22 (br s, 2H), 2.14-1.86 (m, 4H), 1.82-1.64 (m, 1H). MS m/z (M+H)=417.3.

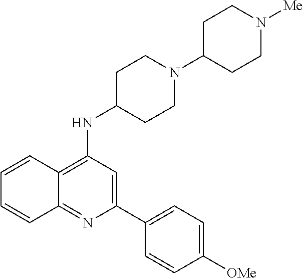 [SMILES: COc1ccc(-c2cc(NC3CCN(C4CCN(C)CC4)CC3)c3ccccc3n2)cc1]

Example 69: 2-(4-Methoxyphenyl)-N-(1′-methyl-[1,4′-bipiperidine]-4-yl)quinolin-4-amine. To a solution of 4-chloro-2-(4-methoxyphenyl)quinoline (0.1 g, 0.37 mmol) with 1-(1-methylpiperidin-4-yl)piperidin-4-amine (0.366 g, 1.85 mmol) was added two drops of tin tetrachloride. The reaction was carried out following the same procedure as step 2 of the synthesis of intermediate A (IA). 2-(4-methoxyphenyl)-N-(1′-methyl-[1,4′-bipiperidin]-4-yl)quinolin-4-amine was obtained as pale yellow powder (58 mg, 37%). ¹H NMR (300 MHz, DMSO-d₆) δ 8.26 (d, J=8.4 Hz, 1H), 8.15 (d, J=8.8 Hz, 2H), 7.80 (dd, J=8.5, 1.3 Hz, 1H), 7.59 (ddd, J=8.3, 6.9, 1.2 Hz, 1H), 7.42-7.30 (m, 1H), 7.04 (d, J=8.9 Hz, 2H), 6.95 (s, 1H), 6.70 (d, J=7.8 Hz, 1H), 3.83 (s, 3H), 3.70 (br d, J=8.7 Hz, 1H), 2.91 (br d, J=11.1 Hz, 2H), 2.79 (br d, J=10.9 Hz, 2H), 2.36 (br t, J=11.3 Hz, 2H), 2.28-2.17 (m, 1H), 2.13 (s, 3H), 2.00 (br d, J=12.2 Hz, 2H), 1.84 (br t, J=11.3 Hz, 2H), 1.77-1.58 (m, 4H), 1.58-1.38 (m, 2H). MS m/z (M+H)=431.5.

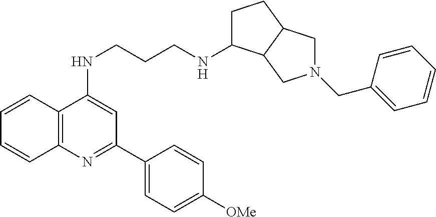 [SMILES: COc1ccc(-c2cc(NCCCNC3CCC4CN(Cc5ccccc5)CC43)c3ccccc3n2)cc1]

Example 70: N¹-(2-Benzyloctahydrocyclopenta[c]pyrrol-4-yl)-N³-(2-(4-methoxyphenyl) quinolin-4-yl)propane-1,3-diamine trihydrochloride. Into a vial with IA (0.06 g, 0.16 mmol) dissolved in dichloromethane (0.5 mL) was added 2-benzyloctahydrocyclopenta[c]pyrrol-4-amine (0.175 mg, 0.81 mmol). The reaction was carried out following the same procedure as Example 1. The obtained oil was dissolved in diethyl ether and excess of hydrogen chloride solution in diethyl ether was added. The solvent was evaporated and the resulting powder was dried under vacuum to give N¹-(2-benzyloctahydrocyclopenta[c]pyrrol-4-yl)-N³-(2-(4-methoxyphenyl)quinolin-4-yl)propane-1,3-diamine trihydrochloride as a pale yellow powder (12 mg, 15%). ¹H NMR (300 MHz, DMSO-d₆) δ 13.60 (br s, 1H), 11.95-11.04 (m, 1H), 9.80-9.31 (m, 3H), 8.69 (t, J=7.6 Hz, 1H), 8.24 (br d, J=8.4 Hz, 1H), 8.13 (d, J=8.6 Hz, 2H), 7.92 (t, J=7.7 Hz, 1H), 7.74-7.60 (m, 2H), 7.56 (dd, J=6.3, 2.8 Hz, 1H), 7.47-7.33 (m, 3H), 7.18 (d, J=8.6 Hz, 2H), 7.07 (d, J=3.1 Hz, 1H), 4.44-4.14 (m, 2H), 3.87 (s, 3H), 3.83-3.68 (m, 3H), 3.23-2.69 (m, 7H), 2.33-2.02 (m, 4H), 1.90 and 1.50 (m, 1H), 1.64-1.40 (m, 1H), 1.21 (s, 1H). MS m/z (M+H)=507.4.

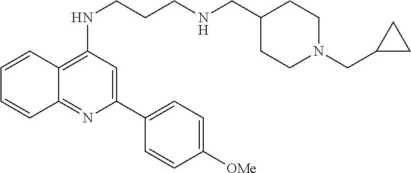 [SMILES: COc1ccc(-c2cc(NCCCNCC3CCN(CC4CC4)CC3)c3ccccc3n2)cc1]

Example 71: N¹-((1-(Cyclopropylmethyl)piperidin-4-yl)methyl)-N³-(2-(4-methoxy phenyl) quinolin-4-yl)propane-1,3-diamine trihydrochloride. Into a vial with IB (0.06 g, 0.16 mmol) dissolved in dichloromethane (0.5 mL) was added 1[1-(cyclopropylmethyl)-4-piperidinyl]methanamine (0.136 mg, 0.81 mmol). The reaction was carried out following the same procedure as Example 1. The obtained oil was dissolved in diethyl ether and excess of hydrogen chloride solution in diethyl ether was added. The solvent was evaporated and the resulting powder was dried under vacuum to give N¹-((1-(cyclopropylmethyl)piperidin-4-yl)methyl)-N³-(2-(4-methoxyphenyl)quinolin-4-yl)propane-1,3-diamine trihydrochloride as a white powder (36 mg, 49%). ¹H NMR (300 MHz, DMSO-d₆) δ 13.59 (s, 1H), 10.63-10.35 (m, 1H), 9.64 (s, 1H), 9.32 (br s, 2H), 8.70 (d, J=8.4 Hz, 1H), 8.23 (d, J=8.5 Hz, 1H), 8.13 (d, J=8.7 Hz, 2H), 7.93 (t, J=7.7 Hz, 1H), 7.64 (t, J=9 Hz, 1H), 7.20 (d, J=8.8 Hz, 2H), 7.07 (s, 1H), 3.88 (s, 3H), 3.81 (m, 2H), 3.48 (m, 2H), 3.03 (br s, 2H), 2.93-2.75 (m, 5H), 2.28-2.11 (m, 2H), 2.02 (br d, J=13.7 Hz, 2H), 1.59 (br d, J=13.6 Hz, 2H), 1.59 (m, 2H), 1.16-1.10 (m, 1H), 0.71-0.53 (m, 2H), 0.36 (q, J=5.0 Hz, 2H). MS m/z (M+H)=459.5.

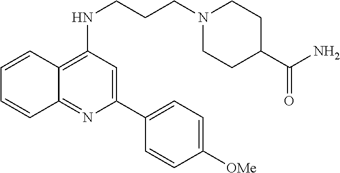 [SMILES: COc1ccc(-c2cc(NCCCN3CCC(C(N)=O)CC3)c3ccccc3n2)cc1]

Example 72: 1-(3-((2-(4-Methoxyphenyl)quinolin-4-yl)amino)propyl)piperidine-4-carboxamide dihydrochloride. To a solution of 4-chloro-2-(4-methoxyphenyl)quinoline (0.1 g, 0.37 mmol) with 1-(3-aminopropyl)piperidine-4-carboxamide (0.330 mL, 1.85 mmol) was added two drops of tin tetrachloride. The reaction was carried out following the same procedure as step 2 of the synthesis of intermediate A (IA). The obtained oil was dissolved in diethyl ether and excess of hydrogen chloride solution in diethyl ether was added. The solvent was evaporated and the resulting powder was dried under vacuum to give 1-(3-((2-(4-methoxyphenyl)quinolin-4-yl)amino)propyl)piperidine-4-carboxamide dihydrochloride as a pale yellow powder (10 mg, 7%). ¹H NMR (300 MHz, DMSO-d₆) δ 13.58 (s, 1H), 10.68-10.26 (m, 1H), 8.63 (d, J=8.2 Hz, 1H), 8.22 (d, J=8.5 Hz, 1H), 8.16-8.07 (m, 2H), 7.95 (t, J=7.7 Hz, 1H), 7.69 (t, J=7.7 Hz, 1H), 7.40 (s, 1H), 7.22 (d, J=8.9 Hz, 2H), 7.07 (s, 1H), 6.92 (s, 1H), 3.89 (s, 3H), 3.78 (br d, J=6.8 Hz, 2H), 3.56-3.42 (m, 2H), 3.24-3.07 (m, 3H), 3.02-2.73 (m, 2H), 2.42-2.25 (m, 1H), 2.25-2.08 (m, 2H), 2.04-1.96 (m, 1H), 1.89 (m, 3H). MS m/z (M+H)=419.4.

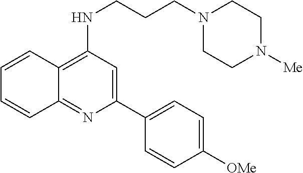 [SMILES: COc1ccc(-c2cc(NCCCN3CCN(C)CC3)c3ccccc3n2)cc1]

Example 73: 2-(4-Methoxyphenyl)-N-(3-(4-methylpiperazin-1-yl)propyl)quinolin-4-amine. To a solution of 4-chloro-6-methoxy-2-(4-methoxyphenyl)quinoline (0.1 g, 0.37 mmol) with 1-(3-aminopropyl)-4-methylpiperazine (0.757 mL, 4.45 mmol) was added two drops of tin tetrachloride. The reaction mixture was stirred at 130° C. over a period of 12 hours. The reaction mixture was directly purified through silica gel cartridge eluting with dichloromethane/methanol with 5% ammonium hydroxide to give 2-(4-methoxyphenyl)-N-(3-(4-methylpiperazin-1-yl)propyl)quinolin-4-amine as a white powder (75 mg, 52%). ¹H NMR (CDCl₃) δ: 7.99-8.10 (m, 3H), 7.93 (d, J=8.3 Hz, 1H), 7.57-7.67 (m, 1H), 7.34-7.41 (m, 1H), 7.33 (br d, J=2.4 Hz, 1H), 6.97-7.04 (m, 2H), 6.75 (s, 1H), 3.86 (s, 3H), 3.46 (q, J=5.3 Hz, 2H), 2.49-2.72 (m, 9H), 2.39 (s, 3H), 2.22-2.31 (m, 1H), 1.90-2.04 (m, 2H). MS m/z (M+H)=391.2.

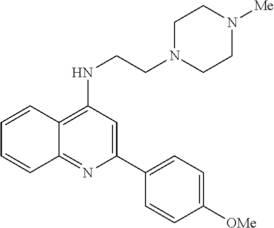 [SMILES: COc1ccc(-c2cc(NCCN3CCN(C)CC3)c3ccccc3n2)cc1]

Example 74: 2-(4-Methoxyphenyl)-N-(2-(4-methylpiperazin-1-yl)ethyl)quinolin-4-amine. To a solution of 4-chloro-6-methoxy-2-(4-methoxyphenyl)quinoline (0.1 g, 0.37 mmol) with 1 2-(4-methylpiperazin-1-yl)ethanamine (0.666 mL, 4.45 mmol) was added two drops of tin tetrachloride. The reaction mixture was stirred at 130° C. over a period of 12 hours. The reaction mixture was directly purified through silica gel cartridge eluting with dichloromethane/methanol with 5% ammonium hydroxide to give 2-(4-methoxyphenyl)-N-(2-(4-methylpiperazin-1-yl)ethyl)quinolin-4-amine as a white powder (29 mg, 21%). ¹H NMR (CDC₃) δ: 8.00-8.14 (m, 3H), 7.73 (d, J=8.3 Hz, 1H), 7.63 (ddd, J=8.4, 6.9, 1.4 Hz, 1H), 7.37-7.47 (m, 1H), 7.01 (q, J=4.9 Hz, 2H), 6.80 (s, 1H), 5.92 (br s, 1H), 3.87 (s, 3H), 3.34-3.49 (m, 2H), 2.82 (t, J=5.9 Hz, 2H), 2.39-2.69 (m, 8H), 2.32 (s, 3H). MS m/z (M+H)=377.3.

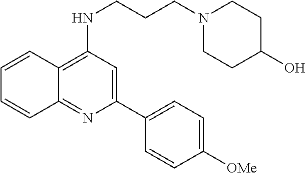 [SMILES: COc1ccc(-c2cc(NCCCN3CCC(O)CC3)c3ccccc3n2)cc1]

Example 75: 1-(3-((2-(4-Methoxyphenyl)quinolin-4-yl)amino)propyl)piperidin-4-ol. Into a vial with N-(3-bromopropyl)-2-(4-methoxyphenyl)quinolin-4-amine (0.1 g, 0.27 mmol) dissolved in dichloromethane (0.5 mL) was added 4-hydroxypiperidine (136 mg, 1.35 mmol). The reaction mixture was stirred at 23° C. over a period of 12 hours. The reaction mixture was then directly purified through silica gel cartridge eluting with dichloromethane/methanol with 5% ammonium hydroxide to give 1-(3-((2-(4-methoxyphenyl)quinolin-4-yl)amino)propyl)piperidin-4-ol as pale yellow powder (74 mg, 63%). ¹H NMR (CDCl₃) δ: 7.99-8.11 (m, 3H), 7.85 (dd, J=8.3, 1.4 Hz, 1H), 7.61 (ddd, J=8.4, 6.9, 1.4 Hz, 1H), 7.37 (ddd, J=8.3, 6.9, 1.2 Hz, 1H), 6.97-7.05 (m, 2H), 6.74 (s, 1H), 3.86 (s, 3H), 3.76-3.84 (m, 1H), 3.41-3.54 (m, 2H), 2.82-2.97 (m, 2H), 2.54-2.67 (m, 2H), 2.22 (br t, J=10.6 Hz, 2H), 1.92-2.07 (m, 4H), 1.86 (br s, 1H), 1.64-1.80 (m, 2H). MS m/z (M+H)=392.4.

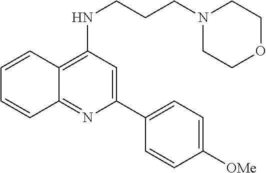 [SMILES: COc1ccc(-c2cc(NCCCN3CCOCC3)c3ccccc3n2)cc1]

Example 76: 2-(4-Methoxyphenyl)-N-(3-morpholinopropyl)quinolin-4-amine. To a solution of 4-chloro-6-methoxy-2-(4-methoxyphenyl)quinoline (0.1 g, 0.37 mmol) with 3-morpholinopropylamine (0.650 mL, 4.45 mmol) was added two drops of tin tetrachloride. The reaction mixture was stirred at 130° C. over a period of 12 hours. The reaction mixture was directly purified through silica gel cartridge eluting with dichloromethane/methanol with 5% ammonium hydroxide to give 2-(4-methoxyphenyl)-N-(3-morpholinopropyl)quinolin-4-amine as a yellow powder (112 mg, 82%). ¹H NMR (CDCl₃) δ: 7.98-8.09 (m, 3H), 7.84 (d, J=8.3 Hz, 1H), 7.60 (ddd, J=8.4, 6.9, 1.4 Hz, 1H), 7.37 (ddd, J=8.3, 6.9, 1.3 Hz, 1H), 6.99 (d, J=8.8 Hz, 2H), 6.92-6.97 (m, 1H), 6.74 (s, 1H), 3.84 (s, 3H), 3.79-3.83 (m, 3H), 3.66-3.72 (m, 1H), 3.42 (q, J=5.4 Hz, 2H), 2.48-2.64 (m, 4H), 2.37-2.45 (m, 2H), 1.92 (quin, J=5.8 Hz, 2H). MS m/z (M+H)=378.3.

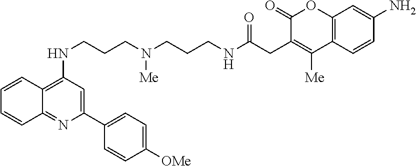 [SMILES: COc1ccc(-c2cc(NCCCN(C)CCCNC(=O)Cc3c(C)c4ccc(N)cc4oc3=O)c3ccccc3n2)cc1]

Example 77: 2-(7-Amino-4-methyl-2-oxo-2H-chromen-3-yl)-N-(3-((3-((2-(4-methoxyphenyl) quinolin-4-yl)amino)propyl)(methyl)amino)propyl)acetamide trihydrochloride. Into a vial was added N¹-(3-aminopropyl)-N³-(2-(4-methoxyphenyl)quinolin-4-yl)-N¹-methylpropane-1,3-diamine (75 mg, 0.2 mmol), 7-amino-4-methylcoumarinylacetic acid (42 mg, 0.18 mmol), 1-ethyl-3-(3-dimethylaminopropyl)carbodiimide (34.5 mg, 0.18 mmol) and 1-hydroxy-7-azabenzotriazole (24.5 mg, 0.18 mmol), together with dimethylformamide (2 mL) and diisopropylethylamine (78 μL, 0.43 mmol). The reaction was stirred overnight at 23° C. The reaction mixture was diluted with ethyl acetate, washed with saturated aqueous ammonium chloride, water (2×), 1N sodium hydroxide, brine, dried over magnesium sulfate and filtered. The resultant crude product was purified through silica gel cartridge eluting with dichloromethane/methanol with 5% ammonium hydroxide to give 2-(7-amino-4-methyl-2-oxo-2H-chromen-3-yl)-N-(3-((3-((2-(4-methoxyphenyl)quinolin-4-yl)amino)propyl)methylamino)propyl)acetamide. The obtained oil was dissolved in diethyl ether and excess of hydrogen chloride solution in diethyl ether was added. The solvent was evaporated and the resulting powder was dried under vacuum to give 2-(7-amino-4-methyl-2-oxo-2H-chromen-3-yl)-N-(3-((3-((2-(4-methoxyphenyl)quinolin-4-yl)amino)propyl)methylamino)propyl)acetamide trihydrochloride as a pale brown powder (60 mg, 57%). ¹H NMR (300 MHz, DMSO-d₆) δ13.66 (s, 1H), 10.93-10.55 (m, 1H), 9.55 (s, 1H), 8.69 (d, J=8.5 Hz, 1H), 8.26 (d, J=8.5 Hz, 1H), 8.12 (d, J=8.9 Hz, 2H), 8.08 (s, 1H), 7.97-7.86 (m, 1H), 7.65 (t, J=7.7 Hz, 1H), 7.44 (d, J=8.7 Hz, 1H), 7.17 (d, J=8.8 Hz, 2H), 7.05 (s, 1H), 6.77 (s, 3H), 6.62 (dd, J=8.7, 2.3 Hz, 1H), 6.46 (d, J=2.1 Hz, 1H), 3.85 (s, 3H), 3.82-3.70 (m, 2H), 3.54 (s, 3H), 3.13 (s, 2H), 3.12-3.03 (m, 2H), 2.7 (d, J=4.6 Hz, 2H), 2.22 (s, 3H), 2.20-2.11 (m, 2H), 1.92-1.76 (m, 2H), 1.33-1.18 (m, 2H). MS m/z (M+H)=594.1.

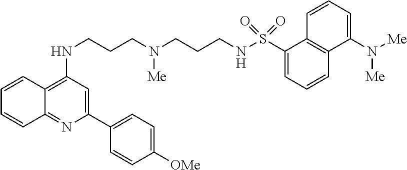 [SMILES: COc1ccc(-c2cc(NCCCN(C)CCCNS(=O)(=O)c3cccc4c(N(C)C)cccc34)c3ccccc3n2)cc1]

Example 78: Synthesis of 5-(dimethylamino)-N-{3-[(3-{[2-(4-methoxyphenyl)quinolin-4 yl]amino}propyl)(methyl)amino]propyl}naphthalene-1-sulfonamide. To a solution of N-{3-[(3-Aminopropyl)amino]propyl}-2-(4-methoxyphenyl)quinolin-4-amine (30 mg, 0.08 mmol) in dichloromethane was added triethylamine (33 μL, 0.24 mmol). After 5 minutes of stirring 5-(dimethylamino)naphthalene-1-sulfonyl chloride (24 mg, 0.087 mmol) was added and the reaction was stirred for a period of 16 hours at room temperature. The reaction was purified on silica gel cartridge eluting with dichloromethane and methanol with 5% ammonium hydroxide to afford 5-(dimethylamino)-N-{3-[(3-{[2-(4-methoxyphenyl)quinolin-4-yl]amino}propyl)(methyl)amino] propyl}naphthalene-1-sulfonamide 19 mg Yield 39%. ¹H NMR (CDCl₃) δ: 8.48 (d, J=8.6 Hz, 1H), 8.28 (d, J=8.7 Hz, 1H), 8.18 (dd, J=7.3, 1.3 Hz, 1H), 8.06 (d, J=8.8 Hz, 2H), 8.01 (s, 1H), 7.73 (d, J=8.3 Hz, 1H), 7.60 (ddd, J=8.4, 6.9, 1.3 Hz, 1H), 7.46 (ddd, J=14.5, 8.6, 7.4 Hz, 2H), 7.30-7.38 (m, 1H), 7.11 (d, J=7.5 Hz, 1H), 7.00 (d, J=8.8 Hz, 1H), 6.80 (s, 1H), 3.87 (s, 3H), 3.43-3.54 (m, 2H), 3.00 (t, J=5.9 Hz, 2H), 2.84 (s, 6H), 2.50 (t, J=6.4 Hz, 2H), 2.41 (t, J=6.2 Hz, 2H), 2.23 (s, 3H), 1.94 (t, J=6.4 Hz, 2H), 1.50-1.71 (m, 4H). MS m/z (M+H)==612.1.

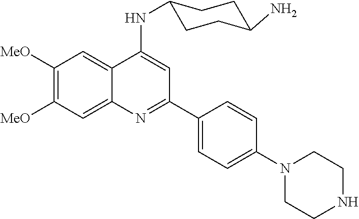 [SMILES: COc1cc2nc(-c3ccc(N4CCNCC4)cc3)cc(NC3CCC(N)CC3)c2cc1OC]

Example 81: (1R,4R)—N¹-(6,7-dimethoxy-2-(4-(piperazin-1-yl)phenyl)quinolin-4-yl)cyclohexane-1,4-diamine: To a stirring solution of tert-butyl 4-(4-(4-chloro-6,7-dimethoxyquinolin-2-yl)phenyl)piperazine-1-carboxylate (1E, 200 mg, 0.41 mmol) in dioxane (4.1 mL), tert-butyl ((1R,4R)-4-aminocyclohexyl)carbamate (176 mg, 0.82 mmol), palladium(II) acetate (9 mg, 0.04 mmol), 2,2′-bis(diphenylphosphino)-1,1′-binaphthalene (50 mg, 0.08 mmol), sodium tert-butoxide (118 mg, 1.23 mmol) were added. The resulting mixture was purged with nitrogen for 10 minutes then heated to 70° C. for 15 hours. The reaction was cooled down to room temperature, diluted with methylene chloride and water, filtered through celite. Extracted with methylene chloride (3×3 mL), dried over sodium sulfate and concentrated by reduced pressure. The crude was dissolved in methylene chloride (5 mL) and trifluoroacetic acid (0.47 mL, 6.15 mmol). The resulting mixture was stirred at room temperature for 1 hour. The excess solvent was removed under reduced pressure and purified through HPLC (0-50% acetonitrile in water) to give (1R,4R)—N¹-(6,7-dimethoxy-2-(4-(piperazin-1-yl)phenyl)quinolin-4-yl)cyclohexane-1,4-diamine as yellow solid (100 mg, 43%). ¹H NMR (300 MHz, Methanol-d4) δ 9.05-8.63 (m, 2H), 8.31 (d, J=3.8 Hz, 1H), 7.99 (d, J=1.5 Hz, 1H), 7.81 (d, J=8.9 Hz, 2H), 7.61 (s, 1H), 4.71 (d, J=5.3 Hz, 6H), 4.15 (d, J=6.1 Hz, 4H), 3.94 (t, J=5.0 Hz, 4H), 3.67 (dd, J=6.2, 3.9 Hz, 4H), 3.32 (p, J=1.8 Hz, 2H), 2.96-2.79 (m, 1H), 2.67 (d, J=12.6 Hz, 1H), 2.25 (t, J=11.9 Hz, 1H), 2.11 (dd, J=23.4, 10.8 Hz, 1H). MS m/z (M+H)=462.62.

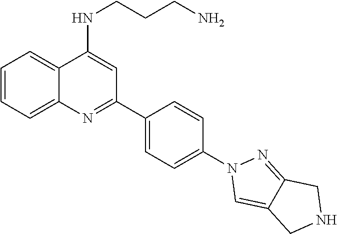 [SMILES: NCCCNc1cc(-c2ccc(-n3cc4c(n3)CNC4)cc2)nc2ccccc12]

### Example 84: Synthesis of N¹-(2-(4-(5,6-dihydropyrrolo[3,4-c]pyrazol-2(4H)-yl)phenyl)quinolin-4-yl)propane-1,3-diamine

Step 1: To a stirring solution of 4-fluoro-bromobenzene (175 mg, 1 mmol) and tert-Butyl 2H, 4H, 5H, 6H-pyrrolo[3,4-c]pyrazole-5-carboxylate (209 mg, 1 mmol) in N,N-dimethyl formamide (1.75 mL) was added sodium hydride (˜60% dispersion in mineral oil) (40 mg, 1 mmol) at 0° C. After stirring 30 min, the ice bath was removed and the reaction was headed to 130° C. for 5 h After cooling to ambient temperature, the reaction was quenched by the addition of water and the organic layer was extracted three times with dichloromethane (˜10 mL), dried over sodium sulfate, filtered, and concentrated in vacuo. The crude was purified via column chromatography using 0-25% EtOAc/hexanes to provide 170 mg (46%) of the title compound. MS m/z (M+H)=366.05.

Step 2: tert-butyl 2-(4-bromophenyl)-4,6-dihydropyrrolo[3,4-c]pyrazole-5(2H)-carboxylate (170 mg, 0.47 mmol), bis(pinacolato)diboron (178 mg, 0.7 mmol), potassium acetate (137 mg, 1.4 mmol), and [1,1′-Bis(diphenylphosphino)ferrocene]dichloropalladium(II) dichloromethane complex (20 mg, 0.02 mmol) were dissolved in dioxane (2.30 mL). The reaction mixture was placed under vacuum and backfilled with nitrogen three times before being heated to 100° C. and stirred overnight. After cooling to ambient temperature, the reaction was filtered and concentrated in vacuo. The crude was purified via column chromatography using 0-25% EtOAc/hexanes to provide 171 mg (88%) of (4-(5-(tert-butoxycarbonyl)-5,6-dihydropyrrolo[3,4-c]pyrazol-2(4H)-yl)phenyl)boronic acid pinacol ester. MS m/z (M+H)=412.23.

Step 3: tert-butyl 2-(4-(4-chloroquinolin-2-yl)phenyl)-4,6-dihydropyrrolo[3,4-c]pyrazole-5(2H)-carboxylate. 2,4-Dichloroquinoline (78 mg, 0.4 mmol), (4-(5-(tert-butoxycarbonyl)-5,6-dihydropyrrolo[3,4-c]pyrazol-2(4H)-yl)phenyl)boronic acid pinacol ester (170 mg, 0.41 mmol), tetrakis triphenylphosphine-palladium (14 mg, 0.012 mmol), aqueous 2M sodium carbonate (0.4 mL, 0.8 mmol) were dissolved in toluene (0.8 mL) and ethanol (0.8 mL). The reaction mixture was placed under vacuum and backfilled with nitrogen three times before being heated to 90° C. and stirred overnight. After cooling to ambient temperature, the reaction was quenched by the addition of water and the organic layer was extracted three times with dichloromethane (10 mL), dried over sodium sulfate, filtered, and concentrated in vacuo. The crude was purified via column chromatography using 0-100% EtOAc/hexanes to provide 80 mg (45%) of tert-butyl 2-(4-(4-chloroquinolin-2-yl)phenyl)-4,6-dihydropyrrolo[3,4-c]pyrazole-5(2H)-carboxylate. MS m/z (M+H)=447.25. Step 4: tert-butyl 2-(4-(4-chloroquinolin-2-yl)phenyl)-4,6-dihydropyrrolo[3,4-c]pyrazole-5(2H)-carboxylate (70 mg, 0.16 mmol), tert-butyl(3-aminopropyl)carbamate (82 mg, 0.47 mmol), palladium acetate (2 mg, 0.008 mmol), BINAP (11 mg, 0.02 mmol), and potassium phosphate tribasic (16 mg, 0.31 mmol) were dissolved in dioxane (1.0 mL) and water (0.1 mL). The reaction mixture was placed under vacuum and backfilled with nitrogen three times before being heated to reflux and stirred overnight. After cooling to ambient temperature, water was added and the organic layer was extracted three times with dichloromethane (10 mL), dried over sodium sulfate, filtered, and concentrated in vacuo. The crude was purified via column chromatography using 0-100% EtOAc/hexanes to provide 80 mg (86%) of tert-butyl 2-(4-(4-((3-((tert-butoxycarbonyl)amino)propyl)amino)quinolin-2-yl)phenyl)-4,6-dihydropyrrolo[3,4-c]pyrazole-5(2H)-carboxylate. ¹H NMR (300 MHz, Chloroform-d) δ ppm 8.15-8.21 (m, 2H) 8.02-8.09 (m, 1H) 7.86-7.95 (m, 1H) 7.73-7.80 (m, 2H) 7.61-7.73 (m, 2H) 7.39-7.54 (m, 1H) 6.83-6.89 (s, 1H), 4.43-4.88 (m, 4H), 3.46-3.58 (m, 2H), 3.27-3.39 (m, 2H), 1.84-1.98 (m, 2H), 1.53 (s, 9H), 1.49 (s, 9H). MS m/z (M+H)=585.44.

Step 5: N¹-(2-(4-(5,6-dihydropyrrolo[3,4-c]pyrazol-2(4H)-yl)phenyl)quinolin-4-yl)propane-1,3-diamine. tert-butyl 2-(4-(4-((3-((tert-butoxycarbonyl)amino)propyl)amino)quinolin-2-yl)phenyl)-4,6-dihydropyrrolo[3,4-c]pyrazole-5(2H)-carboxylate (50 mg, 0.09 mmol) was dissolved in dichloromethane (0.5 mL) and treated with hydrogen chloride, 4N in dioxane (0.43 mL, 1.7 mmol). The reaction was stirred for 1 h at ambient temperature and then concentrated in vacuo. The mixture was then treated with ammonium hydroxide (˜4 mL) and the organic phase was extracted twice with 10% isopropanol/dichloromethane (10 mL) and dried over sodium sulfate, filtered, and concentrated in vacuo to provide the free base. A solution of 2 N HCl in diethyl ether was added and concentrated in vacuo to give 42 mg of N-(3-aminopropyl)-2-(4-(5,6-dihydropyrrolo[3,4-c]pyrazol-2(4H)-yl)phenyl)quinolin-4-amine as an HCl salt. ¹H NMR (300 MHz, DMSO-d₆) δ ppm 13.76-13.94 (m, 1H), 10.85-11.06 (m, 1H), 10.55-10.77 (m, 1H), 9.70 (br s, 1H), 8.69 (t, J=7.5 Hz, 1H), 8.50 (s, 1H), 8.16-8.35 (m, 2H), 7.99 (d, J=8.79 Hz, 1H), 7.92 (t, J=7.62 Hz, 1H), 7.80 (d, J=8.79 Hz, 1H), 7.69 (s, 1H), 7.64 (t, J=7.62 Hz, 1H), 7.10 (s, 1H), 4.80 (br s, 1H), 4.38 (br d, J=4.10 Hz, 2H), 4.31 (s, 1H), 3.73-3.86 (m, 2H), 2.90-2.98 (m, 2H), 1.96-2.15 (m, 2H). MS m/z (M+H)=385.23.

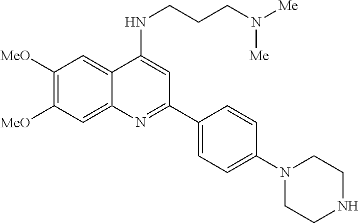 [SMILES: COc1cc2nc(-c3ccc(N4CCNCC4)cc3)cc(NCCCN(C)C)c2cc1OC]

### Example 87: Synthesis of N-(3-(dimethylamino)propyl)-6,7-dimethoxy-2-(4-(piperazin-1-yl)phenyl)quinolin-4-amine

Step 1: Common Intermediate IC: To a solution of 2,4-dichloro-6,7-dimethoxyquinoline (680 mg, 2.63 mmol) in 1,4 dioxane and water (1:1, 20 mL) was added tert-butyl 4-(4-(4,4,5,5-tetramethyl-1,3-dioxolan-2-yl)phenyl)piperazine-1-carboxylate (1028 mg, 2.63 mmol) and cesium carbonate (1.29 g, 3.95 mmol) degassed for 10 minutes with a stream of nitrogen, was added tetrakisstriphenylphosphine palladium (0) (91 mg, 0.08 mmol). The resulting mixture was heated to 90° C. for 3 hours. The reaction was cooled to room temperature and partitioned between ethyl acetate and water, purified on silica gel cartridge eluting with ethyl acetate in hexanes to afford tert-butyl 4-(4-(4-chloro-6,7-dimethoxyquinolin-2-yl)phenyl)piperazine-1-carboxylate 535 mg Yield 42%. MS m/z (M+H)=612.1.

Step 2: To a degassed solution of N1,N1-dimethylpropane-1,3-diamine (127 mg, 1.24 mmol) and tert-butyl 4-(4-(4-chloro-6,7-dimethoxyquinolin-2-yl)phenyl)piperazine-1-carboxylate (200 mg, 0.41 mmol) in dioxane and water (10:1, 11 mL) was added potassium phosphate tribasic (39 mg, 0.06 mmol), palladium(II) acetate (4.6 mg, 0.02 mmol) and BINAP (39 mg, 0.06 mmol). The resulting reaction mixture was stirred at 90° C. for 18 hours. Phases were separated and the aqueous phase was extracted with ethyl acetate (3 times 10 mL), the solvent was removed in vacuo. Purified on silica gel with methylene chloride methanol with 5% ammonium hydroxide. Recovered tert-butyl 4-(4-(4-(3-(dimethylamino)propylamino)-6,7-dimethoxyquinolin-2-yl)phenyl)piperazine-1-carboxylate as a foam (200 mg, 89%).

Step 3: tert-butyl 4-(4-(4-(3-(dimethylamino)propylamino)-6,7-dimethoxyquinolin-2-yl)phenyl)piperazine-1-carboxylate (200 mg, 0.36 mmol) was dissolved in dichloromethane (5 mL), hereto was added a solution of hydrogen chloride in dioxane, resulting in a heterogenous mixture. Stirred for 1 hour, concentrated under reduced pressure, and dried under high vacuum over night to afford tert-butyl 4-(4-(4-(3-(dimethylamino)propylamino)-6,7-dimethoxyquinolin-2-yl)phenyl)piperazine-1-carboxylate (148 mg, 89%). ¹H NMR (300 MHz, DMSO-d₆) δ ppm 10.56-10.86 (m, 1H) 9.44-9.62 (m, 2H) 9.19-9.39 (m, 1H) 7.98-8.12 (m, 3H) 7.76-7.88 (m, 1H) 7.05-7.26 (m, 2H) 6.83-6.95 (m, 1H) 3.92 (d, J=15.24 Hz, 6H) 3.64-3.77 (m, 2H) 3.53-3.62 (m, 4H) 3.12-3.25 (m, 6H) 2.69-2.79 (m, 6) 2.42-2.53 (m, 4H) 2.02-2.23 (m, 2H). (m,2H). MS m/z (M+) 450.65.

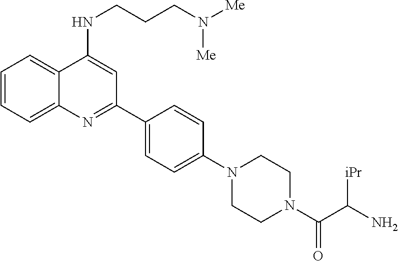 [SMILES: CC(C)C(N)C(=O)N1CCN(c2ccc(-c3cc(NCCCN(C)C)c4ccccc4n3)cc2)CC1]

### Example 91: Synthesis of 2-amino-1-(4-(4-(4-((3-(dimethylamino)propyl)amino)quinolin-2-yl)phenyl)piperazin-1-yl)-3-methylbutan-1-one

Step 1: N¹,N¹-dimethyl-N3-(2-(4-(piperazin-1-yl)phenyl)quinolin-4-yl)propane-1,3-diamine HCl (230 mg, 0.4 mmol), N-Boc-valine (109 mg, 0.5 mmol), N-(3-Dimethylaminopropyl)-N′-ethylcarbodiimide hydrochloride (116 mg, 0.6 mmol), 1-Hydroxy-7-azabenzotriazole (110 mg, 0.8 mmol), and N,N-diisopropylethylamine (0.42 mL, 2.4 mmol) was dissolved in N,N-dimethylformamide (2 mL). The reaction stirred overnight at ambient temperature before being quenched by the addition of water (5 mL). The organic phase was extracted three times with dichloromethane (˜10 mL), dried over sodium sulfate, filtered, and concentrated in vacuo. The crude was purified via column chromatography using 0-100% EtOAc/hexanes to provide 134 mg (57%) of tert-butyl(1-(4-(4-(4-((3-(dimethylamino)propyl)amino)quinolin-2-yl)phenyl)piperazin-1-yl)-3-methyl-1-oxobutan-2-yl)carbamate. ¹H NMR (300 MHz, Chloroform-d) δ ppm 8.07 (d, J=8.79 Hz, 2H), 7.57-7.70 (m, 2H), 7.32-7.41 (m, 1H), 7.02 (d, J=8.79 Hz, 2H), 6.73 (s, 1H), 5.34 (d, J=8.79 Hz, 1H), 4.47-4.55 (m, 1H), 3.66-3.90 (m, 4H), 3.43-3.59 (m, 2H), 3.19-3.38 (m, 4H), 2.52-2.66 (m, 2H), 2.39 (s, 6H), 1.90-2.03 (m, 2H), 1.44 (s, 9H), 0.99 (d, J=6.45 Hz, 3H), 0.92 (d, J=7.03 Hz, 2H). MS m/z (M+H)=589.74.

Step 2: tert-butyl(1-(4-(4-(4-((3-(dimethylamino)propyl)amino)quinolin-2-yl)phenyl)piperazin-1-yl)-3-methyl-1-oxobutan-2-yl)carbamate (132 mg, 0.22 mmol) was dissolved in dichloromethane (0.5 mL) and treated with hydrogen chloride, 4N in dioxane (0.56 mL, 2.2 mmol). The reaction was stirred for 1 h at ambient temperature and then concentrated in vacuo to give 140 mg of the 2-amino-1-(4-(4-(4-((3-(dimethylamino)propyl)amino)quinolin-2-yl)phenyl)piperazin-1-yl)-3-methylbutan-1-one. ¹H NMR (300 MHz, DMSO-d₆) δ ppm 13.44-13.65 (br m, 1H), 10.70-11.08 (br s, 1H), 9.37-9.55 (br s, 1H), 8.58-8.75 (m, 1H), 8.27-8.38 (m, 2H), 8.06-8.17 (m, 2H), 7.79-7.95 (m, 1H), 7.54-7.70 (m, 1H), 7.06-7.20 (m, 2H), 6.98 (s, 1H), 4.17-4.38 (m, 1H), 3.69-3.88 (m, 4H), 3.38-3.50 (m, 4H), 3.08-3.23 (m, 2H), 2.63-2.81 (m, 6H), 2.09-2.21 (m, 2H), 1.14-1.33 (m, 1H), 0.95-1.01 (m, 2H), 0.87-0.95 (m, 2H). MS m/z (M+H)=489.62.

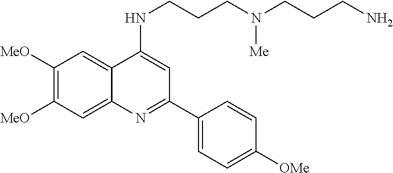 [SMILES: COc1ccc(-c2cc(NCCCN(C)CCCN)c3cc(OC)c(OC)cc3n2)cc1]

Example 94: Synthesis of N-(3-(dimethylamino)propyl)-6-fluoro-2-(4-(piperazin-1-yl)phenyl)quinolin-4-amine: To a solution of intermediate IC (100 mg, 0.3 mmol) was added 3,3′-Diamino-N-methyldipropylamine (528 mg, 3.63 mmol) and tin tetrachloride (2 drops). The reaction was heated to 135° C. over night. Purified on silica gel eluting with dichloromethane/methanol with 5% ammonium hydroxide, recovered a white foam, dissolved in dichloromethane and treated with two equivalent of hydrogen chloride in dioxane. Isolated N-{3-[(3-aminopropyl)(methyl)amino]propyl}-6,7-dimethoxy-2-(4-methoxyphenyl)quinolin-4-amine dihydrochloride 14 mg, yield 11%. ¹H NMR (300 MHz, DMSO-d₆) δ ppm 10.73-10.92 (m, 1H) 9.59-9.87 (m, 2H) 9.40-9.56 (m, 1H) 8.59-8.77 (m, 1H) 8.36-8.53 (m, 1H) 8.07 (br d, J=8.21 Hz, 2H) 7.70-7.87 (m, 1H) 7.07 (br d, J=8.79 Hz, 2H) 6.82-6.97 (m, 1H) 3.72 (br d, J=4.69 Hz, 2H) 3.57 (br s, 6H) 3.45 (br s, 6H) 2.72 (br d, J=3.52 Hz, 6H) 2.01-2.32 (m, 2H). MS m/z (M+) 439.32.

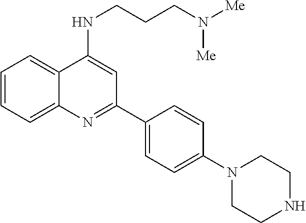 [SMILES: CN(C)CCCNc1cc(-c2ccc(N3CCNCC3)cc2)nc2ccccc12]

### Example 99: Synthesis of N¹,N¹-dimethyl-N3-(2-(4-(piperazin-1-yl)phenyl)quinolin-4-yl)propane-1,3-diamine

Step 1: 2,4-Dichloroquinoline (500 mg, 2.5 mmol), (4-(4-(tert-butoxycarbonyl)piperazin-1-yl)phenyl)boronic acid pinacol ester (1.03 g, 2.65 mmol), tetrakis triphenylphosphine-palladium (87 mg, 0.07 mmol), aqueous 2M sodium carbonate (2.5 mL, 5 mmol) were dissolved in toluene (5 mL) and ethanol (5 mL). The reaction mixture was placed under vacuum and backfilled with nitrogen three times before being heated to 90° C. and stirred overnight. After cooling to ambient temperature, the reaction was quenched by the addition of water and the organic layer was extracted three times with dichloromethane (˜10 mL), dried over sodium sulfate, filtered, and concentrated in vacuo. The crude was purified via column chromatography using 0-100% EtOAc/hexanes to provide 1.92 g (91%) of tert-butyl 4-(4-(4-chloroquinolin-2-yl)phenyl)piperazine-1-carboxylate Intermediate ID.

Step 2: Intermediate ID (500 mg, 1.2 mmol), N¹,N¹-dimethylpropane-1,3-diamine (0.446 mL, 3.5 mmol), palladium acetate (13 mg, 0.06 mmol), BINAP (81 mg, 0.13 mmol), and potassium phosphate tribasic (501 mg, 2.4 mmol) were dissolved in dioxane (4.4 mL) and water (0.5 mL). The reaction mixture was placed under vacuum and backfilled with nitrogen three times before being heated to reflux and stirred overnight. After cooling to ambient temperature, the reaction was partitioned between water and the organic layer was extracted three times with dichloromethane (10 mL), dried over sodium sulfate, filtered, and concentrated in vacuo. The crude was purified via column chromatography using 0-100% EtOAc/hexanes to provide 560 mg (95%) of tert-butyl 4-(4-(4-(3-(dimethylamino)propylamino)quinolin-2-yl)phenyl)piperazine-1-carboxylate.

Step 3: tert-butyl 4-(4-(4-((3-(dimethylamino)propyl)amino)quinolin-2-yl)phenyl)piperazine-1-carboxylate (560 mg, 1.14 mmol) was dissolved in dichloromethane (0.5 mL) and treated with hydrogen chloride, 4N in dioxane (2.8 mL, 11.2 mmol). The reaction was stirred for 1 h at ambient temperature and then concentrated in vacuo. The mixture was then treated with ammonium hydroxide (˜4 mL) and the organic phase was extracted twice with 10% isopropanol/dichloromethane (10 mL) and dried over sodium sulfate, filtered, and concentrated in vacuo to provide the free base. A solution of 2 N HCl in diethyl ether was added and concentrated in vacuo to give 484 mg of the title compound as a bis HCl salt. ¹H NMR (300 MHz, DMSO-d₆) δ ppm 9.24-11.63 (br s, 1H), 8.63 (br s, 1H), 8.54 (d, J=8.79 Hz, 1H), 8.16 (s, 1H), 8.09-8.15 (m, 1H), 7.76 (t, J=8.5 Hz, 1H), 7.51 (t, J=8.5 Hz, 1H), 7.13 (d, J=8.79 Hz, 2H), 6.99 (s, 1H) 3.62-3.70 (m, 2H), 3.52-3.60 (m, 4H), 3.10-3.26 (m, 7H), 2.71 (s, 6H), 2.14 (quin, J=7.03 Hz, 2H). MS m/z (M+H)=390.78.

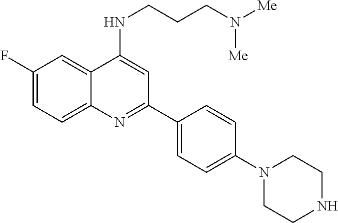 [SMILES: CN(C)CCCNc1cc(-c2ccc(N3CCNCC3)cc2)nc2ccc(F)cc12]

### Example 103: Synthesis of N-(3-(dimethylamino)propyl)-6-fluoro-2-(4-(piperazin-1-yl)phenyl)quinolin-4-amine

Step 1: To a solution of 2,4-dichloro-6-fluoroquinoline (500 mg, 2.31 mmol) in toluene, ethanol and water (5:5:1.2 mL) was added tert-butyl 4-(4-(4,4,5,5-tetramethyl-1,3-dioxolan-2-yl)phenyl)piperazine-1-carboxylate (1028 mg, 2.63 mmol) and sodium carbonate (1.3 mL, 2 m, 4.6 mL) degassed for 10 minutes with a stream of nitrogen, was added tetrakisstriphenylphosphine palladium (0) (80 mg, 0.03 mmol). The resulting mixture was heated to 70° C. for 3 hours. The reaction was cooled to room temperature and concentrated, loaded straight on silica gel cartridge eluting with ethyl acetate in hexanes to afford tert-butyl 4-(4-(4-chloro-6-fluoroquinolin-2-yl)phenyl)piperazine-1-carboxylate 600 mg Yield 59%.

Step 2: To a degassed solution of N¹,N¹-dimethylpropane-1,3-diamine (139 mg, 1.36 mmol) and tert-butyl 4-(4-(4-chloro-6-fluoroquinolin-2-yl)phenyl)piperazine-1-carboxylate (200 mg, 0.45 mmol) in dioxane and water (10:1, 11 mL) was added potassium phosphate tribasic (42 mg, 0.07 mmol), palladium(II) acetate (5.08 mg, 0.05 mmol) and BINAP (42 mg, 0.07 mmol). The resulting reaction mixture was stirred at 90° C. for 18 hours. Phases were separated and the aqueous phase was extracted with ethyl acetate (3 times 10 mL), the solvent was removed in vacuo. Purified with methylene chloride methanol with 5% ammonium hydroxide. Recovered tert-butyl 4-(4-(4-(3-(dimethylamino)propylamino)-6-fluoroquinolin-2-yl)phenyl)piperazine-1-carboxylate (200 mg, 89%).

Step 3: tert-butyl 4-(4-(4-(3-(dimethylamino)propylamino)-6-fluoroquinolin-2-yl)phenyl)piperazine-1-carboxylate (200 mg, 0.45 mmol) was dissolved in dichloromethane (5 mL), hereto was added a solution of hydrogen chloride in dioxane, resulting in a heterogenous mixture. Stirred for 1 hour, concentrated under reduced pressure, and dried under high vacuum over night to afford N-(3-(dimethylamino)propyl)-6-fluoro-2-(4-(piperazin-1-yl)phenyl)quinolin-4-amine (200 mg, 87%). ¹H NMR (300 MHz, DMSO-d₆) δ ppm 10.73-10.92 (m, 1H) 9.59-9.87 (m, 2H) 9.40-9.56 (m, 1H) 8.59-8.77 (m, 1H) 8.36-8.53 (m, 1H) 8.07 (br d, J=8.21 Hz, 2H) 7.70-7.87 (m, 1H) 7.07 (br d, J=8.79 Hz, 2 H) 6.82-6.97 (m, 1H) 3.72 (br d, J=4.69 Hz, 2H) 3.57 (br s, 6H) 3.45 (br s, 6H) 2.72 (br d, J=3.52 Hz, 6H) 2.01-2.32 (m, 2H). MS m/z (M+H)=408.42.

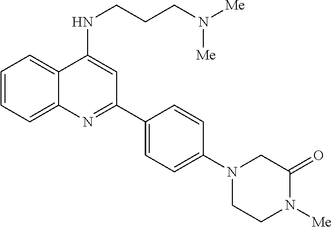 [SMILES: CN(C)CCCNc1cc(-c2ccc(N3CCN(C)C(=O)C3)cc2)nc2ccccc12]

### Example 115: Synthesis of 4-(4-(4-((3-(dimethylamino)propyl)amino)quinolin-2-yl)phenyl)-1-methylpiperazin-2-one

Step 1: 1-Methyl-2-piperazinone (228 mg, 2 mmol), 1,4-dibromobenzene (0.769 mL, 6 mmol), cesium carbonate (1.95 g, 5.98 mmol), palladium acetate (13 mg, 0.06 mmol), and XantPhos (69 mg, 0.12 mmol) were dissolved in toluene (10 mL). The reaction mixture was placed under vacuum and backfilled with nitrogen three times before being heated to reflux and stirred overnight. After cooling to ambient temperature, the reaction was quenched by the addition of water and the organic layer was extracted three times with dichloromethane (10 mL), dried over sodium sulfate, filtered, and concentrated in vacuo. The crude was purified via column chromatography using 0-50% EtOAc/hexanes to provide 200 mg (37%) of 4-(4-bromophenyl)-1-methylpiperazin-2-one MS m/z (M+H)=269.27.

Step 2: 4-(4-bromophenyl)-1-methylpiperazin-2-one (200 mg, 0.74 mmol), bis(pinacolato)diboron (283 mg, 1.11 mmol), potassium acetate (219 mg, 2.22 mmol), and [1,1′-bis(diphenylphosphino)ferrocene]dichloropalladium(II) dichloromethane complex (30 mg, 0.04 mmol) were dissolved in dioxane (3.7 mL). The reaction mixture was placed under vacuum and backfilled with nitrogen three times before being heated to 100° C. and stirred overnight. After cooling to ambient temperature, the reaction was filtered and concentrated in vacuo. The crude was purified via column chromatography using 0-25% EtOAc/hexanes to provide 175 mg (75%) of (4-(4-methyl-3-oxopiperazin-1-yl)phenyl)boronic acid pinacol ester. MS m/z (M+H)=317.43.

Step 3: 2,4-Dichloroquinoline (102 mg, 0.51 mmol), (4-(4-methyl-3-oxopiperazin-1-yl)phenyl)boronic acid pinacol ester (173 mg, 0.54 mmol), tetrakis triphenylphosphine-palladium (14 mg, 0.012 mmol), aqueous 2M sodium carbonate (0.4 mL, 0.8 mmol) were dissolved in toluene (0.8 mL) and ethanol (0.8 mL). The reaction mixture was placed under vacuum and backfilled with nitrogen three times before being heated to 90° C. and stirred overnight. After cooling to ambient temperature, the reaction was quenched by the addition of water and the organic layer was extracted three times with dichloromethane (˜10 mL), dried over sodium sulfate, filtered, and concentrated in vacuo. The crude was purified via column chromatography using 0-100% EtOAc/hexanes to provide 137 mg (76%) of 4-(4-(4-chloroquinolin-2-yl)phenyl)-1-methylpiperazin-2-one. ¹H NMR (300 MHz, Chloroform-d) δ ppm 8.20 (d, J=1.76 Hz, 1H), 8.08-8.15 (m, 3H), 7.92 (s, 1H), 7.74 (ddd, J=8.50, 7.03, 1.47 Hz, 1H), 7.53-7.63 (m, 1H), 6.98 (d, J=8.79 Hz, 2H), 4.00 (s, 2H), 3.57-3.66 (m, 2H), 3.47-3.56 (m, 2H), 3.06 (s, 3H). MS m/z (M+H)=352.25.

Step 4: 4-(4-(4-chloroquinolin-2-yl)phenyl)-1-methylpiperazin-2-one (135 mg, 0.38 mmol), N¹,N¹-dimethylpropane-1,3-diamine (0.145 mL, 1.15 mmol), palladium acetate (4 mg, 0.05 mmol), BINAP (26 mg, 0.11 mmol), and potassium phosphate tribasic (163 mg, 0.77 mmol) were dissolved in dioxane (1.5 mL) and water (0.16 mL). The reaction mixture was placed under vacuum and backfilled with nitrogen three times before being heated to reflux and stirred overnight. After cooling to ambient temperature, the reaction was quenched by the addition of water and the organic layer was extracted three times with dichloromethane (10 mL), dried over sodium sulfate, filtered, and concentrated in vacuo. The crude was purified via column chromatography using 0-10% methanol/dichloromethane. The pooled fractions were concentrated and then treated with 2 N HCl in diethyl ether and concentrated in vacuo to give 161 mg (86%) of 4-(4-(4-((3-(dimethylamino)propyl)amino)quinolin-2-yl)phenyl)-1-methylpiperazin-2-one HCl salt. ¹H NMR (300 MHz, DMSO-d₆) δ ppm 13.11-13.61 (m, 1H), 10.83 (br s, 1H), 9.46 (br t, J=5.28 Hz, 1H), 8.68 (d, J=8.21 Hz, 1H), 8.28 (d, J=8.21 Hz, 1H), 8.11 (d, J=9.38 Hz, 2H), 7.91 (t, J=7.91 Hz, 1H), 7.13 (d, J=8.79 Hz, 2H), 7.03 (s, 1H), 3.99 (s, 2H), 3.74-3.83 (m, 2H), 3.70 (t, J=4.5 Hz, 2H), 3.5 (t, J=4.75 Hz, 2H), 3.14-3.26 (m, 2H), 2.93 (s, 3H), 2.75 (d, J=4.69 Hz, 6H), 2.09-2.24 (m, 2H). MS m/z (M+H)=418.66.

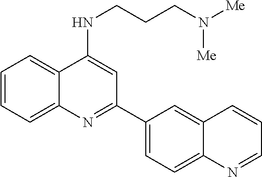 [SMILES: CN(C)CCCNc1cc(-c2ccc3ncccc3c2)nc2ccccc12]

### Example 119: Synthesis of N¹-([2,6′-biquinolin]-4-yl)-N³,N³-dimethylpropane-1,3-diamine

Step 1: 2,4-Dichloroquinoline (100 mg, 0.5 mmol), quinolin-6-ylboronic acid pinacol ester (135 mg, 0.53 mmol), tetrakis triphenylphosphine-palladium (17 mg, 0.015 mmol), aqueous 2M sodium carbonate (0.5 mL, 1 mmol) were dissolved in toluene (1 mL) and ethanol (1 mL). The reaction mixture was placed under vacuum and backfilled with nitrogen three times before being heated to 90° C. and stirred overnight. After cooling to ambient temperature, the reaction partitioned with water and the organic layer was extracted three times with dichloromethane (˜10 mL), dried over sodium sulfate, filtered, and concentrated in vacuo. The crude was purified via column chromatography using 0-100% EtOAc/hexanes to provide 139 mg (95%) of 4-chloro-2,6′-biquinoline. ¹H NMR (300 MHz, Chloroform-d) δ ppm 8.98 (dd, J=4.10, 1.17 Hz, 1H), 8.60-8.64 (m, 1H), 8.58 (dd, J=9.0, 3.0 Hz, 1H), 8.30-8.36 (m, 1H), 8.20-8.30 (m, 3H), 8.15 (s, 1H), 7.83 (t, J=7.62 Hz, 1H), 7.67 (t, J=7.72 Hz, 1H), 7.49 (dd, J=8.21, 4.10 Hz, 1H). MS m/z (M+H)=291.32.

Step 2: 4-chloro-2,6′-biquinoline (139 mg, 0.48 mmol), N¹,N¹-dimethylpropane-1,3-diamine (0.18 mL, 1.4 mmol), palladium acetate (5 mg, 0.02 mmol), BINAP (32 mg, 0.05 mmol), and potassium phosphate tribasic (203 mg, 0.96 mmol) were dissolved in dioxane (1.8 mL) and water (0.2 mL). The reaction mixture was placed under vacuum and backfilled with nitrogen three times before being heated to reflux and stirred overnight. After cooling to ambient temperature, the reaction was partitioned between water and organic layer was extracted three times with dichloromethane (10 mL), dried over sodium sulfate, filtered, and concentrated in vacuo. The crude was purified via column chromatography using 0-10% MeOH/dichloromethane. The pooled fractions were concentrated and treated with 2N HCl in diethyl ether and concentrated to provide 195 mg (95%) of Synthesis of N¹-([2,6′-biquinolin]-4-yl)-N³,N³-dimethylpropane-1,3-diamine HCl salt. ¹H NMR (300 MHz, DMSO-d₆) δ ppm 10.77-11.15 (br s, 1H), 9.88 (br t, J=5.57 Hz, 1H), 9.18 (dd, J=4.40, 1.47 Hz, 1H), 9.08 (d, J=8.50, 4.40 Hz, 1H), 8.86 (d, J=7.5 Hz, 1H), 8.78 (d, 7.3 Hz, 1H), 8.59 (dd, J=8.79, 1.76 Hz, 1H), 8.31-8.43 (m, 2H), 7.99 (t, J=7.91 Hz, 1H), 7.86 (dd, J=8.50, 4.40 Hz, 1H), 7.72 (t, J=7.33 Hz, 1H), 7.31 (s, 1H), 3.76-3.93 (m, 2H), 3.15-3.31 (m, 2H), 2.77 (d, J=4.69 Hz, 6H), 2.14-2.29 (m, 2H). MS m/z (M+H)=357.61.

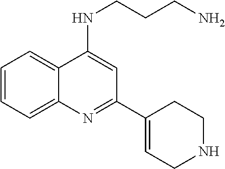 [SMILES: NCCCNc1cc(C2=CCNCC2)nc2ccccc12]

### Example 121: N¹-(2-(1,2,3,6-tetrahydropyridin-4-yl)quinolin-4-yl)propane-1,3-diamine

Step 1: 2,4-dichloroquinoline (500 mg, 2.5 mmol), 1-methyl-piperdine-4-boronic acid pinacol ester hydrochloride (780 mg, 2.5 mmol), tetrakis(triphenylphosphine)palladium(0) (87 mg, 0.075 mmol), potassium carbonate aqueous solution (691 mg in 4 mL water), toluene (8 mL) and ethanol (8 mL) were added into a 150 mL round bottom flask. The resulting mixture was purged with nitrogen for 20 minutes. The reaction was stirred at room temperature for 16 h. Toluene and ethanol were removed by reduced pressure and the aqueous layer was extract with methylene chloride three times (10 mL×3). The crude product was purified with silica gel chromatography (0-50% ethyl acetate in hexane) to give tert-butyl 4-(4-chloroquinolin-2-yl)-3,6-dihydropyridine-1(2H)-carboxylate as yellow solid (390 mg, 48%). ¹H NMR (300 MHz, Chloroform-d) δ 8.02 (d, J=0.7 Hz, 1H), 7.82-7.70 (m, 1H), 7.60 (ddd, J=8.4, 6.9, 1.3 Hz, 1H), 7.40-7.32 (m, 1H), 6.55 (s, 1H), 4.15 (q, J=3.0 Hz, 1H), 3.67 (dt, J=10.9, 5.9 Hz, 2H), 2.82-2.63 (m, 2H), 2.42 (t, J=6.2 Hz, 2H), 1.47 (d, J=2.5 Hz, 9H). MS m/z (M+H)=345.50.

Step 2: To a stirring solution of tert-butyl 4-(4-chloroquinolin-2-yl)-3,6-dihydropyridine-1(2H)-carboxylate (390 mg, 1.2 mmol) in dioxane (12 mL), tert-butyl(3-aminopropyl)carbamate (1.04 g, 5.97 mmol), palladium(II) acetate (13 mg, 0.06 mmol), 2,2′-bis(diphenylphosphino)-1,1′-binaphthalene (75 mg, 0.012 mmol), sodium tert-butoxide (346 mg, 3.6 mmol) were added. The resulting mixture was purged with nitrogen for 15 minutes then heated to 70° C. for 15 hours. The reaction was cooled down to room temperature, diluted with methylene chloride and water, filtered through celite. Extracted with methylene chloride (3×10 mL), dried over sodium sulfate and concentrated by reduced pressure. The crude was dissolved in methylene chloride (10 mL) and trifluoroacetic acid (1.38 mL, 18 mmol). The resulting mixture was stirred at room temperature for 1 hour. The excess solvent was removed under reduced pressure and purified through HPLC (0-50% acetonitrile in water) to give N¹-(2-(1,2,3,6-tetrahydropyridin-4-yl)quinolin-4-yl)propane-1,3-diamine as yellow solid (125 mg, 37%). ¹H NMR (300 MHz, Chloroform-d) δ 8.03-7.88 (m, 1H), 7.85-7.66 (m, 1H), 7.67-7.51 (m, 1H), 7.47-7.32 (m, 1H), 6.69 (dt, J=3.4, 1.7 Hz, 1H), 6.51 (s, 1H), 3.61 (q, J=2.9 Hz, 1H), 3.46 (t, J=6.2 Hz, 2H), 3.15 (t, J=5.7 Hz, 2H), 3.11-2.98 (m, 2H), 2.81-2.66 (m, 2H), 1.91 (dq, J=12.7, 6.4 Hz, 2H), 1.55 (s, 2H). MS m/z (M+H)=283.62.

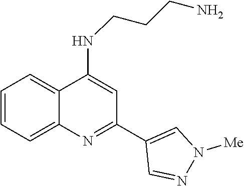 [SMILES: Cn1cc(-c2cc(NCCCN)c3ccccc3n2)cn1]

### Example 126: Synthesis of N¹-(2-(1-methyl-1H-pyrazol-4-yl)quinolin-4-yl)propane-1,3-diamine

Step 1: 2,4-dichloroquinoline (500 mg, 2.5 mmol), 1-methyl-piperdine-4-boronic acid pinacol ester hydrochloride (780 mg, 2.5 mmol), tetrakis(triphenylphosphine)palladium(0) (87 mg, 0.075 mmol), potassium carbonate aqueous solution (691 mg in 4 mL water), toluene (8 mL) and ethanol (8 mL) were added into a 150 mL round bottom flask. The resulting mixture was purged with nitrogen for 20 minutes. The reaction was stirred at room temperature for 16 h. Toluene and ethanol were removed by reduced pressure and the aqueous layer was extract with methylene chloride three times (10 mL×3). The crude product was purified with silica gel chromatography (0-50% ethyl acetate in hexane) to 4-chloro-2-(1-methyl-1H-pyrazol-4-yl)quinoline as yellow solid (380 mg, 63%). ¹H NMR (300 MHz, Chloroform-d) δ 8.17 (ddd, J=8.4, 1.4, 0.6 Hz, 1H), 8.12-8.07 (m, 2H), 8.04 (ddd, J=8.4, 1.3, 0.6 Hz, 1H), 7.73 (ddd, J=8.5, 6.9, 1.5 Hz, 1H), 7.69 (s, 1H), 7.62-7.51 (m, 1H), 4.00 (s, 3H). MS m/z (M+H)=244.77.

Step 2: To a stirring solution of 4-chloro-2-(1-methyl-1H-pyrazol-4-yl)quinoline (290 mg, 1.2 mmol) in dioxane (12 mL), tert-butyl(3-aminopropyl)carbamate (415 mg, 2.38 mmol), palladium(II) acetate (13 mg, 0.06 mmol),2,2′-bis(diphenylphosphino)-1,1′-binaphthalene (75 mg, 0.012 mmol), sodium tert-butoxide (346 mg, 3.6 mmol) were added. The resulting mixture was purged with nitrogen for 15 minutes then heated to 70° C. for 15 hours. The reaction was cooled down to room temperature, diluted with methylene chloride and water, filtered through celite. Extracted with methylene chloride (3×10 mL), dried over sodium sulfate and concentrated by reduced pressure. The crude was dissolved in methylene chloride (10 mL) and trifluoroacetic acid (1.38 mL, 18 mmol). The resulting mixture was stirred at room temperature for 1 hour. The excess solvent was removed under reduced pressure and purified through HPLC (0-50% acetonitrile in water) to give N¹-(2-(1-methyl-1H-pyrazol-4-yl)quinolin-4-yl)propane-1,3-diamine (235 mg, 52%). ¹H NMR (300 MHz, DMSO-d₆) δ 8.35 (d, J=0.7 Hz, 1H), 8.24-7.93 (m, 2H), 7.71 (ddd, J=8.4, 1.3, 0.6 Hz, 1H), 7.55 (ddd, J=8.3, 6.8, 1.4 Hz, 1H), 7.31 (ddd, J=8.3, 6.8, 1.4 Hz, 1H), 6.74 (s, 1H), 3.90 (s, 3H), 3.39 (t, J=6.9 Hz, 2H), 2.71 (t, J=6.5 Hz, 2H), 1.77 (p, J=6.7 Hz, 2H). MS m/z (M+H)=282.57.

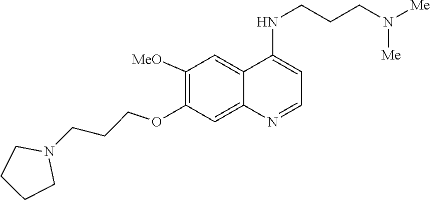 [SMILES: COc1cc2c(NCCCN(C)C)ccnc2cc1OCCCN1CCCC1]

### Example 145: Synthesis of N¹-(6-methoxy-7-(3-(pyrrolidin-1-yl)propoxy)quinolin-4-yl)-N³,N³-dimethylpropane-1,3-diamine

Step 1: 4-chloro-6-methoxyquinolin-7-ol (200 mg, 0.95 mmol), 1-(3-chloropropyl)pyrrolidine (423 mg, 2.86 mmol), potassium carbonate (263 mg, 1.9 mmol) and dimethylformamide (9.5 ml) were added into a 150 ml round bottom flask. The reaction was stirred at room temperature for 16 hours. The resulting mixture was dilute with water and extract with ethyl acetate three times. The organic layer was combined and washed with brine. The crude product was purified with column chromatography (0-100% ethyl acetate in hexane) to give 4-chloro-6-methoxy-7-(3-(pyrrolidin-1-yl)propoxy)quinoline as brown solid (225 mg, 74%). ¹H NMR (CDCl₃) δ 8.56 (d, J=4.9 Hz, 1H), 7.41 (s, 1H), 7.38 (s, 1H), 7.33 (d, J=4.8 Hz, 1H), 4.25 (t, J=6.6 Hz, 2H), 4.03 (s, 3H), 2.75 (dd, J=8.3, 6.7 Hz, 2H), 2.70-2.57 (m, 4H), 2.20 (dq, J=8.6, 6.6 Hz, 2H), 1.91-1.68 (m, 4H). MS m/z (M+H)=321.63.

Step 2: To a stirring solution of 4-chloro-6-methoxy-7-(3-(pyrrolidin-1-yl)propoxy)quinoline (100 mg, 0.31 mmol) in dioxane (7 ml), N,N-dimethylpropane-1,3-diamine (63 mg, 0.62 mmol), palladium(II) acetate (3 mg, 0.015 mmol), 2,2′-bis(diphenylphosphino)-1,1′-binaphthalene (19 mg, 0.03 mmol), sodium tert-butoxide (89 mg, 0.93 mmol) were added. The resulting mixture was purged with nitrogen for 10 minutes then heated to 70° C. for 15 hours. The reaction was cooled down to room temperature, diluted with methylene chloride and water, filtered through celite, extracted with methylene chloride three times. The organic layer was combined, dried over sodium sulfate, and concentrated by reduced pressure. The crude product was purified by HPLC (0-50% acetonitrile in water) to give N¹-(6-methoxy-7-(3-(pyrrolidin-1-yl)propoxy)quinolin-4-yl)-N³,N³-dimethylpropane-1,3-diamine as yellow solid (105 mg, 88%). ¹H NMR (CDCl₃) δ 8.32 (d, J=5.4 Hz, 1H), 7.56 (t, J=4.3 Hz, 1H), 7.29 (s, 1H), 6.91 (s, 1H), 6.23 (d, J=5.4 Hz, 1H), 4.17 (t, J=6.7 Hz, 2H), 3.91 (s, 3H), 3.34 (td, J=5.9, 4.3 Hz, 2H), 2.67-2.57 (m, 2H), 2.56 (d, J=0.7 Hz, 2H), 2.53-2.44 (m, 4H), 2.32 (s, 6H), 2.10 (dt, J=8.4, 6.8 Hz, 2H), 1.96-1.82 (m, 2H), 1.80-1.69 (m, 4H). MS m/z (M+H)=387.78.

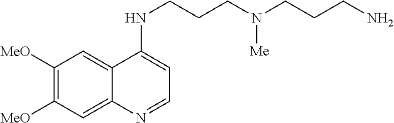 [SMILES: COc1cc2nccc(NCCCN(C)CCCN)c2cc1OC]

### Example 147: N¹-(3-aminopropyl)-N³-(6,7-dimethoxyquinolin-4-yl)-N¹-methylpropane-1,3-diamine

Step 1: To a stirring solution of 4-chloro-6,7-dimethoxyquinoline (200 mg, 0.89 mmol) in dioxane (9 ml), N¹-(3-aminopropyl)-N¹-methylpropane-1,3-diamine (260 mg, 1.79 mmol), palladium(II) acetate (10 mg, 0.04 mmol), 2,2′-bis(diphenylphosphino)-1,1′-binaphthalene (55 mg, 0.09 mmol), sodium tert-butoxide (257 mg, 2.67 mmol) were added. The resulting mixture was purged with nitrogen for 10 minutes then heated to 70° C. for 15 hours. The reaction was cooled down to room temperature, diluted with methylene chloride and water, filtered through celite, extracted with methylene chloride three times. The organic layer was combined, dried over sodium sulfate, and concentrated by reduced pressure. The crude product was purified through HPLC (0-50% acetonitrile in water) to give N¹-(3-aminopropyl)-N³-(6,7-dimethoxyquinolin-4-yl)-N¹-methylpropane-1,3-diamine as yellow solid (121 mg, 41%). ¹H NMR (CDCl₃) δ 8.28 (d, J=5.4 Hz, 1H), 7.22 (s, 2H), 6.96 (s, 1H), 6.20 (d, J=5.5 Hz, 1H), 3.86 (d, J=6.7 Hz, 6H), 3.26 (t, J=5.3 Hz, 2H), 2.61 (t, J=6.8 Hz, 2H), 2.55-2.44 (m, 2H), 2.44-2.33 (m, 2H), 2.25 (s, 3H), 2.08 (d, J=10.0 Hz, 2H), 1.92-1.71 (m, 2H), 1.55 (dd, J=8.2, 6.4 Hz, 2H). MS m/z (M+H)=333.60.

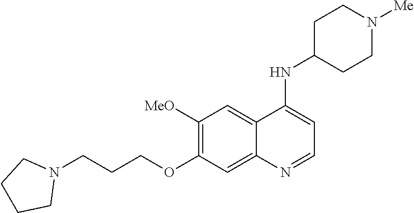 [SMILES: COc1cc2c(NC3CCN(C)CC3)ccnc2cc1OCCCN1CCCC1]

### Example 148: Synthesis of 6-methoxy-N-(1-methylpiperidin-4-yl)-7-(3-(pyrrolidin-1-yl)propoxy)quinolin-4-amine

Step 1: 4-chloro-6-methoxyquinolin-7-ol (200 mg, 0.95 mmol), 1-(3-chloropropyl)pyrrolidine (423 mg, 2.86 mmol), potassium carbonate (263 mg, 1.9 mmol) and dimethylformamide (9.5 ml) were added into a 150 ml round bottom flask. The reaction was stirred at room temperature for 16 hours. The resulting mixture was dilute with water and extract with ethyl acetate three times. The organic layer was combined and washed with brine. The crude product was purified with column chromatography (0-100% ethyl acetate in hexane) to give 4-chloro-6-methoxy-7-(3-(pyrrolidin-1-yl)propoxy)quinoline as brown solid (225 mg, 74%). ¹H NMR (CDCl₃) δ 8.56 (d, J=4.9 Hz, 1H), 7.41 (s, 1H), 7.38 (s, 1H), 7.33 (d, J=4.8 Hz, 1H), 4.25 (t, J=6.6 Hz, 2H), 4.03 (s, 3H), 2.75 (dd, J=8.3, 6.7 Hz, 2H), 2.70-2.57 (m, 4H), 2.20 (dq, J=8.6, 6.6 Hz, 2H), 1.91-1.68 (m, 4H). MS m/z (M+H)=321.63.

Step 2: To a stirring solution of 4-chloro-6-methoxy-7-(3-(pyrrolidin-1-yl)propoxy)quinoline (100 mg, 0.31 mmol) in dioxane (7 ml), 1-methylpiperidin-4-amine (71 mg, 0.62 mmol), palladium(II) acetate (3 mg, 0.015 mmol), 2,2′-bis(diphenylphosphino)-1,1′-binaphthalene (19 mg, 0.03 mmol), sodium tert-butoxide (89 mg, 0.93 mmol) were added. The resulting mixture was purged with nitrogen for 10 minutes then heated to 70° C. for 15 hours. The reaction was cooled down to room temperature diluted with methylene chloride and water, filtered through celite, extracted with methylene chloride three times. The organic layer was combined, dried over sodium sulfate, and concentrated by reduced pressure. The crude product was purified by HPLC to give 6-methoxy-N-(1-methylpiperidin-4-yl)-7-(3-(pyrrolidin-1-yl)propoxy)quinolin-4-amine as brown solid (55 mg, 44%). ¹H NMR (CDCl₃) δ 8.69 (dd, J=4.4, 1.7 Hz, 1H), 7.98 (ddd, J=8.3, 1.8, 0.6 Hz, 1H), 7.41 (d, J=0.7 Hz, 1H), 7.26-7.10 (m, 1H), 7.03 (s, 1H), 4.25 (t, J=6.6 Hz, 2H), 3.99 (s, 3H), 2.80-2.67 (m, 2H), 2.61 (td, J=6.8, 5.5, 2.8 Hz, 5H), 2.37-2.01 (m, 6H), 1.82 (ddt, J=6.8, 5.2, 2.3 Hz, 6H), 1.24 (s, 2H), 0.93-0.75 (m, 1H). MS m/z (M+H)=399.75.

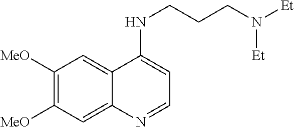 [SMILES: CCN(CC)CCCNc1ccnc2cc(OC)c(OC)cc12]

Example 149: Synthesis of N¹-(6,7-dimethoxyquinolin-4-yl)-N³,N³-diethylpropane-1,3-diamine. To a stirring solution of 4-chloro-6,7-dimethoxyquinoline (200 mg, 0.89 mmol) in dioxane (9 ml, 0.1 M), N,N-diethylpropane-1,3-diamine (233 mg, 1.79 mmol), palladium(II) acetate (10 mg, 0.04 mmol), 2,2′-bis(diphenylphosphino)-1,1′-binaphthalene (55 mg, 0.09 mmol), sodium tert-butoxide (257 mg, 2.67 mmol) were added. The resulting mixture was purged with nitrogen for 10 minutes then heated to 70° C. for 15 hours. The reaction was cooled down to room temperature diluted with methylene chloride and water, filtered through celite, extracted with methylene chloride three times. The organic layer was combined, dried over sodium sulfate, and concentrated by reduced pressure. The crude product was purified by HPLC (0-50% acetonitrile in water) to give N¹-(6,7-dimethoxyquinolin-4-yl)-N³,N³-diethylpropane-1,3-diamine as yellowish solid (230 mg, 72%). ¹H NMR (CDCl₃) δ 8.32 (dd, J=5.4, 0.5 Hz, 1H), 7.53-7.38 (m, 1H), 7.28 (s, 1H), 6.95 (s, 1H), 6.24 (d, J=5.5 Hz, 1H), 3.92 (d, J=7.9 Hz, 6H), 3.32 (td, J=5.7, 4.2 Hz, 2H), 2.73-2.35 (m, 6H), 2.00-1.65 (m, 2H), 1.03 (t, J=7.2 Hz, 6H). MS m/z (M+H)=318.62.

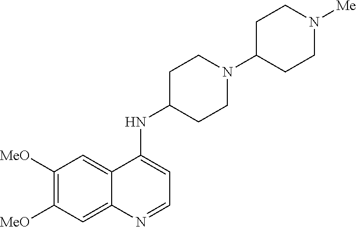 [SMILES: COc1cc2nccc(NC3CCN(C4CCN(C)CC4)CC3)c2cc1OC]

Example 150: Synthesis of 6,7-dimethoxy-N-(1′-methyl-[1,4′-bipiperidin]-4-yl)quinolin-4-amine: To a stirring solution of 4-chloro-6,7-dimethoxyquinoline (200 mg, 0.89 mmol) in dioxane (9 ml), 1′-methyl-[1,4′-bipiperidin]-4-amine (353 mg, 1.79 mmol), palladium(II) acetate (10 mg, 0.04 mmol), 2,2′-bis(diphenylphosphino)-1,1′-binaphthalene (55 mg, 0.09 mmol), sodium tert-butoxide (257 mg, 2.67 mmol) were added. The resulting mixture was purged with nitrogen for 10 minutes then heated to 70° C. for 15 hours. The reaction was cooled down to room temperature diluted with methylene chloride and water, filtered through celite, extracted with methylene chloride three times. The organic layer was combined, dried over sodium sulfate, and concentrated by reduced pressure. The crude product was purified by HPLC (0-50% acetonitrile in water) to give 6,7-dimethoxy-N-(1′-methyl-[1,4′-bipiperidin]-4-yl)quinolin-4-amine as yellow solid (202 mg, 59%). ¹H NMR (CDCl₃) δ 8.23 (d, J=5.5 Hz, 1H), 7.18 (s, 1H), 7.02 (s, 1H), 6.28 (d, J=5.7 Hz, 1H), 5.62-5.37 (m, 1H), 3.77 (d, J=14.0 Hz, 6H), 3.39 (dq, J=14.1, 6.8, 4.9 Hz, 1H), 2.90-2.69 (m, 4H), 2.27 (td, J=11.5, 2.1 Hz, 2H), 2.16 (s, 3H), 2.10-2.00 (m, 2H), 1.83 (td, J=11.7, 2.3 Hz, 2H), 1.75-1.62 (m, 2H), 1.53 (ddd, J=15.6, 12.0, 3.5 Hz, 4H). MS m/z (M+H)=385.82.

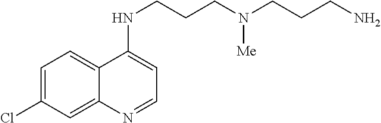 [SMILES: CN(CCCN)CCCNc1ccnc2cc(Cl)ccc12]

Example 151: Synthesis of N¹-(3-aminopropyl)-N³-(7-chloroquinolin-4-yl)-N¹-methylpropane-1,3-diamine: To a solution of 7-chloro-4-iodoquinoline (200 mg, 0.69 mmol) in dioxane (7 ml), N¹-(3-aminopropyl)-N¹-methylpropane-1,3-diamine (200 mg, 1.38 mmol), palladium(II) acetate (8 mg, 0.03 mmol), 2,2′-bis(diphenylphosphino)-1,1′-binaphthalene (43 mg, 0.07 mmol), sodium tert-butoxide (199 mg, 2.07 mmol) were added. The resulting mixture was purged with nitrogen for 10 minutes then heated to 70° C. for 15 hours. The reaction was cooled down to room temperature diluted with methylene chloride and water, filtered through celite, extracted with methylene chloride three times. The organic layer was combined, dried over sodium sulfate, and concentrated by reduced pressure. The crude product was purified by HPLC (0-50% acetonitrile in water) to give N¹-(3-aminopropyl)-N³-(7-chloroquinolin-4-yl)-N¹-methylpropane-1,3-diamine as yellow solid (105 mg, 50%). ¹H NMR (CDCl₃) δ 8.44 (d, J=5.4 Hz, 1H), 7.87 (d, J=2.1 Hz, 1H), 7.66 (d, J=8.9 Hz, 1H), 7.36-7.04 (m, 1H), 6.26 (d, J=5.4 Hz, 1H), 3.33 (t, J=6.0 Hz, 2H), 2.75 (t, J=6.8 Hz, 2H), 2.66-2.41 (m, 6H), 2.32 (s, 3H), 2.07-1.78 (m, 2H), 1.68 (p, J=6.9 Hz, 2H). MS m/z (M+H)=307.70.

### FORMULATIONS

The present invention also relates to compositions or formulations which comprise the TDP-43 binding agents according to the present invention. In general, the compositions of the present invention comprise an effective amount of one or more TDP-43 binding agents of the present invention and salts thereof according to the present invention which are effective for providing treatment or prevention of diseases that involve TDP-43, including, for example, ALS, FTLD, CTE, hippocampal sclerosis of aging (CARTS), Alzheimer's disease, or an Alzheimer's disease related disorder and one or more excipients.

For the purposes of the present invention the term “excipient” and “carrier” are used interchangeably throughout the description of the present invention and said terms are defined herein as, “ingredients which are used in the practice of formulating a safe and effective pharmaceutical composition.”

The formulator will understand that excipients are used primarily to serve in delivering a safe, stable, and functional pharmaceutical, serving not only as part of the overall vehicle for delivery but also as a means for achieving effective absorption by the recipient of the active ingredient. An excipient may fill a role as simple and direct as being an inert filler, or an excipient as used herein may be part of a pH stabilizing system or coating to insure delivery of the ingredients safely to the stomach. The formulator can also take advantage of the fact the compounds of the present invention have improved cellular potency, pharmacokinetic properties, as well as improved oral bioavailability.

The present teachings also provide pharmaceutical compositions that include at least one compound described herein and one or more pharmaceutically acceptable carriers, excipients, or diluents. Examples of such carriers are well known to those skilled in the art and can be prepared in accordance with acceptable pharmaceutical procedures, such as, for example, those described in Remington's Pharmaceutical Sciences, 17th edition, ed. Alfonoso R. Gennaro, Mack Publishing Company, Easton, Pa. (1985), the entire disclosure of which is incorporated by reference herein for all purposes. As used herein, “pharmaceutically acceptable” refers to a substance that is acceptable for use in pharmaceutical applications from a toxicological perspective and does not adversely interact with the active ingredient. Accordingly, pharmaceutically acceptable carriers are those that are compatible with the other ingredients in the formulation and are biologically acceptable. Supplementary active ingredients can also be incorporated into the pharmaceutical compositions.

Compounds of the present teachings can be administered orally or parenterally, neat or in combination with conventional pharmaceutical carriers. Applicable solid carriers can include one or more substances which can also act as flavoring agents, lubricants, solubilizers, suspending agents, fillers, glidants, compression aids, binders or tablet-disintegrating agents, or encapsulating materials. The compounds can be formulated in conventional manner, for example, in a manner similar to that used for known therapeutic agents. Oral formulations containing a compound disclosed herein can comprise any conventionally used oral form, including tablets, capsules, buccal forms, troches, lozenges and oral liquids, suspensions or solutions. In powders, the carrier can be a finely divided solid, which is an admixture with a finely divided compound. In tablets, a compound disclosed herein can be mixed with a carrier having the necessary compression properties in suitable proportions and compacted in the shape and size desired. The powders and tablets can contain up to 99% of the compound.

Capsules can contain mixtures of one or more compound(s) disclosed herein with inert filler(s) and/or diluent(s) such as pharmaceutically acceptable starches (e.g., corn, potato or tapioca starch), sugars, artificial sweetening agents, powdered celluloses (e.g., crystalline and microcrystalline celluloses), flours, gelatins, gums, and the like.

Useful tablet formulations can be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Surface modifying agents include nonionic and anionic surface modifying agents. Representative examples of surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecyl sulfate, magnesium aluminum silicate, and triethanolamine. Oral formulations herein can utilize standard delay or time-release formulations to alter the absorption of the compound(s). The oral formulation can also consist of administering a compound disclosed herein in water or fruit juice, containing appropriate solubilizers or emulsifiers as needed.

Liquid carriers can be used in preparing solutions, suspensions, emulsions, syrups, elixirs, and for inhaled delivery. A compound of the present teachings can be dissolved or suspended in a pharmaceutically acceptable liquid carrier such as water, an organic solvent, or a mixture of both, or a pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, and osmo-regulators. Examples of liquid carriers for oral and parenteral administration include, but are not limited to, water (particularly containing additives as described herein, e.g., cellulose derivatives such as a sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the carrier can be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellants.

Liquid pharmaceutical compositions, which are sterile solutions or suspensions, can be utilized by, for example, intramuscular, intraperitoneal or subcutaneous injection. Sterile solutions can also be administered intravenously. Compositions for oral administration can be in either liquid or solid form.

Preferably the pharmaceutical composition is in unit dosage form, for example, as tablets, capsules, powders, solutions, suspensions, emulsions, granules, or suppositories. In such form, the pharmaceutical composition can be sub-divided in unit dose(s) containing appropriate quantities of the compound. The unit dosage forms can be packaged compositions, for example, packeted powders, vials, ampoules, prefilled syringes or sachets containing liquids. Alternatively, the unit dosage form can be a capsule or tablet itself, or it can be the appropriate number of any such compositions in package form. Such unit dosage form can contain from about 1 mg/kg of compound to about 500 mg/kg of compound and can be given in a single dose or in two or more doses. Such doses can be administered in any manner useful in directing the compound(s) to the recipient's bloodstream, including orally, via implants, parenterally (including intravenous, intraperitoneal and subcutaneous injections), rectally, vaginally, and transdermally.

When administered for the treatment or inhibition of a particular disease state or disorder, it is understood that an effective dosage can vary depending upon the particular compound utilized, the mode of administration, and severity of the condition being treated, as well as the various physical factors related to the individual being treated. In therapeutic applications, a compound of the present teachings can be provided to a patient already suffering from a disease in an amount sufficient to cure or at least partially ameliorate the symptoms of the disease and its complications. The dosage to be used in the treatment of a specific individual typically must be subjectively determined by the attending physician. The variables involved include the specific condition and its state as well as the size, age and response pattern of the patient.

In some cases, it may be desirable to administer a compound directly to the airways of the patient, using devices such as, but not limited to, metered dose inhalers, breath-operated inhalers, multidose dry-powder inhalers, pumps, squeeze-actuated nebulized spray dispensers, aerosol dispensers, and aerosol nebulizers. For administration by intranasal or intrabronchial inhalation, the compounds of the present teachings can be formulated into a liquid composition, a solid composition, or an aerosol composition. The liquid composition can include, by way of illustration, one or more compounds of the present teachings dissolved, partially dissolved, or suspended in one or more pharmaceutically acceptable solvents and can be administered by, for example, a pump or a squeeze-actuated nebulized spray dispenser. The solvents can be, for example, isotonic saline or bacteriostatic water. The solid composition can be, by way of illustration, a powder preparation including one or more compounds of the present teachings intermixed with lactose or other inert powders that are acceptable for intrabronchial use, and can be administered by, for example, an aerosol dispenser or a device that breaks or punctures a capsule encasing the solid composition and delivers the solid composition for inhalation. The aerosol composition can include, by way of illustration, one or more compounds of the present teachings, propellants, surfactants, and co-solvents, and can be administered by, for example, a metered device. The propellants can be a chlorofluorocarbon (CFC), a hydrofluoroalkane (HFA), or other propellants that are physiologically and environmentally acceptable.

Compounds described herein can be administered parenterally or intraperitoneally. Solutions or suspensions of these compounds or a pharmaceutically acceptable salts, hydrates, or esters thereof can be prepared in water suitably mixed with a surfactant such as hydroxyl-propylcellulose. Dispersions can also be prepared in glycerol, liquid polyethylene glycols, and mixtures thereof in oils. Under ordinary conditions of storage and use, these preparations typically contain a preservative to inhibit the growth of microorganisms.

The pharmaceutical forms suitable for injection can include sterile aqueous solutions or dispersions and sterile powders for the extemporaneous preparation of sterile injectable solutions or dispersions. In some embodiments, the form can sterile and its viscosity permits it to flow through a syringe. The form preferably is stable under the conditions of manufacture and storage and can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Compounds described herein can be administered transdermally, i.e., administered across the surface of the body and the inner linings of bodily passages including epithelial and mucosal tissues. Such administration can be carried out using the compounds of the present teachings including pharmaceutically acceptable salts, hydrates, or esters thereof, in lotions, creams, foams, patches, suspensions, solutions, and suppositories (rectal and vaginal).

Transdermal administration can be accomplished through the use of a transdermal patch containing a compound, such as a compound disclosed herein, and a carrier that can be inert to the compound, can be non-toxic to the skin, and can allow delivery of the compound for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the compound can also be suitable. A variety of occlusive devices can be used to release the compound into the blood stream, such as a semi-permeable membrane covering a reservoir containing the compound with or without a carrier, or a matrix containing the compound. Other occlusive devices are known in the literature.

Compounds described herein can be administered rectally or vaginally in the form of a conventional suppository. Suppository formulations can be made from traditional materials, including cocoa butter, with or without the addition of waxes to alter the suppository's melting point, and glycerin. Water-soluble suppository bases, such as polyethylene glycols of various molecular weights, can also be used.

Lipid formulations or nanocapsules can be used to introduce compounds of the present teachings into host cells either in vitro or in vivo. Lipid formulations and nanocapsules can be prepared by methods known in the art.

To increase the effectiveness of compounds of the present teachings, it can be desirable to combine a compound with other agents effective in the treatment of the target disease. For example, other active compounds (i.e., other active ingredients or agents) effective in treating the target disease can be administered with compounds of the present teachings. The other agents can be administered at the same time or at different times than the compounds disclosed herein.

Compounds of the present teachings can be useful for the treatment or inhibition of a pathological condition or disorder in a mammal, for example, a human subject. The present teachings accordingly provide methods of treating or inhibiting a pathological condition or disorder by providing to a mammal a compound of the present teachings including its pharmaceutically acceptable salt) or a pharmaceutical composition that includes one or more compounds of the present teachings in combination or association with pharmaceutically acceptable carriers. Compounds of the present teachings can be administered alone or in combination with other therapeutically effective compounds or therapies for the treatment or inhibition of the pathological condition or disorder.

Non-limiting examples of compositions according to the present invention include from about 0.001 mg to about 1000 mg of one or more TDP-43 binding agents according to the present invention and one or more excipients; from about 0.01 mg to about 100 mg of one or more TDP-43 binding agents according to the present invention and one or more excipients; and from about 0.1 mg to about 10 mg of one or more TDP-43 binding agents according to the present invention; and one or more excipients.

### PROCEDURES

The following procedures can be utilized in evaluating and selecting compounds as TDP-43 binding agents.

Alpha-screen assay: Displacement of bt-TG6 binding was measured using AlphaScreen technology as previously described (Cassel, J. A. et al., Journal of Biomolecular Screening, 2010, 15, 1099-1106; Cassel, J. A.; Reitz, A. B. et al. The effects of small molecule inhibitors of nucleic acid binding to TDP-43 on TDP-43 metabolism and function. Biochimie, 2012, 94, 1974-1981).

Test compounds were diluted in assay buffer (25 mM Tris [pH 7.4], 0.1% bovine serum albumin [BSA], 0.1% Triton X-100) at 4× the desired final concentration. Dilutions of test compound (50 to 0.02 μM in half-log dilutions with a no compound control well) were pre-incubated with TDP-43 (5 μl of 60 ng/mL) pre-bound to AlphaScreen® anti-GST acceptor beads (5 μl of 40 μg/mL) in 15 μl volume for 30 minutes at 23° C. with shaking. Assays were initiated by the addition of 5 μl bt-TAR-32 (5′-CTG CTT TTT GCC TGT ACT GGG TCT CTG TGG TT-3′) 0.5 nM pre-bound to AlphaScreen® strepavidin donor beads (40 μg/mL). After incubation in the dark at 23° C. for 90 minutes with shaking, the AlphaScreen® signal was measured on a Synergy 2 plate reader (BioTek, Winooski, Vt.). IC₅₀ values were determined from nonlinear regression fits of the data to a Sigmoidal dose response variable slope model in GraphPad Prism. Final assay conditions are 25 mM Tris [pH 7.4], 0.1% bovine serum albumin [BSA], 0.100 Triton X-100, 15 ng/ml TDP-43, 0.125 nM bTAR32, 10 ug/ml GST donor bead, 10 ug/ml streptavidin acceptor bead.

Results for non-limiting representative compounds of the disclosure are listed in Table 1.

TABLE 1
Activity of test compounds in the TDP-43-DNA alpha-screen assay.

| Example | | Activity |
|---|---|---|
| number | Structure | (μM) |
| 1 | 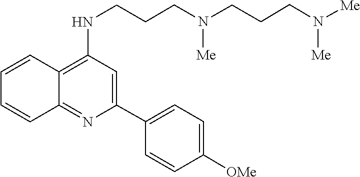 [SMILES: COc1ccc(-c2cc(NCCCN(C)CCCN(C)C)c3ccccc3n2)cc1] | 0.28 |
| 2 | 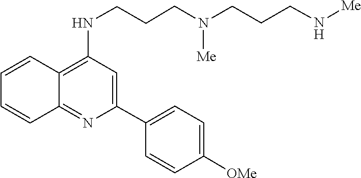 [SMILES: CNCCCN(C)CCCNc1cc(-c2ccc(OC)cc2)nc2ccccc12] | 0.11 |
| 3 | 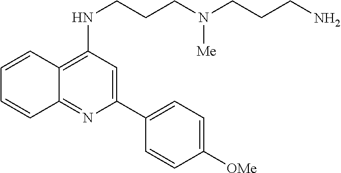 [SMILES: COc1ccc(-c2cc(NCCCN(C)CCCN)c3ccccc3n2)cc1] | 0.08 |
| 4 | 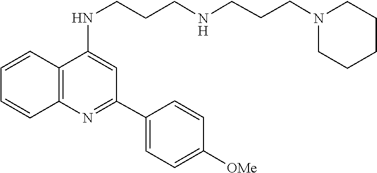 [SMILES: COc1ccc(-c2cc(NCCCNCCCN3CCCCC3)c3ccccc3n2)cc1] | 0.22 |
| 5 | 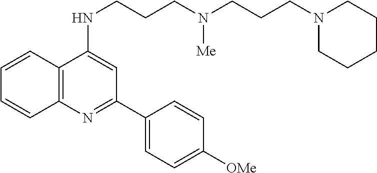 [SMILES: COc1ccc(-c2cc(NCCCN(C)CCCN3CCCCC3)c3ccccc3n2)cc1] | 0.24 |
| 6 | 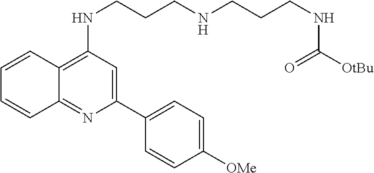 [SMILES: COc1ccc(-c2cc(NCCCNCCCNC(=O)OC(C)(C)C)c3ccccc3n2)cc1] | 0.76 |
| 7 | 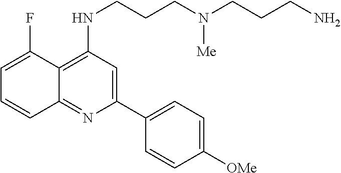 [SMILES: COc1ccc(-c2cc(NCCCN(C)CCCN)c3c(F)cccc3n2)cc1] | 0.22 |
| 8 | 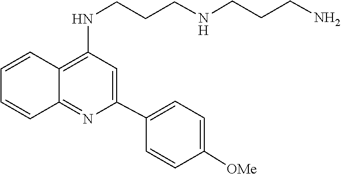 [SMILES: COc1ccc(-c2cc(NCCCNCCCN)c3ccccc3n2)cc1] | 0.08 |
| 9 | 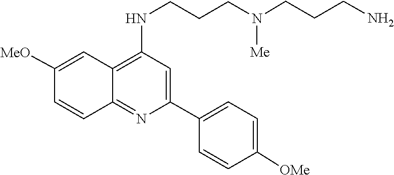 [SMILES: COc1ccc(-c2cc(NCCCN(C)CCCN)c3cc(OC)ccc3n2)cc1] | 0.05 |
| 10 | 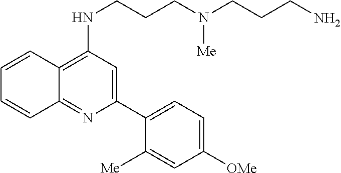 [SMILES: COc1ccc(-c2cc(NCCCN(C)CCCN)c3ccccc3n2)c(C)c1] | 0.65 |
| 11 | 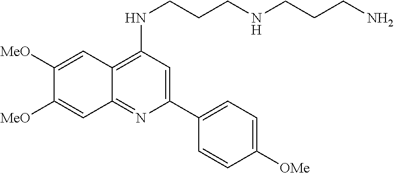 [SMILES: COc1ccc(-c2cc(NCCCNCCCN)c3cc(OC)c(OC)cc3n2)cc1] | 0.05 |
| 12 | 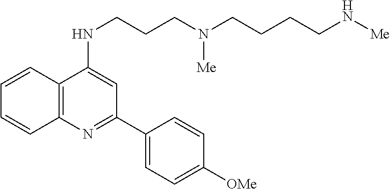 [SMILES: CNCCCCN(C)CCCNc1cc(-c2ccc(OC)cc2)nc2ccccc12] | 0.08 |
| 13 | 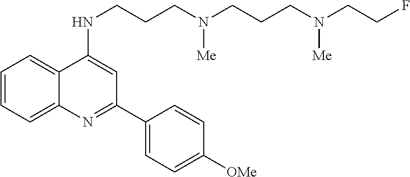 [SMILES: COc1ccc(-c2cc(NCCCN(C)CCCN(C)CCF)c3ccccc3n2)cc1] | 0.33 |
| 14 | 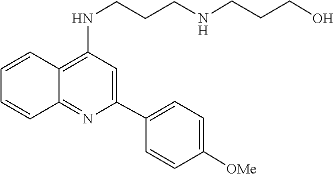 [SMILES: COc1ccc(-c2cc(NCCCNCCCO)c3ccccc3n2)cc1] | 0.90 |
| 15 | 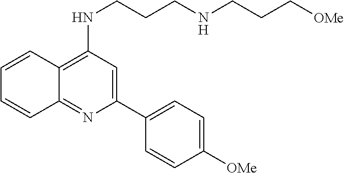 [SMILES: COCCCNCCCNc1cc(-c2ccc(OC)cc2)nc2ccccc12] | 0.80 |
| 16 | 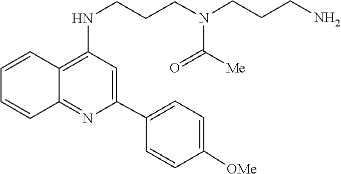 [SMILES: COc1ccc(-c2cc(NCCCN(CCCN)C(C)=O)c3ccccc3n2)cc1] | 1.42 |
| 17 | 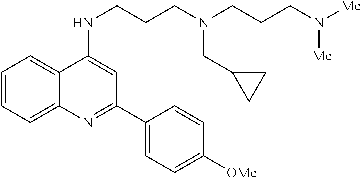 [SMILES: COc1ccc(-c2cc(NCCCN(CCCN(C)C)CC3CC3)c3ccccc3n2)cc1] | 0.18 |
| 18 | 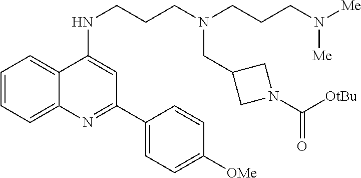 [SMILES: COc1ccc(-c2cc(NCCCN(CCCN(C)C)CC3CN(C(=O)OC(C)(C)C)C3)c3ccccc3n2)cc1] | 1.41 |
| 19 | 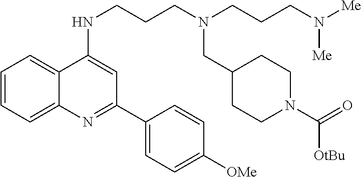 [SMILES: COc1ccc(-c2cc(NCCCN(CCCN(C)C)CC3CCN(C(=O)OC(C)(C)C)CC3)c3ccccc3n2)cc1] | 0.95 |
| 20 | 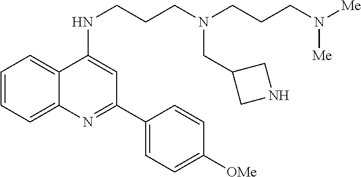 [SMILES: COc1ccc(-c2cc(NCCCN(CCCN(C)C)CC3CNC3)c3ccccc3n2)cc1] | 0.13 |
| 21 | 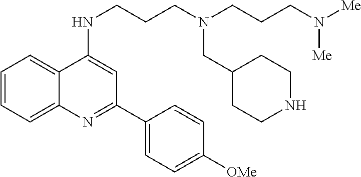 [SMILES: COc1ccc(-c2cc(NCCCN(CCCN(C)C)CC3CCNCC3)c3ccccc3n2)cc1] | 0.12 |
| 22 | 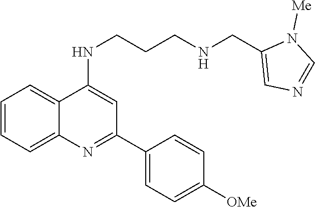 [SMILES: COc1ccc(-c2cc(NCCCNCc3cncn3C)c3ccccc3n2)cc1] | 0.68 |
| 23 | 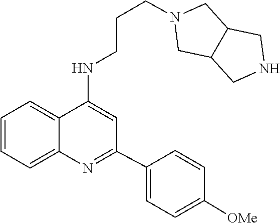 [SMILES: COc1ccc(-c2cc(NCCCN3CC4CNCC4C3)c3ccccc3n2)cc1] | 0.24 |
| 24 | 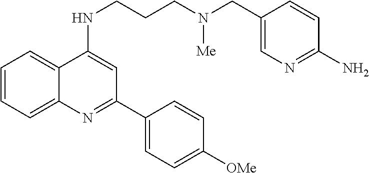 [SMILES: COc1ccc(-c2cc(NCCCN(C)Cc3ccc(N)nc3)c3ccccc3n2)cc1] | 1.11 |
| 25 | 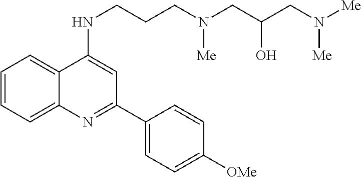 [SMILES: COc1ccc(-c2cc(NCCCN(C)CC(O)CN(C)C)c3ccccc3n2)cc1] | 0.22 |
| 26 | 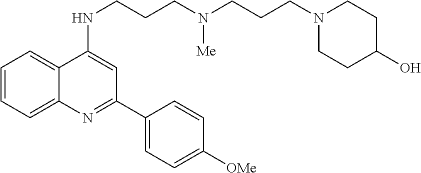 [SMILES: COc1ccc(-c2cc(NCCCN(C)CCCN3CCC(O)CC3)c3ccccc3n2)cc1] | 0.15 |
| 27 | 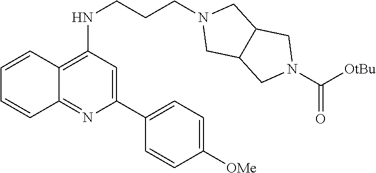 [SMILES: COc1ccc(-c2cc(NCCCN3CC4CN(C(=O)OC(C)(C)C)CC4C3)c3ccccc3n2)cc1] | 2.08 |
| 28 | 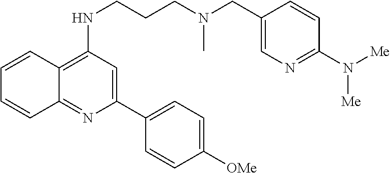 [SMILES: COc1ccc(-c2cc(NCCCN(C)Cc3ccc(N(C)C)nc3)c3ccccc3n2)cc1] | 1.21 |
| 29 | 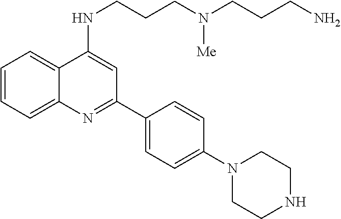 [SMILES: CN(CCCN)CCCNc1cc(-c2ccc(N3CCNCC3)cc2)nc2ccccc12] | 0.08 |
| 30 | 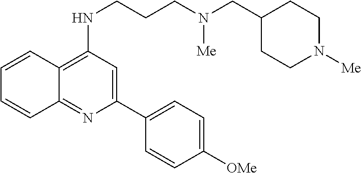 [SMILES: COc1ccc(-c2cc(NCCCN(C)CC3CCN(C)CC3)c3ccccc3n2)cc1] | 0.35 |
| 31 | 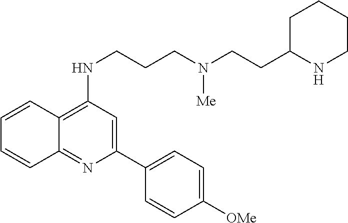 [SMILES: COc1ccc(-c2cc(NCCCN(C)CCC3CCCCN3)c3ccccc3n2)cc1] | 0.35 |
| 32 | 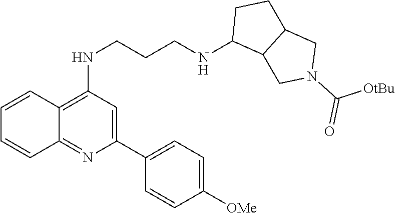 [SMILES: COc1ccc(-c2cc(NCCCNC3CCC4CN(C(=O)OC(C)(C)C)CC43)c3ccccc3n2)cc1] | 2.21 |
| 33 | 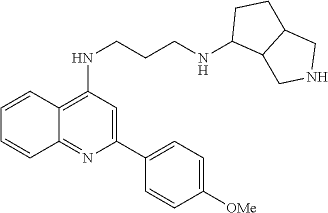 [SMILES: COc1ccc(-c2cc(NCCCNC3CCC4CNCC43)c3ccccc3n2)cc1] | 0.18 |
| 34 | 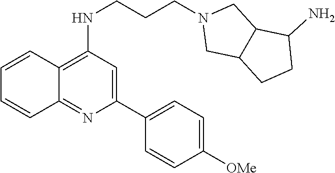 [SMILES: COc1ccc(-c2cc(NCCCN3CC4CCC(N)C4C3)c3ccccc3n2)cc1] | 0.21 |
| 35 | 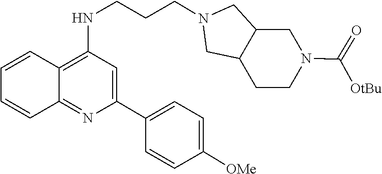 [SMILES: COc1ccc(-c2cc(NCCCN3CC4CCN(C(=O)OC(C)(C)C)CC4C3)c3ccccc3n2)cc1] | 2.67 |
| 36 | 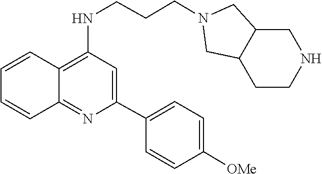 [SMILES: COc1ccc(-c2cc(NCCCN3CC4CCNCC4C3)c3ccccc3n2)cc1] | 0.15 |
| 37 | 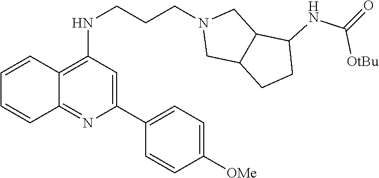 [SMILES: COc1ccc(-c2cc(NCCCN3CC4CCC(NC(=O)OC(C)(C)C)C4C3)c3ccccc3n2)cc1] | 2.21 |
| 38 | 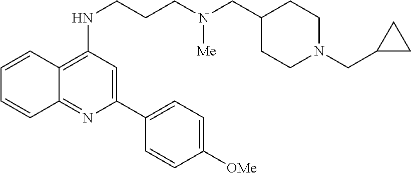 [SMILES: COc1ccc(-c2cc(NCCCN(C)CC3CCN(CC4CC4)CC3)c3ccccc3n2)cc1] | 0.22 |
| 39 | 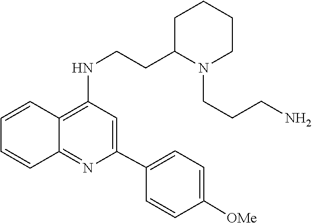 [SMILES: COc1ccc(-c2cc(NCCC3CCCCN3CCCN)c3ccccc3n2)cc1] | 0.13 |
| 40 | 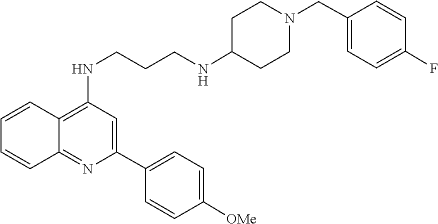 [SMILES: COc1ccc(-c2cc(NCCCNC3CCN(Cc4ccc(F)cc4)CC3)c3ccccc3n2)cc1] | 0.30 |
| 41 | 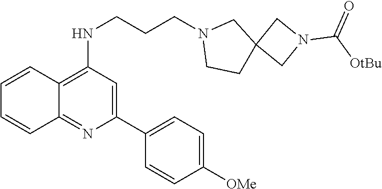 [SMILES: COc1ccc(-c2cc(NCCCN3CCC4(C3)CN(C(=O)OC(C)(C)C)C4)c3ccccc3n2)cc1] | 1.47 |
| 42 | 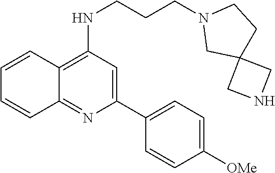 [SMILES: COc1ccc(-c2cc(NCCCN3CCC4(CNC4)C3)c3ccccc3n2)cc1] | 0.23 |
| 43 | 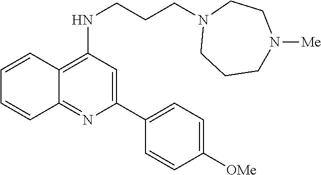 [SMILES: COc1ccc(-c2cc(NCCCN3CCCN(C)CC3)c3ccccc3n2)cc1] | 1.19 |
| 44 | 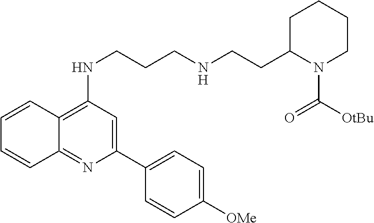 [SMILES: COc1ccc(-c2cc(NCCCNCCC3CCCCN3C(=O)OC(C)(C)C)c3ccccc3n2)cc1] | 2.07 |
| 45 | 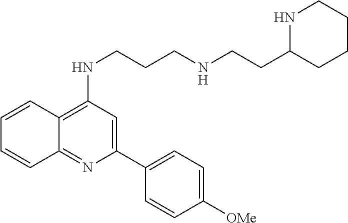 [SMILES: COc1ccc(-c2cc(NCCCNCCC3CCCCN3)c3ccccc3n2)cc1] | 0.71 |
| 46 | 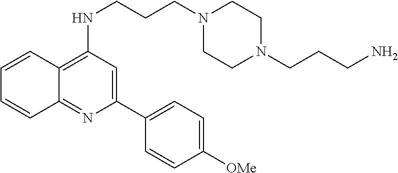 [SMILES: COc1ccc(-c2cc(NCCCN3CCN(CCCN)CC3)c3ccccc3n2)cc1] | 0.36 |
| 47 | 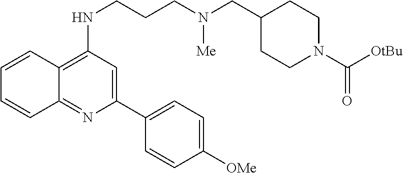 [SMILES: COc1ccc(-c2cc(NCCCN(C)CC3CCN(C(=O)OC(C)(C)C)CC3)c3ccccc3n2)cc1] | 4.05 |
| 48 | 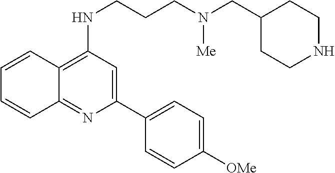 [SMILES: COc1ccc(-c2cc(NCCCN(C)CC3CCNCC3)c3ccccc3n2)cc1] | 0.10 |
| 49 | 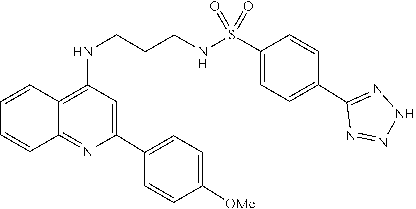 [SMILES: COc1ccc(-c2cc(NCCCNS(=O)(=O)c3ccc(-c4nn[nH]n4)cc3)c3ccccc3n2)cc1] | 7.20 |
| 50 | 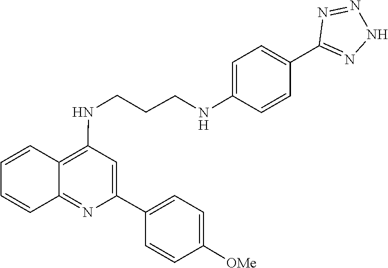 [SMILES: COc1ccc(-c2cc(NCCCNc3ccc(-c4nn[nH]n4)cc3)c3ccccc3n2)cc1] | 15.26 |
| 51 | 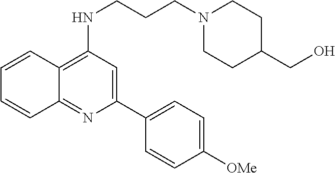 [SMILES: COc1ccc(-c2cc(NCCCN3CCC(CO)CC3)c3ccccc3n2)cc1] | 1.49 |
| 52 | 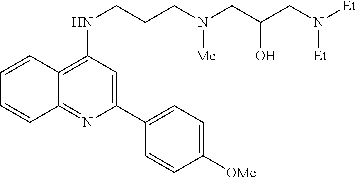 [SMILES: CCN(CC)CC(O)CN(C)CCCNc1cc(-c2ccc(OC)cc2)nc2ccccc12] | 0.17 |
| 53 | 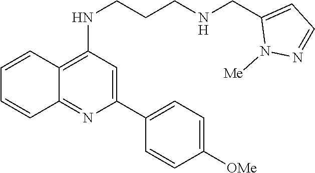 [SMILES: COc1ccc(-c2cc(NCCCNCc3ccnn3C)c3ccccc3n2)cc1] | 3.27 |
| 54 | 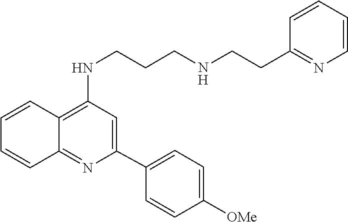 [SMILES: COc1ccc(-c2cc(NCCCNCCc3ccccn3)c3ccccc3n2)cc1] | 1.92 |
| 55 | 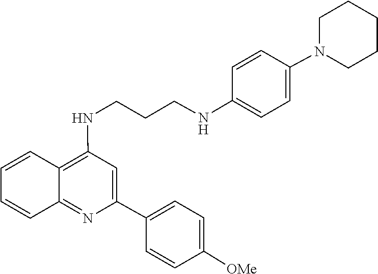 [SMILES: COc1ccc(-c2cc(NCCCNc3ccc(N4CCCCC4)cc3)c3ccccc3n2)cc1] | 0.97 |
| 56 | 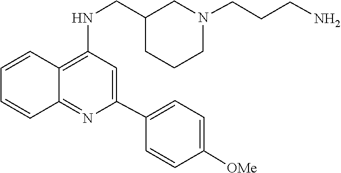 [SMILES: COc1ccc(-c2cc(NCC3CCCN(CCCN)C3)c3ccccc3n2)cc1] | 0.3 |
| 57 | 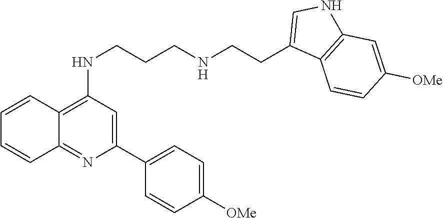 [SMILES: COc1ccc(-c2cc(NCCCNCCc3c[nH]c4cc(OC)ccc34)c3ccccc3n2)cc1] | 0.26 |
| 58 | 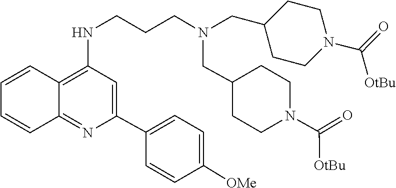 [SMILES: COc1ccc(-c2cc(NCCCN(CC3CCN(C(=O)OC(C)(C)C)CC3)CC3CCN(C(=O)OC(C)(C)C)CC3)c3ccccc3n2)cc1] | 26.73 |
| 59 | 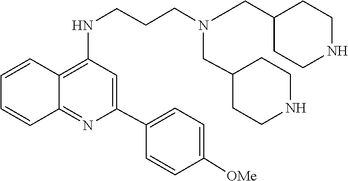 [SMILES: COc1ccc(-c2cc(NCCCN(CC3CCNCC3)CC3CCNCC3)c3ccccc3n2)cc1] | 0.10 |
| 60 | 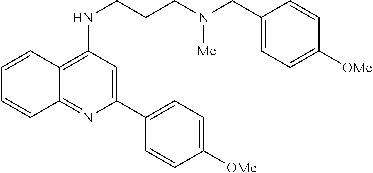 [SMILES: COc1ccc(CN(C)CCCNc2cc(-c3ccc(OC)cc3)nc3ccccc23)cc1] | 1.97 |
| 61 | 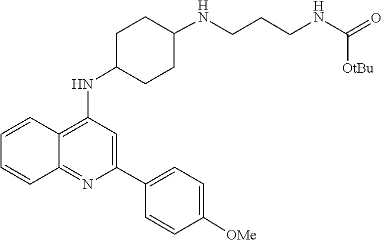 [SMILES: COc1ccc(-c2cc(NC3CCC(NCCCNC(=O)OC(C)(C)C)CC3)c3ccccc3n2)cc1] | 1.35 |
| 62 | 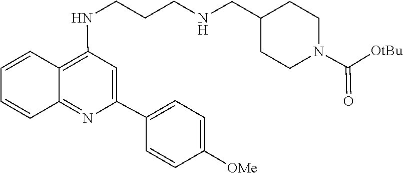 [SMILES: COc1ccc(-c2cc(NCCCNCC3CCN(C(=O)OC(C)(C)C)CC3)c3ccccc3n2)cc1] | 1.16 |
| 63 | 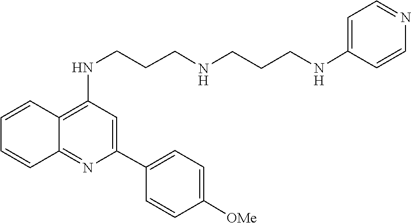 [SMILES: COc1ccc(-c2cc(NCCCNCCCNc3ccncc3)c3ccccc3n2)cc1] | 0.10 |
| 64 | 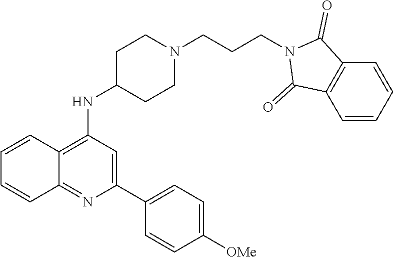 [SMILES: COc1ccc(-c2cc(NC3CCN(CCCN4C(=O)c5ccccc5C4=O)CC3)c3ccccc3n2)cc1] | 3.6 |
| 65 | 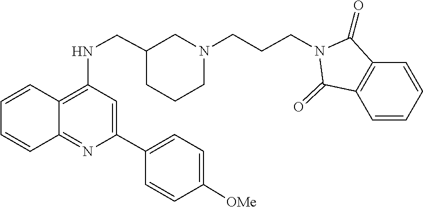 [SMILES: COc1ccc(-c2cc(NCC3CCCN(CCCN4C(=O)c5ccccc5C4=O)C3)c3ccccc3n2)cc1] | 0.73 |
| 66 | 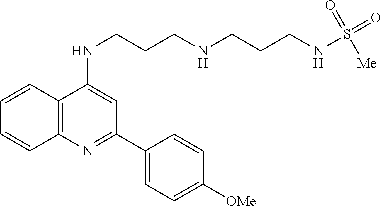 [SMILES: COc1ccc(-c2cc(NCCCNCCCNS(C)(=O)=O)c3ccccc3n2)cc1] | 1.41 |
| 67 | 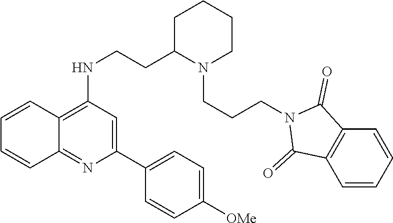 [SMILES: COc1ccc(-c2cc(NCCC3CCCCN3CCCN3C(=O)c4ccccc4C3=O)c3ccccc3n2)cc1] | 0.29 |
| 68 | 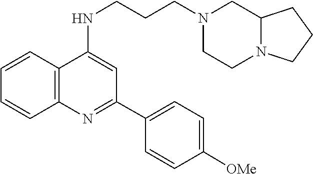 [SMILES: COc1ccc(-c2cc(NCCCN3CCN4CCCC4C3)c3ccccc3n2)cc1] | 2.16 |
| 69 | 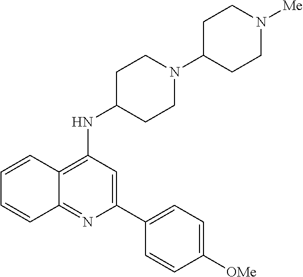 [SMILES: COc1ccc(-c2cc(NC3CCN(C4CCN(C)CC4)CC3)c3ccccc3n2)cc1] | 0.47 |
| 70 | 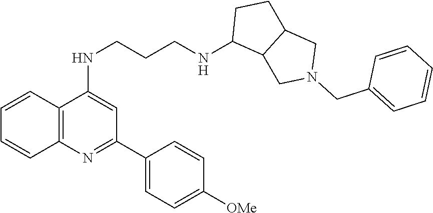 [SMILES: COc1ccc(-c2cc(NCCCNC3CCC4CN(Cc5ccccc5)CC43)c3ccccc3n2)cc1] | 0.17 |
| 71 | 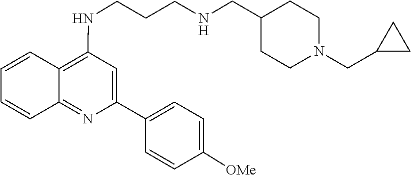 [SMILES: COc1ccc(-c2cc(NCCCNCC3CCN(CC4CC4)CC3)c3ccccc3n2)cc1] | 0.10 |
| 72 | 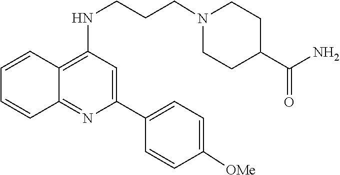 [SMILES: COc1ccc(-c2cc(NCCCN3CCC(C(N)=O)CC3)c3ccccc3n2)cc1] | 0.93 |
| 73 | 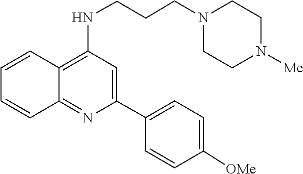 [SMILES: COc1ccc(-c2cc(NCCCN3CCN(C)CC3)c3ccccc3n2)cc1] | 1.71 |
| 74 | 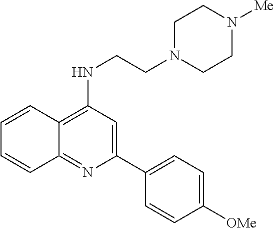 [SMILES: COc1ccc(-c2cc(NCCN3CCN(C)CC3)c3ccccc3n2)cc1] | 1.85 |
| 75 | 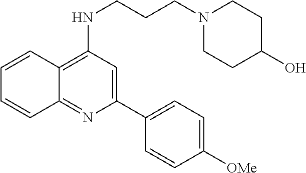 [SMILES: COc1ccc(-c2cc(NCCCN3CCC(O)CC3)c3ccccc3n2)cc1] | 0.59 |
| 76 | 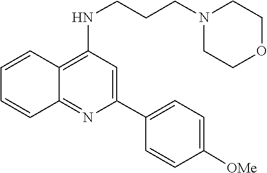 [SMILES: COc1ccc(-c2cc(NCCCN3CCOCC3)c3ccccc3n2)cc1] | 6.28 |
| 77 | 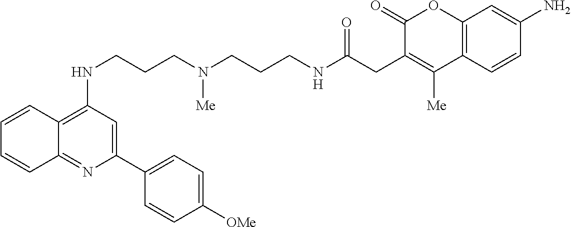 [SMILES: COc1ccc(-c2cc(NCCCN(C)CCCNC(=O)Cc3c(C)c4ccc(N)cc4oc3=O)c3ccccc3n2)cc1] | 0.25 |
| 78 | 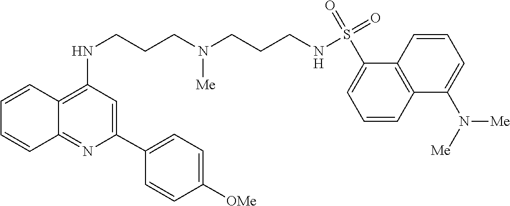 [SMILES: COc1ccc(-c2cc(NCCCN(C)CCCNS(=O)(=O)c3cccc4c(N(C)C)cccc34)c3ccccc3n2)cc1] | 1.26 |
| 79 | 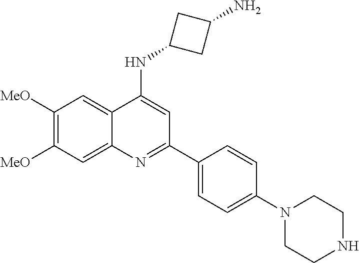 [SMILES: COc1cc2nc(-c3ccc(N4CCNCC4)cc3)cc(N[C@H]3C[C@@H](N)C3)c2cc1OC] | 0.02 |
| 80 | 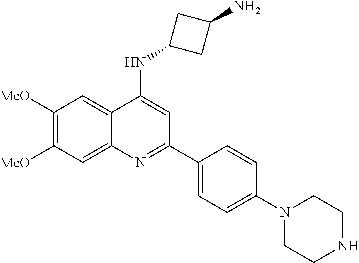 [SMILES: COc1cc2nc(-c3ccc(N4CCNCC4)cc3)cc(N[C@H]3C[C@H](N)C3)c2cc1OC] | 0.02 |
| 81 | 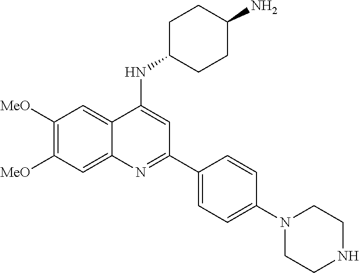 [SMILES: COc1cc2nc(-c3ccc(N4CCNCC4)cc3)cc(N[C@H]3CC[C@H](N)CC3)c2cc1OC] | 0.03 |
| 82 | 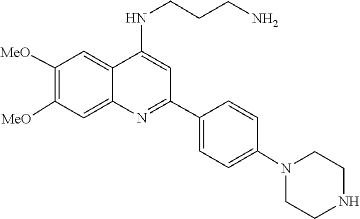 [SMILES: COc1cc2nc(-c3ccc(N4CCNCC4)cc3)cc(NCCCN)c2cc1OC] | 0.03 |
| 83 | 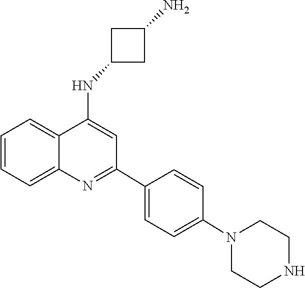 [SMILES: N[C@H]1C[C@@H](Nc2cc(-c3ccc(N4CCNCC4)cc3)nc3ccccc23)C1] | 0.03 |
| 84 | 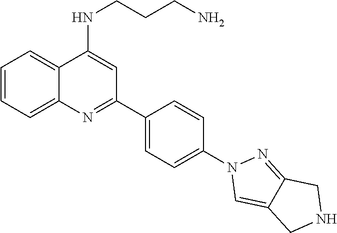 [SMILES: NCCCNc1cc(-c2ccc(-n3cc4c(n3)CNC4)cc2)nc2ccccc12] | 0.04 |
| 85 | 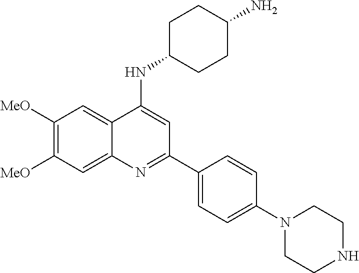 [SMILES: COc1cc2nc(-c3ccc(N4CCNCC4)cc3)cc(N[C@H]3CC[C@@H](N)CC3)c2cc1OC] | 0.04 |
| 86 | 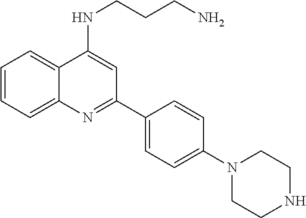 [SMILES: NCCCNc1cc(-c2ccc(N3CCNCC3)cc2)nc2ccccc12] | 0.05 |
| 87 | 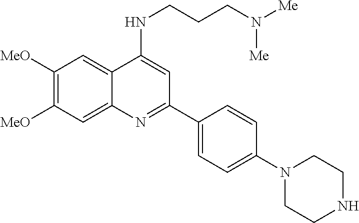 [SMILES: COc1cc2nc(-c3ccc(N4CCNCC4)cc3)cc(NCCCN(C)C)c2cc1OC] | 0.05 |
| 88 | 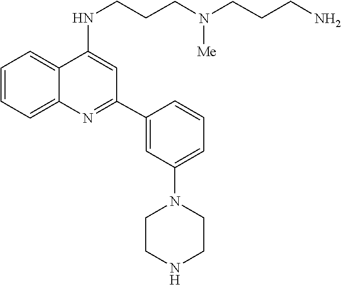 [SMILES: CN(CCCN)CCCNc1cc(-c2cccc(N3CCNCC3)c2)nc2ccccc12] | 0.06 |
| 89 | 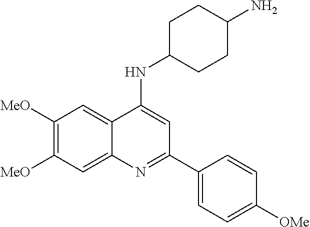 [SMILES: COc1ccc(-c2cc(NC3CCC(N)CC3)c3cc(OC)c(OC)cc3n2)cc1] | 0.08 |
| 90 | 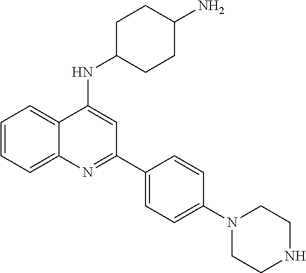 [SMILES: NC1CCC(Nc2cc(-c3ccc(N4CCNCC4)cc3)nc3ccccc23)CC1] | 0.08 |
| 91 | 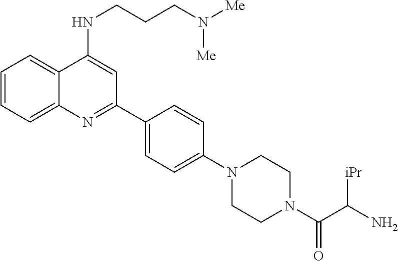 [SMILES: CC(C)C(N)C(=O)N1CCN(c2ccc(-c3cc(NCCCN(C)C)c4ccccc4n3)cc2)CC1] | 0.09 |
| 92 | 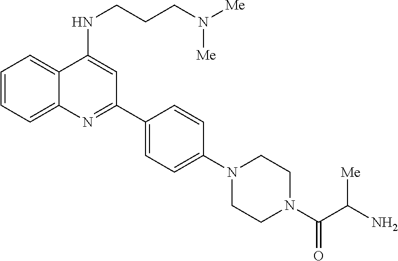 [SMILES: CC(N)C(=O)N1CCN(c2ccc(-c3cc(NCCCN(C)C)c4ccccc4n3)cc2)CC1] | 0.09 |
| 93 | 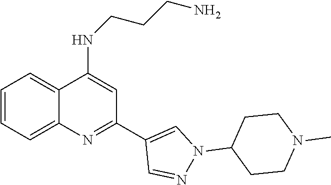 [SMILES: CN1CCC(n2cc(-c3cc(NCCCN)c4ccccc4n3)cn2)CC1] | 0.09 |
| 94 | 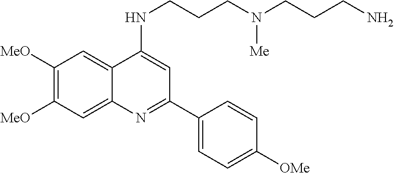 [SMILES: COc1ccc(-c2cc(NCCCN(C)CCCN)c3cc(OC)c(OC)cc3n2)cc1] | 0.10 |
| 95 | 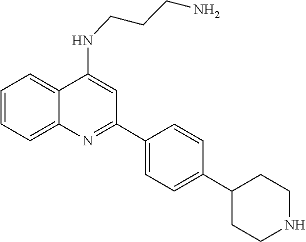 [SMILES: NCCCNc1cc(-c2ccc(C3CCNCC3)cc2)nc2ccccc12] | 0.11 |
| 96 | 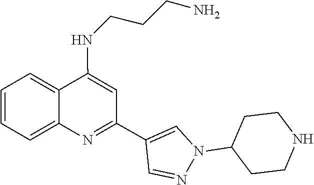 [SMILES: NCCCNc1cc(-c2cnn(C3CCNCC3)c2)nc2ccccc12] | 0.12 |
| 97 | 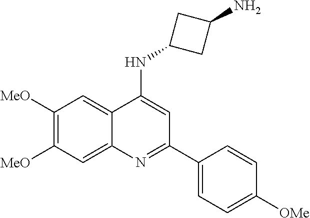 [SMILES: COc1ccc(-c2cc(N[C@H]3C[C@H](N)C3)c3cc(OC)c(OC)cc3n2)cc1] | 0.13 |
| 98 | 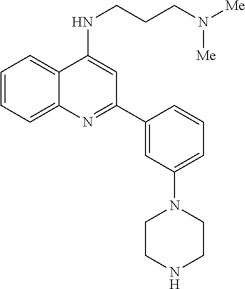 [SMILES: CN(C)CCCNc1cc(-c2cccc(N3CCNCC3)c2)nc2ccccc12] | 0.14 |
| 99 | 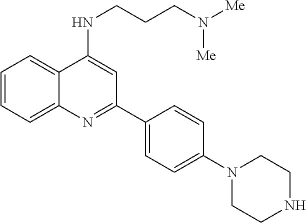 [SMILES: CN(C)CCCNc1cc(-c2ccc(N3CCNCC3)cc2)nc2ccccc12] | 0.15 |
| 100 | 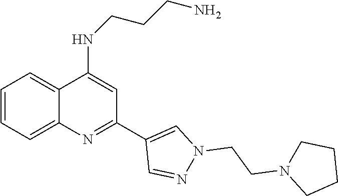 [SMILES: NCCCNc1cc(-c2cnn(CCN3CCCC3)c2)nc2ccccc12] | 0.16 |
| 101 | 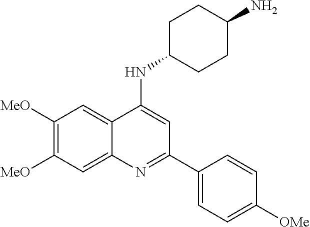 [SMILES: COc1ccc(-c2cc(N[C@H]3CC[C@H](N)CC3)c3cc(OC)c(OC)cc3n2)cc1] | 0.17 |
| 102 | 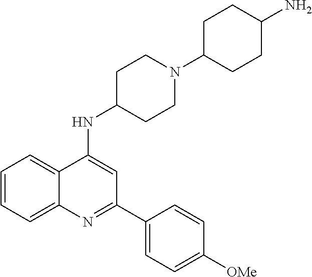 [SMILES: COc1ccc(-c2cc(NC3CCN(C4CCC(N)CC4)CC3)c3ccccc3n2)cc1] | 0.18 |
| 103 | 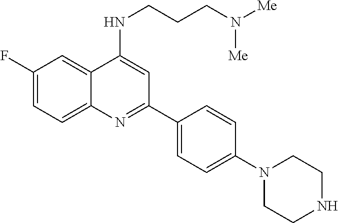 [SMILES: CN(C)CCCNc1cc(-c2ccc(N3CCNCC3)cc2)nc2ccc(F)cc12] | 0.22 |
| 104 | 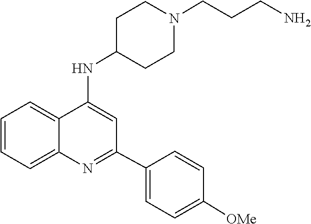 [SMILES: COc1ccc(-c2cc(NC3CCN(CCCN)CC3)c3ccccc3n2)cc1] | 0.22 |
| 105 | 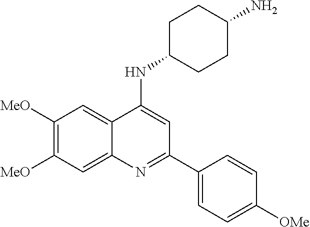 [SMILES: COc1ccc(-c2cc(N[C@H]3CC[C@@H](N)CC3)c3cc(OC)c(OC)cc3n2)cc1] | 0.23 |
| 106 | 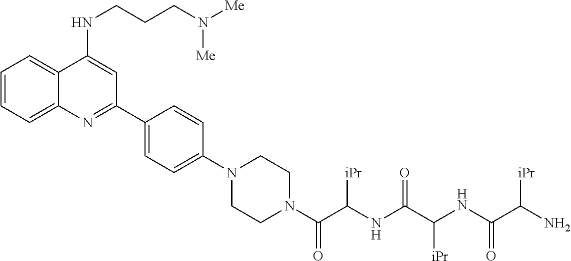 [SMILES: CC(C)C(N)C(=O)NC(C(=O)NC(C(=O)N1CCN(c2ccc(-c3cc(NCCCN(C)C)c4ccccc4n3)cc2)CC1)C(C)C)C(C)C] | 0.24 |
| 107 | 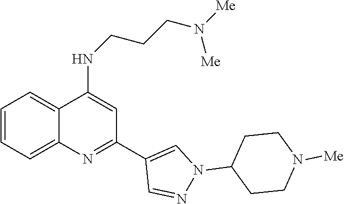 [SMILES: CN(C)CCCNc1cc(-c2cnn(C3CCN(C)CC3)c2)nc2ccccc12] | 0.26 |
| 108 | 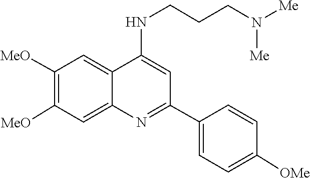 [SMILES: COc1ccc(-c2cc(NCCCN(C)C)c3cc(OC)c(OC)cc3n2)cc1] | 0.27 |
| 109 | 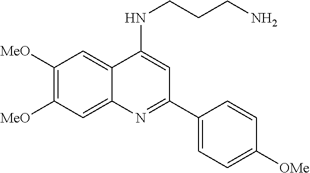 [SMILES: COc1ccc(-c2cc(NCCCN)c3cc(OC)c(OC)cc3n2)cc1] | 0.29 |
| 110 | 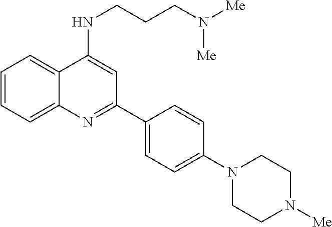 [SMILES: CN(C)CCCNc1cc(-c2ccc(N3CCN(C)CC3)cc2)nc2ccccc12] | 0.31 |
| 111 | 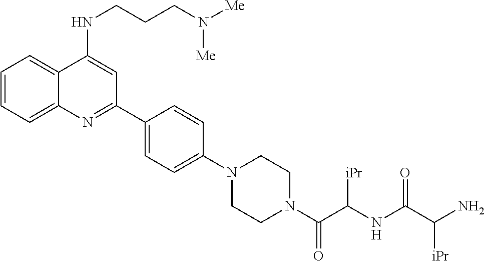 [SMILES: CC(C)C(N)C(=O)NC(C(=O)N1CCN(c2ccc(-c3cc(NCCCN(C)C)c4ccccc4n3)cc2)CC1)C(C)C] | 0.42 |
| 112 | 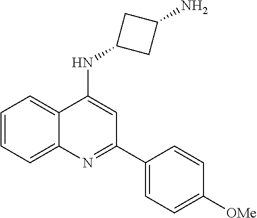 [SMILES: COc1ccc(-c2cc(N[C@H]3C[C@@H](N)C3)c3ccccc3n2)cc1] | 0.49 |
| 113 | 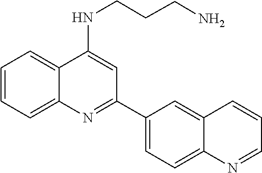 [SMILES: NCCCNc1cc(-c2ccc3ncccc3c2)nc2ccccc12] | 0.57 |
| 114 | 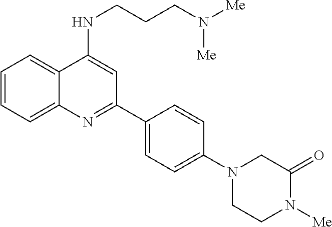 [SMILES: CN(C)CCCNc1cc(-c2ccc(N3CCN(C)C(=O)C3)cc2)nc2ccccc12] | 0.60 |
| 115 | 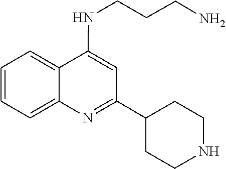 [SMILES: NCCCNc1cc(C2CCNCC2)nc2ccccc12] | 0.61 |
| 116 | 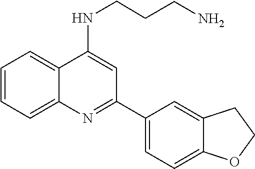 [SMILES: NCCCNc1cc(-c2ccc3c(c2)CCO3)nc2ccccc12] | 0.64 |
| 117 | 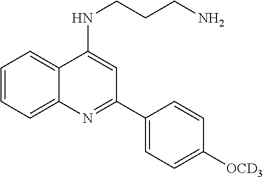 | 0.76 |
| 118 | 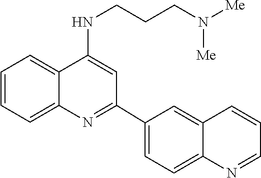 [SMILES: CN(C)CCCNc1cc(-c2ccc3ncccc3c2)nc2ccccc12] | 0.80 |
| 119 | 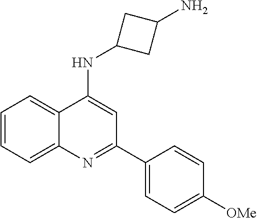 [SMILES: COc1ccc(-c2cc(NC3CC(N)C3)c3ccccc3n2)cc1] | 0.82 |
| 120 | 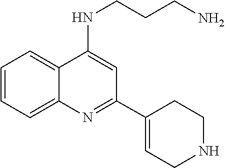 [SMILES: NCCCNc1cc(C2=CCNCC2)nc2ccccc12] | 0.87 |
| 121 | 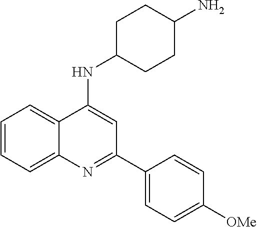 [SMILES: COc1ccc(-c2cc(NC3CCC(N)CC3)c3ccccc3n2)cc1] | 1.07 |
| 122 | 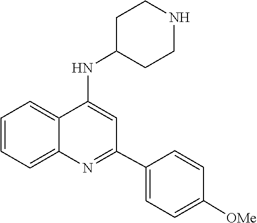 [SMILES: COc1ccc(-c2cc(NC3CCNCC3)c3ccccc3n2)cc1] | 1.17 |
| 123 | 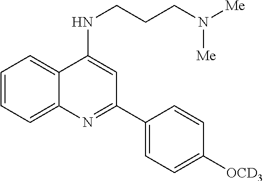 | 1.21 |
| 124 | 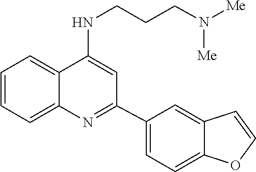 [SMILES: CN(C)CCCNc1cc(-c2ccc3occc3c2)nc2ccccc12] | 1.28 |
| 125 | 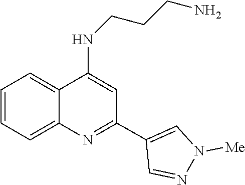 [SMILES: Cn1cc(-c2cc(NCCCN)c3ccccc3n2)cn1] | 1.30 |
| 126 | 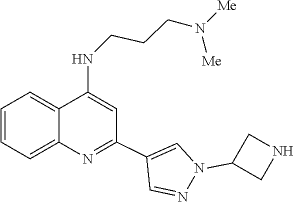 [SMILES: CN(C)CCCNc1cc(-c2cnn(C3CNC3)c2)nc2ccccc12] | 1.49 |
| 127 | 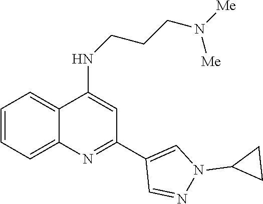 [SMILES: CN(C)CCCNc1cc(-c2cnn(C3CC3)c2)nc2ccccc12] | 1.51 |
| 128 | 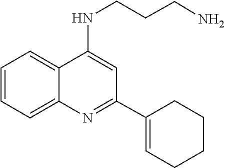 [SMILES: NCCCNc1cc(C2=CCCCC2)nc2ccccc12] | 1.57 |
| 129 | 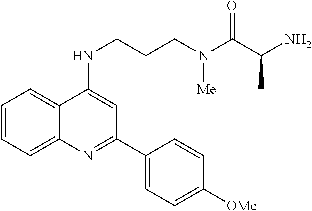 [SMILES: COc1ccc(-c2cc(NCCCN(C)C(=O)[C@H](C)N)c3ccccc3n2)cc1] | 1.66 |
| 130 | 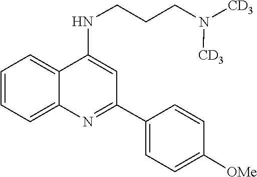 [SMILES: [2H]C([2H])([2H])N(CCCNc1cc(-c2ccc(OC)cc2)nc2ccccc12)C([2H])([2H])[2H]] | 1.78 |
| 131 | 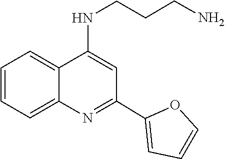 [SMILES: NCCCNc1cc(-c2ccco2)nc2ccccc12] | 2.55 |
| 132 | 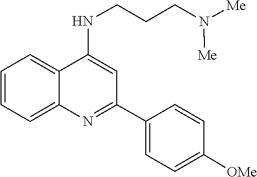 [SMILES: COc1ccc(-c2cc(NCCCN(C)C)c3ccccc3n2)cc1] | 2.72 |
| 133 | 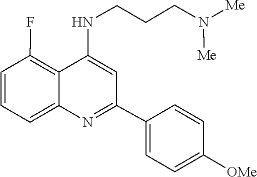 [SMILES: COc1ccc(-c2cc(NCCCN(C)C)c3c(F)cccc3n2)cc1] | 2.90 |
| 134 | 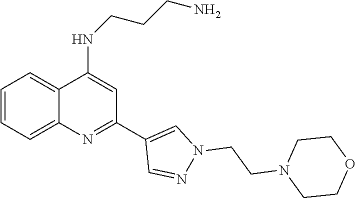 [SMILES: NCCCNc1cc(-c2cnn(CCN3CCOCC3)c2)nc2ccccc12] | 2.94 |
| 135 | 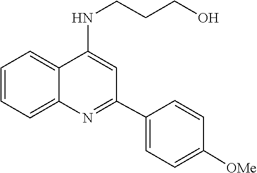 [SMILES: COc1ccc(-c2cc(NCCCO)c3ccccc3n2)cc1] | 31.7 |
| 136 | 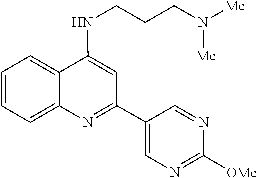 [SMILES: COc1ncc(-c2cc(NCCCN(C)C)c3ccccc3n2)cn1] | 38.5 |
| 137 | 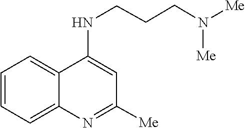 [SMILES: Cc1cc(NCCCN(C)C)c2ccccc2n1] | 45.6 |
| 138 | 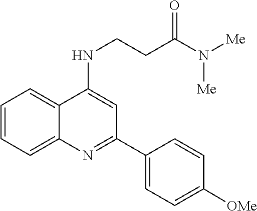 [SMILES: COc1ccc(-c2cc(NCCC(=O)N(C)C)c3ccccc3n2)cc1] | 52.6 |
| 139 | 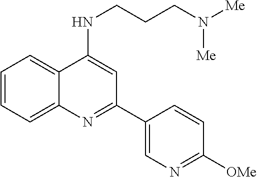 [SMILES: COc1ccc(-c2cc(NCCCN(C)C)c3ccccc3n2)cn1] | 6.51 |
| 140 | 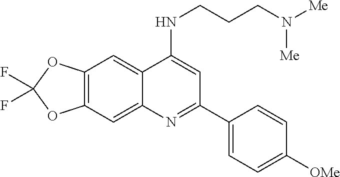 [SMILES: COc1ccc(-c2cc(NCCCN(C)C)c3cc4c(cc3n2)OC(F)(F)O4)cc1] | 6.56 |
| 141 | 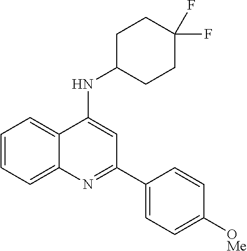 [SMILES: COc1ccc(-c2cc(NC3CCC(F)(F)CC3)c3ccccc3n2)cc1] | 64.5 |
| 142 | 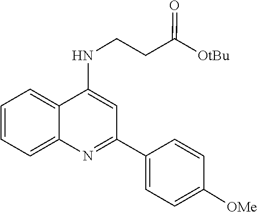 [SMILES: COc1ccc(-c2cc(NCCC(=O)OC(C)(C)C)c3ccccc3n2)cc1] | 65.3 |
| 143 | 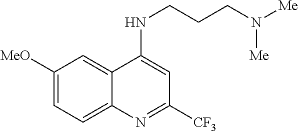 [SMILES: COc1ccc2nc(C(F)(F)F)cc(NCCCN(C)C)c2c1] | 79.4 |
| 144 | 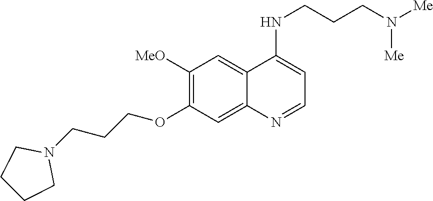 [SMILES: COc1cc2c(NCCCN(C)C)ccnc2cc1OCCCN1CCCC1] | 0.01 |
| 145 | 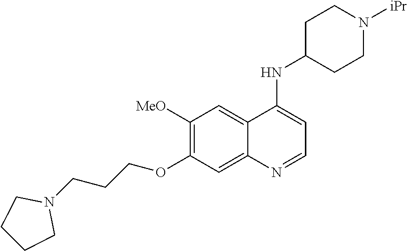 [SMILES: COc1cc2c(NC3CCN(C(C)C)CC3)ccnc2cc1OCCCN1CCCC1] | 1.30 |
| 146 | 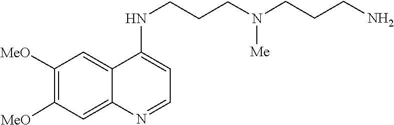 [SMILES: COc1cc2nccc(NCCCN(C)CCCN)c2cc1OC] | 0.41 |
| 147 | 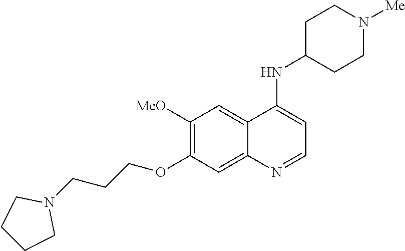 [SMILES: COc1cc2c(NC3CCN(C)CC3)ccnc2cc1OCCCN1CCCC1] | 0.84 |
| 148 | 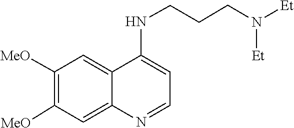 [SMILES: CCN(CC)CCCNc1ccnc2cc(OC)c(OC)cc12] | 0.26 |
| 149 | 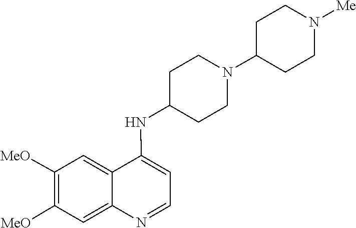 [SMILES: COc1cc2nccc(NC3CCN(C4CCN(C)CC4)CC3)c2cc1OC] | 1.16 |
| 150 | 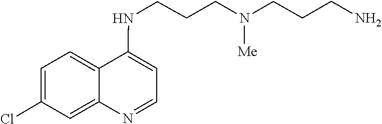 [SMILES: CN(CCCN)CCCNc1ccnc2cc(Cl)ccc12] | 0.22 |
| 151 | 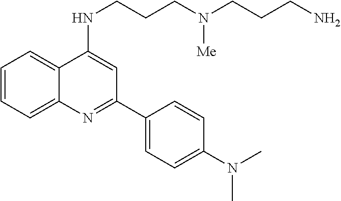 [SMILES: CN(CCCN)CCCNc1cc(-c2ccc(N(C)C)cc2)nc2ccccc12] | TBD |

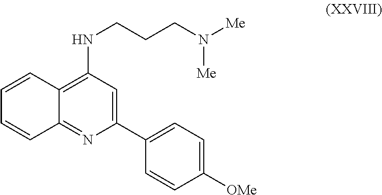 [SMILES: COc1ccc(-c2cc(NCCCN(C)C)c3ccccc3n2)cc1]

The activity of (XXVIII), listed as 1 in Biochimie, 2012, 94, 1974-1981 has been previously reported. The compound was found active in a TDP-43 alpha-screen, however, using bt-(TG)₆ and not bt-TAR32. In addition, (XXVIII) promoted caspase-7 cleavage of TDP-43. However, unexpectedly significant boost in activity was found when the terminal amino group is appended with further substitution of either an aminoalkyl or alkoxyalkyl. For example, Example 3 exhibits an increase in potency of 11-18× (14× average) in the same alpha-screen assay using bt-TAR32 when the two compounds are compared side-by-side in three independent assays.

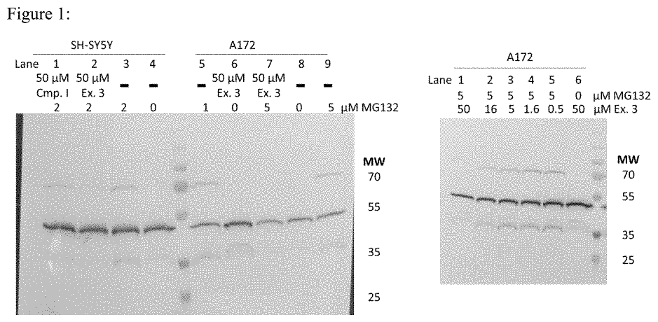

It has been reported (Eersel et al. PLoS ONE, 2011, 6, e22850) that inhibition of proteasome activity in neuronal cells results in accumulation and aggregation of TDP-43 in the cytoplasm that recapitulates major pathological features of neurodegenerative disease including amyotrophic lateral sclerosis (ALS), frontotemporal lobar degeneration (FTLD) and Alzheimer's disease (AD). To investigate the consequences of inhibition of nucleic acid binding by TDP-43 on these disease-related pathologies, human neuroblastoma (SH-SY5Y) and glioblastoma (A172) cells were treated with the proteasome inhibitor benzyloxycarbonyl-1-leucyl-1-leucyl-1-leucinal (MG-132) in the presence and absence of Example 3 (FIG. 1). Cytoplasmic protein extracts were prepared after a 20 hour treatment period and resolved on a denaturing gel to identify TDP-43 immuno-reactive products by Western analysis. In FIG. 1A, full-length 43 kD TDP-43 species was evident in all samples and treatment with MG-132 resulted in the appearance of a higher MW 70 kD band indicative of aggregation (lane 3 for SH-SY5Y cells and lanes 5 and 9 for A172 cells). Importantly, the 70 kD reactive product was absent in both cell types co-treated with MG-132 and Example 3 (lane 2 for SH-SY5Y cells and lane 7 for A172 cells). There was only a smaller impact on levels of the 70 kD product when SH-SY5Y cells were co-treated with MG132 and (XXVIII) (lane 1). A dose response study in A172 cells (FIGURE IB) showed a progressive decline in levels of the 70 kD band relative to full-length TDP-43 as concentrations of Example 3 were increased from 0.5 to 50 μM. Together, these results indicate that inhibition of nucleic acid binding reduces levels of TDP-43 aggregation that is associated with and implicated in mediating neuronal pathologies of several neurodegenerative diseases.

Throughout this application, various publications are referenced by author name and date, or by patent number or patent publication number. The disclosures of these publications are hereby incorporated in their entireties by reference into this application in order to more fully describe the state of the art as known to those skilled therein as of the date of the invention described and claimed herein. However, the citation of a reference herein should not be construed as an acknowledgement that such reference is prior art to the present invention.

Those skilled in the art will recognize or be able to ascertain using no more than routine experimentation, numerous equivalents to the specific procedures described herein. Such equivalents are considered to be within the scope of this invention and are covered by the following claims. Furthermore, it is intended that specific items within lists of items, or subset groups of items within larger groups of items, can be combined with other specific items, subset groups of items or larger groups of items whether or not there is a specific disclosure herein identifying such a combination.

What is claimed is:

1. A compound having formula (I): 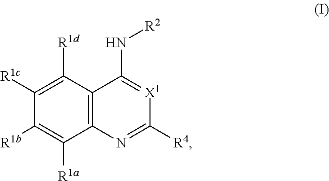
an enantiomer, a diastereomer, a hydrate, a solvate, a pharmaceutically acceptable salt, an isotopic analog, a prodrug or a complex thereof, wherein:
X¹ is selected from the group consisting of nitrogen and CH;
R^{1a} is selected from the group consisting of hydrogen, halogen, CF₃, OCF₃, C₁₋₄ linear alkyl, C₃₋₄ branched alkyl, (C₂₋₈ dialkylamino)(C₂₋₄ alkyl), (C₃₋₆ alkyleneamino)(C₂₋₄ alkyl), (C₂₋₈ dialkylamino)(C₂₋₄ alkoxy), (C₃₋₆ alkyleneamino)(C₂₋₄ alkoxy), C₁₋₄ linear alkoxy, and C₃₋₄ branched alkoxy;
R^{1b} is selected from the group consisting of hydrogen, halogen, CF₃, OCF₃, C₁₋₄ linear alkyl, C₃₋₄ branched alkyl, (C₂₋₈ dialkylamino)(C₂₋₄ alkyl), (C₃₋₆ alkyleneamino)(C₂₋₄ alkyl), (C₂₋₈ dialkylamino)(C₂₋₄ alkoxy), (C₃₋₆ alkyleneamino)(C₂₋₄ alkoxy), C₁₋₄ linear alkoxy, and C₃₋₄ branched alkoxy;
R^{1c} is selected from the group consisting of hydrogen, halogen, CF₃, OCF₃, C₁₋₄ linear alkyl, C₃₋₄ branched alkyl, (C₂₋₈ dialkylamino)(C₂₋₄ alkyl), (C₃₋₆ alkyleneamino)(C₂₋₄ alkyl), (C₂₋₈ dialkylamino)(C₂₋₄ alkoxy), (C₃₋₆ alkyleneamino)(C₂₋₄ alkoxy), C₁₋₄ linear alkoxy, and C₃₋₄ branched alkoxy;
R^{1d} is selected from the group consisting of hydrogen, halogen, CF₃, OCF₃, C₁₋₄ linear alkyl, C₃₋₄ branched alkyl, (C₂₋₈ dialkylamino)(C₂₋₄ alkyl), (C₃₋₆ alkyleneamino)(C₂₋₄ alkyl), (C₂₋₈ dialkylamino)(C₂₋₄ alkoxy), (C₃₋₆ alkyleneamino)(C₂₋₄ alkoxy), C₁₋₄ linear alkoxy, and C₃₋₄ branched alkoxy;
wherein any two selected from the group consisting of R^{1a}, R^{1b}, R^{1c}, and R^{1d} are optionally connected to form a ring;
R² is selected from the group consisting of —(CH₂)ₙ—NR⁵R⁶, —(CH₂)ₙC(O)—NR⁵R⁶, 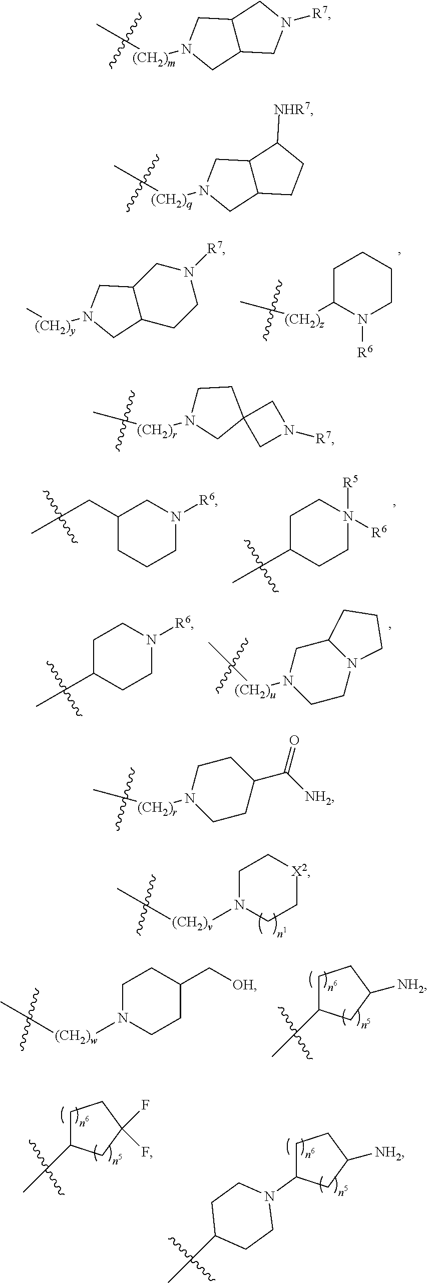
n¹ is 1 or 2;
R⁴ is hydrogen; CF₃; a five-membered monocyclic heteroaryl ring comprising at least one heteroatom selected from the group consisting from O, N, and S that is optionally substituted with up to 2 groups selected from C₁₋₄ linear alkyl, C₃₋₄ branched alkyl, C₁₋₄ linear alkoxy, C₃₋₄ branched alkoxy, CF₃, CF₃O, halogen, 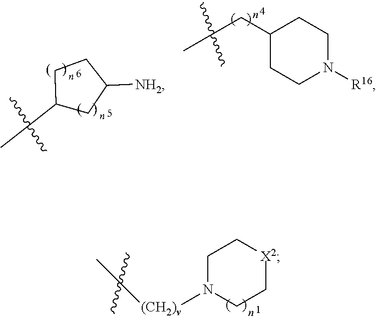
a five-membered monocyclic heteroaryl ring comprising at least one heteroatom selected from the group consisting from O, N, and S; 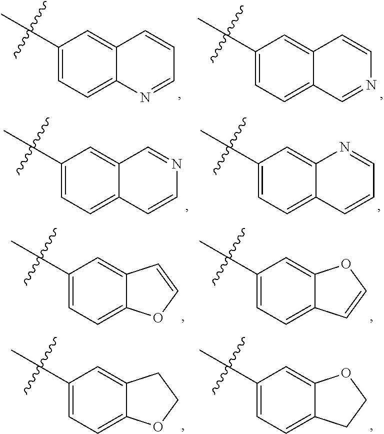
a phenyl ring that is optionally substituted with up to 2 groups selected from C₁₋₄ linear alkyl, C₃₋₄ branched alkyl, C₁₋₄ linear alkoxy, C₃₋₄ branched alkoxy, C₃₋₆ cycloalkyl, C₃₋₆ cycloalkoxy, CF₃, CF₃O, halogen, NR^{3a}R^{3b}, SO₂NR^{3a}R^{3b}, NHSO₂R^{3a}, 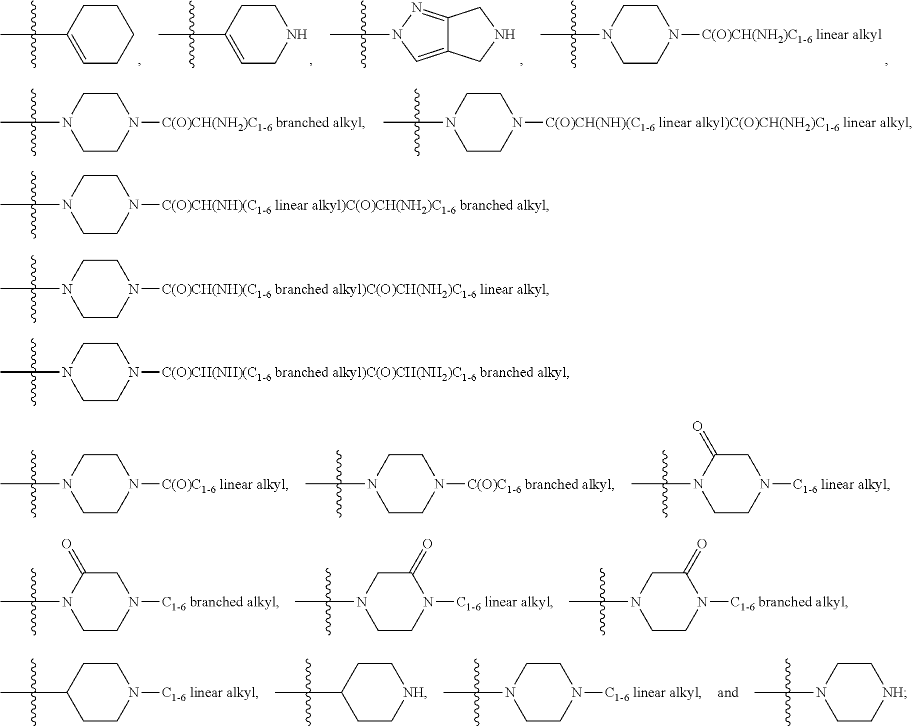
R^{3a} is at each occurrence independently selected from the group consisting of C₁₋₄ linear alkyl and C₃₋₄ branched alkyl;
R^{3b} is at each occurrence independently selected from the group consisting of C₁₋₄ linear alkyl and C₃₋₄ branched alkyl;
R⁵ is selected from the group consisting of hydrogen, C₁₋₄ linear alkyl, C₃₋₄ branched alkyl, —CH₂—(C₁₋₆ cycloalkyl), C(O)C₁₋₆ linear alkyl, C(O)C₃₋₆ branched alkyl, C(O)CH(NH₂)C₁₋₆ linear alkyl, C(O)CH(NH₂)C₃₋₆ branched alkyl, 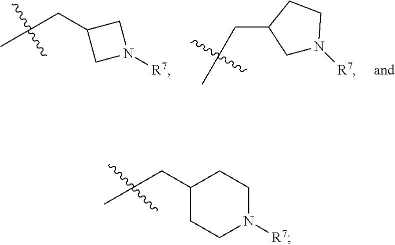
R⁶ is selected from the group consisting of C₁₋₄ linear alkyl C₃₋₄ branched alkyl, 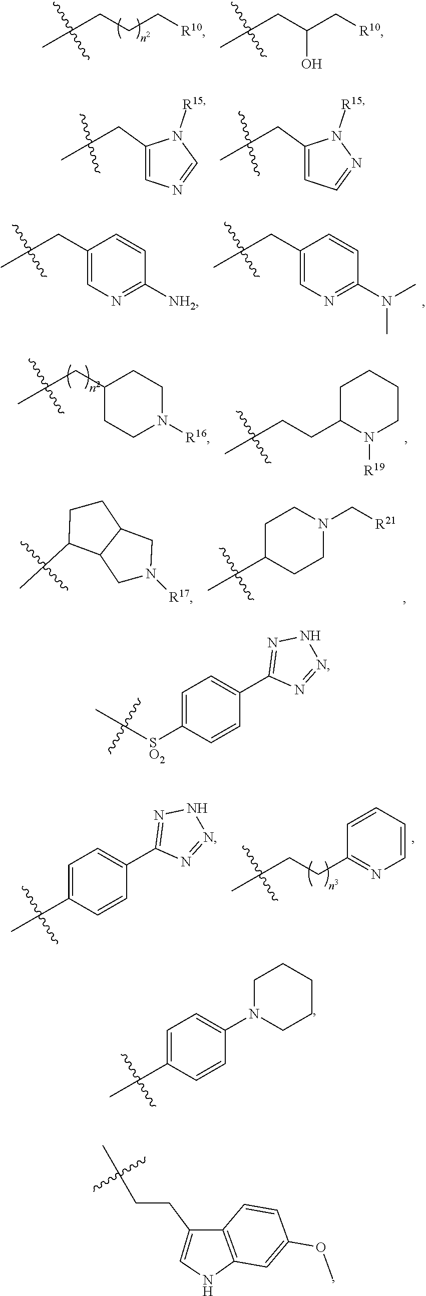
and an optionally substituted benzyl group wherein the optionally substituted benzyl group is substituted with 0 to 2 groups selected from the group consisting of halogen, —CN, —NO₂, —OH, —NH₂, C₁₋₆ alkyl, C₃₋₇ branched alkyl, C₁₋₆ linear haloalkyl, C₃₋₇ branched haloalkyl, C₁₋₆ linear alkoxy, C₃₋₇ branched alkoxy, C₁₋₆ linear haloalkoxy, C₃₋₇ branched haloalkoxy, C₃₋₇ cycloalkyl, aryl, heterocycle, and heteroaryl, provided that when R² is 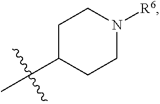
R⁶ is not hydrogen, C₁₋₄ linear alkyl, C₃₋₄ branched alkyl, or benzyl;
n² is 1 or 2;
n³ is 0 or 1;
n⁴ is 0 or 1;
n⁵ is 0, 1, or 2;
n⁶ is 0 or 1;
R⁷ is selected from the group consisting of hydrogen and C(O)OR⁸;
R⁸ is selected from the group consisting of C₁₋₄ linear alkyl and C₃₋₄ branched alkyl;
X² is selected from the group consisting of a single bond, oxygen, CH₂, CHOH, and NR⁹;
R⁹ is C₁₋₄ linear alkyl that is optionally substituted with an NH₂;
R¹⁰ is selected from the group consisting of OH, OR¹¹, NR¹²R¹³, NHSO₂R²², and 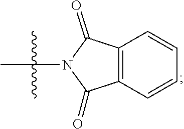 [SMILES: *N1C(=O)c2ccccc2C1=O]
R¹¹ is selected from the group consisting of C₁₋₄ linear alkyl and C₃₋₄ branched alkyl;
R¹² is selected from the group consisting of hydrogen, C₁₋₄ linear alkyl, C₃₋₄ branched alkyl, C₁₋₄ linear fluoroalkyl, C(O)R¹⁴;
R¹³ is selected from the group consisting of hydrogen, C₁₋₄ linear alkyl, C₃₋₄ branched alkyl, and heteroaryl;
R¹² and R¹³ are optionally taken together with the atoms to which they are connected to form a ring with 3 to 6 atoms;
R¹⁴ is selected from the group consisting of C₁₋₄ linear alkyl C₃₋₄ branched alkyl, and 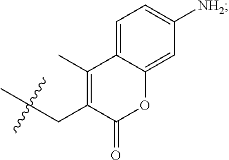 [SMILES: *Cc1c(C)c2ccc(N)cc2oc1=O]
R¹⁵ is selected from the group consisting of C₁₋₄ linear alkyl and C₃₋₄ branched alkyl;
R¹⁶ is selected from the group consisting of hydrogen, C₁₋₄ linear alkyl, C₃₋₄ branched alkyl, CH₂—(C₁₋₆ cycloalkyl), and C(O)R¹⁸;
R⁷ is selected from the group consisting of hydrogen, benzyl and C(O)R¹⁸;
R⁸ is selected from the group consisting of C₁₋₄ linear alkyl and C₃₋₄ branched alkyl;
R¹⁹ is selected from the group consisting of hydrogen and C(O)R²⁰;
R²⁰ is selected from the group consisting of C₁₋₄ linear alkyl and C₃₋₄ branched alkyl;
R²¹ is a benzene ring that is optionally substituted with 0 to 2 groups selected from the group consisting of halogen, —CN, —NO₂, —OH, —NH₂, C₁₋₆ alkyl, C₃₋₇ branched alkyl, C₁₋₆ linear haloalkyl, C₃₋₇ branched haloalkyl, C₁₋₆ linear alkoxy, C₃₋₇ branched alkoxy, C₁₋₆ linear haloalkoxy, C₃₋₇ branched haloalkoxy, C₃₋₇ cycloalkyl, aryl, heterocycle, and heteroaryl;
R²² is selected from the group consisting of C₁₋₄ linear alkyl, C₃₋₄ branched alkyl, 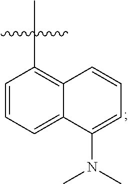 [SMILES: *c1cccc2c(N(C)C)cccc12]
n is 2, 3, or 4;
m is 2, 3, or 4;
q is 2, 3, or 4;
y is 2, 3, or 4;
u is 2, 3, or 4;
v is 2, 3, or 4;
w is 2, 3, or 4;
z is 1, 2 or 3;
r is 2, 3, or 4; and
x is 2, 3, or 4.

2. The compound of claim 1 having formula (II): 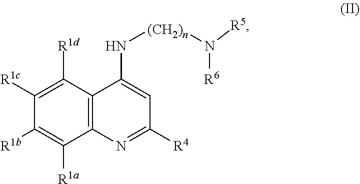
an enantiomer, a diastereomer, a hydrate, a solvate, a pharmaceutically acceptable salt, an isotopic analog, a prodrug, or a complex thereof.

3. The compound of claim 1 having formula (III): 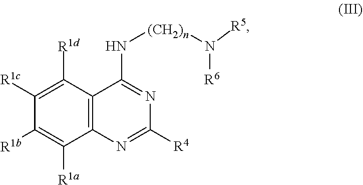
an enantiomer, a diastereomer, a hydrate, a solvate, a pharmaceutically acceptable salt, an isotopic analog, a prodrug, or a complex thereof.

4. The compound of claim 1 having formula (IV): 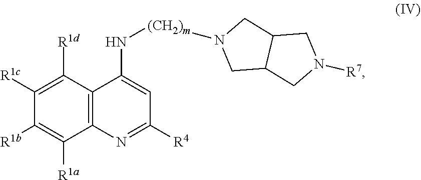
an enantiomer, a diastereomer, a hydrate, a solvate, a pharmaceutically acceptable salt, an isotopic analog, a prodrug, or a complex thereof.

5. The compound of claim 1 having formula (V): 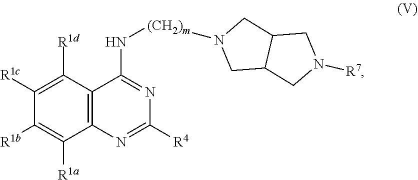
an enantiomer, a diastereomer, a hydrate, a solvate, a pharmaceutically acceptable salt, an isotopic analog, a prodrug, or a complex thereof.

6. The compound of claim 1 having formula (VI): 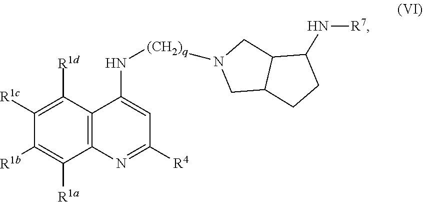
an enantiomer, a diastereomer, a hydrate, a solvate, a pharmaceutically acceptable salt, an isotopic analog, a prodrug, or a complex thereof.

7. The compound of claim 1 having formula (VII): 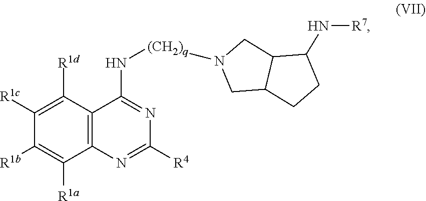
an enantiomer, a diastereomer, a hydrate, a solvate, a pharmaceutically acceptable salt, an isotopic analog, a prodrug, or a complex thereof.

8. The compound of claim 1 having formula (VIII): 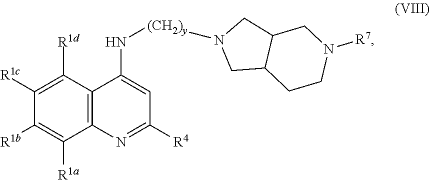
an enantiomer, a diastereomer, a hydrate, a solvate, a pharmaceutically acceptable salt, an isotopic analog, a prodrug, or a complex thereof.

9. The compound of claim 1 having formula (IX): 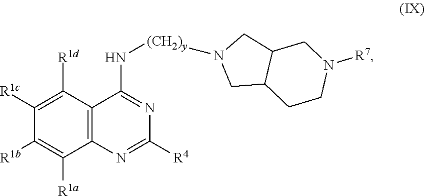
an enantiomer, a diastereomer, a hydrate, a solvate, a pharmaceutically acceptable salt, an isotopic analog, a prodrug, or a complex thereof.

10. The compound of claim 1 having formula (X): 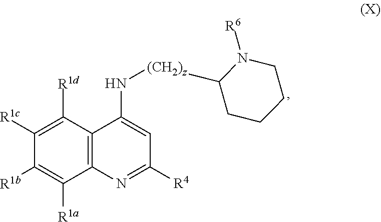
an enantiomer, a diastereomer, a hydrate, a solvate, a pharmaceutically acceptable salt, an isotopic analog, a prodrug, or a complex thereof.

11. The compound of claim 1 having formula (XI): 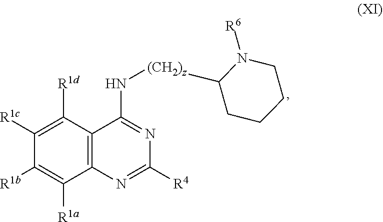
an enantiomer, a diastereomer, a hydrate, a solvate, a pharmaceutically acceptable salt, an isotopic analog, a prodrug, and a complex thereof.

12. The compound of claim 1 having formula (XII): 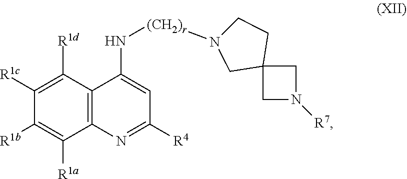
an enantiomer, a diastereomer, a hydrate, a solvate, a pharmaceutically acceptable salt, an isotopic analog, a prodrug, or a complex thereof.

13. The compound of claim 1 having formula (XIII): 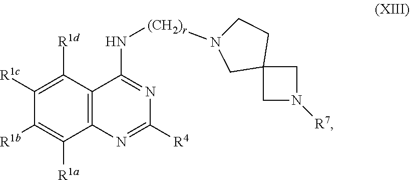
an enantiomer, a diastereomer, a hydrate, a solvate, a pharmaceutically acceptable salt, an isotopic analog, a prodrug, or a complex thereof.

14. The compound of claim 1 having formula (XIV): 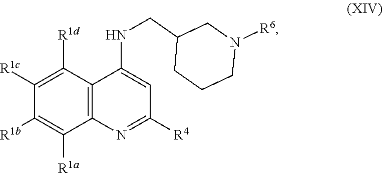
an enantiomer, a diastereomer, a hydrate, a solvate, a pharmaceutically acceptable salt, an isotopic analog, a prodrugs, or a complex thereof.

15. The compound of claim 1 having formula (XV): 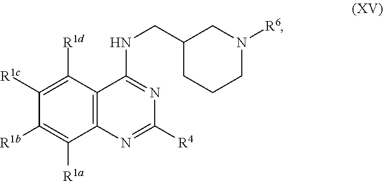
an enantiomer, a diastereomer, a hydrate, a solvate, a pharmaceutically acceptable salt, an isotopic analog, a prodrug, or a complex thereof.

16. The compound of claim 1 having formula (XVI): 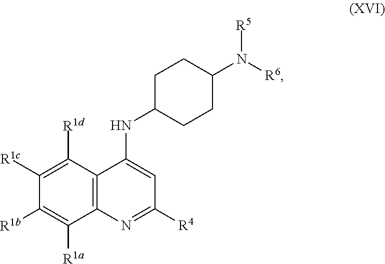
an enantiomer, a diastereomer, a hydrate, a solvate, a pharmaceutically acceptable salt, an isotopic analog, a prodrug, or a complex thereof.

17. The compound of claim 1 having formula (XVII): 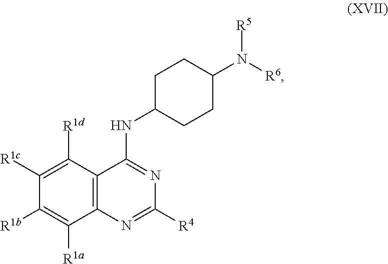
an enantiomer, a diastereomer, a hydrate, a solvate, a pharmaceutically acceptable salt, an isotopic analog, a prodrug, or a complex thereof.

18. The compound of claim 1 having formula (XVIII): 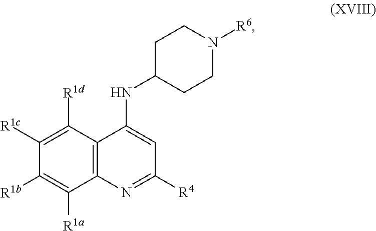
an enantiomer, a diastereomer, a hydrate, a solvate, a pharmaceutically acceptable salts, an isotopic analog, a prodrug, or a complex thereof.

19. The compound of claim 1 having formula (XIX): 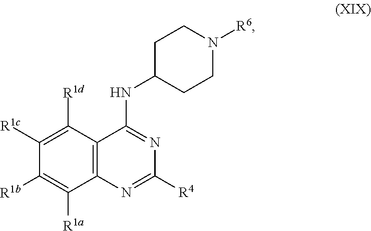
an enantiomer, a diastereomer, a hydrate, a solvate, a pharmaceutically acceptable salt, an isotopic analog, a prodrug, or a complex thereof.

20. The compound of claim 1 having formula (XX): 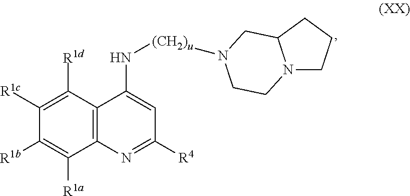
an enantiomer, a diastereomer, a hydrate, a solvate, a pharmaceutically acceptable salt, an isotopic analog, a prodrug, or a complex thereof.

21. The compound of claim 1 having formula (XXI): 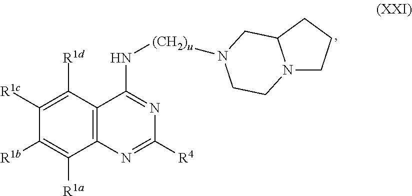
an enantiomer, a diastereomer, a hydrate, a solvate, a pharmaceutically acceptable salt, an isotopic analog, a prodrug, or a complex thereof.

22. The compound of claim 1 having formula (XXII): 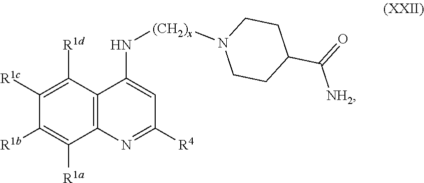
an enantiomer, a diastereomer, a hydrate, a solvate, a pharmaceutically acceptable salt, an isotopic analog, a prodrug, or a complexes thereof.

23. The compound of claim 1 having formula (XXIII): 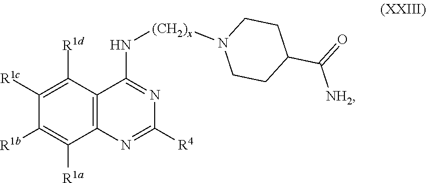
an enantiomer, a diastereomer, a hydrate, a solvate, a pharmaceutically acceptable salt, an isotopic analog, a prodrug, or a complex thereof.

24. The compound of claim 1 having formula (XXIV): 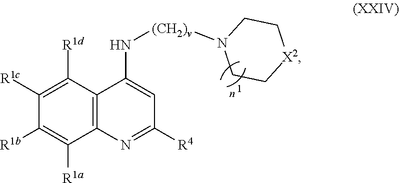
an enantiomer, a diastereomer, a hydrate, a solvate, a pharmaceutically acceptable salt, an isotopic analog, a prodrug, or a complex thereof.

25. The compound of claim 1 having formula (XXV): 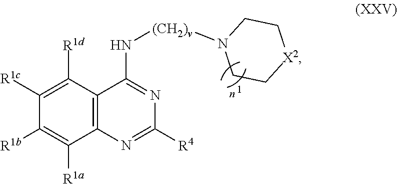
an enantiomer, a diastereomer, a hydrate, a solvate, a pharmaceutically acceptable salt, an isotopic analogs, a prodrug, or a complex thereof.

26. The compound of claim 1 having formula (XXVI): 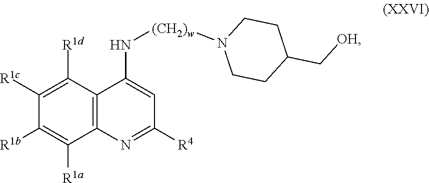
an enantiomer, a diastereomer, a hydrate, a solvate, a pharmaceutically acceptable salt, an isotopic analog, a prodrug, or a complex thereof.

27. The compound of claim 1 having formula (XXVII): 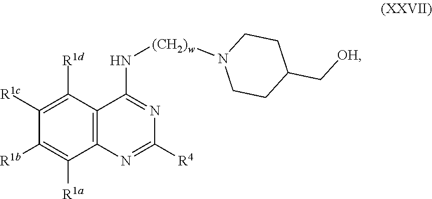
an enantiomer, a diastereomer, a hydrate, a solvate, a pharmaceutically acceptable salt, an isotopic analog, a prodrug, or a complex thereof.

28. The compound according to claim 1 that is:
N¹-(3-((2-(4-Methoxyphenyl)quinolin-4-yl)amino)propyl)-N¹,N³,N³-trimethyl propane-1,3-diamine;
2-(4-Methoxyphenyl)-N-(3-f{methyl[3-(methylamino)propyl]-amino}propyl)quinolin-4-amine;
N¹-(3-Aminopropyl)-N³-(2-(4-methoxyphenyl)quinolin-4-yl)-N¹-methylpropane-1,3-diamine;
N¹-(2-(4-Methoxyphenyl)quinolin-4-yl)-N³-(3-(piperidin-1-yl)propyl)propane-1,3-diamine;
N¹-(2-(4-methoxyphenyl)quinolin-4-yl)-N³-methyl-N³-(3-(piperidin-1-yl)propyl)propane-1,3-diamine;
tert-Butyl N-{3-[(3-{[2-(4-methoxyphenyl)quinolin-4yl]amino}propyl)amino]propyl}carbamate;
N¹-(3-aminopropyl)-N³-(5-fluoro-2-(4-methoxyphenyl)quinolin-4-yl)-N¹-methylpropane-1,3-diamine:
N-{3-[(3-Aminopropyl)amino]propyl}-2-(4-methoxyphenyl)quinolin-4-amine;
N¹-(3-Aminopropyl)-N³-(6-methoxy-2-(4-methoxyphenyl)quinolin-4-yl)-N¹-methylpropane-1,3-diamine;
N¹-(3-Aminopropyl)-N³-(2-(2-methyl-4-methoxyphenyl)quinolin-4-yl)-N¹-methylpropane-1,3-diamine;
N¹-(3-Aminopropyl)-N³-(6,7-dimethoxy-2-(4-methoxyphenyl)quinolin-4-yl)-N¹-methylpropane-1,3-diamine;
N¹-(3-((2-(4-Methoxyphenyl)quinolin-4-yl)amino)propyl)-N¹,N⁴-dimethylbutane-1,4-diamine;
N¹-(2-fluoroethyl)-N³-(3-((2-(4-methoxyphenyl)quinolin-4-yl)amino)propyl)-N¹,N³-dimethylpropane-1,3-diamine;
3-((3-((2-(4-Methoxyphenyl)quinolin-4-yl)amino)propyl)amino)-propan-1-ol;
N-(3-(3-methoxypropylamino)propyl)-2-(4-methoxyphenyl)quinolin-4-amine;
N-(3-(2-(4-Methoxyphenyl)quinolin-4-ylamino)propyl)-N-(3-aminopropyl)acetamide;
N¹-(Cyclopropylmethyl)-N¹-(3-((2-(4-methoxyphenyl)quinolin-4-yl)amino)propyl)-N³,N³-dimethyl propane-1,3-diamine:
tert-Butyl 3-(((3-(dimethylamino)propyl)(3-((2-(4-methoxyphenyl)quinolin-4-yl)amino)propyl)amino)methyl)azetidine-1-carboxylate;
tert-Butyl 4-(((3-(dimethylamino)propyl)(3-((2-(4-methoxyphenyl)quinolin-4-yl)amino)propyl)amino)methyl)piperidine-1-carboxylate:
N¹-(Azetidin-3-ylmethyl)-N¹-(3-((2-(4-methoxyphenyl)quinolin-4-yl)amino)propyl)-N³,N³-dimethylpropane-1,3-diamine;
N¹-(3-((2-(4-Methoxyphenyl)quinolin-4-yl)amino)propyl)-N³,N³-dimethyl-N¹-(piperidin-4-ylmethyl)propane-1,3-diamine;
N¹-(2-(4-Methoxyphenyl)quinolin-4-yl)-N³-((1-methyl-1H-imidazol-5-yl)methyl)propane-1,3-diamine;
N-(3-(Hexahydropyrrolo[3,4-c]pyrrol-2(1H)-yl)propyl)-2-(4-methoxyphenyl)quinolin-4-amine;
N¹-((6-Aminopyridin-3-yl)methyl)-N³-(2-(4-methoxyphenyl)-quinolin-4-yl)-N¹-methylpropane-1,3-diamine;
1-(Dimethylamino)-3-((3-((2-(4-methoxyphenyl)quinolin-4-yl)-amino)propyl)(methyl)amino)propan-2-ol;
1-(3-((3-((2-(4-Methoxyphenyl)quinolin-4-yl)amino)propyl)-methylamino)propyl)piperidin-4-ol;
tert-butyl 5-(3-((2-(4-methoxyphenyl)quinolin-4-yl)amino)propyl)hexahydro-pyrrolo[3,4-c]pyrrole-2(1H)-carboxylate;
N¹-((6-(dimethylamino)pyridin-3-yl)methyl)-N³-(2-(4-methoxyphenyl)quinolin-4-yl)-N¹-methyl propane-1,3-diamine;
N¹-(3-aminopropyl)-N¹-methyl-N³-(2-(4-(piperazin-1-yl)phenyl)quinolin-4-yl)propane-1,3-diamine;
N¹-(2-(4-methoxyphenyl)quinolin-4-yl)-N³-methyl-N³-((1-methylpiperidin-4-yl)methyl)propane-1,3-diamine:
N¹-(2-(4-methoxyphenyl)quinolin-4-yl)-N³-methyl-N³-(2-(piperidin-2-yl)ethyl)propane-1,3-diamine;
tert-Butyl(2-(3-((2-(4-methoxyphenyl)quinolin-4-yl)amino)propyl)octahydrocyclopenta[c]pyrrol-4-yl)carbamate:
N¹-(2-(4-Methoxyphenyl)quinolin-4-yl)-N³-(octahydrocyclopenta[c]pyrrol-4-yl)propane-1,3-diamine;
N-(3-(4-Aminohexahydrocyclopenta[c]pyrrol-2(1H)-yl)propyl)-2-(4-methoxyphenyl)quinolin-4-amine;
tert-Butyl 2-(3-((2-(4-methoxyphenyl)quinolin-4-yl)amino)propyl)hexahydro-1H-pyrrolo[3,4-c]pyridine-5(6H)-carboxylate;
N-(3-(Hexahydro-1H-pyrrolo[3,4-c]pyridin-2(3H)-yl)propyl)-2-(4-methoxyphenyl)quinolin-4-amine;
tert-Butyl 4-((3-((2-(4-methoxyphenyl)quinolin-4-yl)amino)propyl)amino)hexahydrocyclopenta[c] pyrrole-2(1H)-carboxylate;
N¹-((1-(Cyclopropylmethyl)piperidin-4-yl)methyl)-N³-(2-(4-methoxyphenyl)quinolin-4-yl)-N¹-methyl propane-1,3-diamine;
N-(2-(1-(3-Aminopropyl)piperidin-2-yl)ethyl)-2-(4-methoxyphenyl)quinolin-4-amine:
N¹-(1-(4-Fluorobenzyl)piperidin-4-yl)-N³-(2-(4-methoxyphenyl)quinolin-4-yl)propane-1,3-diamine:
tert-Butyl 6-(3-((2-(4-methoxyphenyl)quinolin-4-yl)amino)propyl)-2,6-diazaspiro[3.4]octane-2-carboxylate;
N-(3-(2,6-Diazaspiro[3.4]octan-6-yl)propyl)-2-(4-methoxyphenyl)quinolin-4-amine:
2-(4-Methoxyphenyl)-N-(3-(4-methyl-1,4-diazepan-1-yl)propyl)quinolin-4-amine:
tert-Butyl 2-(2-((3-((2-(4-methoxyphenyl)quinolin-4-yl)amino)propyl)amino)ethyl)piperidine-1-carboxylate;
N¹-(2-(4-methoxyphenyl)quinolin-4-yl)-N³-(2-(piperidin-2-yl)ethyl)propane-1,3-diamine;
N-(3-(4-(3-Aminopropyl)piperazin-1-yl)propyl)-2-(4-methoxyphenyl)quinolin-4-amine;
tert-Butyl 4-{[(3-{[2-(4-methoxyphenyl)quinolin-4-yl]amino}propyl)(methyl)amino]methyl}piperidine-1-carboxylate;
N-(3-(N-Methyl-N-((piperidin-4-yl)methyl)amino)propyl)-2-(4-methoxyphenyl)quinolin-4-amine;
N-(3-((2-(4-Methoxyphenyl)quinolin-4-yl)amino)propyl)-4-(2H-tetrazol-5-yl)benzenesulfonamide;
N¹-(4-(2H-Tetrazol-5-yl)phenyl)-N³-(2-(4-methoxyphenyl)quinolin-4-yl)propane-1,3-diamine;
(1-(3-((2-(4-Methoxyphenyl)quinolin-4-yl)amino)propyl)piperidin-4-yl)methanol;
1-(Diethylamino)-3-((3-((2-(4-methoxyphenyl)quinolin-4-yl)amino)propyl)amino)propan-2-ol;
N¹-(2-(4-Methoxyphenyl)quinolin-4-yl)-N³-((1-methyl-1H-pyrazol-5-yl)methyl)propane-1,3-diamine;
N¹-(2-(4-methoxyphenyl)quinolin-4-yl)-N³-(2-(pyridin-2-yl)ethyl)propane-1,3-diamine;
N¹-(2-(4-Methoxyphenyl)quinolin-4-yl)-N³-(4-(piperidin-1-yl)phenyl)propane-1,3-diamine;
N-((1-(3-Aminopropyl)piperidin-3-yl)methyl)-2-(4-methoxyphenyl)quinolin-4-amine;
N-(3-(2-(6-Methoxy-1H-indol-3-yl)ethylamino)propyl)-2-(4-methoxyphenyl)quinolin-4-amine:
tert-Butyl 4-{[({1-[(tert-butoxy)carbonyl]piperidin-4-yl}methyl)(3-{[2-(4-methoxyphenyl)-quinolin-4-yl]amino}propyl)amino]methyl}piperidine-1-carboxylate;
N-(3-(Bis((piperidin-4-yl)methyl)amino)propyl)-2-(4-methoxyphenyl)quinolin-4-amine;
N¹-(4-Methoxybenzyl)-N³-(2-(4-methoxyphenyl)quinolin-4-yl)-N¹-methylpropane-1,3-diamine;
tert-Butyl(3-((4-((2-(4-methoxyphenyl)quinolin-4-yl)amino)cyclohexyl)amino)propyl)carbamate;
tert-Butyl 4-{[(3-{[2-(4-methoxyphenyl)quinolin-4-yl]amino}propyl)amino]methyl}piperidine-1-carboxylate;
N¹-(2-(4-methoxyphenyl)quinolin-4-yl)-N³-(3-(pyridin-4-ylamino)propyl)propane-1,3-diamine;
2-(3-(4-((2-(4-Methoxyphenyl)quinolin-4-yl)amino)piperidin-1-yl)propyl)isoindoline-1,3-dione;
2-(3-(3-(((2-(4-Methoxyphenyl)quinolin-4-yl)amino)methyl)piperidin-1-yl)propyl)isoindoline-1,3-dione;
N-(3-((3-((2-(4-Methoxyphenyl)quinolin-4-yl)amino)propyl)amino)propyl) methanesulfonamide;
2-(3-(2-(2-((2-(4-Methoxyphenyl)quinolin-4-yl)amino)ethyl)piperidin-1-yl)propyl)isoindoline-1,3-dione;
N-(3-(Hexahydropyrrolo[1,2-a]pyrazin-2(1H)-yl)propyl)-2-(4-methoxy phenyl)quinolin-4-amine;
2-(4-Methoxyphenyl)-N-(1′-methyl-[1,4′-bipiperidine]-4-yl)quinolin-4-amine;
N¹-(2-Benzyloctahydrocyclopenta[c]pyrrol-4-yl)-N³-(2-(4-methoxyphenyl)quinolin-4-yl)propane-1,3-diamine;
N¹-((1-(Cyclopropylmethyl)piperidin-4-yl)methyl)-N³-(2-(4-methoxyphenyl)quinolin-4-yl)propane-1,3-diamine;
1-(3-((2-(4-Methoxyphenyl)quinolin-4-yl)amino)propyl)piperidine-4-carboxamide;
2-(4-Methoxyphenyl)-N-(3-(4-methylpiperazin-1-yl)propyl)quinolin-4-amine;
2-(4-Methoxyphenyl)-N-(2-(4-methylpiperazin-1-yl)ethyl)quinolin-4-amine;
2-(4-Methoxyphenyl)-N-(3-morpholinopropyl)quinolin-4-amine;
2-(7-Amino-4-methyl-2-oxo-2H-chromen-3-yl)-N-(3-((3-((2-(4-methoxyphenyl)quinolin-4-yl)amino) propyl)(methyl)amino)propyl)acetamide;
5-(dimethylamino)-N-{3-[(3-{[2-(4-methoxyphenyl)quinolin-4yl]amino}propyl)(methyl)amino]propyl}naphthalene-1-sulfonamide;
cis-N¹-(6,7-dimethoxy-2-(4-(piperazin-1-yl)phenyl)quinolin-4-yl)cyclobutane-1,3-diamine;
trans-N¹-(6,7-dimethoxy-2-(4-(piperazin-1-yl)phenyl)quinolin-4-yl)cyclobutane-1,3-diamine;
trans-N¹-(6,7-dimethoxy-2-(4-(piperazin-1-yl)phenyl)quinolin-4-yl)cyclohexane-1,4-diamine;
N¹-(6,7-dimethoxy-2-(4-(piperazin-1-yl)phenyl)quinolin-4-yl)propane-1,3-diamine;
cis-N¹-(2-(4-(piperazin-1-yl)phenyl)quinolin-4-yl)cyclobutane-1,3-diamine;
N¹-(2-(4-(5,6-dihydropyrrolo[3,4-c]pyrazol-2(4H)-yl)phenyl)quinolin-4-yl)propane-1,3-diamine;
cis-N¹-(6,7-dimethoxy-2-(4-(piperazin-1-yl)phenyl)quinolin-4-yl)cyclohexane-1,4-diamine;
N¹-(2-(4-(piperazin-1-yl)phenyl)quinolin-4-yl)propane-1,3-diamine;
N¹-(6,7-dimethoxy-2-(4-(piperazin-1-yl)phenyl)quinolin-4-yl)-N³,N³-dimethylpropane-1,3-diamine;
N¹-(3-aminopropyl)-N¹-methyl-N³-(2-(3-(piperazin-1-yl)phenyl)quinolin-4-yl)propane-1,3-diamine;
N¹-(6,7-dimethoxy-2-(4-methoxyphenyl)quinolin-4-yl)cyclohexane-1,4-diamine;
N¹-(2-(4-(piperazin-1-yl)phenyl)quinolin-4-yl)cyclohexane-1,4-diamine;
2-amino-1-(4-(4-(4-((3-(dimethylamino)propyl)amino)quinolin-2-yl)phenyl)piperazin-1-yl)-3-methylbutan-1-one;
2-amino-1-(4-(4-(4-((3-(dimethylamino)propyl)amino)quinolin-2-yl)phenyl)piperazin-1-yl)propan-1-one;
N¹-(2-(1-(1-methylpiperidin-4-yl)-1H-pyrazol-4-yl)quinolin-4-yl)propane-1,3-diamine;
N¹-(3-aminopropyl)-N³-(6,7-dimethoxy-2-(4-methoxyphenyl)quinolin-4-yl)-N¹-methylpropane-1,3-diamine;
N¹-(2-(4-(piperidin-4-yl)phenyl)quinolin-4-yl)propane-1,3-diamine;
N¹-(2-(1-(piperidin-4-yl)-1H-pyrazol-4-yl)quinolin-4-yl)propane-1,3-diamine;
trans-N¹-(6,7-dimethoxy-2-(4-methoxyphenyl)quinolin-4-yl)cyclobutane-1,3-diamine;
N¹,N¹-dimethyl-N³-(2-(3-(piperazin-1-yl)phenyl)quinolin-4-yl)propane-1,3-diamine;
N¹,N¹-dimethyl-N³-(2-(4-(piperazin-1-yl)phenyl)quinolin-4-yl)propane-1,3-diamine;
N¹-(2-(1-(2-(pyrrolidin-1-yl)ethyl)-1H-pyrazol-4-yl)quinolin-4-yl)propane-1,3-diamine;
trans-N¹-(6,7-dimethoxy-2-(4-methoxyphenyl)quinolin-4-yl)cyclohexane-1,4-diamine;
N-(1-(4-aminocyclohexyl)piperidin-4-yl)-2-(4-methoxyphenyl)quinolin-4-amine;
N¹-(6-fluoro-2-(4-(piperazin-1-yl)phenyl)quinolin-4-yl)-N³,N³-dimethylpropane-1,3-diamine;
N-(1-(3-aminopropyl)piperidin-4-yl)-2-(4-methoxyphenyl)quinolin-4-amine;
cis-N1-(6,7-dimethoxy-2-(4-methoxyphenyl)quinolin-4-yl)cyclohexane-1,4-diamine;
2-amino-N-(1-((1-(4-(4-(4-((3-(dimethylamino)propyl)amino)quinolin-2-yl)phenyl)piperazin-1-yl)-3-methyl-1-oxobutan-2-yl)amino)-3-methyl-1-oxobutan-2-yl)-3-methylbutanamide;
N¹,N¹-dimethyl-N³-(2-(1-(1-methylpiperidin-4-yl)-1H-pyrazol-4-yl)quinolin-4-yl)propane-1,3-diamine;
N¹-(6,7-dimethoxy-2-(4-methoxyphenyl)quinolin-4-yl)-N³,N³-dimethylpropane-1,3-diamine;
N¹-(6,7-dimethoxy-2-(4-methoxyphenyl)quinolin-4-yl)propane-1,3-diamine;
N¹,N¹-dimethyl-N³-(2-(4-(4-methylpiperazin-1-yl)phenyl)quinolin-4-yl)propane-1,3-diamine;
2-amino-N-(1-(4-(4-(4-((3-(dimethylamino)propyl)amino)quinolin-2-yl)phenyl)piperazin-1-yl)-3-methyl-1-oxobutan-2-yl)-3-methylbutanamide;
cis-N¹-(2-(4-methoxyphenyl)quinolin-4-yl)cyclobutane-1,3-diamine;
N¹-([2,6′-biquinolin]-4-yl)propane-1,3-diamine;
4-(4-(4-((3-(dimethylamino)propyl)amino)quinolin-2-yl)phenyl)-1-methylpiperazin-2-one;
N¹-(2-(piperidin-4-yl)quinolin-4-yl)propane-1,3-diamine;
N¹-(2-(2,3-dihydrobenzofuran-5-yl)quinolin-4-yl)propane-1,3-diamine;
N¹-(2-(4-(methoxy-d3)phenyl)quinolin-4-yl)propane-1,3-diamine;
N¹-([2,6′-biquinolin]-4-yl)-N³,N³-dimethylpropane-1,3-diamine;
N¹-(2-(4-methoxyphenyl)quinolin-4-yl)cyclobutane-1,3-diamine;
N¹-(2-(1,2,3,6-tetrahydropyridin-4-yl)quinolin-4-yl)propane-1,3-diamine;
N¹-(2-(4-methoxyphenyl)quinolin-4-yl)cyclohexane-1,4-diamine;
2-(4-methoxyphenyl)-N-(piperidin-4-yl)quinolin-4-amine;
N¹-(2-(4-(methoxy-d₃)phenyl)quinolin-4-yl)-N³,N³-dimethylpropane-1,3-diamine;
N¹-(2-(benzofuran-5-yl)quinolin-4-yl)-N³,N³-dimethylpropane-1,3-diamine;
N¹-(2-(1-methyl-1H-pyrazol-4-yl)quinolin-4-yl)propane-1,3-diamine;
N¹-(2-(1-(azetidin-3-yl)-1H-pyrazol-4-yl)quinolin-4-yl)-N³,N³-dimethylpropane-1,3-diamine;
N¹-(2-(1-cyclopropyl-1H-pyrazol-4-yl)quinolin-4-yl)-N³,N³-dimethylpropane-1,3-diamine;
N¹-(2-(cyclohex-1-en-1-yl)quinolin-4-yl)propane-1,3-diamine;
(S)-2-amino-N-(3-((2-(4-methoxyphenyl)quinolin-4-yl)amino)propyl)-N-methylpropanamide;
N¹-(2-(4-methoxyphenyl)quinolin-4-yl)-N³,N³-bis(methyl-d3)propane-1,3-diamine;
N¹-(2-(furan-2-yl)quinolin-4-yl)propane-1,3-diamine;
N¹-(5-fluoro-2-(4-methoxyphenyl)quinolin-4-yl)-N³,N³-dimethylpropane-1,3-diamine;
N¹-(2-(1-(2-morpholinoethyl)-1H-pyrazol-4-yl)quinolin-4-yl)propane-1,3-diamine;
3-((2-(4-methoxyphenyl)quinolin-4-yl)amino)propan-1-ol;
N¹-(2-(2-methoxypyrimidin-5-yl)quinolin-4-yl)-N³,N³-dimethylpropane-1,3-diamine;
N¹,N¹-dimethyl-N³-(2-methylquinolin-4-yl)propane-1,3-diamine;
3-((2-(4-methoxyphenyl)quinolin-4-yl)amino)-N,N-dimethylpropanamide;
N¹-(2-(6-methoxypyridin-3-yl)quinolin-4-yl)-N³,N³-dimethylpropane-1,3-diamine;
N¹-(2,2-difluoro-6-(4-methoxyphenyl)-[1,3]dioxolo[4,5-g]quinolin-8-yl)-N³,N³-dimethylpropane-1,3-diamine;
N-(4,4-difluorocyclohexyl)-2-(4-methoxyphenyl)quinolin-4-amine;
tert-butyl 3-((2-(4-methoxyphenyl)quinolin-4-yl)amino)propanoate;
N¹-(6-methoxy-2-(trifluoromethyl)quinolin-4-yl)-N³,N³-dimethylpropane-1,3-diamine;
N¹-(6-methoxy-7-(3-(pyrrolidin-1-yl)propoxy)quinolin-4-yl)-N³,N³-dimethylpropane-1,3-diamine;
2-cyclohexyl-N-(1-isopropylpiperidin-4-yl)-6-methoxy-7-(3-(pyrrolidin-1-yl)propoxy)quinolin-4-amine;
N¹-(3-aminopropyl)-N³-(6,7-dimethoxyquinolin-4-yl)-N¹-methylpropane-1,3-diamine;
6-methoxy-N-(1-methylpiperidin-4-yl)-7-(3-(pyrrolidin-1-yl)propoxy)quinolin-4-amine;
N¹-(6,7-dimethoxyquinolin-4-yl)-N³,N³-diethylpropane-1,3-diamine;
6,7-dimethoxy-N-(1′-methyl-[1,4′-bipiperidin]-4-yl)quinolin-4-amine;
N¹-(3-aminopropyl)-N³-(7-chloroquinolin-4-yl)-N¹-methylpropane-1,3-diamine;
N¹-(3-aminopropyl)-N³-(2-(4-(dimethylamino)phenyl)quinolin-4-yl)-N-methylpropane-1,3-diamine,
an enantiomer, a diastereomer, a hydrate, a solvate, a pharmaceutically acceptable salt, an isotopic analog, a prodrug, or a complex thereof.

29. A composition comprising an effective amount of at least one compound according to claim 1.

30. The composition according to claim 29, further comprising at least one excipient.

31. The composition according to claim 30, wherein the at least one compound is at least one selected from the group consisting of:
N¹-(3-((2-(4-Methoxyphenyl)quinolin-4-yl)amino)propyl)-N¹,N³,N³-trimethyl propane-1,3-diamine;
2-(4-Methoxyphenyl)-N-(3-f{methyl[3-(methylamino)propyl]-amino}propyl)quinolin-4-amine;
N¹-(3-Aminopropyl)-N³-(2-(4-methoxyphenyl)quinolin-4-yl)-N¹-methylpropane-1,3-diamine;
N¹-(2-(4-Methoxyphenyl)quinolin-4-yl)-N³-(3-(piperidin-1-yl)propyl)propane-1,3-diamine;
N¹-(2-(4-methoxyphenyl)quinolin-4-yl)-N³-methyl-N³-(3-(piperidin-1-yl)propyl)propane-1,3-diamine;
tert-Butyl N-{3-[(3-{[2-(4-methoxyphenyl)quinolin-4yl]amino}propyl)amino]propyl}carbamate;
N¹-(3-aminopropyl)-N³-(5-fluoro-2-(4-methoxyphenyl)quinolin-4-yl)-N¹-methylpropane-1,3-diamine:
N-{3-[(3-Aminopropyl)amino]propyl}-2-(4-methoxyphenyl)quinolin-4-amine;
N¹-(3-Aminopropyl)-N³-(6-methoxy-2-(4-methoxyphenyl)quinolin-4-yl)-N¹-methylpropane-1,3-diamine;
N¹-(3-Aminopropyl)-N³-(2-(2-methyl-4-methoxyphenyl)quinolin-4-yl)-N¹-methylpropane-1,3-diamine;
N¹-(3-Aminopropyl)-N³-(6,7-dimethoxy-2-(4-methoxyphenyl)quinolin-4-yl)-N¹-methylpropane-1,3-diamine;
N¹-(3-((2-(4-Methoxyphenyl)quinolin-4-yl)amino)propyl)-N¹,N⁴-dimethylbutane-1,4-diamine;
N¹-(2-fluoroethyl)-N³-(3-((2-(4-methoxyphenyl)quinolin-4-yl)amino)propyl)-N¹,N³-dimethylpropane-1,3-diamine;
3-((3-((2-(4-Methoxyphenyl)quinolin-4-yl)amino)propyl)amino)-propan-1-ol;
N-(3-(3-methoxypropylamino)propyl)-2-(4-methoxyphenyl)quinolin-4-amine;
N-(3-(2-(4-Methoxyphenyl)quinolin-4-ylamino)propyl)-N-(3-aminopropyl)acetamide;
N¹-(Cyclopropylmethyl)-N¹-(3-((2-(4-methoxyphenyl)quinolin-4-yl)amino)propyl)-N³,N³-dimethyl propane-1,3-diamine:
tert-Butyl 3-(((3-(dimethylamino)propyl)(3-((2-(4-methoxyphenyl)quinolin-4-yl)amino)propyl)amino)methyl)azetidine-1-carboxylate;
tert-Butyl 4-(((3-(dimethylamino)propyl)(3-((2-(4-methoxyphenyl)quinolin-4-yl)amino)propyl)amino)methyl)piperidine-1-carboxylate:
N¹-(Azetidin-3-ylmethyl)-N¹-(3-((2-(4-methoxyphenyl)quinolin-4-yl)amino)propyl)-N³,N³-dimethylpropane-1,3-diamine;
N¹-(3-((2-(4-Methoxyphenyl)quinolin-4-yl)amino)propyl)-N³,N³-dimethyl-N¹-(piperidin-4-ylmethyl)propane-1,3-diamine;
N¹-(2-(4-Methoxyphenyl)quinolin-4-yl)-N³-((1-methyl-1H-imidazol-5-yl)methyl)propane-1,3-diamine;
N-(3-(Hexahydropyrrolo[3,4-c]pyrrol-2(1H)-yl)propyl)-2-(4-methoxyphenyl)quinolin-4-amine;
N¹-((6-Aminopyridin-3-yl)methyl)-N³-(2-(4-methoxyphenyl)-quinolin-4-yl)-N¹-methylpropane-1,3-diamine;
1-(Dimethylamino)-3-((3-((2-(4-methoxyphenyl)quinolin-4-yl)-amino)propyl)(methyl)amino)propan-2-ol;
1-(3-((3-((2-(4-Methoxyphenyl)quinolin-4-yl)amino)propyl)-methylamino)propyl)piperidin-4-ol;
tert-butyl 5-(3-((2-(4-methoxyphenyl)quinolin-4-yl)amino)propyl)hexahydro-pyrrolo[3,4-c]pyrrole-2(1H)-carboxylate;
N¹-((6-(dimethylamino)pyridin-3-yl)methyl)-N³-(2-(4-methoxyphenyl)quinolin-4-yl)-N¹-methyl propane-1,3-diamine;
N¹-(3-aminopropyl)-N¹-methyl-N³-(2-(4-(piperazin-1-yl)phenyl)quinolin-4-yl)propane-1,3-diamine;
N¹-(2-(4-methoxyphenyl)quinolin-4-yl)-N³-methyl-N³-((1-methylpiperidin-4-yl)methyl)propane-1,3-diamine:
N¹-(2-(4-methoxyphenyl)quinolin-4-yl)-N³-methyl-N³-(2-(piperidin-2-yl)ethyl)propane-1,3-diamine;
tert-Butyl(2-(3-((2-(4-methoxyphenyl)quinolin-4-yl)amino)propyl)octahydrocyclopenta[c]pyrrol-4-yl)carbamate:
N¹-(2-(4-Methoxyphenyl)quinolin-4-yl)-N³-(octahydrocyclopenta[c]pyrrol-4-yl)propane-1,3-diamine;
N-(3-(4-Aminohexahydrocyclopenta[c]pyrrol-2(1H)-yl)propyl)-2-(4-methoxyphenyl)quinolin-4-amine;
tert-Butyl 2-(3-((2-(4-methoxyphenyl)quinolin-4-yl)amino)propyl)hexahydro-1H-pyrrolo[3,4-c]pyridine-5(6H)-carboxylate;
N-(3-(Hexahydro-1H-pyrrolo[3,4-c]pyridin-2(3H)-yl)propyl)-2-(4-methoxyphenyl)quinolin-4-amine;
tert-Butyl 4-((3-((2-(4-methoxyphenyl)quinolin-4-yl)amino)propyl)amino)hexahydrocyclopenta[c]pyrrole-2(1H)-carboxylate;
N¹-((1-(Cyclopropylmethyl)piperidin-4-yl)methyl)-N³-(2-(4-methoxyphenyl)quinolin-4-yl)-N¹-methyl propane-1,3-diamine;
N-(2-(1-(3-Aminopropyl)piperidin-2-yl)ethyl)-2-(4-methoxyphenyl)quinolin-4-amine:
N¹-(1-(4-Fluorobenzyl)piperidin-4-yl)-N³-(2-(4-methoxyphenyl)quinolin-4-yl)propane-1,3-diamine:
tert-Butyl 6-(3-((2-(4-methoxyphenyl)quinolin-4-yl)amino)propyl)-2,6-diazaspiro[3.4]octane-2-carboxylate;
N-(3-(2,6-Diazaspiro[3.4]octan-6-yl)propyl)-2-(4-methoxyphenyl)quinolin-4-amine:
2-(4-Methoxyphenyl)-N-(3-(4-methyl-1,4-diazepan-1-yl)propyl)quinolin-4-amine:
tert-Butyl 2-(2-((3-((2-(4-methoxyphenyl)quinolin-4-yl)amino)propyl)amino)ethyl)piperidine-1-carboxylate;
N¹-(2-(4-methoxyphenyl)quinolin-4-yl)-N³-(2-(piperidin-2-yl)ethyl)propane-1,3-diamine;
N-(3-(4-(3-Aminopropyl)piperazin-1-yl)propyl)-2-(4-methoxyphenyl)quinolin-4-amine;
tert-Butyl 4-{[(3-{[2-(4-methoxyphenyl)quinolin-4-yl]amino}propyl)(methyl)amino]methyl}piperidine-1-carboxylate;
N-(3-(N-Methyl-N-((piperidin-4-yl)methyl)amino)propyl)-2-(4-methoxyphenyl)quinolin-4-amine;
N-(3-((2-(4-Methoxyphenyl)quinolin-4-yl)amino)propyl)-4-(2H-tetrazol-5-yl)benzenesulfonamide;
N¹-(4-(2H-Tetrazol-5-yl)phenyl)-N³-(2-(4-methoxyphenyl)quinolin-4-yl)propane-1,3-diamine;
(1-(3-((2-(4-Methoxyphenyl)quinolin-4-yl)amino)propyl)piperidin-4-yl)methanol;
1-(Diethylamino)-3-((3-((2-(4-methoxyphenyl)quinolin-4-yl)amino)propyl)amino)propan-2-ol;
N¹-(2-(4-Methoxyphenyl)quinolin-4-yl)-N³-((1-methyl-1H-pyrazol-5-yl)methyl)propane-1,3-diamine;
N¹-(2-(4-methoxyphenyl)quinolin-4-yl)-N³-(2-(pyridin-2-yl)ethyl)propane-1,3-diamine;
N¹-(2-(4-Methoxyphenyl)quinolin-4-yl)-N³-(4-(piperidin-1-yl)phenyl)propane-1,3-diamine;
N-((1-(3-Aminopropyl)piperidin-3-yl)methyl)-2-(4-methoxyphenyl)quinolin-4-amine;
N-(3-(2-(6-Methoxy-1H-indol-3-yl)ethylamino)propyl)-2-(4-methoxyphenyl)quinolin-4-amine:
tert-Butyl 4-{[({1-[(tert-butoxy)carbonyl]piperidin-4-yl}methyl)(3-{[2-(4-methoxyphenyl)-quinolin-4-yl]amino}propyl)amino]methyl}piperidine-1-carboxylate;
N-(3-(Bis((piperidin-4-yl)methyl)amino)propyl)-2-(4-methoxyphenyl)quinolin-4-amine;
N¹-(4-Methoxybenzyl)-N³-(2-(4-methoxyphenyl)quinolin-4-yl)-N¹-methylpropane-1,3-diamine;
tert-Butyl(3-((4-((2-(4-methoxyphenyl)quinolin-4-yl)amino)cyclohexyl)amino)propyl)carbamate;
tert-Butyl 4-{[(3-{[2-(4-methoxyphenyl)quinolin-4-yl]amino}propyl)amino]methyl}piperidine-1-carboxylate;
N¹-(2-(4-methoxyphenyl)quinolin-4-yl)-N³-(3-(pyridin-4-ylamino)propyl)propane-1,3-diamine;
2-(3-(4-((2-(4-Methoxyphenyl)quinolin-4-yl)amino)piperidin-1-yl)propyl)isoindoline-1,3-dione;
2-(3-(3-(((2-(4-Methoxyphenyl)quinolin-4-yl)amino)methyl)piperidin-1-yl)propyl)isoindoline-1,3-dione;
N-(3-((3-((2-(4-Methoxyphenyl)quinolin-4-yl)amino)propyl)amino)propyl) methanesulfonamide;
2-(3-(2-(2-((2-(4-Methoxyphenyl)quinolin-4-yl)amino)ethyl)piperidin-1-yl)propyl)isoindoline-1,3-dione;
N-(3-(Hexahydropyrrolo[1,2-a]pyrazin-2(1H)-yl)propyl)-2-(4-methoxy phenyl)quinolin-4-amine;
2-(4-Methoxyphenyl)-N-(1′-methyl-[1,4′-bipiperidine]-4-yl)quinolin-4-amine;
N¹-(2-Benzyloctahydrocyclopenta[c]pyrrol-4-yl)-N³-(2-(4-methoxyphenyl)quinolin-4-yl)propane-1,3-diamine;
N¹-((1-(Cyclopropylmethyl)piperidin-4-yl)methyl)-N³-(2-(4-methoxyphenyl)quinolin-4-yl)propane-1,3-diamine;
1-(3-((2-(4-Methoxyphenyl)quinolin-4-yl)amino)propyl)piperidine-4-carboxamide;
2-(4-Methoxyphenyl)-N-(3-(4-methylpiperazin-1-yl)propyl)quinolin-4-amine;
2-(4-Methoxyphenyl)-N-(2-(4-methylpiperazin-1-yl)ethyl)quinolin-4-amine;
2-(4-Methoxyphenyl)-N-(3-morpholinopropyl)quinolin-4-amine;
2-(7-Amino-4-methyl-2-oxo-2H-chromen-3-yl)-N-(3-((3-((2-(4-methoxyphenyl)quinolin-4-yl)amino) propyl)(methyl)amino)propyl)acetamide;
5-(dimethylamino)-N-{3-[(3-{[2-(4-methoxyphenyl)quinolin-4yl]amino}propyl)(methyl)amino]propyl}naphthalene-1-sulfonamide, cis-N¹-(6,7-dimethoxy-2-(4-(piperazin-1-yl)phenyl)quinolin-4-yl)cyclobutane-1,3-diamine;
trans-N¹-(6,7-dimethoxy-2-(4-(piperazin-1-yl)phenyl)quinolin-4-yl)cyclobutane-1,3-diamine;
trans-N¹-(6,7-dimethoxy-2-(4-(piperazin-1-yl)phenyl)quinolin-4-yl)cyclohexane-1,4-diamine;
N¹-(6,7-dimethoxy-2-(4-(piperazin-1-yl)phenyl)quinolin-4-yl)propane-1,3-diamine;
cis-N¹-(2-(4-(piperazin-1-yl)phenyl)quinolin-4-yl)cyclobutane-1,3-diamine;
N¹-(2-(4-(5,6-dihydropyrrolo[3,4-c]pyrazol-2(4H)-yl)phenyl)quinolin-4-yl)propane-1,3-diamine;
cis-N¹-(6,7-dimethoxy-2-(4-(piperazin-1-yl)phenyl)quinolin-4-yl)cyclohexane-1,4-diamine;
N¹-(2-(4-(piperazin-1-yl)phenyl)quinolin-4-yl)propane-1,3-diamine;
N¹-(6,7-dimethoxy-2-(4-(piperazin-1-yl)phenyl)quinolin-4-yl)-N³,N³-dimethylpropane-1,3-diamine;
N¹-(3-aminopropyl)-N¹-methyl-N³-(2-(3-(piperazin-1-yl)phenyl)quinolin-4-yl)propane-1,3-diamine;
N¹-(6,7-dimethoxy-2-(4-methoxyphenyl)quinolin-4-yl)cyclohexane-1,4-diamine;
N¹-(2-(4-(piperazin-1-yl)phenyl)quinolin-4-yl)cyclohexane-1,4-diamine;
2-amino-1-(4-(4-(4-((3-(dimethylamino)propyl)amino)quinolin-2-yl)phenyl)piperazin-1-yl)-3-methylbutan-1-one;
2-amino-1-(4-(4-(4-((3-(dimethylamino)propyl)amino)quinolin-2-yl)phenyl)piperazin-1-yl)propan-1-one;
N¹-(2-(1-(1-methylpiperidin-4-yl)-1H-pyrazol-4-yl)quinolin-4-yl)propane-1,3-diamine;
N¹-(3-aminopropyl)-N³-(6,7-dimethoxy-2-(4-methoxyphenyl)quinolin-4-yl)-N¹-methylpropane-1,3-diamine;
N¹-(2-(4-(piperidin-4-yl)phenyl)quinolin-4-yl)propane-1,3-diamine;
N¹-(2-(1-(piperidin-4-yl)-1H-pyrazol-4-yl)quinolin-4-yl)propane-1,3-diamine;
trans-N¹-(6,7-dimethoxy-2-(4-methoxyphenyl)quinolin-4-yl)cyclobutane-1,3-diamine;
N¹,N¹-dimethyl-N³-(2-(3-(piperazin-1-yl)phenyl)quinolin-4-yl)propane-1,3-diamine;
N¹,N¹-dimethyl-N³-(2-(4-(piperazin-1-yl)phenyl)quinolin-4-yl)propane-1,3-diamine;
N¹-(2-(1-(2-(pyrrolidin-1-yl)ethyl)-1H-pyrazol-4-yl)quinolin-4-yl)propane-1,3-diamine;
trans-N¹-(6,7-dimethoxy-2-(4-methoxyphenyl)quinolin-4-yl)cyclohexane-1,4-diamine;
N-(1-(4-aminocyclohexyl)piperidin-4-yl)-2-(4-methoxyphenyl)quinolin-4-amine;
N¹-(6-fluoro-2-(4-(piperazin-1-yl)phenyl)quinolin-4-yl)-N³,N³-dimethylpropane-1,3-diamine;
N-(1-(3-aminopropyl)piperidin-4-yl)-2-(4-methoxyphenyl)quinolin-4-amine;
cis-N¹-(6,7-dimethoxy-2-(4-methoxyphenyl)quinolin-4-yl)cyclohexane-1,4-diamine;
2-amino-N-(1-((1-(4-(4-(4-((3-(dimethylamino)propyl)amino)quinolin-2-yl)phenyl)piperazin-1-yl)-3-methyl-1-oxobutan-2-yl)amino)-3-methyl-1-oxobutan-2-yl)-3-methylbutanamide;
N¹,N¹-dimethyl-N³-(2-(1-(1-methylpiperidin-4-yl)-1H-pyrazol-4-yl)quinolin-4-yl)propane-1,3-diamine;
N¹-(6,7-dimethoxy-2-(4-methoxyphenyl)quinolin-4-yl)-N³,N³-dimethylpropane-1,3-diamine;
N¹-(6,7-dimethoxy-2-(4-methoxyphenyl)quinolin-4-yl)propane-1,3-diamine;
N¹,N¹-dimethyl-N³-(2-(4-(4-methylpiperazin-1-yl)phenyl)quinolin-4-yl)propane-1,3-diamine;
2-amino-N-(1-(4-(4-(4-((3-(dimethylamino)propyl)amino)quinolin-2-yl)phenyl)piperazin-1-yl)-3-methyl-1-oxobutan-2-yl)-3-methylbutanamide;
cis-N¹-(2-(4-methoxyphenyl)quinolin-4-yl)cyclobutane-1,3-diamine;
N¹-([2,6′-biquinolin]-4-yl)propane-1,3-diamine;
4-(4-(4-((3-(dimethylamino)propyl)amino)quinolin-2-yl)phenyl)-1-methylpiperazin-2-one;
N¹-(2-(piperidin-4-yl)quinolin-4-yl)propane-1,3-diamine;
N¹-(2-(2,3-dihydrobenzofuran-5-yl)quinolin-4-yl)propane-1,3-diamine;
N¹-(2-(4-(methoxy-d3)phenyl)quinolin-4-yl)propane-1,3-diamine;
N¹-([2,6′-biquinolin]-4-yl)-N³,N³-dimethylpropane-1,3-diamine;
N¹-(2-(4-methoxyphenyl)quinolin-4-yl)cyclobutane-1,3-diamine;
N¹-(2-(1,2,3,6-tetrahydropyridin-4-yl)quinolin-4-yl)propane-1,3-diamine;
N¹-(2-(4-methoxyphenyl)quinolin-4-yl)cyclohexane-1,4-diamine;
2-(4-methoxyphenyl)-N-(piperidin-4-yl)quinolin-4-amine;
N¹-(2-(4-(methoxy-d3)phenyl)quinolin-4-yl)-N³,N³-dimethylpropane-1,3-diamine;
N¹-(2-(benzofuran-5-yl)quinolin-4-yl)-N³,N³-dimethylpropane-1,3-diamine;
N¹-(2-(1-methyl-1H-pyrazol-4-yl)quinolin-4-yl)propane-1,3-diamine;
N¹-(2-(1-(azetidin-3-yl)-1H-pyrazol-4-yl)quinolin-4-yl)-N³,N³-dimethylpropane-1,3-diamine;
N¹-(2-(1-cyclopropyl-1H-pyrazol-4-yl)quinolin-4-yl)-N³,N³-dimethylpropane-1,3-diamine;
N¹-(2-(cyclohex-1-en-1-yl)quinolin-4-yl)propane-1,3-diamine;
(S)-2-amino-N-(3-((2-(4-methoxyphenyl)quinolin-4-yl)amino)propyl)-N-methylpropanamide;
N¹-(2-(4-methoxyphenyl)quinolin-4-yl)-N³,N³-bis(methyl-d3)propane-1,3-diamine;
N¹-(2-(furan-2-yl)quinolin-4-yl)propane-1,3-diamine;
N¹-(5-fluoro-2-(4-methoxyphenyl)quinolin-4-yl)-N³,N³-dimethylpropane-1,3-diamine;
N¹-(2-(1-(2-morpholinoethyl)-1H-pyrazol-4-yl)quinolin-4-yl)propane-1,3-diamine;
3-((2-(4-methoxyphenyl)quinolin-4-yl)amino)propan-1-ol;
N¹-(2-(2-methoxypyrimidin-5-yl)quinolin-4-yl)-N³,N³-dimethylpropane-1,3-diamine;
N¹,N¹-dimethyl-N³-(2-methylquinolin-4-yl)propane-1,3-diamine;
3-((2-(4-methoxyphenyl)quinolin-4-yl)amino)-N,N-dimethylpropanamide;
N¹-(2-(6-methoxypyridin-3-yl)quinolin-4-yl)-N³,N³-dimethylpropane-1,3-diamine;
N¹-(2,2-difluoro-6-(4-methoxyphenyl)-[1,3]dioxolo[4,5-g]quinolin-8-yl)-N³,N³-dimethylpropane-1,3-diamine;
N-(4,4-difluorocyclohexyl)-2-(4-methoxyphenyl)quinolin-4-amine;
tert-butyl 3-((2-(4-methoxyphenyl)quinolin-4-yl)amino)propanoate;
N¹-(6-methoxy-2-(trifluoromethyl)quinolin-4-yl)-N³,N³-dimethylpropane-1,3-diamine;
N¹-(6-methoxy-7-(3-(pyrrolidin-1-yl)propoxy)quinolin-4-yl)-N³,N³-dimethylpropane-1,3-diamine,
N¹-(6-methoxy-7-(3-(pyrrolidin-1-yl)propoxy)quinolin-4-yl)-N³,N³-dimethylpropane-1,3-diamine;
2-cyclohexyl-N-(1-isopropylpiperidin-4-yl)-6-methoxy-7-(3-(pyrrolidin-1-yl)propoxy)quinolin-4-amine;
N¹-(3-aminopropyl)-N³-(6,7-dimethoxyquinolin-4-yl)-N¹-methylpropane-1,3-diamine;
6-methoxy-N-(1-methylpiperidin-4-yl)-7-(3-(pyrrolidin-1-yl)propoxy)quinolin-4-amine;
N¹-(6,7-dimethoxyquinolin-4-yl)-N³,N³-diethylpropane-1,3-diamine;
6,7-dimethoxy-N-(1′-methyl-[1,4′-bipiperidin]-4-yl)quinolin-4-amine;
N¹-(3-aminopropyl)-N³-(7-chloroquinolin-4-yl)-N¹-methylpropane-1,3-diamine;
N¹-(3-aminopropyl)-N³-(2-(4-(dimethylamino)phenyl)quinolin-4-yl)-N¹-methylpropane-1,3-diamine;
an enantiomer, a diastereomer, a hydrate, a solvate, a pharmaceutically acceptable salt, an isotopic analog, a prodrug, or a complex thereof.

32. A method for treating or preventing a disease that involves TDP-43, the method comprising administering to a subject an effective amount of at least one compound having formula (I): 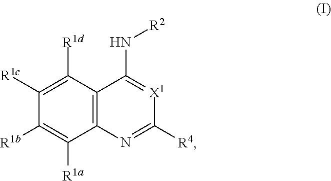
an enantiomer, a diastereomer, a hydrate, a solvate, a pharmaceutically acceptable salt, an isotopic analog, a prodrug or a complex thereof, wherein:
X¹ is selected from the group consisting of nitrogen and CH;
R^{1a} is selected from the group consisting of hydrogen, halogen, C₁₋₂₀ linear alkyl, C₃₋₂₀ branched alkyl, C₁₋₂₀ linear heteroalkyl, C₃₋₂₀ branched heteroalkyl, each of which except hydrogen and halogen are optionally substituted;
R^{1b} is selected from the group consisting of hydrogen, halogen, C₁₋₂₀ linear alkyl, C₃₋₂₀ branched alkyl, C₁₋₂₀ linear heteroalkyl, C₃₋₂₀ branched heteroalkyl, each of which except hydrogen and halogen are optionally substituted;
R^{1c} is selected from the group consisting of hydrogen, halogen, C₁₋₂₀ linear alkyl, C₃₋₂₀ branched alkyl, C₁₋₂₀ linear heteroalkyl, C₃₋₂₀ branched heteroalkyl, each of which except hydrogen and halogen are optionally substituted; and
R^{1d} is selected from the group consisting of hydrogen, halogen, C₁₋₂₀ linear alkyl, C₃₋₂₀ branched alkyl, C₁₋₂₀ linear heteroalkyl, C₃₋₂₀ branched heteroalkyl, each of which except hydrogen and halogen are optionally substituted,
R² is a substituted or unsubstituted C₁-C₂₀ linear, branched, or cyclic organic group including at least one nitrogen; and
R⁴ is a hydrogen, halogen, a hydroxyl group, a cyano group, a nitro group, a substituted or unsubstituted C₀-C₁₀ amino group, a substituted or unsubstituted C₁-C₁₀ alkyl group, a substituted or unsubstituted C₂-C₁₀ alkenyl group, a substituted or unsubstituted C₂-C₁₀ alkynyl group, and a substituted or unsubstituted C₁-C₁₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₂₀ aryl group, a substituted or unsubstituted C₆-C₂₀ aryloxy group, a substituted or unsubstituted C₆-C₂₀ arylthio group, a substituted or unsubstituted C₂-C₂₀ heteroaryl group, a substituted or unsubstituted C₂-C₂₀ heteroaryloxy group, or a substituted or unsubstituted C₂-C₂₀ heteroarylthio group,
provided that the following compounds in which each hydrogen is a proton are excluded:

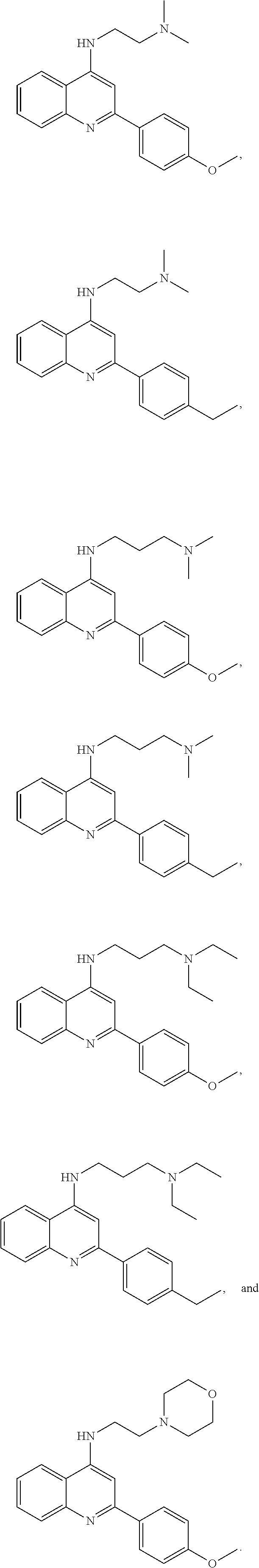

33. The method of claim 32, wherein the at least one compound is administered in a composition further comprising at least one excipient.

34. The method of claim 33, wherein the diseases that involves TDP-43 is selected from the group consisting of ALS, FTLD, CTE, hippocampal sclerosis of aging (CARTS), Alzheimer's disease, and Alzheimer's disease related disorders.

35. A method for treating or preventing a disease associated with TDP-43 proteinopathies, the method comprising administering to a subject an effective amount of at least one compound according to claim 1.

36. The method of claim 35, wherein the at least one compound is administered in a composition further comprising at least one excipient.

37. A method for treating or preventing diseases that involve excess amounts of TDP-43 in the cytosol, the method comprising administering to a subject an effective amount of at least one compound according to claim 1.

38. The method of claim 37, wherein the at least one compound is administered in a composition further comprising at least one excipient.

39. The method of use of the TDP-43 binders of the present invention as positron emission tomography (PET) imaging agents, wherein the method comprises administering to a subject an effective amount of an isotopically labeled compound according to claim 1.

40. The method of use of the TDP-43 binders of the present invention as single-photon emission computed tomography (SPECT) imaging agents, wherein the method comprises administering to a subject an effective amount of an isotopically labeled compound according to claim 1.

41. The method of claim 32, wherein, in formula (I),
R^{1a} is selected from the group consisting of hydrogen, halogen, C₁₋₂₀ linear alkyl, C₃₋₂₀ branched alkyl, C₁₋₂₀ linear alkoxy, C₃₋₂₀ branched alkoxy, C₁₋₂₀ linear aminoalkyl, C₃₋₂₀ branched aminoalkyl, C₁₋₂₀ linear aminoalkoxy, and C₃₋₂₀ branched aminoalkoxy, each of which except hydrogen and halogen are optionally substituted;
R^{1b} is selected from the group consisting of hydrogen, halogen, C₁₋₂₀ linear alkyl, C₃₋₂₀ branched alkyl, C₁₋₂₀ linear alkoxy, C₃₋₂₀ branched alkoxy, C₁₋₂₀ linear aminoalkyl, C₃₋₂₀ branched aminoalkyl, C₁₋₂₀ linear aminoalkoxy, and C₃₋₂₀ branched aminoalkoxy, each of which except hydrogen and halogen are optionally substituted;
R^{1c} is selected from the group consisting of hydrogen, halogen, C₁₋₂₀ linear alkyl, C₃₋₂₀ branched alkyl, C₁₋₂₀ linear alkoxy, C₃₋₂₀ branched alkoxy, C₁₋₂₀ linear aminoalkyl, C₃-20 branched aminoalkyl, C₁₋₂₀ linear aminoalkoxy, and C₃₋₂₀ branched aminoalkoxy, each of which except hydrogen and halogen are optionally substituted; and
R^{1d} is selected from the group consisting of hydrogen, halogen, C₁₋₂₀ linear alkyl, C₃₋₂₀ branched alkyl, C₁₋₂₀ linear alkoxy, C₃₋₂₀ branched alkoxy, C₁₋₂₀ linear aminoalkyl, C₃-20 branched aminoalkyl, C₁₋₂₀ linear aminoalkoxy, and C₃₋₂₀ branched aminoalkoxy, each of which except hydrogen and halogen are optionally substituted.

42. The method of claim 41, wherein, in formula (I), at least one selected from the group consisting of R^{1a}, R^{1b}, R^{1c}, and R^{1d} is a substituted or unsubstituted C₁₋₂₀ aminoalkoxy group.

43. The method of claim 41, wherein, in formula (I), at least one selected from the group consisting of R^{1a}, R^{1b}, R^{1c}, and R^{1d} is a substituted or unsubstituted (C₂₋₈ dialkylamino)(C₂₋₄ alkoxy) group or a substituted or unsubstituted (C₃₋₆ alkyleneamino)(C₂₋₄ alkoxy) group.

44. The method of claim 32, wherein the compound is:
N¹-(3-((2-(4-Methoxyphenyl)quinolin-4-yl)amino)propyl)-N¹,N³,N³-trimethyl propane-1,3-diamine;
2-(4-Methoxyphenyl)-N-(3-f{methyl[3-(methylamino)propyl]-amino}propyl)quinolin-4-amine;
N¹-(3-Aminopropyl)-N³-(2-(4-methoxyphenyl)quinolin-4-yl)-N¹-methylpropane-1,3-diamine;
N¹-(2-(4-Methoxyphenyl)quinolin-4-yl)-N³-(3-(piperidin-yl)propyl)propane-1,3-diamine;
N¹-(2-(4-methoxyphenyl)quinolin-4-yl)-N³-methyl-N³-(3-(piperidin-1-yl)propyl)propane-1,3-diamine;
tert-Butyl N-{3-[(3-{[2-(4-methoxyphenyl)quinolin-4yl]amino}propyl)amino]propyl}carbamate;
N¹-(3-aminopropyl)-N³-(5-fluoro-2-(4-methoxyphenyl)quinolin-4-yl)-N¹-methylpropane-1,3-diamine:
N-{3-[(3-Aminopropyl)amino]propyl}-2-(4-methoxyphenyl)quinolin-4-amine;
N¹-(3-Aminopropyl)-N³-(6-methoxy-2-(4-methoxyphenyl)quinolin-4-yl)-N¹-methylpropane-1,3-diamine;
N¹-(3-Aminopropyl)-N³-(2-(2-methyl-4-methoxyphenyl)quinolin-4-yl)-N¹-methylpropane-1,3-diamine;
N¹-(3-Aminopropyl)-N³-(6,7-dimethoxy-2-(4-methoxyphenyl)quinolin-4-yl)-N¹-methylpropane-1,3-diamine;
N¹-(3-((2-(4-Methoxyphenyl)quinolin-4-yl)amino)propyl)-N¹,N⁴-dimethylbutane-1,4-diamine;
N¹-(2-fluoroethyl)-N³-(3-((2-(4-methoxyphenyl)quinolin-4-yl)amino)propyl)-N¹,N³-dimethylpropane-1,3-diamine;
3-((3-((2-(4-Methoxyphenyl)quinolin-4-yl)amino)propyl)amino)-propan-1-ol;
N-(3-(3-methoxypropylamino)propyl)-2-(4-methoxyphenyl)quinolin-4-amine;
N-(3-(2-(4-Methoxyphenyl)quinolin-4-ylamino)propyl)-N-(3-aminopropyl)acetamide;
N¹-(Cyclopropylmethyl)-N¹-(3-((2-(4-methoxyphenyl)quinolin-4-yl)amino)propyl)-N³,N³-dimethyl propane-1,3-diamine:
tert-Butyl 3-(((3-(dimethylamino)propyl)(3-((2-(4-methoxyphenyl)quinolin-4-yl)amino)propyl)amino)methyl)azetidine-1-carboxylate;
tert-Butyl 4-(((3-(dimethylamino)propyl)(3-((2-(4-methoxyphenyl)quinolin-4-yl)amino)propyl)amino)methyl)piperidine-1-carboxylate:
N¹-(Azetidin-3-ylmethyl)-N¹-(3-((2-(4-methoxyphenyl)quinolin-4-yl)amino)propyl)-N³,N³-dimethylpropane-1,3-diamine;
N¹-(3-((2-(4-Methoxyphenyl)quinolin-4-yl)amino)propyl)-N³,N³-dimethyl-N¹-(piperidin-4-ylmethyl)propane-1,3-diamine;
N¹-(2-(4-Methoxyphenyl)quinolin-4-yl)-N³-((1-methyl-1H-imidazol-5-yl)methyl)propane-1,3-diamine;
N-(3-(Hexahydropyrrolo[3,4-c]pyrrol-2(1H)-yl)propyl)-2-(4-methoxyphenyl)quinolin-4-amine;
N¹-((6-Aminopyridin-3-yl)methyl)-N³-(2-(4-methoxyphenyl)-quinolin-4-yl)-N¹-methylpropane-1,3-diamine;
1-(Dimethylamino)-3-((3-((2-(4-methoxyphenyl)quinolin-4-yl)-amino)propyl)(methyl)amino)propan-2-ol;
1-(3-((3-((2-(4-Methoxyphenyl)quinolin-4-yl)amino)propyl)-methylamino)propyl)piperidin-4-ol;
tert-butyl 5-(3-((2-(4-methoxyphenyl)quinolin-4-yl)amino)propyl)hexahydro-pyrrolo[3,4-c]pyrrole-2(1H)-carboxylate;
N¹-((6-(dimethylamino)pyridin-3-yl)methyl)-N³-(2-(4-methoxyphenyl)quinolin-4-yl)-N¹-methyl propane-1,3-diamine;
N¹-(3-aminopropyl)-N¹-methyl-N³-(2-(4-(piperazin-1-yl)phenyl)quinolin-4-yl)propane-1,3-diamine;
N¹-(2-(4-methoxyphenyl)quinolin-4-yl)-N³-methyl-N³-((1-methylpiperidin-4-yl)methyl)propane-1,3-diamine:
N¹-(2-(4-methoxyphenyl)quinolin-4-yl)-N³-methyl-N³-(2-(piperidin-2-yl)ethyl)propane-1,3-diamine;
tert-Butyl(2-(3-((2-(4-methoxyphenyl)quinolin-4-yl)amino)propyl)octahydrocyclopenta[c]pyrrol-4-yl)carbamate:
N¹-(2-(4-Methoxyphenyl)quinolin-4-yl)-N³-(octahydrocyclopenta[c]pyrrol-4-yl)propane-1,3-diamine;
N-(3-(4-Aminohexahydrocyclopenta[c]pyrrol-2(1H)-yl)propyl)-2-(4-methoxyphenyl)quinolin-4-amine;
tert-Butyl 2-(3-((2-(4-methoxyphenyl)quinolin-4-yl)amino)propyl)hexahydro-1H-pyrrolo[3,4-c]pyridine-5(6H)-carboxylate;
N-(3-(Hexahydro-1H-pyrrolo[3,4-c]pyridin-2(3H)-yl)propyl)-2-(4-methoxyphenyl)quinolin-4-amine;
tert-Butyl 4-((3-((2-(4-methoxyphenyl)quinolin-4-yl)amino)propyl)amino)hexahydrocyclopenta[c]pyrrole-2(1H)-carboxylate;
N¹-((1-(Cyclopropylmethyl)piperidin-4-yl)methyl)-N³-(2-(4-methoxyphenyl)quinolin-4-yl)-N¹-methyl propane-1,3-diamine;
N-(2-(1-(3-Aminopropyl)piperidin-2-yl)ethyl)-2-(4-methoxyphenyl)quinolin-4-amine:
N¹-(1-(4-Fluorobenzyl)piperidin-4-yl)-N³-(2-(4-methoxyphenyl)quinolin-4-yl)propane-1,3-diamine:
tert-Butyl 6-(3-((2-(4-methoxyphenyl)quinolin-4-yl)amino)propyl)-2,6-diazaspiro[3.4]octane-2-carboxylate;
N-(3-(2,6-Diazaspiro[3.4]octan-6-yl)propyl)-2-(4-methoxyphenyl)quinolin-4-amine:
2-(4-Methoxyphenyl)-N-(3-(4-methyl-1,4-diazepan-1-yl)propyl)quinolin-4-amine:
tert-Butyl 2-(2-((3-((2-(4-methoxyphenyl)quinolin-4-yl)amino)propyl)amino)ethyl)piperidine-1-carboxylate;
N¹-(2-(4-methoxyphenyl)quinolin-4-yl)-N³-(2-(piperidin-2-yl)ethyl)propane-1,3-diamine;
N-(3-(4-(3-Aminopropyl)piperazin-1-yl)propyl)-2-(4-methoxyphenyl)quinolin-4-amine;
tert-Butyl 4-{[(3-{[2-(4-methoxyphenyl)quinolin-4-yl]amino}propyl)(methyl)amino]methyl}piperidine-1-carboxylate;
N-(3-(N-Methyl-N-((piperidin-4-yl)methyl)amino)propyl)-2-(4-methoxyphenyl)quinolin-4-amine;
N-(3-((2-(4-Methoxyphenyl)quinolin-4-yl)amino)propyl)-4-(2H-tetrazol-5-yl)benzenesulfonamide;
N¹-(4-(2H-Tetrazol-5-yl)phenyl)-N³-(2-(4-methoxyphenyl)quinolin-4-yl)propane-1,3-diamine;
(1-(3-((2-(4-Methoxyphenyl)quinolin-4-yl)amino)propyl)piperidin-4-yl)methanol;
1-(Diethylamino)-3-((3-((2-(4-methoxyphenyl)quinolin-4-yl)amino)propyl)amino)propan-2-ol;
N¹-(2-(4-Methoxyphenyl)quinolin-4-yl)-N³-((1-methyl-1H-pyrazol-5-yl)methyl)propane-1,3-diamine;
N¹-(2-(4-methoxyphenyl)quinolin-4-yl)-N³-(2-(pyridin-2-yl)ethyl)propane-1,3-diamine;
N¹-(2-(4-Methoxyphenyl)quinolin-4-yl)-N³-(4-(piperidin-1-yl)phenyl)propane-1,3-diamine;
N-((1-(3-Aminopropyl)piperidin-3-yl)methyl)-2-(4-methoxyphenyl)quinolin-4-amine;
N-(3-(2-(6-Methoxy-1H-indol-3-yl)ethylamino)propyl)-2-(4-methoxyphenyl)quinolin-4-amine:
tert-Butyl 4-{[({1-[(tert-butoxy)carbonyl]piperidin-4-yl}methyl)(3-{[2-(4-methoxyphenyl)-quinolin-4-yl]amino}propyl)amino]methyl}piperidine-1-carboxylate;
N-(3-(Bis((piperidin-4-yl)methyl)amino)propyl)-2-(4-methoxyphenyl)quinolin-4-amine;
N¹-(4-Methoxybenzyl)-N³-(2-(4-methoxyphenyl)quinolin-4-yl)-N¹-methylpropane-1,3-diamine;
tert-Butyl(3-((4-((2-(4-methoxyphenyl)quinolin-4-yl)amino)cyclohexyl)amino)propyl)carbamate;
tert-Butyl 4-{[(3-{[2-(4-methoxyphenyl)quinolin-4-yl]amino}propyl)amino]methyl}piperidine-1-carboxylate;
N¹-(2-(4-methoxyphenyl)quinolin-4-yl)-N³-(3-(pyridin-4-ylamino)propyl)propane-1,3-diamine;
2-(3-(4-((2-(4-Methoxyphenyl)quinolin-4-yl)amino)piperidin-1-yl)propyl)isoindoline-1,3-dione;
2-(3-(3-(((2-(4-Methoxyphenyl)quinolin-4-yl)amino)methyl)piperidin-1-yl)propyl)isoindoline-1,3-dione;
N-(3-((3-((2-(4-Methoxyphenyl)quinolin-4-yl)amino)propyl)amino)propyl) methanesulfonamide;
2-(3-(2-(2-((2-(4-Methoxyphenyl)quinolin-4-yl)amino)ethyl)piperidin-1-yl)propyl)isoindoline-1,3-dione;
N-(3-(Hexahydropyrrolo[1,2-a]pyrazin-2(1H)-yl)propyl)-2-(4-methoxy phenyl)quinolin-4-amine;
2-(4-Methoxyphenyl)-N-(1′-methyl-[1,4′-bipiperidine]-4-yl)quinolin-4-amine;
N¹-(2-Benzyloctahydrocyclopenta[c]pyrrol-4-yl)-N³-(2-(4-methoxyphenyl)quinolin-4-yl)propane-1,3-diamine;
N¹-((1-(Cyclopropylmethyl)piperidin-4-yl)methyl)-N³-(2-(4-methoxyphenyl)quinolin-4-yl)propane-1,3-diamine;
1-(3-((2-(4-Methoxyphenyl)quinolin-4-yl)amino)propyl)piperidine-4-carboxamide;
2-(4-Methoxyphenyl)-N-(3-(4-methylpiperazin-1-yl)propyl)quinolin-4-amine;
2-(4-Methoxyphenyl)-N-(2-(4-methylpiperazin-1-yl)ethyl)quinolin-4-amine;
2-(4-Methoxyphenyl)-N-(3-morpholinopropyl)quinolin-4-amine;
2-(7-Amino-4-methyl-2-oxo-2H-chromen-3-yl)-N-(3-((3-((2-(4-methoxyphenyl)quinolin-4-yl)amino) propyl)(methyl)amino)propyl)acetamide;
5-(dimethylamino)-N-{3-[(3-{[2-(4-methoxyphenyl)quinolin-4yl]amino}propyl)(methyl)amino]propyl}naphthalene-1-sulfonamide;
cis-N¹-(6,7-dimethoxy-2-(4-(piperazin-1-yl)phenyl)quinolin-4-yl)cyclobutane-1,3-diamine;
trans-N¹-(6,7-dimethoxy-2-(4-(piperazin-1-yl)phenyl)quinolin-4-yl)cyclobutane-1,3-diamine;
trans-N¹-(6,7-dimethoxy-2-(4-(piperazin-1-yl)phenyl)quinolin-4-yl)cyclohexane-1,4-diamine;
N¹-(6,7-dimethoxy-2-(4-(piperazin-1-yl)phenyl)quinolin-4-yl)propane-1,3-diamine;
cis-N¹-(2-(4-(piperazin-1-yl)phenyl)quinolin-4-yl)cyclobutane-1,3-diamine;
N¹-(2-(4-(5,6-dihydropyrrolo[3,4-c]pyrazol-2(4H)-yl)phenyl)quinolin-4-yl)propane-1,3-diamine;
cis-N¹-(6,7-dimethoxy-2-(4-(piperazin-1-yl)phenyl)quinolin-4-yl)cyclohexane-1,4-diamine;
N¹-(2-(4-(piperazin-1-yl)phenyl)quinolin-4-yl)propane-1,3-diamine;
N¹-(6,7-dimethoxy-2-(4-(piperazin-1-yl)phenyl)quinolin-4-yl)-N³,N³-dimethylpropane-1,3-diamine;
N¹-(3-aminopropyl)-N¹-methyl-N³-(2-(3-(piperazin-1-yl)phenyl)quinolin-4-yl)propane-1,3-diamine;
N¹-(6,7-dimethoxy-2-(4-methoxyphenyl)quinolin-4-yl)cyclohexane-1,4-diamine;
N¹-(2-(4-(piperazin-1-yl)phenyl)quinolin-4-yl)cyclohexane-1,4-diamine;
2-amino-1-(4-(4-(4-((3-(dimethylamino)propyl)amino)quinolin-2-yl)phenyl)piperazin-1-yl)-3-methylbutan-1-one;
2-amino-1-(4-(4-(4-((3-(dimethylamino)propyl)amino)quinolin-2-yl)phenyl)piperazin-1-yl)propan-1-one;
N¹-(2-(1-(1-methylpiperidin-4-yl)-1H-pyrazol-4-yl)quinolin-4-yl)propane-1,3-diamine;
N¹-(3-aminopropyl)-N³-(6,7-dimethoxy-2-(4-methoxyphenyl)quinolin-4-yl)-N¹-methylpropane-1,3-diamine;
N¹-(2-(4-(piperidin-4-yl)phenyl)quinolin-4-yl)propane-1,3-diamine;
N¹-(2-(1-(piperidin-4-yl)-1H-pyrazol-4-yl)quinolin-4-yl)propane-1,3-diamine;
trans-N¹-(6,7-dimethoxy-2-(4-methoxyphenyl)quinolin-4-yl)cyclobutane-1,3-diamine;
N¹,N¹-dimethyl-N³-(2-(3-(piperazin-1-yl)phenyl)quinolin-4-yl)propane-1,3-diamine;
N¹,N¹-dimethyl-N³-(2-(4-(piperazin-1-yl)phenyl)quinolin-4-yl)propane-1,3-diamine;
N¹-(2-(1-(2-(pyrrolidin-1-yl)ethyl)-1H-pyrazol-4-yl)quinolin-4-yl)propane-1,3-diamine;
trans-N¹-(6,7-dimethoxy-2-(4-methoxyphenyl)quinolin-4-yl)cyclohexane-1,4-diamine;
N-(1-(4-aminocyclohexyl)piperidin-4-yl)-2-(4-methoxyphenyl)quinolin-4-amine;
N¹-(6-fluoro-2-(4-(piperazin-1-yl)phenyl)quinolin-4-yl)-N³,N³-dimethylpropane-1,3-diamine;
N-(1-(3-aminopropyl)piperidin-4-yl)-2-(4-methoxyphenyl)quinolin-4-amine;
cis-N¹-(6,7-dimethoxy-2-(4-methoxyphenyl)quinolin-4-yl)cyclohexane-1,4-diamine;
2-amino-N-(1-((1-(4-(4-(4-((3-(dimethylamino)propyl)amino)quinolin-2-yl)phenyl)piperazin-1-yl)-3-methyl-1-oxobutan-2-yl)amino)-3-methyl-1-oxobutan-2-yl)-3-methylbutanamide;
N¹,N¹-dimethyl-N³-(2-(1-(1-methylpiperidin-4-yl)-1H-pyrazol-4-yl)quinolin-4-yl)propane-1,3-diamine;
N¹-(6,7-dimethoxy-2-(4-methoxyphenyl)quinolin-4-yl)-N³,N³-dimethylpropane-1,3-diamine;
N¹-(6,7-dimethoxy-2-(4-methoxyphenyl)quinolin-4-yl)propane-1,3-diamine;
N¹,N¹-dimethyl-N³-(2-(4-(4-methylpiperazin-1-yl)phenyl)quinolin-4-yl)propane-1,3-diamine;
2-amino-N-(1-(4-(4-(4-((3-(dimethylamino)propyl)amino)quinolin-2-yl)phenyl)piperazin-1-yl)-3-methyl-1-oxobutan-2-yl)-3-methylbutanamide;
cis-N¹-(2-(4-methoxyphenyl)quinolin-4-yl)cyclobutane-1,3-diamine;
N¹-([2,6′-biquinolin]-4-yl)propane-1,3-diamine;
4-(4-(4-((3-(dimethylamino)propyl)amino)quinolin-2-yl)phenyl)-1-methylpiperazin-2-one;
N¹-(2-(piperidin-4-yl)quinolin-4-yl)propane-1,3-diamine;
N¹-(2-(2,3-dihydrobenzofuran-5-yl)quinolin-4-yl)propane-1,3-diamine;
N¹-(2-(4-(methoxy-d₃)phenyl)quinolin-4-yl)propane-1,3-diamine;
N¹-([2,6′-biquinolin]-4-yl)-N³,N³-dimethylpropane-1,3-diamine;
N¹-(2-(4-methoxyphenyl)quinolin-4-yl)cyclobutane-1,3-diamine;
N¹-(2-(1,2,3,6-tetrahydropyridin-4-yl)quinolin-4-yl)propane-1,3-diamine;
N¹-(2-(4-methoxyphenyl)quinolin-4-yl)cyclohexane-1,4-diamine;
2-(4-methoxyphenyl)-N-(piperidin-4-yl)quinolin-4-amine;
N¹-(2-(4-(methoxy-d₃)phenyl)quinolin-4-yl)-N³,N³-dimethylpropane-1,3-diamine;
N¹-(2-(benzofuran-5-yl)quinolin-4-yl)-N³,N³-dimethylpropane-1,3-diamine;
N¹-(2-(1-methyl-1H-pyrazol-4-yl)quinolin-4-yl)propane-1,3-diamine;
N¹-(2-(1-(azetidin-3-yl)-1H-pyrazol-4-yl)quinolin-4-yl)-N³,N³-dimethylpropane-1,3-diamine;
N¹-(2-(1-cyclopropyl-1H-pyrazol-4-yl)quinolin-4-yl)-N³,N³-dimethylpropane-1,3-diamine;
N¹-(2-(cyclohex-1-en-1-yl)quinolin-4-yl)propane-1,3-diamine;
(S)-2-amino-N-(3-((2-(4-methoxyphenyl)quinolin-4-yl)amino)propyl)-N-methylpropanamide;
N¹-(2-(4-methoxyphenyl)quinolin-4-yl)-N³,N³-bis(methyl-d₃)propane-1,3-diamine;
N¹-(2-(furan-2-yl)quinolin-4-yl)propane-1,3-diamine;
N¹-(5-fluoro-2-(4-methoxyphenyl)quinolin-4-yl)-N³,N³-dimethylpropane-1,3-diamine;
N¹-(2-(1-(2-morpholinoethyl)-1H-pyrazol-4-yl)quinolin-4-yl)propane-1,3-diamine;
3-((2-(4-methoxyphenyl)quinolin-4-yl)amino)propan-1-ol;
N¹-(2-(2-methoxypyrimidin-5-yl)quinolin-4-yl)-N³,N³-dimethylpropane-1,3-diamine;
N¹,N¹-dimethyl-N³-(2-methylquinolin-4-yl)propane-1,3-diamine;
3-((2-(4-methoxyphenyl)quinolin-4-yl)amino)-N,N-dimethylpropanamide;
N¹-(2-(6-methoxypyridin-3-yl)quinolin-4-yl)-N³,N³-dimethylpropane-1,3-diamine;
N¹-(2,2-difluoro-6-(4-methoxyphenyl)-[1,3]dioxolo[4,5-g]quinolin-8-yl)-N³,N³-dimethylpropane-1,3-diamine;
N-(4,4-difluorocyclohexyl)-2-(4-methoxyphenyl)quinolin-4-amine;
tert-butyl 3-((2-(4-methoxyphenyl)quinolin-4-yl)amino)propanoate;
N¹-(6-methoxy-2-(trifluoromethyl)quinolin-4-yl)-N³,N³-dimethylpropane-1,3-diamine;
N¹-(6-methoxy-7-(3-(pyrrolidin-1-yl)propoxy)quinolin-4-yl)-N³,N³-dimethylpropane-1,3-diamine;
2-cyclohexyl-N-(1-isopropylpiperidin-4-yl)-6-methoxy-7-(3-(pyrrolidin-1-yl)propoxy)quinolin-4-amine;
N¹-(3-aminopropyl)-N³-(6,7-dimethoxyquinolin-4-yl)-N¹-methylpropane-1,3-diamine;
6-methoxy-N-(1-methylpiperidin-4-yl)-7-(3-(pyrrolidin-1-yl)propoxy)quinolin-4-amine;
N¹-(6,7-dimethoxyquinolin-4-yl)-N³,N³-diethylpropane-1,3-diamine;
6,7-dimethoxy-N-(1′-methyl-[1,4′-bipiperidin]-4-yl)quinolin-4-amine;
N¹-(3-aminopropyl)-N³-(7-chloroquinolin-4-yl)-N¹-methylpropane-1,3-diamine;
N¹-(3-aminopropyl)-N³-(2-(4-(dimethylamino)phenyl)quinolin-4-yl)-N-methylpropane-1,3-diamine;
N¹-(6-methoxy-7-(2-(pyrrolidin-1-yl)ethoxy)quinolin-4-yl)-N³,N³-dimethylpropane-1,3-diamine;
N¹-(2-(4-(2-(1H-pyrazol-1-yl)ethoxy)phenyl)quinolin-4-yl)-N³,N³-dimethylpropane-1,3-diamine,
an enantiomer, a diastereomer, a hydrate, a solvate, a pharmaceutically acceptable salt, an isotopic analog, a prodrug, or a complex thereof.
